(19) United States
(12) Patent Application Publication
(10) Pub. No.: US 2023/0227449 A1
(43) Pub. Date: Jul. 20, 2023

(54) AMIDOPYRIMIDONE DERIVATIVES

(71) Applicant: Hoffmann-La Roche Inc., Little Falls, NJ (US)

(72) Inventors: Andrew Simon Bell, Dundee (GB); Jérémy Besnard, Dundee (GB); Anthony Richard Bradley, Dundee (GB); Luke Green, Basel (CH); Wolfgang Haap, Loerrach (DE); Buelent Kocer, Maulburg (DE); Andreas Kuglstatter, Loerrach (DE); Xavier Lucas, Basel (CH); Patrizio Mattei, Riehen (CH); Dmitry Mazunin, Grenzach-Wyhlen (DE); Hasane Ratni, Habsheim (FR); Claus Riemer, Freiburg (DE); Willem Paul Van Hoorn, Dundee (GB)

(73) Assignee: Hoffmann-La Roche Inc., Little Falls, NJ (US)

(21) Appl. No.: 18/068,407

(22) Filed: Dec. 19, 2022

Related U.S. Application Data
(63) Continuation of application No. PCT/EP2021/066725, filed on Jun. 21, 2021.

(30) Foreign Application Priority Data
Jun. 22, 2020 (EP) 20181341.7

(51) Int. Cl. C07D 471/04; C07D 405/04; C07D 519/00; C07D 401/04; C07D 239/95

(52) U.S. Cl. CPC C07D 471/04; C07D 405/04; C07D 519/00; C07D 401/04; C07D 239/95

(57) ABSTRACT

The present invention provides compounds of formula I or II:

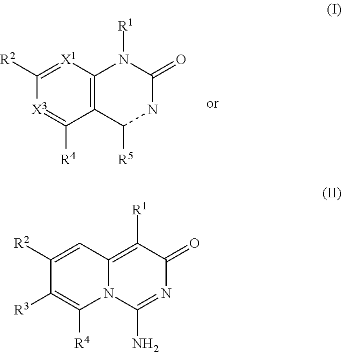

wherein X¹, X³, R¹, R², R³, R⁴ and R⁵ are as described herein, as well as pharmaceutically acceptable salts thereof. Further the present invention is concerned with the manufacture of the compounds of formula I, pharmaceutical compositions comprising them and their use as medicaments.

This application is a Continuation of International Application No. PCT/EP2021/066725, filed on Jun. 21, 2021, which claims benefit of priority to European Application No. 20181341.7 filed Jun. 22, 2020, each of which is incorporated herein by reference in its entirety.

The present invention provides compounds which are inhibitors of the Human methionine adenosyltransferase 2A (Mat2A), for use in the treatment, prevention and/or delay of progression of Cancer.

The present invention relates to compounds of formula I or II:

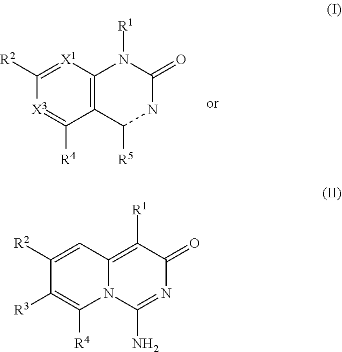

wherein

X¹ is either N or CH;

X³ is either N or CR³;

the dotted line represents a single bond when R⁵ is oxo or a double bond when R⁵ is —NH₂, R¹ is (C₁-C₆)alkyl optionally substituted with one or more, particularly one to three, more particularly one or two substituents R^{1a}, (C₁-C₆)alkoxy optionally substituted with one or more, particularly one to three, more particularly one or two substituents R^{1b}, halo(C₁-C₆)alkyl optionally substituted with one or more, particularly one to three, more particularly one or two substituents R^{1a}, halo(C₁-C₆)alkoxy optionally substituted with one or more, particularly one to three, more particularly one or two substituents R^{1b}, (C₃-C₈)cycloalkyl optionally substituted with one or more, particularly one to three, more particularly one or two substituents R^{1c}, heteroaryl optionally substituted with one or more, particularly one to three, more particularly one or two substituents R^{1d}, heterocycloalkyl optionally substituted with one or more, particularly one to three, more particularly one or two substituents R^{1e} or phenyl optionally substituted with one or more, particularly one to three, more particularly one or two substituents R^{1f};

R^{1a} and R^{1b} are each independently selected from (C₃-C₆)cycloalkyl, hydroxyl, heteroaryl, heterocycloalkyl and phenyl, wherein heteroaryl, heterocycloalkyl or phenyl are optionally substituted with one or more, particularly one to three, more particularly one or two substituents R^{1g};

R^{1c}, R^{1d}, R^{1e} and R^{1f} are each independently selected from halogen, oxo, cyano, hydroxyl, (C₁-C₆)alkyl, (C₁-C₆)alkoxy, (C₃-C₆)cycloalkyl, (C₃-C₆)cycloalkyl-(C₁-C₆)alkyl, halo(C₁-C₆)alkyl, halo(C₁-C₆)alkoxy, hydroxy(C₁-C₆)alkyl, (C₁-C₆)alkoxy-(C₁-C₆)alkyl, heteroaryl, heterocycloalkyl and phenyl;

R^{1g} are each independently selected from halogen, cyano, hydroxyl, (C₁-C₆)alkyl, (C₁-C₆)alkoxy, (C₃-C₆)cycloalkyl, (C₁-C₆)alkoxy, (C₃-C₆)cycloalkyl-(C₁-C₆)alkyl, halo(C₁-C₆)alkyl, halo(C₁-C₆)alkoxy, hydroxy(C₁-C₆)alkyl, and (C₁-C₆)alkoxy-(C₁-C₆)alkyl;

R² is hydrogen, halogen, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{2a}, (C₃-C₆)cycloalkyl-(C₁-C₆)alkyl optionally substituted with one or more, particularly one to three, more particularly one or two substituents R^{2b}, (C₃-C₆)cycloalkyl-(C₁-C₆)alkoxy optionally substituted with one or more, particularly one to three, more particularly one or two substituents R^{2c}, heterocycloalkyl optionally substituted with one or more, particularly one to three, more particularly one or two substituents R^{2a}, NR^{2f}R^{2g} or phenyl optionally substituted with one or more, particularly one to three, more particularly one or two substituents R^{2e};

R^{2a}, R^{2b}, R^{2c}, R^{2d} and R^{2e} are each independently selected from halogen, (C₁-C₆)alkyl, (C₁-C₆)alkoxy, halo(C₁-C₆)alkyl and halo(C₁-C₆)alkoxy;

R^{2f} and R^{2g} are each independently selected from hydrogen or (C₁-C₆)alkyl;

R³ is hydrogen, halogen, cyano, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{3a}, (C₃-C₆)cycloalkyl-(C₁-C₆)alkyl optionally substituted with one or more, particularly one to three, more particularly one or two substituents R^{3b}, (C₃-C₆)cycloalkyl-(C₁-C₆)alkoxy optionally substituted with one or more, particularly one to three, more particularly one or two substituents R^{3c}, heterocycloalkyl optionally substituted with one or more, particularly one to three, more particularly one or two substituents R^{3d} or phenyl optionally substituted with one or more, particularly one to three, more particularly one or two substituents R^{3e};

R^{3a}, R^{3b}, R^{3c}, R^{3d} and R^{3e} are each independently selected from halogen, (C₁-C₆)alkyl, (C₁-C₆)alkoxy, halo(C₁-C₆)alkyl and halo(C₁-C₆)alkoxy;

R⁴ is hydrogen, cyano, hydroxy, halogen, amino, (C₁-C₆)alkyl, (C₁-C₆)alkoxy, halo(C₁-C₆)alkyl, halo(C₁-C₆)alkoxy, (C₁-C₆)alkoxy-(C₁-C₆)alkyl, (C₃-C₆)cycloalkyl, (C₃-C₆)cycloalkyl-(C₁-C₆)alkyl, (C₃-C₆)cycloalkyl optionally substituted with one or more, particularly one to three, more particularly one or two substituents R^{4a}, (C₃-C₆)cycloalkyl-(C₁-C₆)alkyl optionally substituted with one or more, particularly one to three, more particularly one or two substituents R^{4b}, (C₃-C₆)cycloalkyl-(C₁-C₆)alkoxy optionally substituted with one or more, particularly one to three, more particularly one or two substituents R^{4c}, heterocycloalkyl optionally substituted with one or more, particularly one to three, more particularly one or two substituents R^{4d}, —CO₂R^{4a} or —CONR^{4b}R^{4c};

R^{4a}, R^{4b}, R^{4c} and R^{4d} are each independently selected from hydrogen and (C₁-C₆)alkyl;

R⁵ is —NH₂ or oxo;

and pharmaceutically acceptable salts thereof.

In particular, the present invention relates to compounds of formula I or II.

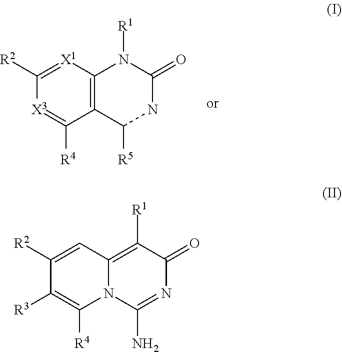

wherein

X¹ is either N or CH;

X³ is either N or CR³;

the dotted line represents a single bond when R⁵ is oxo or a double bond when R⁵ is —NH₂,

R¹ is (C₁-C₆)alkyl optionally substituted with one or more, particularly one to three, more particularly one or two substituents R^{1a}, (C₁-C₆)alkoxy optionally substituted with one or more, particularly one to three, more particularly one or two substituents R^{1b}, halo(C₁-C₆)alkyl optionally substituted with one or more, particularly one to three, more particularly one or two substituents R^{1a}, halo(C₁-C₆)alkoxy optionally substituted with one or more, particularly one to three, more particularly one or two substituents R^{1b}, (C₃-C₈)cycloalkyl optionally substituted with one or more, particularly one to three, more particularly one or two substituents R^{1c}, heteroaryl optionally substituted with one or more, particularly one to three, more particularly one or two substituents R^{1d}, heterocycloalkyl optionally substituted with one or more, particularly one to three, more particularly one or two substituents R^{1e} or phenyl optionally substituted with one or more, particularly one to three, more particularly one or two substituents R^{1f};

R^{1a} and R^{1b} are each independently selected from (C₃-C₆)cycloalkyl, hydroxyl, heteroaryl, heterocycloalkyl and phenyl, wherein heteroaryl, heterocycloalkyl or phenyl are optionally substituted with one or more, particularly one to three, more particularly one or two substituents R^{1g};

R^{1c}, R^{1d}, R^{1c} and R^{1f} are each independently selected from halogen, oxo, cyano, hydroxyl, (C₁-C₆)alkyl, (C₁-C₆)alkoxy, (C₃-C₆)cycloalkyl, (C₃-C₆)cycloalkyl-(C₁-C₆)alkyl, halo(C₁-C₆)alkyl, halo(C₁-C₆)alkoxy, hydroxy(C₁-C₆)alkyl, (C₁-C₆)alkoxy-(C₁-C₆)alkyl, heteroaryl, heterocycloalkyl and phenyl;

R^{1g} are each independently selected from halogen, cyano, hydroxyl, (C₁-C₆)alkyl, (C₁-C₆)alkoxy, (C₃-C₆)cycloalkyl, (C₁-C₆)alkoxy, (C₃-C₆)cycloalkyl-(C₁-C₆)alkyl, halo(C₁-C₆)alkyl, halo(C₁-C₆)alkoxy, hydroxy(C₁-C₆)alkyl, and (C₁-C₆)alkoxy-(C₁-C₆)alkyl; R² is hydrogen, halogen, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{2a}, (C₃-C₆)cycloalkyl-(C₁-C₆)alkyl optionally substituted with one or more, particularly one to three, more particularly one or two substituents R^{2b}, (C₃-C₆)cycloalkyl-(C₁-C₆)alkoxy optionally substituted with one or more, particularly one to three, more particularly one or two substituents R^{2c}, heterocycloalkyl optionally substituted with one or more, particularly one to three, more particularly one or two substituents R^{2a} or phenyl optionally substituted with one or more, particularly one to three, more particularly one or two substituents R^{2e};

R^{2a}, R^{2b}, R^{2c}, R^{2a} and R^{2e} are each independently selected from halogen, (C₁-C₆)alkyl, (C₁-C₆)alkoxy, halo(C₁-C₆)alkyl and halo(C₁-C₆)alkoxy;

R³ is hydrogen, halogen, cyano, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{3a}, (C₃-C₆)cycloalkyl-(C₁-C₆)alkyl optionally substituted with one or more, particularly one to three, more particularly one or two substituents R^{3b}, (C₃-C₆)cycloalkyl-(C₁-C₆)alkoxy optionally substituted with one or more, particularly one to three, more particularly one or two substituents R^{3c}, heterocycloalkyl optionally substituted with one or more, particularly one to three, more particularly one or two substituents R^{3d} or phenyl optionally substituted with one or more, particularly one to three, more particularly one or two substituents R^{3e};

R^{3a}, R^{3b}, R^{3c}, R^{3d} and R^{3e} are each independently selected from halogen, (C₁-C₆)alkyl, (C₁-C₆)alkoxy, halo(C₁-C₆)alkyl and halo(C₁-C₆)alkoxy; R⁴ is hydrogen, cyano, hydroxy, halogen, amino, (C₁-C₆)alkyl, (C₁-C₆)alkoxy, halo(C₁-C₆)alkyl, halo(C₁-C₆)alkoxy, (C₁-C₆)alkoxy-(C₁-C₆)alkyl, (C₃-C₆)cycloalkyl, (C₃-C₆)cycloalkyl-(C₁-C₆)alkyl, (C₃-C₆)cycloalkyl optionally substituted with one or more, particularly one to three, more particularly one or two substituents R^{4a}, (C₃-C₆)cycloalkyl-(C₁-C₆)alkyl optionally substituted with one or more, particularly one to three, more particularly one or two substituents R^{4b}, (C₃-C₆)cycloalkyl-(C₁-C₆)alkoxy optionally substituted with one or more, particularly one to three, more particularly one or two substituents R^{4c}, heterocycloalkyl optionally substituted with one or more, particularly one to three, more particularly one or two substituents R^{4d}, —CO₂R^{4a} or —CONR^{4b}R^{4c};

R^{4a}, R^{4b}, R^{4c} and R^{4d} are each independently selected from hydrogen and (C₁-C₆)alkyl;

R⁵ is —NH₂ or oxo;

and pharmaceutically acceptable salts thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below.

All publications, patent applications, patents, and other references mentioned herein are incorporated by reference in their entirety.

The nomenclature used in this application is based on IUPAC systematic nomenclature, unless indicated otherwise.

Any open valency appearing on a carbon, oxygen, sulfur or nitrogen atom in the structures herein indicates the presence of a hydrogen, unless indicated otherwise.

When indicating the number of substituents, the term “one or more” refers to the range from one substituent to the highest possible number of substitution, i.e. replacement of one hydrogen up to replacement of all hydrogens by substituents, in particular wherein “one or more” refers to one, two or three, most particularly “one or more” refers to one or two.

The term “substituent” denotes an atom or a group of atoms replacing a hydrogen atom on the parent molecule.

The term “substituted” denotes that a specified group bears one or more substituents.

Where any group can carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitution, i.e. replacement of one hydrogen up to replacement of all hydrogens by substituents.

The term “amino” denotes a group of the formula —NR′R″ wherein R′ and R″ are independently hydrogen, (C₁-C₆)alkyl, halo(C₁-C₆)alkyl, or (C₃-C₆)cycloalkyl as described herein. Alternatively, R′ and R″, together with the nitrogen to which they are attached, can form a heterocycloalkyl. The term “primary amino” denotes a group wherein both R′ and R″ are hydrogen. The term “secondary amino” denotes a group wherein R′ is hydrogen and R″ is a group other than hydrogen, particularly wherein R″ is (C₁-C₆)alkyl. The term “tertiary amino” denotes a group wherein both R′ and R″ are other than hydrogen, particularly wherein R′ and R″ are both (C₁-C₆)alkyl. Particular secondary and tertiary amines are methylamine, ethylamine, propylamine, isopropylamine, phenylamine, benzylamine dimethylamine, diethylamine, dipropylamine and diisopropylamine, most particularly amino refers to ethylamine.

“halo” or “halogen” means fluoro, chloro, bromo or iodo, particularly chloro or fluoro.

“hydroxy” refers to a —OH group.

“(C₁-C₆)alkyl” refers to a branched or straight hydrocarbon chain of one to six carbon atoms, such as methyl, ethyl, n-propyl, isopropyl, n-butyl, isobutyl, sec-butyl, t-butyl, pentyl and hexyl.

“(C₁-C₆)alkoxy” means a moiety of the formula —OR^{a}, wherein R^{a} is an (C₁-C₆)alkyl moiety as defined herein. Examples of (C₁-C₆)alkoxy moieties include, but are not limited to, methoxy, ethoxy, isopropoxy, and the like.

The term “(C₃-C₈)cycloalkyl” denotes a saturated monovalent saturated monocyclic hydrocarbon group of 3 to 6 ring carbon atoms. Examples for monocyclic (C₃-C₈)cycloalkyl are cyclopropyl, cyclobutanyl, cyclopentyl, cyclohexyl or cycloheptyl. One particular example of (C₃-C₆)cycloalkyl is cyclopropyl.

“(C₃-C₆)cycloalkyl-(C₁-C₆)alkyl” refers to an (C₁-C₆)alkyl, as defined above, substituted with one or more (C₃-C₆)cycloalkyl group, particularly with one (C₃-C₆)cycloalkyl group. More particularly “(C₃-C₆)cycloalkyl-(C₁-C₆)alkyl refers to

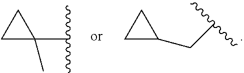

The term “perhalo(C₁-C₃)alkyl” means an (C₁-C₃)alkyl group as defined above wherein all hydrogen atoms have been replaced with halogen atoms. More particularly “(C₁-C₃)perhaloalkyl” is (C₁-C₃)perfluoroalkyl, most preferably trifluoromethyl.

“halo-(C₁-C₆)alkyl” refers to an (C₁-C₆)alkyl, as defined above, substituted with one or more halogen atoms, particularly with one to three halogen atoms. More particularly halo-(C₁-C₆)alkyl is the chloro- and fluoro-(C₁-C₆)alkyl. In some particular embodiment halo-(C₁-C₆)alkyl refers to perhalo(C₁-C₃)alkyl as defined herein. Most particularly halo-(C₁-C₆)alkyl is trifluoromethyl, difluoromethyl or fluoromethyl.

“halo-(C₁-C₆)alkoxy” refers to an (C₁-C₆)alkoxy, as defined above, substituted with one or more halogen atoms, particularly with one to three halogen atoms. More particularly halo-(C₁-C₆) alkoxy is the chloro- and fluoro-(C₁-C₆) alkoxy. In some particular embodiment halo-(C₁-C₆) alkoxy refers to perhalo(C₁-C₃) alkoxy, such as trifluoromethoxy or difluoromethoxy.

“hydroxy-(C₁-C₆)alkyl” refers to an (C₁-C₆)alkyl, as defined above, substituted with one or more hydroxy group, particularly with one hydroxy group. More particularly hydroxy-(C₁-C₆)alkyl refers to methyl-hydroxide or ethyl-hydroxide.

“(C₁-C₆)alkoxy-(C₁-C₆)alkyl” refers to an (C₁-C₆)alkyl, as defined above, substituted with one or more (C₁-C₆)alkoxy group as defined herein, particularly with one (C₁-C₆)alkoxy group. More particularly (C₁-C₆)alkoxy-(C₁-C₆)alkyl refers to —CH₂—O—CH₃ or —CH₂CH₂—O—CH₃.

“halo-(C₁-C₆)alkoxy” refers to an alkoxy, as defined above, substituted with one or more halogen atoms, particularly with one to three halogen atoms. More particularly halo-(C₁-C₆)alkoxy are the chloro- and fluoro-(C₁-C₆)alkoxy.

“Heteroaryl” means a monovalent monocyclic or bicyclic moiety of 5 to 12 ring atoms having at least one aromatic ring containing one, two, or three ring heteroatoms selected each independently from N, O, or S (preferably N or O), the remaining ring atoms being C, with the understanding that the attachment point of the heteroaryl moiety will be on an aromatic ring.

More specifically the term heteroaryl includes, but is not limited to, pyridinyl, furanyl, thienyl, thiazolyl, isothiazolyl, triazolyl, imidazolyl, isoxazolyl, oxazolyl, pyrrolyl, pyrazolyl, pyrimidinyl, pyrazinyl, pyridazinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, imidazo[1,2-a]-pyridinyl, imidazo[2,1-b]thiazolyl, and the derivatives thereof. “N-heteroaryl” in particular refers to heteroaryl as previously defined containing at least one nitrogen atom. The point of attachment of the N-heteroaryl to the rest of the molecule can be through the nitrogen or a carbon ring atom. Example of N-heteroaryl are pyridinyl, pyrazinyl, pyridazinyl, pyrimidinyl.

The term “heterocycloalkyl” or “heterocyclic” denotes a monovalent saturated or partly unsaturated mono- or biclyclic ring system of 4 to 9 ring atoms, comprising 1, 2, or 3 ring heteroatoms selected independently from N, O and S, the remaining ring atoms being carbon. Examples for heterocycloalkyl are pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, dioxolane, 1,4-dioxepanyl, oxepanyl, 1,1-dioxo-thiomorpholin-4-yl, azepanyl, diazepanyl, homopiperazinyl, or oxazepanyl. More particularly heterocycloalkyl refers to dihydrofuryl, 1,3-dioxolyl, dihydropyrryl, dihydrothiophyl, dihydropyrazolyl, dihydroisoxazolyl, tetrahydropyridyl, tetrahydropyranyl, tetrahydrothiopyranyl, piperazinyl, 3,4-dihydro-2H-1,4-oxazinyl, 3,4-dihydro-2H-1,4-thiazyl, 1,2,3,4-tetrahydropyrazyl.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

“Optional” or “optionally” means that the subsequently described event or circumstance may but need not occur, and that the description includes instances where the event or circumstance occurs and instances in which it does not. For example, “aryl group optionally substituted with an alkyl group” means that the alkyl may but need not be present, and the description includes situations where the aryl group is substituted with an alkyl group and situations where the aryl group is not substituted with the alkyl group.

The terms “individual” or “subject” refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The terms “compound(s) of this invention” and “compound(s) of the present invention” refer to compounds as disclosed herein and stereoisomers, tautomers, solvates, and salts (e.g., pharmaceutically acceptable salts) thereof.

When the compounds of the invention are solids, it is understood by those skilled in the art that these compounds, and their solvates and salts, may exist in different solid forms, particularly different crystal forms, all of which are intended to be within the scope of the present invention and specified formulae.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “active pharmaceutical ingredient” (or “API”) denotes the compound or molecule in a pharmaceutical composition that has a particular biological activity.

The terms “pharmaceutical composition” and “pharmaceutical formulation” (or “formulation”) are used interchangeably and denote a mixture or solution comprising a therapeutically effective amount of an active pharmaceutical ingredient together with pharmaceutically acceptable excipients to be administered to a mammal, e.g., a human in need thereof.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

The terms “treating” or “treatment” of a disease state include inhibiting the disease state, i.e., arresting the development of the disease state or its clinical symptoms, or relieving the disease state, i.e., causing temporary or permanent regression of the disease state or its clinical symptoms.

Compounds that have the same molecular formula but differ in the nature or sequence of bonding of their atoms or the arrangement of their atoms in space are termed “isomers.” Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, if a carbon atom is bonded to four different groups, a pair of enantiomers is possible. An enantiomer can be characterized by the absolute configuration of its asymmetric center and is described by the R- and S-sequencing rules of Cahn, Ingold and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of formula I or II can possess one or more asymmetric centers or axes. Unless indicated otherwise, the description or naming of a particular compound in the specification and claims is intended to include both individual enantiomers, atropisomers and mixtures, racemic or otherwise, thereof, as well as individual epimers, atropisomers and mixtures thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

Certain compounds may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—↔—C(—OH)═CH—), amide/imidic acid (—C(═O)—NH—↔—C(—OH)═N—) and amidine (—C(═NR)—NH—↔—C(—NHR)═N—) tautomers. The latter two are particularly common in heteroaryl and heterocyclic rings and the present invention encompasses all tautomeric forms of the compounds.

Now it has been found that the present compounds of formula formula I or II are inhibitors of Mat2A and as such may be of therapeutic use for the treatment of Cancer disorders including Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma.

These compounds are potent inhibitors of the Human methionine adenosyltransferase II alpha (MAT2A). MAT2A and MAT1A (methionine adenosyltransferase I alpha) are two genes that encode for methionine adenosyltransferase activity thereby producing S-adenosylmethionine (SAM), the prinical methyl donor in the cells. MAT1A is the liver specific SAM producing enzyme, whereas MAT2A is broadly expressed, except in the liver. MAT2A is found in complex with MAT2B (methionine adenosyltransferase II beta), the allosteric regulator of MAT2A, and MAT2B acts like a rheostat for MAT2A enzymatic activity. When MAT2B binds to MAT2A, MAT2A undergoes a conformational change that increases its affinity for methionine and SAM. The net effect is that MAT2A, when bound to MAT2B, is more active under low methionine concentrations, but is inhibited under high methionine concentrations.

Loss-of-function mutations in tumor suppressor genes are critical in the molecular pathogenesis of cancer, however successful targeting of tumor suppressors has been elusive mainly because the mutant proteins cannot be directly inhibited for therapeutic benefit, and restoration of mutant function (such as restoring function of mutant p53), has so far not been possible. The recent clinical success of inhibiting PARP in BRCA1/2 deficient patients has shown that targeting conditional synthetic lethalities (CSLs) that arise from loss-of-function mutations in tumor suppressors is a clinically valid approach for the treatment of cancers. The CSL relationship is not only valid for tumor suppressors but can be extended to genes that reside in the same genetic region of a tumor suppressor and are lost when that region is deleted. Methylthioadenosine phosphorylase (MTAP) is one such gene that is in close proximity to the tumor suppressor CDKN2A, and is deleted in ˜15% of all cancers. MTAP is deleted in, but not limited to, ˜53% of glioblastoma multiforme (GBM), ˜25% of pancreatic adenocarcinoma (PDAC), ˜25% of melanoma, ˜23% lung squamous cell carcinoma, ˜20% head and neck squamous cell carcinoma, and ˜15% lung adenocarcinoma. Indeed, this deletion occurs across multiple indications, many of which are areas of high unmet medical need with limited efficacious therapies. In glioblastoma, were the median survival is 14 months, the approval of the most recent therapies has not increased the overall survival (OS) time significantly and the standard of care (SoC) remains the same for over a decade. The same is true for the majority of patients with PDAC where OS is less than 1 year. MTAP deletion is a truncal event that occurs early on in tumor development and would be carried through all evolutions of the tumor including metastasis. Therefore its loss represents an alteration that is not affected by tumor heterogeneity, genetic background, or resistance to any approved agents in the clinic. A CSL relationship identified for MTAP deficiency would represent a true Achilles' heel for multiple tumor indications.

MTAP is located in close proximity to the tumor suppressor CDKN2A on chromosome 9. When CDKN2A is deleted, MTAP is frequently co-deleted. Its loss is thought to be a bystander effect and phenotypically neutral. MTAP is the cornerstone of the adenine and methionine salvage pathways in cells. The methionine salvage pathway feeds into the SAM production pathway, and the levels of SAM are a key regulator of cancer cell growth that needs to be tightly regulated because large changes in SAM concentrations, either increases or decreases, lead to cell cycle arrest. The importance of SAM levels to cancerous growth lies in its central role for protein, DNA, and RNA methylation, acting as a checkpoint for the health of the cell, and can be read out as hypomethylation when SAM is reduced or hypermethylation when SAM is increased. Cells that lack MTAP accumulate methylthioadenosine (MTA) and decarboxylated SAM (dcSAM) without adversely affecting the levels of any salvage metabolites/products including SAM. This accumulation creates a novel stress on the cell where MTA acts as a competitive inhibitor of SAM dependent reactions due to their structural similarity. The loss of MTAP forces the cell to adapt to the new MTA/SAM paradigm without any loss in viability that a MTAP proficient cell would not have to contend with, and this adaptation creates a robust dependence on methionine adenosyltransferase II alpha2 (MAT2A), one of the enzymes that produces SAM, in MTAP deficient cells. This conditional synthetic lethal (CSL) relationship of MTAP loss and MAT2A dependence was identified in three large scale shRNA screens (Marjon Cell Reports 2016, Kryukov Science 2016, and Mavrakis Science 2016).

Targeting MAT2A with a small molecule inhibition would bring benefit to a genetically defined patient population representing many areas of high unmet medical need.

Objects of the present invention are compounds of formula I or II the use of such compounds for the preparation of medicaments for the treatment, prevention and/or delay of progression of Cancer, in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma more particularly for the treatment of cancer including Lung Adenocarcinoma, Lung Squamous Carcinoma, Pancreatic Adenocarcinoma, Glioblastoma Multiforme, and Head and Neck Squamous Carcinoma, their manufacture and medicaments based on a compound of formula I or II in accordance with the invention.

Further objects of the present invention are all forms of optically pure enantiomers, racemates or diastereometric mixtures for compounds of formula I or II.

In particular, the present invention relates to compounds of formula I.

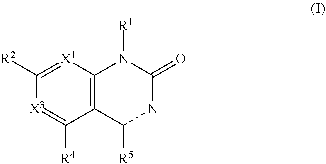

wherein

X¹ is either N or CH;

X³ is either N or CR³;

the dotted line represents a single bond when R⁵ is oxo or a double bond when R⁵ is —NH₂,

R¹ is (C₁-C₆)alkyl optionally substituted with one or more, particularly one to three, more particularly one or two substituents R^{1a}, (C₁-C₆)alkoxy optionally substituted with one or more, particularly one to three, more particularly one or two substituents R^{1b}, (C₃-C₈)cycloalkyl optionally substituted with one or more, particularly one to three, more particularly one or two substituents R^{1c}, heteroaryl optionally substituted with one or more, particularly one to three, more particularly one or two substituents R^{1d}, heterocycloalkyl optionally substituted with one or more, particularly one to three, more particularly one or two substituents R^{1e} or phenyl optionally substituted with one or more, particularly one to three, more particularly one or two substituents R^{1f};

R^{1a} and R^{1b} are each independently selected from (C₃-C₆)cycloalkyl, hydroxyl, heteroaryl, heterocycloalkyl and phenyl, wherein heteroaryl, heterocycloalkyl or phenyl are optionally substituted with one or more, particularly one to three, more particularly one or two substituents R^{1g};

R^{1c}, R^{1d}, R^{1e} and R^{1f} are each independently selected from halogen, cyano, oxo, hydroxy, (C₁-C₆)alkyl, (C₁-C₆)alkoxy, (C₃-C₆)cycloalkyl, (C₃-C₆)cycloalkyl-(C₁-C₆)alkyl, halo(C₁-C₆)alkyl, halo(C₁-C₆)alkoxy, hydroxy(C₁-C₆)alkyl, (C₁-C₆)alkoxy-(C₁-C₆)alkyl, heteroaryl, heterocycloalkyl and phenyl;

R^{1g} are each independently selected from halogen, cyano, hydroxyl, (C₁-C₆)alkyl, (C₁-C₆)alkoxy, (C₃-C₆)cycloalkyl, (C₃-C₆)cycloalkyl-(C₁-C₆)alkyl, halo(C₁-C₆)alkyl, halo(C₁-C₆)alkoxy, hydroxy(C₁-C₆)alkyl and (C₁-C₆)alkoxy-(C₁-C₆)alkyl;

R² is hydrogen, halogen, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{2a}, (C₃-C₆)cycloalkyl-(C₁-C₆)alkyl optionally substituted with one or more, particularly one to three, more particularly one or two substituents R^{2b}, (C₃-C₆)cycloalkyl-(C₁-C₆)alkoxy optionally substituted with one or more, particularly one to three, more particularly one or two substituents R^{2c}, heterocycloalkyl optionally substituted with one or more, particularly one to three, more particularly one or two substituents R^{2d} or phenyl optionally substituted with one or more, particularly one to three, more particularly one or two substituents R^{2e};

R^{2a}, R^{2b}, R^{2c}, R^{2d} and R^{2e} are each independently selected from halogen, (C₁-C₆)alkyl, (C₁-C₆)alkoxy, halo(C₁-C₆)alkyl and halo(C₁-C₆)alkoxy;

R³ is hydrogen, halogen, cyano, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{3a}, (C₃-C₆)cycloalkyl-(C₁-C₆)alkyl optionally substituted with one or more, particularly one to three, more particularly one or two substituents R^{3b}, (C₃-C₆)cycloalkyl-(C₁-C₆)alkoxy optionally substituted with one or more, particularly one to three, more particularly one or two substituents R^{3c}, heterocycloalkyl optionally substituted with one or more, particularly one to three, more particularly one or two substituents R^{3d} or phenyl optionally substituted with one or more, particularly one to three, more particularly one or two substituents R^{3e};

R^{3a}, R^{3b}, R^{3c}, R^{3d} and R^{3e} are each independently selected from halogen, (C₁-C₆)alkyl, (C₁-C₆)alkoxy, halo(C₁-C₆)alkyl and halo(C₁-C₆)alkoxy;

R⁴ is hydrogen, cyano, hydroxy, halogen, amino, (C₁-C₆)alkyl, (C₁-C₆)alkoxy, halo(C₁-C₆)alkyl, halo(C₁-C₆)alkoxy, (C₁-C₆)alkoxy-(C₁-C₆)alkyl, (C₃-C₆)cycloalkyl, (C₃-C₆)cycloalkyl-(C₁-C₆)alkyl, (C₃-C₆)cycloalkyl optionally substituted with one or more, particularly one to three, more particularly one or two substituents R^{4a}, (C₃-C₆)cycloalkyl-(C₁-C₆)alkyl optionally substituted with one or more, particularly one to three, more particularly one or two substituents R^{4b}, (C₃-C₆)cycloalkyl-(C₁-C₆)alkoxy optionally substituted with one or more, particularly one to three, more particularly one or two substituents R^{4c}, heterocycloalkyl optionally substituted with one or more, particularly one to three, more particularly one or two substituents R^{4d};

R^{4a}, R^{4b}, R^{4c} and R^{4d} are each independently selected from hydrogen and (C₁-C₆)alkyl;

R⁵ is —NH₂ or oxo;

and pharmaceutically acceptable salts thereof.

In particular embodiment, the present invention relates to compounds of formula I′:

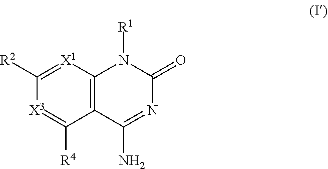

wherein X¹, X³, R¹, R² and R⁴ are as defined herein.

In particular embodiment, the present application relates to compounds of formula I″:

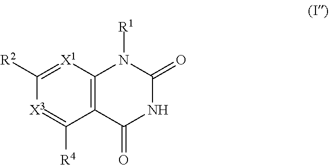

wherein X¹, X³, R¹, R² and R⁴ are as defined herein.

Further, it is to be understood that every embodiment relating to a specific X¹, X³, R¹, R^{1a}, R^{1b}, R^{1c}, R^{1d}, R^{1e}, R^{1f}, R^{1g}, R², R^{2a}, R^{2b}, R^{2c}, R^{2d}, R^{2e}, R³, R^{3a}, R^{3b}, R^{3c}, R^{3d}, R^{3e}, R⁴, R^{4a}, R^{4b}, R^{4c}, R^{4d} and R⁵ as disclosed herein may be combined with any other embodiment relating to another

X¹, X³, R¹, R^{1a}, R^{1b}, R^{1c}, R^{1d}, R^{1e}, R^{1f}, R^{1g}, R², R^{2a}, R^{2b}, R^{2c}, R^{2d}, R^{2e}, R³, R^{3a}, R^{3b}, R^{3c}, R^{3d}, R^{3e}, R⁴, R^{4a}, R^{4b}, R^{4c}, R^{4d} and R⁵ as disclosed herein.

A particular embodiment of the present invention relates to a compound of formula I, I′ or I″, wherein X³ is CR³.

A particular embodiment of the present invention relates to a compound of formula I, I′ or I″, wherein X¹ is N.

An other particular embodiment of the present invention relates to a compound of formula I, I′ or I″, wherein X¹ is CH.

A particular embodiment of the present invention relates to a compound of formula I, I′ or I″, wherein R¹ is (C₁-C₆)alkyl optionally substituted with one R^{1a}, (C₃-C₆)cycloalkyl optionally substituted with one R^{1c}, heteroaryl optionally substituted with one or two R^{1d}, heterocycloalkyl optionally substituted with on R^{1e} or phenyl optionally substituted with one or two R^{1f}; more particularly wherein R¹ is (C₁-C₃)alkyl optionally substituted with one R^{1a}, (C₃—C)cycloalkyl optionally substituted with one R^{1c}, pyrazolyl optionally substituted with one R^{1d}, indazolyl optionally substituted with one R^{1d}, indolyl optionally substituted with one R^{1d}, benzo[d]oxazolyl optionally substituted with one R^{1d}, benzo[d]thiazolyl optionally substituted with one R^{1d}, benzo[d]imidazolyl optionally substituted with one R^{1d}, dioxepanyl optionally substituted with one R^{1d}, oxazolyl optionally substituted with one R^{1d}, thiazolyl optionally substituted with one R^{1d}, pyridinyl optionally substituted with one or two R^{1d}, pyrimidinyl optionally substituted with one R^{1d}, dihydropyrrolo[1,2-c]imidazolyl optionally substituted with one R^{1e}, oxepanyl optionally substituted with one R^{1e}, dihydro-indolyl optionally substituted with one R^{1e}, 1,4-dioxepanyl optionally substituted with one R^{1e}, tetrahydrofuranyl optionally substituted with one R^{1e}, tetrahydropyranyl optionally substituted with one R^{1e}, piperidinyl optionally substituted with one R^{1e}, oxaspiro[2.5]octanyl optionally substituted with one R^{1e}, dihydrobenzofuranyl optionally substituted with one R^{1e} or phenyl optionally substituted with one or two R^{1f}, more particularly wherein R¹ is (C₁-C₃)alkyl optionally substituted by R^{1a}, cyclopentyl optionally substituted with one R^{1c}, indazol-4-yl, pyrazolyl optionally substituted with one R^{1d}, oxazolyl optionally substituted with one R^{1d}, thiazolyl optionally substituted with one R^{1d}, pyridinyl optionally substituted with one or two R^{1d}, pyrimidinyl optionally substituted with one R^{1d}, oxepanyl, tetrahydrofuranyl, tetrahydropyranyl optionally substituted with one R^{1e}, piperidinyl optionally substituted with one R^{1e}, oxaspiro[2.5]octanyl, 2,3-dihydrobenzofuranyl or phenyl optionally substituted with one or two R^{1f}, even more particularly R¹ is pyridinyl optionally substituted with one or two R^{1d}, oxaspiro[2.5]octanyl, 2,3-dihydrobenzofuranyl, tetrahydropyranyl optionally substituted optionally substituted with one R^{1e} or phenyl optionally substituted with one or two R^{1f} most particularly tetrahydropyranyl optionally substituted with one (C₁-C₃)alkyl, more particularly methyl, in alpha.

A particular embodiment of the present invention relates to a compound of formula I, I′ or I″, wherein R¹ is (C₁-C₆)alkyl optionally substituted with one R^{1a}, (C₃-C₆)cycloalkyl optionally substituted with one R^{1c}, heteroaryl optionally substituted with one or two R^{1d}, heterocycloalkyl optionally substituted with one R^{1e} or phenyl optionally substituted with one or two R^{1f}; particularly wherein R¹ is (C₁-C₃)alkyl optionally substituted with one R^{1a}, (C₃-C₆)cycloalkyl optionally substituted with one R^{1c}, pyrazolyl optionally substituted with one R^{1d}, oxazolyl optionally substituted with one R^{1d}, thiazolyl optionally substituted with one R^{1d}, pyridinyl optionally substituted with one or two R^{1d}, pyrimidinyl optionally substituted with one R^{1d}, tetrahydrofuranyl optionally substituted with one R^{1e}, tetrahydropyranyl optionally substituted with one R^{1e}, piperidinyl optionally substituted with one R^{1e}, oxaspiro[2.5]octanyl optionally substituted with one R^{1e}, 2,3-dihydrobenzofuranyl optionally substituted with one R^{1e} or phenyl optionally substituted with one or two R^{1f}, more particularly wherein R¹ is (C₁-C₃)alkyl optionally substituted by R^{1a}, cyclopentyl optionally substituted with one R^{1c}, pyrazolyl optionally substituted with one R^{1d}, oxazolyl optionally substituted with one R^{1d}, thiazolyl optionally substituted with one R^{1d}, pyridinyl optionally substituted with one or two R^{1d}, pyrimidinyl optionally substituted with one R^{1d}, tetrahydrofuranyl, tetrahydropyranyl, piperidinyl optionally substituted with one R″, oxaspiro[2.5]octanyl, 2,3-dihydrobenzofuranyl or phenyl optionally substituted with one or two R^{1f}, most particularly R¹ is pyridinyl optionally substituted with one or two R^{1d}, tetrahydropyranyl or phenyl optionally substituted with one or two R^{1f}.

A particular embodiment of the present invention relates to a compound of formula I, I′ or I″, wherein R¹ is heteroaryl substituted with one or two R^{1d} wherein at least of one R^{1d} is substituted in ortho, heterocycloalkyl substituted with one R^{1e} substituted in alpha or phenyl substituted with one or two R^{1f} wherein at least of one R^{1f} is substituted in ortho, in particular wherein R¹ is pyridinyl substituted with one or two R^{1d} wherein at least of one R^{1d} is substituted in ortho, tetrahydrofuranyl substituted with one R^{1e} wherein at least of one R^{1e} is substituted in alpha, tetrahydropyranyl substituted with one R^{1e} substituted in alpha, oxaspiro[2.5]octanyl or 2,3-dihydrobenzofuranyl substituted with one R^{1e}, more particularly wherein R¹ is tetrahydrofuranyl substituted with one R^{1e} substituted in alpha, tetrahydropyranyl substituted with one R^{1e} substituted in alpha, oxaspiro[2.5]octanyl or 2,3-dihydrobenzofuranyl substituted with one R^{1e}, most particularly wherein R¹ is tetrahydropyranyl substituted with one R^{1e} substituted in alpha.

More particular embodiment of the present invention relates to a compound of formula I, I′ or I″, wherein R¹ is heteroaryl optionally substituted with one or two R^{1d}, heterocycloalkyl optionally substituted with one R^{1e} or phenyl optionally substituted with one or two R^{1f}.

A particular embodiment of the present invention relates to a compound of formula I, I′ or I″, wherein R^{1a} and R^{1b} are each independently selected from heteroaryl, heterocycloalkyl and phenyl, particularly R^{1a} is selected from tetrahydrofuranyl, pyridinyl, oxetanyl or oxazolyl.

A particular embodiment of the present invention relates to a compound of formula I, I′ or I″, wherein R^{1c}, R^{1d}, R^{1e} and R^{1f} are each independently selected from halogen, oxo, cyano, hydroxy, (C₁-C₆)alkyl, (C₁-C₆)alkoxy, halo(C₁-C₆)alkyl and halo(C₁-C₆)alkoxy, particularly R^{1c}, R^{1d}, R^{1e} and R^{1f} are each independently selected from chloro, fluoro, oxo, cyano, hydroxy, (C₁-C₃)alkyl, (C₁-C₃)alkoxy and halo(C₁-C₃)alkyl, more particularly R^{1c}, R^{1d}, R^{1e} and R^{1f} are each independently selected from cyano, cloro and (C₁-C₃)alkyl, most particularly wherein R^{1d} are each independently selected from cyano, chloro and methyl.

A particular embodiment of the present invention relates to a compound of formula I, I′ or I″, wherein R¹ is 2,3-dihydrobenzofuranyl, 2-hydroxycyclopentyl, 3-hydroxycyclopentyl, 1-(tetrahydrofuran-2-yl)ethyl, 1-tetrahydrofuran-3-yl-ethyl, 1-pyridin-2-yl-ethyl, oxepan-3-yl, 1,4-dioxepan-6-yl, dihydro-1H-indol-4-yl, 1-(oxetan-3-yl)ethyl, 1-(oxazol-5-yl)ethyl, indazol-4-yl, oxaspiro[2.5]octanyl, 4-methyloxazol-5-yl, 2-methoxy-phenyl, 3-methyl-phenyl, 2-methyl-phenyl, 3-fluoro-2-methoxyphenyl, 2-methylbenzonitrile, 2-methoxybenzonitrile, 2-ethoxybenzonitrile, 2-chlorophenyl, 3-chlorophenyl, 4-fluoro-2-methylphenyl, 3-fluoro-2-methylphenyl, 3-fluorophenyl, 2-fluorophenyl, 2,6-difluorophenyl, 2,3-dimethylphenyl, phenyl, 2,3-difluorophenyl, 2-fluoro-3-methylphenyl, 3-methoxyphenyl, 3,5-difluorophenyl, 3,4-difluorophenyl, 2-trifluoromethyl-phenyl, 3-(fluoromethyl)-2-methylphenyl, 3-ethylphenyl, 3-chloro-2-fluorophenyl, 2-chloro-5-fluorophenyl, 2-chloro-4-fluorophenyl, 2,3-dichlorophenyl, benzo[d]oxazol-4-yl, benzo[d]imidazolyl, benzo[d]thiazol-7-yl, 2-oxopiperidin-4-yl, 2-methylpyrazol-3-yl, 1-ethyl-1H-pyrazol-5-yl, 2-methylpyridin-3-yl, picolinonitrile, 2-methoxypyridin-3-yl, 2-(trifluoromethyl)pyridin-3-yl, 4-methylpyridin-3-yl, 4-fluoro-2-methoxypyridin-3-yl, indolyl, 2-chloropyridin-3-yl, 6-methoxypyridin-2-yl, 4-methylpyrimidin-5-yl, trifluoromethoxypyridin-2-yl, dihydrobenzofuranyl, tetrahydrofuranyl, 4-methyltetrahydrofuran-3-yl, methyl-tetrahydro-2H-pyran-3-yl, 6,7-dihydro-5H-pyrrolo[1,2-c]imidazol-7-yl, tetrahydro-2H-pyran-3-yl, tetrahydro-2H-pyran-4-yl or 4-methylthiazol-5-yl, in particular wherein R¹ is 2,3-dihydrobenzofuranyl, oxaspiro[2.5]octanyl, oxepan-3-yl, 3-methyl-phenyl, 2-methyl-phenyl, 2-methylbenzonitrile, 2-chlorophenyl, phenyl, 2-methylpyridin-3-yl, 4-methylpyridin-3-yl, 4 2-chloropyridin-3-yl, 2-methyl-tetrahydro-2H-pyran-3-yl or 4-methylpyrimidin-5-yl, most particularly wherein R¹ is 2-methyl-tetrahydro-2H-pyran-3-yl.

A particular embodiment of the present invention relates to a compound of formula I, I′ or I″, wherein R¹ is 2,3-dihydrobenzofuranyl, 2-hydroxycyclopentyl, 3-hydroxycyclopentyl, 1-(tetrahydrofuran-2-yl)ethyl, 1-tetrahydrofuran-3-yl-ethyl, 1-pyridin-2-yl-ethyl, 1-(oxetan-3-yl)ethyl, 1-(oxazol-5-yl)ethyl, oxaspiro[2.5]octanyl, 4-methyloxazol-5-yl, 2-methoxy-phenyl, 3-methyl-phenyl, 2-methyl-phenyl, 3-fluoro-2-methoxyphenyl, 2-methylbenzonitrile, 2-methoxybenzonitrile, 2-ethoxybenzonitrile, 2-chlorophenyl, 4-fluoro-2-methylphenyl, 3-fluorophenyl, 2-fluorophenyl, 2,6-difluorophenyl, phenyl, 2,3-difluorophenyl, 3-methoxyphenyl, 3,5-difluorophenyl, 3-ethylphenyl, 2-oxopiperidin-4-yl, 2-methylpyrazol-3-yl, 1-ethyl-1H-pyrazol-5-yl, 2-methylpyridin-3-yl, picolinonitrile, 2-methoxypyridin-3-yl, 2-(trifluoromethyl)pyridin-3-yl, 4-methylpyridin-3-yl, 4-fluoro-2-methoxypyridin-3-yl, 2-chloropyridin-3-yl, 6-methoxypyridin-2-yl, 4-methylpyrimidin-5-yl, tetrahydrofuranyl, 4-methyltetrahydrofuran-3-yl, tetrahydro-2H-pyran-3-yl, tetrahydro-2H-pyran-4-yl or 4-methylthiazol-5-yl, in particular wherein R¹ is 2,3-dihydrobenzofuranyl, oxaspiro[2.5]octane, 3-methyl-phenyl, 2-methyl-phenyl, 2-methylbenzonitrile, 2-chlorophenyl, phenyl, 2-methylpyridin-3-yl, 4-methylpyridin-3-yl, 4 2-chloropyridin-3-yl or 4-methylpyrimidin-5-yl.

Another embodiment of the present invention relates to a compound of formula I, I′ or I″, wherein R² is halogen, (C₁-C₆)alkyl, (C₁-C₆)alkoxy, halo(C₁-C₆)alkyl, halo(C₁-C₆)alkoxy, (C₃-C₆)cycloalkyl optionally substituted with one R^{2a}, (C₃-C₆)cycloalkyl-(C₁-C₆)alkoxy, heterocycloalkyl optionally substituted with one or two R^{2a}, NR^{2f}R^{2g} or phenyl, in particular R² is halogen, (C₁-C₆)alkyl, (C₁-C₆)alkoxy, halo(C₁-C₆)alkyl, halo(C₁-C₆)alkoxy, (C₃-C₆)cycloalkyl optionally substituted with one R^{2a}, (C₃-C₆)cycloalkyl-(C₁-C₆)alkoxy, heterocycloalkyl optionally substituted with one or two R^{2d} or phenyl, more particularly R² is halogen, (C₁-C₃)alkyl, (C₁-C₃)alkoxy, halo(C₁-C₃)alkyl, halo(C₁-C₃)alkoxy, cyclopropyl optionally substituted with one R^{2a}, cyclobutyl optionally substituted with one R^{2a}, cyclopentyl optionally substituted with one R^{2a}, (C₃-C₆)cycloalkyl-(C₁-C₃)alkoxy, 4,5-dihydrofuran-3-yl, 7-azabicyclo[2.2.1]heptan-7-yl, 3-azabicyclo[2.2.1]heptan-3-yl, tetrahydrofuranyl, tetrahydropyranyl, azetidinyl optionally substituted with one or two R^{2d} or phenyl, more particularly R² is halogen, (C₁-C₃)alkyl, (C₁-C₃)alkoxy, halo(C₁-C₃)alkyl, halo(C₁-C₃)alkoxy, cyclopropyl optionally substituted by halogen or (C₁-C₃)alkyl, cyclobutyl, cyclopentyl, cyclopropyloxy, 4,5-dihydrofuran-3-yl, 7-azabicyclo[2.2.1]heptan-7-yl, 3-azabicyclo[2.2.1]heptan-3-yl, tetrahydrofuranyl, tetrahydropyranyl, azetidinyl optionally substituted by one or two (C₁-C₃)alkyl, even more particularly R² is (halo(C₁-C₃)alkyl, halo(C₁-C₃)alkoxy, cyclopropyl optionally substituted by halogen or (C₁-C₃)alkyl, most particularly R² is trifluoromethyl, difluoromethoxy, trifluoromethoxy or cyclopropyl.

Another embodiment of the present invention relates to a compound of formula I, I′ or I″, wherein R^{2a}, R^{2b}, R^{2c}, R^{2d} and R^{2e} are each independently selected from halogen and (C₁-C₆)alkyl, particularly R^{2a}, R^{2b}, R^{2c}, R^{2d} and R^{2e} are each independently selected from halogen and (C₁-C₃)alkyl, more particularly R^{2a}, R^{2b}, R^{2c}, R^{2a} and R^{2e} are each independently selected from chloro, fluoro and methyl.

Another embodiment of the present invention relates to a compound of formula I, I′ or I″, wherein R^{2f} and R^{2g} are each independently selected from hydrogen or (C₁-C₃)alkyl, particularly wherein one of R^{2f} and R^{2g} is hydrogen while the other is (C₁-C₃)alkyl.

In yet another embodiment of the present invention relates to a compound of formula I, I′ or I″, wherein R³ is hydrogen, halogen or cyano, in particular wherein R³ is hydrogen, chloro, fluoro or cyano, more particularly wherein R³ is hydrogen.

In yet another embodiment of the present invention relates to a compound of formula I, I′ or I″, wherein R^{3a}, R^{3b}, R^{3c}, R^{3d} and R^{3e} are each independently selected from halogen and (C₁-C₃)alkyl.

In a further embodiment of the present invention relates to a compound of formula I, I′ or I″, R⁴ is hydrogen, cyano, halogen, (C₁-C₆)alkyl, (C₁-C₆)alkoxy or —CONR^{4b}R^{4c}, in particular wherein R⁴ is hydrogen, cyano, chloro, fluoro or (C₁-C₃)alkyl, more particularly wherein R⁴ is hydrogen.

In a further embodiment of the present invention relates to a compound of formula I, I′ or I″, wherein R^{4b} or R^{4c} are hydrogen.

In yet another embodiment of the present invention relates to a compound of formula I, I′ or I″, wherein R⁵ is —NH₂.

Particular compounds of formula I of the present invention are those selected from the group consisting of:
4-amino-7-cyclopropyl-1-(o-tolyl)pyrido[2,3-d]pyrimidin-2 (1H)-one
4-amino-7-cyclopropyl-1-(2-methoxyphenyl)pyrido[2,3-d]pyrimidin-2 (1H)-one
4-amino-7-(tert-butyl)-1-(o-tolyl)pyrido[2,3-d]pyrimidin-2 (1H)-one
4-amino-1-(2-methoxyphenyl)-7-phenylpyrido[2,3-d]pyrimidin-2 (1H)-one
4-amino-7-(3,3-difluoroazetidin-1-yl)-1-(o-tolyl)pyrido[2,3-d]pyrimidin-2 (1H)-one
4-amino-7-cyclopropyl-1-(tetrahydrofuran-3-yl)pyrido[2,3-d]pyrimidin-2 (1H)-one
4-amino-7-cyclopropyl-1-(2-methylpyridin-3-yl)pyrido[2,3-d]pyrimidin-2 (1H)-one
4-amino-7-cyclopropyl-1-(2-oxopiperidin-4-yl)pyrido[2,3-d]pyrimidin-2 (1H)-one
4-amino-7-cyclopropyl-1-((cis)-2-methyltetrahydrofuran-3-yl)pyrido[2,3-d]pyrimidin-2 (1H)-one
4-amino-7-cyclopropyl-1-(o-tolyl)pyrido[4,3-d]pyrimidin-2 (1H)-one
4-amino-7-cyclopropyl-1-(2-methylpyridin-3-yl)quinazolin-2-one
4-amino-7-cyclopropyl-1-(2-methylphenyl)quinazolin-2-one
7-cyclopropyl-1-(2-methylphenyl)quinazoline-2,4-dione
4-amino-7-cyclopropyl-1-(o-tolyl)pyrimido[4,5-d]pyrimidin-2 (1H)-one
7-cyclopropyl-1-(2-methylpyridin-3-yl)quinazoline-2,4-dione
4-amino-7-cyclopropyl-1-(2-methoxypyridin-3-yl)pyrido[2,3-d]pyrimidin-2 (1H)-one
4-amino-7-cyclopropyl-6-fluoro-1-(2-methylpyridin-3-yl)pyrido[2,3-d]pyrimidin-2 (1H)-one
7-cyclopropyl-1-(2-methylphenyl)pyrido[2,3-d]pyrimidine-2,4-dione
3-(4-amino-7-cyclopropyl-2-oxopyrido[2,3-d]pyrimidin-1 (2H)-yl)picolinonitrile
4-amino-7-cyclopropyl-1-(oxan-3-yl)pyrido[2,3-d]pyrimidin-2-one
4-amino-7-cyclopropyl-1-[1-(oxolan-3-yl)ethyl]pyrido[2,3-d]pyrimidin-2-one
4-amino-7-cyclopropyl-1-(3-fluoro-2-methoxyphenyl)pyrido[2,3-d]pyrimidin-2 (1H)-one
3-(4-amino-7-cyclopropyl-2-oxopyrido[2,3-d]pyrimidin-1 (2H)-yl)-2-methylbenzonitrile
4-amino-1-(2-methylpyridin-3-yl)-7-propan-2-ylpyrido[2,3-d]pyrimidin-2-one
4-amino-7-cyclopropyl-1-(2,3-dihydrobenzofuran-4-yl)pyrido[2,3-d]pyrimidin-2 (1H)-one
4-amino-6-chloro-7-cyclopropyl-1-(2-methylpyridin-3-yl)pyrido[2,3-d]pyrimidin-2 (1H)-one
3-(4-amino-7-cyclopropyl-2-oxopyrido[2,3-d]pyrimidin-1 (2H)-yl)-2-methoxybenzonitrile
3-(4-amino-7-cyclopropyl-2-oxopyrido[2,3-d]pyrimidin-1 (2H)-yl)-2-ethoxybenzonitrile
4-amino-7-cyclopropyl-1-(1-(tetrahydrofuran-2-yl)ethyl)pyrido[2,3-d]pyrimidin-2 (1H)-one
4-amino-1-(2-methylpyridin-3-yl)-7-(oxetan-3-yl)quinazolin-2 (1H)-one
7-cyclopropyl-1-(2-methylpyridin-3-yl)pyrido[2,3-d]pyrimidine-2,4-dione
4-amino-7-((1RS,2RS)-2-methylcyclopropyl)-1-(2-methylpyridin-3-yl)pyrido[2,3-d]pyrimidin-2 (1H)-one
4-amino-7-cyclopentyl-1-(2-methyl-3-pyridyl)pyrido[2,3-d]pyrimidin-2-one
4-amino-7-cyclopropyl-1-((1SR,2RS)-2-hydroxycyclopentyl)pyrido[2,3-d]pyrimidin-2 (1H)-one
4-amino-2-cyclopentyl-7-(o-tolyl)pyrazolo[3,4-d]pyrimidin-6-one; formic acid
4-amino-7-cyclopentyl-1-(4-methylpyrimidin-5-yl)pyrido[2,3-d]pyrimidin-2-one
4-amino-7-cyclopropyl-1-(3-fluoro-2-methylphenyl)pyrido[2,3-d]pyrimidin-2 (1H)-one
4-amino-7-cyclopropyl-1-[(3R)-oxan-3-yl]pyrido[2,3-d]pyrimidin-2-one
4-amino-7-cyclopropyl-1-[(3S)-oxan-3-yl]pyrido[2,3-d]pyrimidin-2-one
4-amino-7-cyclopropyl-1-(4-methyltetrahydrofuran-3-yl)pyrido[2,3-d]pyrimidin-2 (1H)-one
4-amino-7-cyclopropyl-2-oxo-1-(o-tolyl)-1,2-dihydropyrido[2,3-d]pyrimidine-5-carboxamide
4-amino-1-(2-methoxy-3-pyridyl)-7-tetrahydropyran-2-yl-pyrido[2,3-d]pyrimidin-2-one
4-amino-7-[(1S,4R)-3-azabicyclo[2.2.1]heptan-3-yl]-1-(2-methylpyrazol-3-yl)pyrido[2,3-d]pyrimidin-2-one; formic acid
4-amino-1-(2-methylpyridin-3-yl)-7-(trifluoromethyl)quinazolin-2-one
4-amino-7-cyclopropyl-1-[rac-(2R,3S)-2-methyloxolan-3-yl]pyrido[2,3-d]pyrimidin-2-one
4-amino-7-cyclobutyl-1-(2-methyl-3-pyridyl)pyrido[2,3-d]pyrimidin-2-one; formic acid
4-amino-7-cyclopropyloxy-1-(2-methylpyridin-3-yl)quinazolin-2-one
4-amino-1-(2-methylpyridin-3-yl)-7-(trifluoromethyl)pyrido[2,3-d]pyrimidin-2 (1H)-one
4-amino-7-(7-azabicyclo[2.2.1]heptan-7-yl)-1-(4-methylthiazol-5-yl)pyrido[2,3-d]pyrimidin-2-one; formic acid
4-amino-7-cyclopropyl-1-(3-hydroxycyclopentyl)pyrido[2,3-d]pyrimidin-2 (1H)-one
4-amino-7-(difluoromethoxy)-1-(2-methylpyridin-3-yl)quinazolin-2 (1H)-one
4-amino-7-(difluoromethyl)-1-(2-methylpyridin-3-yl)quinazolin-2 (1H)-one
4-amino-7-[(1R,2S)-2-fluorocyclopropyl]-1-(2-methyl-3-pyridyl)pyrido[2,3-d]pyrimidin-2-one
4-amino-7-cyclopropyl-1-(2-methylpyridin-3-yl)-2-oxo-1,2-dihydropyrido[2,3-d]pyrimidine-6-carbonitrile
3-(4-amino-7-isopropyl-2-oxopyrido[2,3-d]pyrimidin-1 (2H)-yl)-2-methoxybenzonitrile
4-amino-7-cyclopropyl-2-oxo-1-(o-tolyl)-1,2-dihydropyrido[2,3-d]pyrimidine-5-carbonitrile
4-amino-7-methoxy-1-(2-methylpyridin-3-yl)quinazolin-2 (1H)-one
4-amino-1-(2-methylpyridin-3-yl)-7-(trifluoromethoxy)quinazolin-2 (1H)-one
4-amino-7-(4,5-dihydrofuran-3-yl)-1-(2-methylpyridin-3-yl)pyrido[2,3-d]pyrimidin-2 (1H)-one
4-amino-1-(2-methylpyridin-3-yl)-7-(tetrahydrofuran-3-yl)pyrido[2,3-d]pyrimidin-2 (1H)-one
4-amino-7-cyclopropyl-1-(2-methylpyridin-3-yl)pyrido[2,3-d]pyrimidin-2-one
4-amino-7-cyclopropyl-1-(2-methylpyridin-3-yl)pyrido[2,3-d]pyrimidin-2-one
4-amino-7-cyclopropyl-1-(4-methylthiazol-5-yl)pyrido[2,3-d]pyrimidin-2-one
4-amino-7-cyclopropyl-1-(4-methylpyrimidin-5-yl)pyrido[2,3-d]pyrimidin-2 (1H)-one
4-amino-7-cyclopropyl-1-(2-(trifluoromethyl)pyridin-3-yl)pyrido[2,3-d]pyrimidin-2 (1H)-one
4-amino-7-cyclopropyl-1-(2-methylpyrazol-3-yl)pyrido[2,3-d]pyrimidin-2-one
4-amino-7-cyclopropyl-1-(2,3-dihydrobenzofuran-4-yl)quinazolin-2 (1H)-one
(R)-4-amino-1-(tetrahydro-2H-pyran-3-yl)-7-(trifluoromethyl)quinazolin-2 (1H)-one
4-amino-7-cyclopropyl-1-(4-methylpyridin-3-yl)pyrido[2,3-d]pyrimidin-2 (1H)-one
4-amino-7-ethyl-1-(2-methylpyridin-3-yl)quinazolin-2 (1H)-one
4-amino-7-[(1S,2R)-2-fluorocyclopropyl]-1-[(3R)-tetrahydropyran-3-yl]pyrido[2,3-d]pyrimidin-2-one
4-amino-7-cyclopropyl-1-(4-methyloxazol-5-yl)pyrido[2,3-d]pyrimidin-2-one
3-(4-amino-6-chloro-7-isopropyl-2-oxopyrido[2,3-d]pyrimidin-1 (2H)-yl)-2-methoxybenzonitrile
4-amino-7-cyclopropyl-1-((R)-1-((S)-tetrahydrofuran-3-yl)ethyl)pyrido[2,3-d]pyrimidin-2 (1H)-one
4-amino-7-cyclopropyl-1-((R)-1-((R)-tetrahydrofuran-3-yl)ethyl)pyrido[2,3-d]pyrimidin-2 (1H)-one
4-amino-7-(2-fluoropropan-2-yl)-1-(2-methylpyridin-3-yl)quinazolin-2-one
4-amino-5-methoxy-1-(2-methylpyridin-3-yl)-7-(trifluoromethyl)quinazolin-2 (1H)-one
4-amino-7-cyclopropyl-1-(4-fluoro-2-methoxypyridin-3-yl)pyrido[2,3-d]pyrimidin-2 (1H)-one
4-amino-5-fluoro-1-(2-methylpyridin-3-yl)-7-(trifluoromethyl)quinazolin-2 (1H)-one
4-amino-7-cyclopropyl-1-(1-ethyl-1H-pyrazol-5-yl)pyrido[2,3-d]pyrimidin-2 (1H)-one
4-amino-7-chloro-1-(o-tolyl)pyrido[2,3-d]pyrimidin-2 (1H)-one
4-amino-7-cyclopropyl-1-(2,3-dihydrobenzofuran-7-yl)pyrido[2,3-d]pyrimidin-2 (1H)-one
1-amino-4-(2-methoxyphenyl)-6-(trifluoromethyl)-3H-pyrido[1,2-c]pyrimidin-3-one
4-amino-1-(2-chlorophenyl)-7-cyclopropylpyrido[2,3-d]pyrimidin-2-one
4-amino-7-cyclopropyl-1-[(1R)-1-[(3S)-oxolan-3-yl]ethyl]pyrido[2,3-d]pyrimidin-2-one
4-amino-7-cyclopropyl-1-[(1R)-1-[(3R)-oxolan-3-yl]ethyl]pyrido[2,3-d]pyrimidin-2-one
3-(4-amino-6-chloro-7-cyclopropyl-2-oxopyrido[2,3-d]pyrimidin-1 (2H)-yl)-2-methoxybenzonitrile
4-amino-7-cyclopropyl-1-(4-oxaspiro[2.5]octan-8-yl)pyrido[2,3-d]pyrimidin-2 (1H)-one
4-amino-6-chloro-7-cyclopropyl-1-(2-methoxypyridin-3-yl)pyrido[2,3-d]pyrimidin-2 (1H)-one
4-amino-7-cyclopropyl-1-(4-fluoro-2-methylphenyl)pyrido[2,3-d]pyrimidin-2-one
4-amino-7-cyclopropyl-1-(3-ethylphenyl)pyrido[2,3-d]pyrimidin-2 (1H)-one
4-amino-7-cyclopropyl-1-(m-tolyl)pyrido[2,3-d]pyrimidin-2 (1H)-one
4-amino-7-cyclopropyl-1-(3,5-difluorophenyl)pyrido[2,3-d]pyrimidin-2 (1H)-one
4-amino-7-cyclopropyl-1-(6-methoxypyridin-2-yl)pyrido[2,3-d]pyrimidin-2 (1H)-one
4-amino-7-cyclopropyl-1-(3-methoxyphenyl)pyrido[2,3-d]pyrimidin-2 (1H)-one
4-amino-7-cyclopropyl-1-(2,3-difluorophenyl)pyrido[2,3-d]pyrimidin-2 (1H)-one
4-amino-7-cyclopropyl-1-phenylpyrido[2,3-d]pyrimidin-2 (1H)-one
4-amino-7-cyclopropyl-1-(1-(oxazol-5-yl)ethyl)pyrido[2,3-d]pyrimidin-2 (1H)-one
3-(4-amino-2-oxo-7-(trifluoromethyl)quinazolin-1 (2H)-yl)-2-methylbenzonitrile
3-(4-amino-2-oxo-7-(trifluoromethyl)pyrido[2,3-d]pyrimidin-1 (2H)-yl)-2-methylbenzonitrile
4-amino-7-cyclopropyl-1-(2,6-difluorophenyl)pyrido[2,3-d]pyrimidin-2 (1H)-one
4-amino-7-cyclopropyl-1-(2-fluorophenyl)pyrido[2,3-d]pyrimidin-2 (1H)-one
4-amino-7-cyclopropyl-1-(3-fluorophenyl)pyrido[2,3-d]pyrimidin-2 (1H)-one
4-amino-7-cyclopropyl-1-[1-(oxetan-3-yl)ethyl]pyrido[2,3-d]pyrimidin-2-one
4-amino-7-(difluoromethoxy)-1-(2-methylphenyl)pyrido[2,3-d]pyrimidin-2-one
4-amino-7-cyclopropyl-1-(1-pyridin-2-ylethyl)pyrido[2,3-d]pyrimidin-2-one
4-amino-1-(2-methyl-3-pyridyl)-7-(2,2,2-trifluoroethyl)quinazolin-2-one hydrochloride
4-amino-7-cyclopropyl-1-(tetrahydro-2H-pyran-4-yl)pyrido[2,3-d]pyrimidin-2 (1H)-one
4-amino-1-(2-chloropyridin-3-yl)-7-cyclopropylpyrido[2,3-d]pyrimidin-2 (1H)-one
4-amino-2-cyclopentyl-7-(2-methyl-3-pyridyl)pyrazolo[3,4-d]pyrimidin-6-one
4-amino-5-chloro-1-(2-methylpyridin-3-yl)-7-(trifluoromethyl)quinazolin-2 (1H)-one
4-amino-1-(3-fluoro-2-methylphenyl)-7-(trifluoromethyl)pyrido[2,3-d]pyrimidin-2 (1H)-one
4-amino-7-cyclopropyl-1-(1H-indol-4-yl)pyrido[2,3-d]pyrimidin-2-one
4-amino-7-cyclopropyl-1-[6-(trifluoromethoxy)pyridin-2-yl]pyrido[2,3-d]pyrimidin-2-one
4-amino-1-(2,3-dihydrobenzofuran-4-yl)-7-(trifluoromethyl)pyrido[2,3-d]pyrimidin-2 (1H)-one
4-amino-1-(3-chloro-2-fluorophenyl)-7-cyclopropylpyrido[2,3-d]pyrimidin-2-one
4-amino-1-(2,3-dihydro-1-benzofuran-4-yl)-7-(trifluoromethoxy)quinazolin-2-one
4-amino-1-(3-fluoro-2-methylphenyl)-7-(trifluoromethoxy)quinazolin-2-one
3-[4-amino-2-oxo-7-(trifluoromethoxy)quinazolin-1-yl]-2-methylbenzonitrile
4-amino-7-cyclopropyl-1-(2,3-dihydro-1H-indol-4-yl)pyrido[2,3-d]pyrimidin-2-one
4-amino-1-(2-chloro-5-fluorophenyl)-7-cyclopropylpyrido[2,3-d]pyrimidin-2-one
4-amino-1-(2-chloro-4-fluorophenyl)-7-cyclopropylpyrido[2,3-d]pyrimidin-2 (1H)-one
4-amino-7-cyclopropyl-1-(2,5-difluorophenyl)pyrido[2,3-d]pyrimidin-2 (1H)-one
4-amino-7-(ethylamino)-1-(o-tolyl)quinazolin-2-one
4-amino-1-(3-chlorophenyl)-7-cyclopropylpyrido[2,3-d]pyrimidin-2 (1H)-one
3-(4-amino-7-cyclopropyl-2-oxopyrido[2,3-d]pyrimidin-1 (2H)-yl)benzonitrile
4-amino-7-(difluoromethoxy)-1-(4-oxaspiro[2.5]octan-8-yl)quinazolin-2 (1H)-one
1-amino-4-(2-chlorophenyl)-6-(trifluoromethyl)pyrido[1,2-c]pyrimidin-3-one
4-amino-1-(benzo[d]oxazol-4-yl)-7-cyclopropylpyrido[2,3-d]pyrimidin-2 (1H)-one
4-amino-7-cyclopropyl-1-(3,4-difluorophenyl)pyrido[2,3-d]pyrimidin-2 (1H)-one
3-(4-amino-7-cyclopropyl-2-oxopyrido[2,3-d]pyrimidin-1 (2H)-yl)-2-methylbenzonitrile
3-(4-amino-7-cyclopropyl-2-oxopyrido[2,3-d]pyrimidin-1 (2H)-yl)-2-methylbenzonitrile
4-amino-7-(difluoromethoxy)-1-(o-tolyl)quinazolin-2 (1H)-one
3-(4-amino-7-cyclopropyl-2-oxopyrido[2,3-d]pyrimidin-1 (2H)-yl)-2-chlorobenzonitrile
4-amino-7-cyclopropyl-1-[(8S)-4-oxaspiro[2.5]octan-8-yl]pyrido[2,3-d]pyrimidin-2-one
4-amino-7-cyclopropyl-1-[(8R)-4-oxaspiro[2.5]octan-8-yl]pyrido[2,3-d]pyrimidin-2-one
4-amino-1-(1H-benzo[d]imidazol-4-yl)-7-cyclopropylpyrido[2,3-d]pyrimidin-2 (1H)-one
4-amino-7-cyclopropyl-1-(1H-indazol-4-yl)pyrido[2,3-d]pyrimidin-2 (1H)-one
4-amino-7-cyclopropyl-1-(2,3-dimethylphenyl)pyrido[2,3-d]pyrimidin-2 (1H)-one
4-amino-1-(2-chloropyridin-3-yl)-7-(difluoromethoxy)quinazolin-2 (1H)-one
4-amino-1-(2-chloropyridin-3-yl)-7-cyclopropylpyrido[2,3-d]pyrimidin-2 (1H)-one
4-amino-1-(2-chloropyridin-3-yl)-7-cyclopropylpyrido[2,3-d]pyrimidin-2 (1H)-one
4-amino-7-(difluoromethoxy)-1-(m-tolyl)quinazolin-2 (1H)-one
4-amino-7-(difluoromethoxy)-1-(2-fluoro-3-methylphenyl)quinazolin-2 (1H)-one
4-amino-7-cyclopropyl-1-(2-(trifluoromethyl)phenyl)pyrido[2,3-d]pyrimidin-2 (1H)-one
4-amino-7-cyclopropyl-1-(3-(fluoromethyl)-2-methylphenyl)pyrido[2,3-d]pyrimidin-2 (1H)-one
4-amino-1-(2-chloro-3-methylphenyl)-7-cyclopropylpyrido[2,3-d]pyrimidin-2-one
4-amino-1-(3-chloro-2-methylphenyl)-7-cyclopropylpyrido[2,3-d]pyrimidin-2-one
4-amino-7-cyclopropyl-1-(2,3-dichlorophenyl)pyrido[2,3-d]pyrimidin-2 (1H)-one
4-amino-7-cyclopropyl-1-(2-fluoro-3-methylphenyl)pyrido[2,3-d]pyrimidin-2 (1H)-one
3-(4-amino-7-cyclopropyl-2-oxopyrido[2,3-d]pyrimidin-1 (2H)-yl)-2-fluorobenzonitrile
(S)-4-amino-7-cyclopropyl-1-(oxepan-3-yl)pyrido[2,3-d]pyrimidin-2 (1H)-one
(R)-4-amino-7-cyclopropyl-1-(oxepan-3-yl)pyrido[2,3-d]pyrimidin-2 (1H)-one
4-amino-7-cyclopropyl-1-(3-hydroxy-2-methylphenyl)pyrido[2,3-d]pyrimidin-2 (1H)-one
4-amino-7-(difluoromethoxy)-1-(3-fluoro-2-methylphenyl)quinazolin-2 (1H)-one
4-amino-7-cyclopropyl-1-[rac-(2S,3S)-2-methyltetrahydropyran-3-yl]pyrido[2,3-d]pyrimidin-2-one
4-amino-1-(benzo[d]thiazol-7-yl)-7-cyclopropylpyrido[2,3-d]pyrimidin-2 (1H)-one
4-amino-7-cyclopropyl-1-((2S,3R)-2-methyltetrahydro-2H-pyran-3-yl)pyrido[2,3-d]pyrimidin-2 (1H)-one
4-amino-7-cyclopropyl-1-((2R,3S)-2-methyltetrahydro-2H-pyran-3-yl)pyrido[2,3-d]pyrimidin-2 (1H)-one
(−)-4-amino-1-(2-chloro-3-fluorophenyl)-7-cyclopropylpyrido[2,3-d]pyrimidin-2 (1H)-one
(+)-4-amino-1-(2-chloro-3-fluorophenyl)-7-cyclopropylpyrido[2,3-d]pyrimidin-2 (1H)-one
(−)-4-amino-1-(2-chlorophenyl)-7-cyclopropylpyrido[2,3-d]pyrimidin-2 (1H)-one
(+)-4-amino-1-(2-chlorophenyl)-7-cyclopropylpyrido[2,3-d]pyrimidin-2 (1H)-one
4-amino-7-cyclopropyl-6-(difluoromethoxy)-1-(o-tolyl)pyrido[2,3-d]pyrimidin-2-one
4-amino-7-cyclopropyl-1-(6,7-dihydro-5H-pyrrolo[1,2-c]imidazol-7-yl)pyrido[2,3-d]pyrimidin-2-one; formic acid
4-(2-chlorophenyl)-6-cyclopropyl-1-imino-pyrido[1,2-c]pyrimidin-3-one; formic acid
4-amino-7-cyclopropyl-1-[2-(trifluoromethoxy)phenyl]pyrido[2,3-d]pyrimidin-2-one
4-amino-1-(2-chloro-3-pyridyl)-7-(trifluoromethoxy)quinazolin-2-one
4-amino-7-cyclopropyl-1-(6-(difluoromethoxy)pyridin-2-yl)pyrido[2,3-d]pyrimidin-2 (1H)-one
4-amino-7-cyclopropyl-1-(1,4-dioxepan-6-yl)pyrido[2,3-d]pyrimidin-2-one
4-amino-7-cyclopropyl-1-(3-fluoro-2-methylphenyl)quinazolin-2-one

Particular compounds of formula I of the present invention are those selected from the group consisting of:
4-amino-7-cyclopropyl-1-(o-tolyl)pyrido[2,3-d]pyrimidin-2 (1H)-one
4-amino-7-cyclopropyl-1-(2-methylpyridin-3-yl)pyrido[2,3-d]pyrimidin-2 (1H)-one
3-(4-amino-7-cyclopropyl-2-oxopyrido[2,3-d]pyrimidin-1 (2H)-yl)-2-methylbenzonitrile
4-amino-7-cyclopropyl-1-(2,3-dihydrobenzofuran-4-yl)pyrido[2,3-d]pyrimidin-2 (1H)-one
4-amino-7-cyclopropyl-1-(3-fluoro-2-methylphenyl)pyrido[2,3-d]pyrimidin-2 (1H)-one
4-amino-7-cyclopropyl-1-(oxan-3-yl)pyrido[2,3-d]pyrimidin-2-one
4-amino-7-(difluoromethoxy)-1-(2-methylpyridin-3-yl)quinazolin-2 (1H)-one
4-amino-1-(2-methylpyridin-3-yl)-7-(trifluoromethoxy)quinazolin-2 (1H)-one
4-amino-7-cyclopropyl-1-(2,3-dihydrobenzofuran-4-yl)quinazolin-2 (1H)-one
4-amino-7-cyclopropyl-1-(2,3-dihydrobenzofuran-7-yl)pyrido[2,3-d]pyrimidin-2 (1H)-one
4-amino-1-(2-chlorophenyl)-7-cyclopropylpyrido[2,3-d]pyrimidin-2-one
4-amino-7-cyclopropyl-1-(4-oxaspiro[2.5]octan-8-yl)pyrido[2,3-d]pyrimidin-2 (1H)-one
4-amino-1-(2-chloropyridin-3-yl)-7-cyclopropylpyrido[2,3-d]pyrimidin-2 (1H)-one
4-amino-7-(difluoromethoxy)-1-(2-methylphenyl)pyrido[2,3-d]pyrimidin-2-one
3-(4-amino-2-oxo-7-(trifluoromethyl)pyrido[2,3-d]pyrimidin-1 (2H)-yl)-2-methylbenzonitrile
4-amino-7-cyclopropyl-1-(m-tolyl)pyrido[2,3-d]pyrimidin-2 (1H)-one
4-amino-7-cyclopropyl-1-[(8S)-4-oxaspiro[2.5]octan-8-yl]pyrido[2,3-d]pyrimidin-2-one
4-amino-7-cyclopropyl-1-[(8R)-4-oxaspiro[2.5]octan-8-yl]pyrido[2,3-d]pyrimidin-2-one
(R)-4-amino-7-cyclopropyl-1-(oxepan-3-yl)pyrido[2,3-d]pyrimidin-2 (1H)-one
4-amino-7-cyclopropyl-1-((2S,3R)-2-methyltetrahydro-2H-pyran-3-yl)pyrido[2,3-d]pyrimidin-2 (1H)-one

In another embodiment, the present invention provides a compound according to formula I, I′, I″ or II as described herein for use as a therapeutically active substance.

In yet another embodiment, the present invention provides a compound according to formula I, I′, I″ or II as described herein for the treatment, prevention and/or delay of progression of, more 5 particularly for the treatment of Cancer in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, more particularly Lung Adenocarcinoma, Lung Squamous Carcinoma, Pancreatic Adenocarcinoma, Glioblastoma Multiforme, and Head and Neck Squamous Carcinoma.

In another embodiment, the present invention provides the use of a compound according to formula I, I′, I″ or II as described herein for the preparation of a medicament for the treatment, prevention and/or delay of progression of, more particularly for the treatment of, Cancer in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, more particularly Lung Adenocarcinoma, Lung Squamous Carcinoma, Pancreatic Adenocarcinoma, Glioblastoma Multiforme, and Head and Neck Squamous Carcinoma.

In one aspect, the application provides a method of treating a Mat2A disorder in a subject having Mat2A related disorders, said method comprising administering to a subject in need thereof a therapeutically effective amount of any of the above compounds.

In another embodiment, the present invention provides a method of the treatment, prevention and/or delay of progression of, more particularly of the treatment of, Cancer in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, more particularly Lung Adenocarcinoma, Lung Squamous Carcinoma, Pancreatic Adenocarcinoma, Glioblastoma Multiforme, and Head and Neck Squamous Carcinoma which comprises administering an effective amount of a compound according to formula I, I′, I″ or II as described herein.

In particular embodiment, the present invention provides a method of treatment, prevention and/or delay of progression of, more particularly of the treatment of, Cancer in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, more particularly Lung Adenocarcinoma, Lung Squamous Carcinoma, Pancreatic Adenocarcinoma, Glioblastoma Multiforme, and Head and Neck Squamous Carcinoma which comprises administering an effective amount of a compound according to formula I, I′, I″ or II as described herein.

In particular, Mat2A disorders or Mat2A related diseases are Cancer in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, more particularly Lung Adenocarcinoma, Lung Squamous Carcinoma, Pancreatic Adenocarcinoma, Glioblastoma Multiforme, and Head and Neck Squamous Carcinoma.

In some particular embodiments of the invention, atropoisomerism is avoided, leading to chiraly stable compounds.

In one aspect, the application provides a pharmaceutical composition comprising the compound of any one of the above embodiments, admixed with at least one pharmaceutically acceptable carrier, such as excipient or diluent.

In another embodiment, the present invention provides a use of a compound of formula I, I′, I″ or II in the preparation of a medicament for the treatment, prevention and/or delay of progression of, more particularly for the treatment of, diseases associated with Mat2A.

In yet another embodiment, the present invention provides a medicaments containing a compound of formula I, I′, I″ or II as defined herein or a pharmaceutically acceptable salt thereof and a therapeutically inert carrier are also an object of the present invention, as is a process for their production, which comprises bringing one or more compounds of formula I, I′, I″ or II and/or pharmaceutically acceptable acid addition salts and, if desired, one or more other therapeutically valuable substances into a galenical administration form together with one or more therapeutically inert carriers.

Another embodiment provides pharmaceutical compositions or medicaments comprising the compounds of the invention and a therapeutically inert carrier, diluent or pharmaceutically acceptable excipient, as well as methods of using the compounds of the invention to prepare such compositions and medicaments.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. For example, such amount may be below the amount that is toxic to normal cells, or the mammal as a whole.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, coated tablets, dragees, powders, capsules (hard and soft gelatine capsules), solutions (i.e. injection solutions), dispersions, suspensions, syrups, sprays, suppositories, gels, emulsions, patches, eye drops, ear drops etc. Such compositions may contain components conventional in pharmaceutical preparations, e.g., diluents, carriers, pH modifiers, sweeteners, bulking agents, and further active agents.

A typical formulation is prepared by mixing a compound of the present invention and pharmaceutically acceptable carrier or excipient. Suitable pharmaceutically acceptable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The pharmaceutically acceptable carriers may be either solid or liquid. Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatin, tragacanth, methylcellulose, sodium carboxy, methyl, cellulose, a low melting wax, cocoa butter, and the like.

The dosage at which compounds of the invention can be administered can vary within wide limits and will, of course, be fitted to the individual requirements in each particular case. In general, in the case of oral administration a daily dosage of about 0.01 to 1000 mg per person of a compound formula I, I′, I″ or II should be appropriate, although the above upper limit can also be exceeded when necessary.

An example of a suitable oral dosage form is a tablet comprising about 100 mg to 500 mg of the compound of the invention compounded with about 30 to 90 mg anhydrous lactose, about 5 to 40 mg sodium croscarmellose, about 5 to 30 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment.

An example of an aerosol formulation can be prepared by dissolving the compound, for example 10 to 100 mg, of the invention in a suitable buffer solution, e.g. a phosphate buffer, adding a tonicifier, e.g. a salt such as sodium chloride, if desired. The solution may be filtered, e.g., using a 0.2 μm filter, to remove impurities and contaminants.

An embodiment, therefore, includes a pharmaceutical composition comprising a compound according to the invention herein described, or a stereoisomer thereof. In a further embodiment includes a pharmaceutical composition comprising a compound according to the invention herein described, or a stereoisomer thereof, together with a pharmaceutically acceptable carrier or excipient.

The compounds of the present invention can be used, either alone or in combination with other drugs, for the treatment, prevention and/or delay of progression of Mat2A related diseases, in particular Cancer in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, more particularly Lung Adenocarcinoma, Lung Squamous Carcinoma, Pancreatic Adenocarcinoma, Glioblastoma Multiforme, and Head and Neck Squamous Carcinoma.

A particular embodiment of the present invention relates to pharmaceutical compositions comprising compounds of formula I, I′, I″ or II or their pharmaceutically acceptable salts as defined above and one or more pharmaceutically acceptable excipients for use in the treatment, prevention and/or delay of progression of cognitive impairments associated with Cancer in particular Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, more particularly Lung Adenocarcinoma, Lung Squamous Carcinoma, Pancreatic Adenocarcinoma, Glioblastoma Multiforme, and Head and Neck Squamous Carcinoma.

Another embodiment includes a pharmaceutical composition comprising a compound according to the invention herein described for use in the treatment, prevention and/or delay of progression of, more particularly in the treatment of a Mat2A related diseases. Another embodiment includes a pharmaceutical composition comprising a compound according to the invention herein described for use in the treatment, prevention and/or delay of progression of, more particularly in the treatment of Mat2A related diseases.

In another embodiment the present invention provides the manufacture of compounds of formula I, I′, I″ or II as described herein.

The preparation of compounds of formula I, I′, I″ or II of the present invention may be carried out in sequential or convergent synthetic routes. Syntheses of the invention are shown in the following general scheme. The skills required for carrying out the reaction and purification of the resulting products are known to those persons skilled in the art. In case a mixture of enantiomers or diastereoisomers is produced during a reaction, these enantiomers or diastereoisomers can be separated by methods described herein or known to the man skilled in the art such as e.g. chiral chromatography or crystallization.

Furthermore the compounds of the present invention can be prepared from commercially available starting materials or by the use of general synthetic techniques and procedures that are known to those skilled in the art. Outlined below are reaction schemes suitable for the preparation of such compounds. The substituents and indices used in the following description of the processes have the significance given herein. Further exemplification can be found in the specific examples detailed below.

### General Schemes

In more detail, compounds of formula I, I′ or I″ and their intermediates may be prepared by schemes 1 to 2 and by the description of the specific examples.

A subgroup of compounds of formula I or I′ wherein X¹ is N, X³ is CR³, X is halogen (particularly Chloro or Fluoro) and R⁵ is NH₂ and R¹, R², R³ and R⁴ are as defined previously, can be prepared as outlined in scheme 1 below.

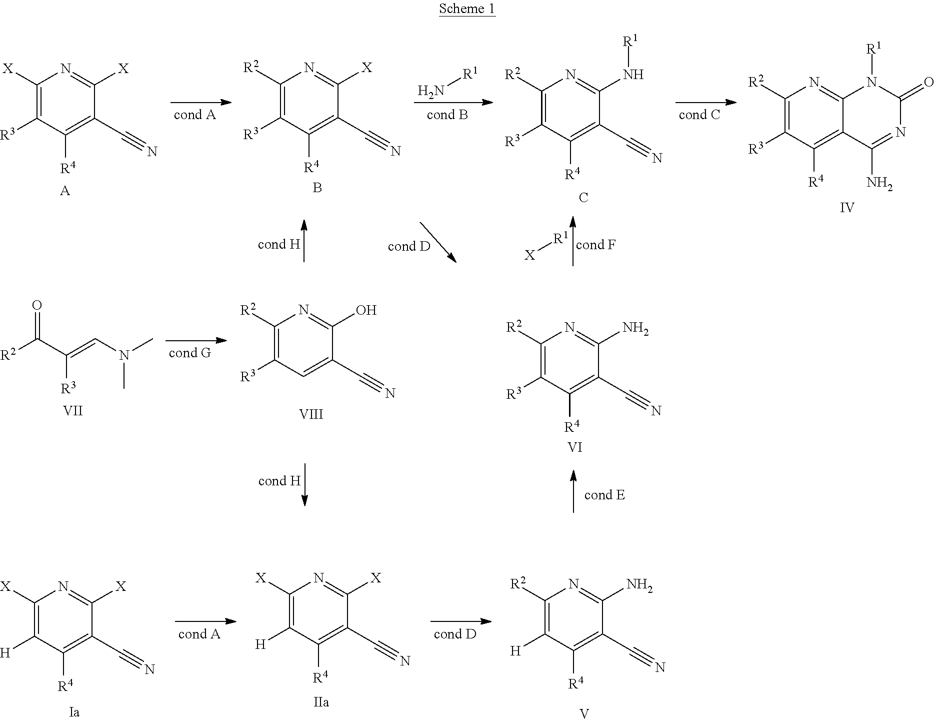

A 2,6-dihalo-3-nitrile pyridine A can be reacted in the 6-position with a boronic acid or boronic ester in a Suzuki-Miyaura type reaction using palladium catalyst such as Pd(dppf)₂Cl₂·CH₂Cl₂ and an excess of a base such as K₂CO₃ at elevated temperatures in solvents such as dioxane and water or with an amine in a SnAr type reaction at elevated temperatures in polar solvents such as DMF, DMA, NMP etc. using an excess of a base (e.g. DIPEA, K₂CO₃) (cond A) to afford 2-halo-3-nitirle pyridine B. Alternatively, pyridine B can be synthesized by cyclizing the intermediate VII with 2-cyanoacetamide using base (e.g. NaOEt) in polar solvents such as DMF at elevated temperatures yielding corresponding hydroxy pyridine VIII (cond G). Hydroxy pyridine VIII can be converted to pyridine B with dehydrating reagent such as POCl₃ at elevated temperatures (cond H). The Halogen in the 2-position of pyridine B can be converted with an amine or aniline in a Hartwig-Buchwald type reaction using palladium catalyst system such as Pd(OAc)₂/xantphos or xphos and an excess of a base such as Cs₂CO₃ at elevated temperatures in solvents such as dioxane or toluene, or in a SnAr type reaction at elevated temperatures in polar solvents such as DMF, DMA, NMP etc. using an excess of a base (e.g. DIPEA, K₂CO₃) (cond B) to afford substituted pyridine C. The NH group of pyridine C can be activated with an isocyanate reagent e.g. trichloroacetyl isocyanate at ambient or elevated temperatures in chlorinated apolar solvents such as DCM to afford aminopyrimidone IV after cyclization using ammonia in a polar solvent such as MeOH at ambient temperature (cond C).

Alternatively, a halogen in the 2-position of pyridine B can be converted to amine moiety using ammonolysis reaction conditions such as ammonia in polar solvents (e.g. MeOH) at elevated temperatures and high pressure to afford the pyridine intermediate VI (cond D) or pyridine VI can be obtained by reacting an intermediate V in the 5-position with an electrophilic halogenation reagent such as NBS in chlorinated solvent (e.g. DCM or CHCl₃) at ambient or elevated temperatures (cond E). Next, pyridine VI can be reacted with haloarenes in a Hartwig-Buchwald type reaction using palladium catalyst system such as Pd(OAc)₂/xantphos or xphos and an excess of a base such as Cs₂CO₃ at elevated temperatures in solvents such as dioxane or toluene, (cond F) or in a SnAr type reaction at elevated temperatures in polar solvents such as DMF, DMA, NMP etc. using an excess of a base (e.g. DIPEA, K₂CO₃) (cond B) to afford substituted pyridine C, which can converted to the final aminopyrimidone IV using previously described conditions.

A subgroup of compounds of formula I or I′ wherein X¹ is CH, X³ is CR³, X is halogen (particularly Chloro or Fluoro) and R⁵ is NH₂ and R¹, R², R³ and R⁴ are as defined previously, can be prepared as outlined in scheme 2 below.

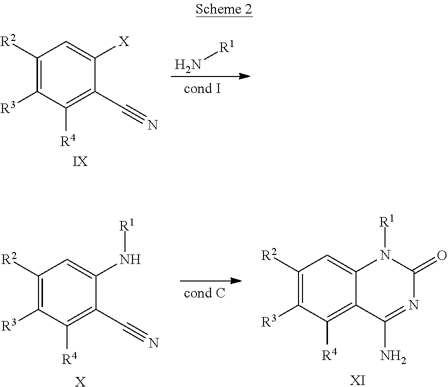

The halogen in the 2-position (X═Br or Cl) of arylnitrile IX can be converted with an amine or aniline in a Hartwig-Buchwald type reaction using palladium catalyst system such as Pd(OAc)₂/xantphos or xphos and an excess of a base such as Cs₂CO₃ at elevated temperatures in solvents such as dioxane or toluene, or in a SnAr type reaction (X═F) at elevated temperatures in polar solvents such as DMF, DMA, NMP etc. using an excess of a base (e.g. DIPEA, K₂CO₃) (cond I) to afford substituted arylnitrile X. The NH group of the intermediate X can be activated with an isocyanate reagent e.g. trichloroacetyl isocyanate at ambient or elevated temperatures in chlorinated apolar solvents such as DCM to afford aminopyrimidone XI after cyclization using ammonia in a polar solvent such as MeOH at ambient temperature (cond C).

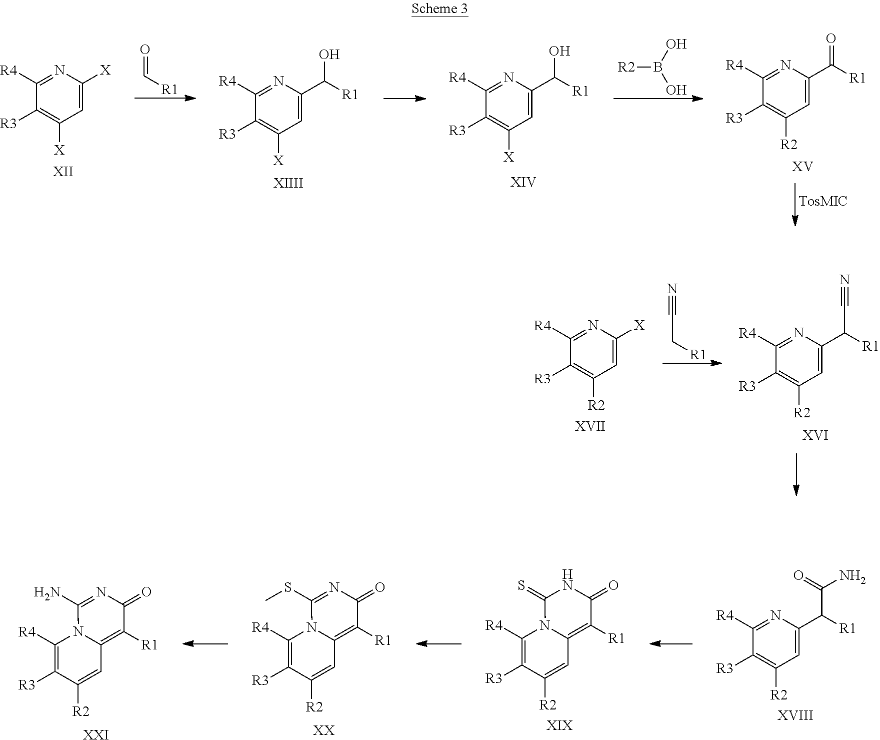

Dihalide XII (X═Br, I) can be selectively metallated by lithium halogen exchange in the 2-position at low temperature (−78° C.) in THF with nBuLi (European Journal of Inorganic Chemistry, 2014, 4734) and the resulting organolithium reacted with an aldehyde to afford XIII. Oxidation to the ketone by standard oxidising reagents (e.g. MnO2, Dess-Martin periodinane) affords ketone XIV which can be further derivitised by Suzuki coupling with boronic acids to install R² in product XV. Reaction with tosMIC in dimethoxyethane and strong base (e.g. potassium tertbutoxide) at ambient temperature affords nitrile XVI. Mild hydrolyis under acidic conditions (e.g. sulfuric acid in acetic acid 1:4) at 40° C. affords carboxamide XVIII. Cyclisation with thiophosgene in ethanol under basic conditions (sodium ethoxide) affords XIX which can be directly alkylated with iodomethane in alcoholic solvents (e.g. ethanol) to afford thioether XX. Reaction with ammonia (e.g. ammonium hydroxide) at elevated temperatures (50° C. in a sealed tube) affords the final product XXI.

Alternatively nitrile XVI can be prepared by reaction of XVII (X═Cl) with the appropriate nitrile in a polar solvent (e.g. DMF) under strongly basic conditions (e.g. Sodium hydride).

### General Procedures

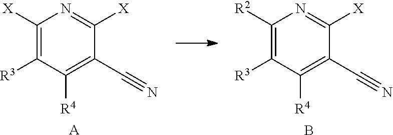

wherein X¹ is N, X³ is CR³ and X is halogen (particularly Chloro or Fluoro) and R², R³ and R⁴ are as defined previously.

### General Procedure A1: Suzuki-Miyaura Type Cross Coupling

To a 2,6-dihalo-3-nitrile pyridine A dissolved in dioxane/water (ration 4:1, 0.1-0.2 M) were added K₂CO₃ (3 eq.) followed by boronic acid or ester (1.5 eq.) and the resulting reaction mixture was degassed by bubbling argon through the mixture with sonication. Pd(dppf)₂Cl₂—CH₂Cl₂ complex (0.05-0.2 eq.) was added and the reaction mixture heated to 100° C. until LCMS showed complete consumption of the pyridine starting material A (0.5 h-16 h). The reaction was then diluted with EtOAc, washed with brine, dried over Na₂SO₄ and concentrated. The crude product B could be purified using flash silica gel chromatography.

General Procedure A2: SnAr Type Reaction

To a solution of a 2,6-dihalo-3-nitrile pyridine A in THF (0.1-0.2 M) were added DIPEA (2 eq.) and a secondary amine (1.1 eq.). Reaction was stirred at ambient or elevated temperature until LCMS showed complete consumption of the pyridine starting material A (up to 16 h). The reaction was then diluted with EtOAc, washed with brine, dried over Na₂SO₄ and concentrated. The crude product B could be purified using flash silica gel chromatography.

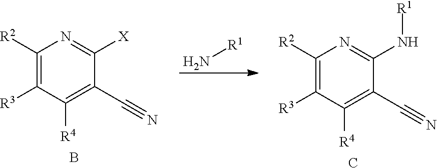

wherein X¹ is N, X³ is CR³, X is halogen (particularly Chloro or Fluoro) and R², R³ and R⁴ are as defined previously.

### General Procedure B1: Hartwig-Buchwald Type Cross Coupling

To a 2-halo-3-nitirle pyridine B dissolved in dioxane (0.1-0.2 M) were added Cs₂CO₃ (3 eq.) followed by amine or aniline (1.5-3 eq.) and the resulting reaction mixture was degassed by bubbling argon through the mixture with sonication. Pd(OAc)₂ (0.1 eq.) and a ligand (xantphos or xphos, 0.2 eq.) were added and the reaction mixture heated to 100° C. until LCMS showed complete consumption of the pyridine starting material B (0.5 h-16 h). The reaction was then diluted with EtOAc, washed with brine, dried over Na₂SO₄ and concentrated. The crude product C could be purified using flash silica gel chromatography.

### General Procedure B2: SnAr Type Reaction

To a 2-halo-3-nitirle pyridine B dissolved in NMP (0.1-0.2 M) were added DIPEA or TEA (3 eq.) and primary amine (1.2-2 eq.). The reaction mixture was heated between 100° C. and 210° C. until LCMS showed complete consumption of the pyridine starting material A (1-8 h). The reaction was then diluted with EtOAc, washed with brine, dried over Na₂SO₄ and concentrated. The crude product C could be purified using flash silica gel chromatography.

### General Procedure C: Formation of Aminopyrimidone

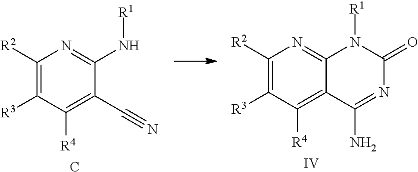

wherein X¹ is N, X³ is CR³, and R⁵ is NH₂ and R², R³ and R⁴ are as defined previously.

To a solution of pyridine intermediate C in DCM or DCE (0.1-0.2 M) was added trichloroacetyl isocyanate (2.2 eq.) and the resulting reaction mixture was stirred at ambient or elevated temperature until LCMS showed complete consumption of the pyridine starting material C and formation of the 2,2,2-trichlorocarbamoyl acetamide intermediate (1-16 h). Next, ammonia in methanol (7 M, 100-200 eq.) was added and the resulting reaction mixture was stirred at ambient temperature until LCMS showed complete conversion to aminopyrimidone product IV (1-16 h). The reaction was then diluted with EtOAc, washed with brine, dried over Na₂SO₄ and concentrated. The crude product IV could be purified using flash silica gel chromatography or preparative HPLC.

General Procedure G and H: Pyridine Synthesis

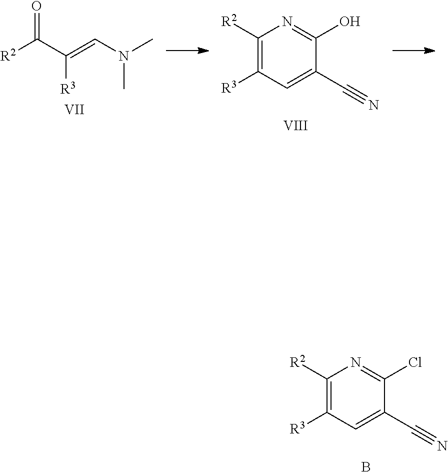

wherein X¹ is N, X³ is CR³ and R⁴ is H and R² and R³ are as defined previously.

To a solution of VII (prepared by a modified procedure from J. Med. Chem. 2011, 54, 7974-7985) in DMF (0.2-0.4 M) were added 2-cyanoacetamide (3 eq.) and NaOEt (3 eq.) as a base. The resulting reaction mixture was heated to 100° C. until LCMS showed complete consumption of the starting material VII (approx. 16 h). The reaction was then diluted with EtOAc, washed with brine, dried over Na₂SO₄ and concentrated. The crude product VIII could be purified using flash silica gel chromatography.

In the subsequent step, hydroxy pyridine VIII was dissolved in POCl₃ (10-20 eq.) and The resulting reaction mixture was heated to 100° C. until LCMS showed complete consumption of the starting material VIII (approx. 16 h). The reaction mixture was then concentrated under reduced pressure, diluted with EtOAc and filtered. The organic layers were diluted with water and extracted several times with EtOAc. The combined organic layers were washed with brine, dried over Na₂SO₄ and concentrated. The crude chloro pyridine product B could be purified using flash silica gel chromatography.

General Procedure D: Ammonolysis

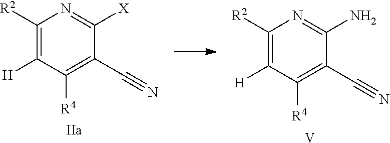

wherein X¹ is N, X is halogen (particularly Chloro or Fluoro) and X³ is CH and R² and R⁴ are as defined previously.

A solution of pyridine IIa in dioxane (0.4-0.6 M) was heated to 100° C. under NH₃ atmosphere and inherent pressure until LCMS showed full consumption of the starting material IIa (approx. 2 days). The reaction was then concentrated to dryness. The crude product V could be purified using flash silica gel chromatography.

General Procedure E: Pyridine Synthesis: Halogenation

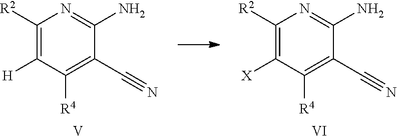

wherein X¹ is N, X is halogen (particularly Chloro or Fluoro) and X³ is CH and R² and R⁴ are as defined previously.

To a solution of amino pyridine V in DMF, DCM or CHCl₃ (0.1-0.2 M) was added NCS or NBS reagent (1.1-1.5 eq.) and the resulting reaction mixture was stirred in the dark at ambient temperature (for NBS) or heated to 60° C. until LCMS showed complete consumption of the starting material V. The reaction was then diluted with EtOAc, washed with brine, dried over Na₂SO₄ and concentrated. The crude product VI could be used in the next step without further purification or be purified using flash silica gel chromatography.

General Procedure F: Inverse Hartwig-Buchwald Type Cross Coupling

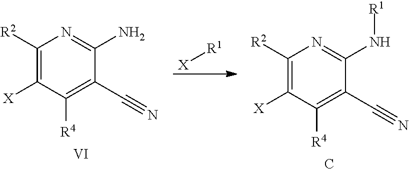

wherein X¹ is N, X is halogen (particularly Chloro or Fluoro) and R² and R⁴ are as defined previously.

To a 2-amino-3-nitirle pyridine VI dissolved in dioxane (0.1-0.2 M) were added Cs₂CO₃ (3 eq.) followed by haloarenes (1.5-3 eq.) and the resulting reaction mixture was degassed by bubbling argon through the mixture with sonication. Pd(OAc)₂ (0.05-0.1 eq.) and a ligand (xantphos or xphos, 0.1-0.2 eq.) were added and the reaction mixture heated to 100° C. until LCMS showed complete consumption of the pyridine starting material VI (0.5 h-16 h). The reaction was then diluted with EtOAc, washed with brine, dried over Na₂SO₄ and concentrated. The crude product C could be purified using flash silica gel chromatography.

General Procedure I1: Hartwig-Buchwald Type Cross Coupling

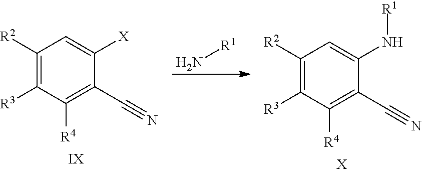

wherein X¹ is CH, X is halogen (particularly Chloro or Fluoro) and X³ is CH and R² and R⁴ are as defined previously.

To a haloarene nitrile XI dissolved in dioxane (0.1-0.2 M) were added Cs₂CO₃ (3 eq.) followed by amine or aniline (1.5-3 eq.) and the resulting reaction mixture was degassed by bubbling argon through the mixture with sonication. Pd(OAc)₂ (0.1 eq.) and a ligand (xantphos or xphos, 0.2 eq.) were added and the reaction mixture heated to 90-100° C. until LCMS showed complete consumption of the starting material XI (0.5 h-16 h). The reaction was then diluted with EtOAc, washed with brine, dried over Na₂SO₄ and concentrated. The crude product X could be purified using flash silica gel chromatography.

General Procedure 12: SnAr Type Reaction

To a haloarene nitrile XI dissolved in NMP (0.1-0.2 M) were added DIPEA or TEA (3 eq.) and primary amine (1.2-2 eq.). The reaction mixture was stirred at ambient temperature or heated between 140° C. and 210° C. until LCMS showed complete consumption of the starting material XI (1-8 h). The reaction was then diluted with EtOAc, washed with brine, dried over Na₂SO₄ and concentrated. The crude product X could be purified using flash silica gel chromatography.

A particular embodiment of the invention relates to a process for the preparation of compounds of formula (I′) wherein X¹, X³, R¹, R² and R⁴ are as defined herein and pharmaceutically acceptable salts thereof as defined in accordance with the present invention, comprising the cyclisation of compound of formula (Ia′) to afford the compound of formula (I′) by activating with an isocyanate reagent e.g. trichloroacetyl isocyanate at ambient or elevated temperatures in chlorinated apolar solvents such as DCM followed by the addition of ammonia in a polar solvent such as MeOH at ambient temperature (cond C), as shown in scheme 4.

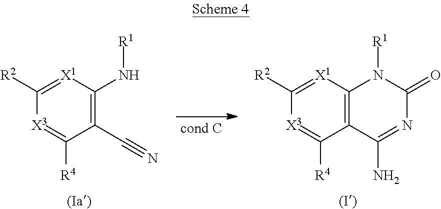

The compounds were investigated in accordance with the test given hereinafter.

Determination of Mat2A activity

Measurement of Mat2A inhibition is performed in 384 well format absorbance-based assay. Recombinant human Mat2a (12.5 nM) and serial diluted compounds in DMSO (range of concentrations from 10 NM to 508 pM) or controls (DMSO) are incubated for 15 minutes at room temperature (RT) in assay buffer containing 50 mM HEPES pH 7.5, 50 mM KCl, 50 mM MgCl2, 0.01% Tween 20 and 10 mM DTT. The reaction is initiated by the addition of the combined substrates ATP and Methionine, each at a final concentration of 100 μM. Final assay condition are 12.5 nM Mat2A, 100 μM ATP and Methionine Substrates and 2% DMSO. After 120 minutes of incubation at RT, the reaction is stopped by the addition of Biomol Green. The absorbance signal is measured at λ=635 nm with a multiplate reader (BMG Pherastar reader or equivalent) after 30 min of equilibration at RT.

| Example number | MAT2A IC50 (uM) |
|---|---|
| 1 | <0.013 |
| 2 | <0.013 |
| 3 | 0.022 |
| 4 | 0.031 |
| 5 | 0.022 |
| 6 | 0.028 |
| 7 | <0.013 |
| 8 | <0.013 |
| 9 | <0.013 |
| 10 | <0.013 |
| 11 | <0.013 |
| 12 | <0.013 |
| 13 | 0.023 |
| 14 | 0.027 |
| 15 | <0.013 |
| 16 | <0.013 |
| 17 | 0.095 |
| 18 | <0.013 |
| 19 | <0.013 |
| 20 | 0.015 |
| 21 | 0.086 |
| 22 | 0.021 |
| 23 | 0.018 |
| 24 | 0.017 |
| 25 | <0.013 |
| 26 | <0.013 |
| 27 | 0.042 |
| 28 | 0.057 |
| 29 | <0.013 |
| 30 | 0.022 |
| 31 | 0.018 |
| 32 | <0.013 |
| 33 | <0.013 |
| 34 | <0.013 |
| 35 | <0.013 |
| 36 | <0.013 |
| 37 | 0.015 |
| 38 | <0.013 |
| 39 | <0.013 |
| 40 | <0.013 |
| 41 | 0.045 |
| 42 | <0.013 |
| 43 | <0.013 |
| 44 | <0.013 |
| 45 | <0.013 |
| 46 | 0.031 |
| 47 | <0.013 |
| 48 | <0.013 |
| 49 | 0.014 |
| 50 | 0.019 |
| 51 | <0.013 |
| 52 | 0.043 |
| 53 | <0.013 |
| 54 | 0.14 |
| 55 | 0.017 |
| 56 | <0.013 |
| 57 | 0.15 |
| 58 | 0.022 |
| 59 | 0.014 |
| 60 | 0.020 |
| 61 | <0.013 |
| 62 | 0.020 |
| 63 | 0.015 |
| 64 | <0.013 |
| 65 | 0.018 |
| 66 | 0.013 |
| 67 | 0.018 |
| 68 | <0.013 |
| 69 | <0.013 |
| 70 | 0.066 |
| 71 | <0.013 |
| 72 | <0.013 |
| 73 | <0.013 |
| 74 | 0.016 |
| 75 | 0.099 |
| 76 | <0.013 |
| 77 | <0.013 |
| 78 | <0.013 |
| 79 | <0.013 |
| 80 | <0.013 |
| 81 | 0.042 |
| 82 | 0.036 |
| 83 | 0.032 |
| 84 | 0.210 |
| 85 | <0.013 |
| 86 | <0.013 |
| 87 | 0.020 |
| 88 | <0.013 |
| 89 | 0.053 |
| 90 | 0.013 |
| 91 | <0.013 |
| 92 | 0.037 |
| 93 | 0.027 |
| 94 | 0.074 |
| 95 | 0.022 |
| 96 | <0.013 |
| 97 | 0.048 |
| 98 | 0.200 |
| 99 | 0.065 |
| 100 | 0.350 |
| 101 | 0.040 |
| 102 | 0.014 |
| 103 | <0.013 |
| 104 | 0.110 |
| 105 | 0.042 |
| 106 | <0.013 |
| 107 | 0.025 |
| 108 | <0.013 |
| 109 | 0.016 |
| 110 | <0.013 |
| 111 | <0.013 |
| 112 | 0.039 |
| 113 | 0.014 |
| 114 | <0.013 |
| 115 | 0.072 |
| 116 | <0.013 |
| 117 | 0.078 |
| 118 | 0.013 |
| 119 | <0.013 |
| 120 | <0.013 |
| 121 | 0.014 |
| 122 | <0.013 |
| 123 | 0.013 |
| 124 | <0.013 |
| 125 | 0.04 |
| 126 | <0.013 |
| 127 | 0.013 |
| 128 | <0.013 |
| 129 | <0.013 |
| 130 | <0.013 |
| 131 | <0.013 |
| 132 | <0.013 |
| 133 | 0.026 |
| 134 | 0.02 |
| 135 | <0.013 |
| 136 | 0.15 |
| 137 | <0.013 |
| 138 | <0.013 |
| 139 | <0.013 |
| 140 | 0.13 |
| 141 | <0.013 |
| 142 | <0.013 |
| 143 | <0.013 |
| 144 | <0.013 |
| 145 | <0.013 |
| 146 | 0.058 |
| 147 | 0.031 |
| 148 | 0.033 |
| 149 | 0.024 |
| 150 | 0.13 |
| 151 | <0.013 |
| 152 | 0.014 |
| 153 | 0.017 |
| 154 | 0.02 |
| 155 | 0.016 |
| 156 | 0.021 |
| 157 | 0.015 |
| 158 | 0.025 |
| 159 | 0.019 |
| 160 | 0.018 |
| 161 | <0.013 |
| 162 | <0.013 |
| 163 | 0.031 |
| 164 | <0.013 |
| 165 | <0.013 |

### Experimental Part

The following examples are provided for illustration of the invention. They should not be considered as limiting the scope of the invention, but merely as being representative thereof.

### General

Analytical Methods

HPLC (method LCMS_fastgradient)

Column: Agilent Zorbax Eclipse Plus C18, Rapid Resolution HT, 2.1×30 mm, 1.8 μm, Part. no. 959731-902

Solvent A: Water 0.01% Formic Acid; Solvent B: acetonitrile (MeCN)

### Gradients:

| Time [min] | Flow Rate [ml/min] | % A | % B |
|---|---|---|---|
| Initial | 0.8 | 97 | 3 |
| 0.2 | 1.0 | 97 | 3 |
| 1.7 | 1.0 | 3 | 97 |
| 2.0 | 1.0 | 3 | 97 |
| 2.1 | 1.0 | 97 | 3 |

Abbreviations

The following abbreviations were used in the experimental part:
Ar=argon;
nBuLi=n-butyl lithium;
DCM=dichloromethane;
DIPEA=diisopropylethylamine;
DMSO=dimethylsufoxide;
DMF=dimethylformamide;
EtOH=ethanol;
EtOAc=ethyl acetate;
HCl=hydrochloric acid;
HPLC=high peformance liquid chromatography;
LDA=lithium diisopropylamide;
LiHMDS=lithium bis (trimethylsilyl)amide;
mCPBA=metachloroperbenzoic acid;
MOM=methoxymethyl;
NMP=N-methyl-2-pyrolidone;
SEM=[2-(trimethylsilyl)ethoxy)methyl]acetal;
TBTU=2-(1H-Benzotriazole-1-yl)-1,1,3,3-tetramethylaminium tetrafluoroborate;
THF=tetrahydrofuran;
TEMPO=2,2,6,6-tetramethylpiperidinyloxyl;
TBAF=tetra-n-butyl ammonium fluorideTLC=thin layer chromatography;

### Starting Materials

Basic chemicals and solvents were purchased and used as is without further purification. Some intermediates are commercially available, or they can be synthesized using methods known in the art.

### Intermediates

### Intermediate 1: 6-cyclopropyl-2-(o-tolylamino)nicotinonitrile

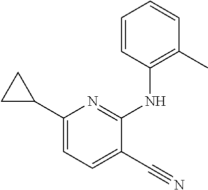 [SMILES: Cc1ccccc1Nc1nc(C2CC2)ccc1C#N]

The title compound ([M+H]⁺ 250.2) was prepared from 2-chloro-6-cyclopropylnicotinonitrile (CAS [1198475-35-2]) by reaction with o-toluidine (CAS [95-35-4]) using Pd(OAc)₂ as a catalyst and xphos as a ligand (General procedure B1).

### Intermediate 2: 6-cyclopropyl-2-((2-methoxyphenyl)amino)nicotinonitrile

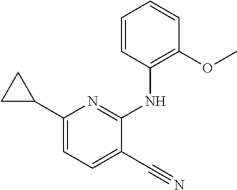 [SMILES: COc1ccccc1Nc1nc(C2CC2)ccc1C#N]

The title compound ([M+H]⁺ 266.2) was prepared from 2-chloro-6-cyclopropylnicotinonitrile (CAS [1198475-35-2]) by reaction with o-anisidine (CAS [90-04-0]) using Pd(OAc)₂ as a catalyst and xphos as a ligand (General procedure B1).

### Intermediate 3: 6-(tert-butyl)-2-(o-tolylamino)nicotinonitrile

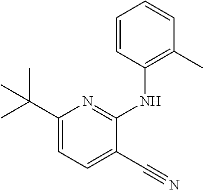 [SMILES: Cc1ccccc1Nc1nc(C(C)(C)C)ccc1C#N]

The title compound ([M+H]⁺ 266.2) was prepared from 6-(tert-butyl)-2-chloronicotinonitrile (CAS [4138-20-9]) by reaction with o-toluidine (CAS [95-35-4]) using Pd(OAc)₂ as a catalyst and xphos as a ligand (General procedure B1).

### Intermediate 4: 2-((2-methoxyphenyl)amino)-6-phenylnicotinonitrile

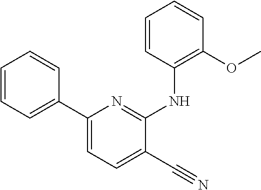 [SMILES: COc1ccccc1Nc1nc(-c2ccccc2)ccc1C#N]

The title compound ([M+H]⁺ 302.2) was prepared from 2-chloro-6-phenylnicotinonitrile (CAS [43083-14-3]) by reaction with o-anisidine (CAS [90-04-0]) using Pd(OAc)₂ as a catalyst and xphos as a ligand (General procedure B1).

### Intermediate 5: 6-(3,3-difluoroazetidin-1-yl)-2-(o-tolylamino)nicotinonitrile

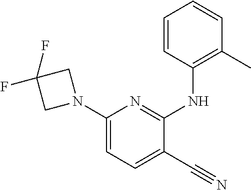 [SMILES: Cc1ccccc1Nc1nc(N2CC(F)(F)C2)ccc1C#N]

### Step 1: 2-chloro-6-(3,3-difluoroazetidin-1-yl)nicotinonitrile

2-chloro-6-(3,3-difluoroazetidin-1-yl)nicotinonitrile ([M+H]⁺ 230.0) was prepared by reaction of 2,6-dichloronicotinonitrile (CAS [40381-90-6]) and 3,3-difluoroazetidine hydrochloride (CAS 5 [288315-03-7]) using DIPEA as a base at 80° C. (General procedure A2).

### Step 2: 6-(3,3-difluoroazetidin-1-yl)-2-(o-tolylamino)nicotinonitrile

The title compound ([M+H]⁺ 301.1) was prepared from 2-chloro-6-(3,3-difluoroazetidin-1-yl)nicotinonitrile by reaction with o-toluidine (CAS [95-35-4]) using Pd(OAc)₂ as a catalyst and xphos as a ligand (General procedure B1).

### Intermediate 6: 6-cyclopropyl-2-((tetrahydrofuran-3-yl)amino)nicotinonitrile

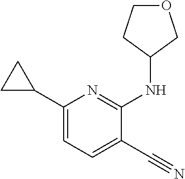 [SMILES: N#Cc1ccc(C2CC2)nc1NC1CCOC1]

The title compound ([M+H]⁺ 230.3) was prepared from 2-chloro-6-cyclopropylnicotinonitrile (CAS [1198475-35-2]) by reaction with tetrahydrofuran-3-amine (CAS [88675-24-5]) at 120° C. using DIPEA as a base (General procedure B2).

### Intermediate 7: 6-cyclopropyl-2-((2-methylpyridin-3-yl)amino)nicotinonitrile

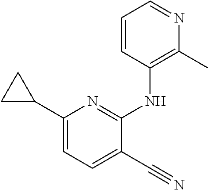 [SMILES: Cc1ncccc1Nc1nc(C2CC2)ccc1C#N]

The title compound ([M+H]⁺ 251.2) was prepared from 2-chloro-6-cyclopropylnicotinonitrile (CAS [1198475-35-2]) by reaction with 3-amino-2-methylpyridine (CAS [3430-10-2]) using Pd(OAc)₂ as a catalyst and xphos as a ligand (General procedure B1).

### Intermediate 8: 4-cyclopropyl-2-(2-methylanilino)benzonitrile

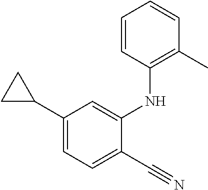 [SMILES: Cc1ccccc1Nc1cc(C2CC2)ccc1C#N]

The title compound ([M+H]⁺ 249.2) was prepared from 2-bromo-4-cyclopropylbenzonitrile (CAS [1237130-18-5]) by reaction with o-toluidine (CAS [95-35-4]) at 120° C. using Pd(OAc)₂ as catalyst, DPPF as a ligand and KOtBu as a base in toluene (General procedure I1).

### Intermediate 9: 6-cyclopropyl-2-((2-methoxypyridin-3-yl)amino)nicotinonitrile

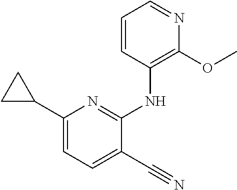 [SMILES: COc1ncccc1Nc1nc(C2CC2)ccc1C#N]

The title compound ([M+H]⁺ 267.2) was prepared from 2-chloro-6-cyclopropylnicotinonitrile (CAS [1198475-35-2]) by reaction with o-anisidine (CAS [90-04-0]) using Pd(OAc)₂ as a catalyst and xphos as a ligand (General procedure B1).

### Intermediate 10: 6-cyclopropyl-5-fluoro-2-((2-methylpyridin-3-yl)amino)nicotinonitrile

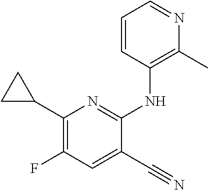 [SMILES: Cc1ncccc1Nc1nc(C2CC2)c(F)cc1C#N]

### Step 1: 2-chloro-6-cyclopropyl-5-fluoronicotinonitrile

2-chloro-6-cyclopropyl-5-fluoronicotinonitrile ([M+H]⁺ 197.0) was prepared by reaction of 2,6-dichloro-5-fluoronicotinonitrile (CAS [82671-02-1]) and cyclopropylboronic acid (CAS [411235-57-9]) using Pd(dppf)₂Cl₂·CH₂Cl₂ as a catalyst and K₂CO₃ as a base (General procedure A1).

### Step 2: 6-cyclopropyl-5-fluoro-2-((2-methylpyridin-3-yl)amino)nicotinonitrile

The title compound ([M+H]⁺ 269.2) was prepared from 2-chloro-6-cyclopropyl-5-fluoronicotinonitrile by reaction with 3-amino-2-methylpyridine (CAS [3430-10-2]) using Pd(OAc)₂ as a catalyst and xphos as a ligand (General procedure B1).

### Intermediate 11: 3-((3-cyano-6-cyclopropylpyridin-2-yl)amino)picolinonitrile

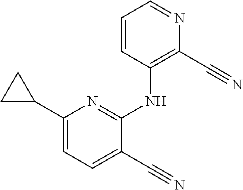 [SMILES: N#Cc1ccc(C2CC2)nc1Nc1cccnc1C#N]

The title compound ([M+H]⁺ 262.3) was prepared from 2-chloro-6-cyclopropylnicotinonitrile (CAS [1198475-35-2]) by reaction with 3-amino-2-cyanopyridine (CAS [42242-11-5]) using Pd(OAc)₂ as a catalyst and xphos as a ligand (General procedure B1).

### Intermediate 12: 6-cyclopropyl-2-(oxan-3-ylamino)pyridine-3-carbonitrile

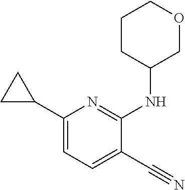 [SMILES: N#Cc1ccc(C2CC2)nc1NC1CCCOC1]

The title compound ([M+H]⁺ 244.3) was prepared from 2-chloro-6-cyclopropylnicotinonitrile (CAS [1198475-35-2]) by reaction with tetrahydro-2H-pyran-3-amine at 200° C. using DIPEA as a base (General procedure B2).

### Intermediate 13: 6-cyclopropyl-2-[1-(oxolan-3-yl)ethylamino]pyridine-3-carbonitrile

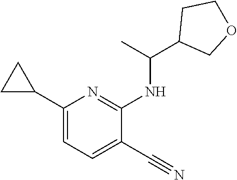 [SMILES: CC(Nc1nc(C2CC2)ccc1C#N)C1CCOC1]

The title compound ([M+H]⁺ 258.2) was prepared from 2-chloro-6-cyclopropylnicotinonitrile (CAS [1198475-35-2]) by reaction with 1-(tetrahydrofuran-3-yl)ethan-1-amine hydrochloride (CAS [1803592-17-7]) using NMP as solvent, DIPEA as abase at 210° C./MW (General procedure B2).

### Intermediate 14: 6-cyclopropyl-2-((3-fluoro-2-methoxyphenyl)amino)nicotinonitrile

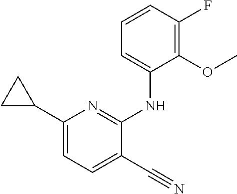 [SMILES: COc1c(F)cccc1Nc1nc(C2CC2)ccc1C#N]

The title compound ([M+H]⁺ 284.2) was prepared from 2-chloro-6-cyclopropylnicotinonitrile (CAS [1198475-35-2]) by reaction with 3-fluoro-2-methoxyaniline (CAS [437-83-2]) using Pd(OAc)₂ as a catalyst and xphos as a ligand (General procedure B1).

### Intermediate 15: 2-((3-cyano-2-methylphenyl)amino)-6-cyclopropylnicotinonitrile

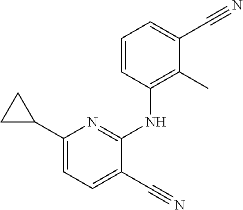 [SMILES: Cc1c(C#N)cccc1Nc1nc(C2CC2)ccc1C#N]

The title compound ([M+H]⁺ 275.3) was prepared from 2-chloro-6-cyclopropylnicotinonitrile (CAS [1198475-35-2]) by reaction with 3-amino-2-methylbenzonitrile (CAS [69022-35-1]) using Pd(OAc)₂ as a catalyst and xphos as a ligand (General procedure B1).

### Intermediate 16: 2-[(2-methylpyridin-3-yl)amino]-6-propan-2-ylpyridine-3-carbonitrile

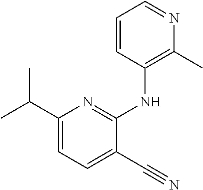 [SMILES: Cc1ncccc1Nc1nc(C(C)C)ccc1C#N]

The title compound ([M+H]⁺ 253.2) was prepared from 2-chloro-6-isopropylnicotinonitrile (CAS [108244-44-6]) by reaction with 3-amino-2-methylpyridine (CAS [3430-10-2]) at 120° C. using Pd(OAc)₂ as catalyst, DPPF as a ligand and KOtBu as a base in toluene (General procedure B1).

### Intermediate 17: 6-cyclopropyl-2-((2,3-dihydrobenzofuran-4-yl)amino) nicotinonitrile

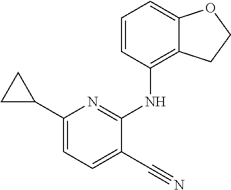 [SMILES: N#Cc1ccc(C2CC2)nc1Nc1cccc2c1CCO2]

The title compound ([M+H]⁺ 278.3) was prepared from 2-chloro-6-cyclopropylnicotinonitrile (CAS [1198475-35-2]) by reaction with 3-amino-2-methylbenzonitrile (CAS [69022-35-1]) using Pd(OAc)₂ as a catalyst and xphos as a ligand (General procedure B1).

### Intermediate 18: 5-chloro-6-cyclopropyl-2-((2-methylpyridin-3-yl)amino)nicotinonitrile

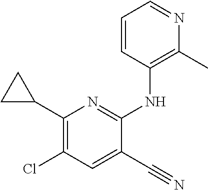 [SMILES: Cc1ncccc1Nc1nc(C2CC2)c(Cl)cc1C#N]

### Step 1: 2,5-dichloro-6-cyclopropyl-pyridine-3-carbonitrile

2,5-dichloro-6-cyclopropyl-pyridine-3-carbonitrile ([M+H]⁺ 213.1) was prepared by reaction of 2,5,6-trichloronicotinonitrile (CAS [40381-92-8]) and cyclopropylboronic acid (CAS [411235-57-9]) using Pd(dppf)₂Cl₂·CH₂Cl₂ as a catalyst and K₂CO₃ as a base (General procedure A1).

### Step 2: 5-chloro-6-cyclopropyl-2-((2-methylpyridin-3-yl)amino)nicotinonitrile

The title compound ([M+H]⁺ 285.2) was prepared from 2,5-dichloro-6-cyclopropyl-pyridine-3-carbonitrile by reaction with 3-amino-2-methylpyridine (CAS [3430-10-2]) using Pd(OAc)₂ as a catalyst and xphos as a ligand (General procedure B1).

### Intermediate 19: 2-((3-cyano-2-methoxyphenyl)amino)-6-cyclopropylnicotinonitrile

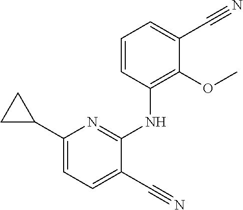 [SMILES: COc1c(C#N)cccc1Nc1nc(C2CC2)ccc1C#N]

The title compound ([M+H]⁺ 291.2) was prepared from 2-chloro-6-cyclopropylnicotinonitrile (CAS [1198475-35-2]) by reaction with 3-amino-2-methoxybenzonitrile (CAS [725718-10-5]) using Pd(OAc)₂ as a catalyst and xphos as a ligand (General procedure B1).

### Intermediate 20: 2-((3-cyano-2-ethoxyphenyl)amino)-6-cyclopropylnicotinonitrile

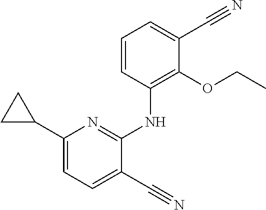 [SMILES: CCOc1c(C#N)cccc1Nc1nc(C2CC2)ccc1C#N]

The title compound ([M+H]⁺ 305.2) was prepared from 2-chloro-6-cyclopropylnicotinonitrile (CAS [1198475-35-2]) by reaction with 3-amino-2-ethoxybenzonitrile (CAS [1823514-97-1]) using Pd(OAc)₂ as a catalyst and xphos as a ligand (General procedure B1).

### Intermediate 21: 6-cyclopropyl-2-(1-tetrahydrofuran-2-ylethylamino)pyridine-3-carbonitrile

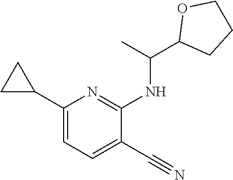 [SMILES: CC(Nc1nc(C2CC2)ccc1C#N)C1CCCO1]

The title compound ([M+H]⁺ 258.4) was prepared from 2-chloro-6-cyclopropylnicotinonitrile (CAS [1198475-35-2]) by reaction with 1-(tetrahydrofuran-2-yl)ethanamine (CAS [92071-57-3]) using NMP as solvent, DIPEA as a base at 210° C./MW (General procedure B2).

### Intermediate 22: 2-((2-methylpyridin-3-yl)amino)-4-(oxetan-3-yl)benzonitrile

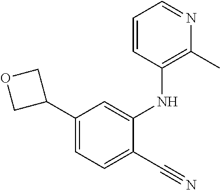 [SMILES: Cc1ncccc1Nc1cc(C2COC2)ccc1C#N]

### Step 1: 2-chloro-4-(oxetan-3-yl)benzonitrile

To a mixture of (3-chloro-4-cyanophenyl)boronic acid (197 mg, 1.09 mmol) and trans-2-aminocyclohexanol hydrochloride (5 mg, 0.03 mmol) in 2-propanol (2 mL) was added sodium bis(trimethylsilyl)amide 2M in THF (544 μL, 1.09 mmol). The reaction mixture was degassed and 3-iodooxetane (47 μL, 0.54 mmol) and nickel(II) iodide (10 mg, 0.03 mmol) were added. The resulting reaction was stirred at 80° C. in a sealed tube, before it was diluted with water and extracted with EtOAc. The combined organic layers were washed with brine, dried over Na₂SO₄, filtered and evaporated to dryness. The crude reaction mixture was purified by flash column chromatography to yield product (16 mg, 14%) as a white solid. ([M+H]⁺ 192.9)

### Step 2: 2-((2-methylpyridin-3-yl)amino)-4-(oxetan-3-yl)benzonitrile

The title compound ([M+H]⁺ 266.2) was prepared from 2-chloro-4-(oxetan-3-yl)benzonitrile by reaction with 3-amino-2-methylpyridine (CAS [3430-10-2]) at 100° C. using Pd₂(dba)₃ as catalyst, xantphos as a ligand and Cs₂CO₃ as a base in dioxane (General procedure I1).

### Intermediate 23: 6-((1RS,2RS)-2-methylcyclopropyl)-2-((2-methylpyridin-3-yl)amino)nicotinonitrile

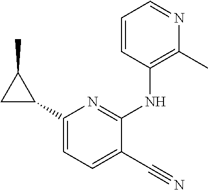 [SMILES: Cc1ncccc1Nc1nc([C@@H]2C[C@H]2C)ccc1C#N]

### Step 1: 2-hydroxy-6-(2-methylcyclopropyl)nicotinonitrile

2-hydroxy-6-(2-methylcyclopropyl)nicotinonitrile ([M+H]⁺ 175.0) was prepared by reaction of (E)-3-(dimethylamino)-1-(2-methylcyclopropyl)prop-2-en-1-one (prepared by a modified procedure from J. Med. Chem. 2011, 54, 7974-7985) and 2-cyanoacetamide (CAS [107-91-5]) using NaOMe as a base (General procedure G and H).

### Step 2: 2-chloro-6-((1RS,2RS)-2-methylcyclopropyl)nicotinonitrile

2-chloro-6-((1RS,2RS)-2-methylcyclopropyl)nicotinonitrile ([M+H]⁺ 193.1) was prepared by reaction of 2-hydroxy-6-(2-methylcyclopropyl)nicotinonitrile with POCl₃ (General procedure G and H).

### Step 3: 6-((1RS,2RS)-2-methylcyclopropyl)-2-((2-methylpyridin-3-yl)amino)nicotinonitrile

The title compound ([M+H]⁺ 265.3) was prepared from 2-chloro-6-((1RS,2RS)-2-methylcyclopropyl)nicotinonitrile by reaction with 3-amino-2-methylpyridine (CAS [3430-10-2]) using Pd(OAc)₂ as a catalyst and xphos as a ligand (General procedure B1).

### Intermediate 24: 6-cyclopropyl-2-[[(1SR,2RS)-2-triethylsilyloxycyclopentyl]amino]pyridine-3-carbonitrile

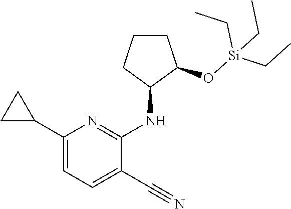 [SMILES: CC[Si](CC)(CC)O[C@@H]1CCC[C@@H]1Nc1nc(C2CC2)ccc1C#N]

### Step 1: 6-cyclopropyl-2-(((1SR,2RS)-2-hydroxycyclopentyl)amino)nicotinonitrile

The title compound ([M+H]⁺ 244.3) was prepared from 2-chloro-6-cyclopropylnicotinonitrile (CAS [1198475-35-2]) by reaction with (1SR,2RS)-2-aminocyclopentanol hydrochloride (CAS [137254-03-6]) using NMP as solvent, DIPEA as a base at 210° C./MW (General procedure B2).

### Step 2: 6-cyclopropyl-2-[[(1SR,2RS)-2-triethylsilyloxycyclopentyl]amino]pyridine-3-carbonitrile

To a solution of 6-cyclopropyl-2-(((1SR,2RS)-2-hydroxycyclopentyl)amino)nicotinonitrile (79 mg, 325 μmol) in DCE (2 ml) were added DIPEA (57 μl, 325 μmol), chlorotriethylsilane (60 μl, 357 μmol) and DMAP (48 mg, 390 μmol) and the reaction mixture was stirred at rt for 1 h. The next step was performed directly without isolation of 6-cyclopropyl-2-[[(1SR,2RS)-2-triethylsilyloxycyclopentyl]amino]pyridine-3-carbonitrile intermediate. ([M+H]⁺ 358.5)

### Intermediate 25: 6-cyclopropyl-2-((3-fluoro-2-methylphenyl)amino)nicotinonitrile

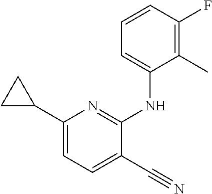 [SMILES: Cc1c(F)cccc1Nc1nc(C2CC2)ccc1C#N]

The title compound ([M+H]⁺ 268.3) was prepared from 2-chloro-6-cyclopropylnicotinonitrile (CAS [1198475-35-2]) by reaction with 3-fluoro-2-methylaniline (CAS [443-86-7]) using Pd(OAc)₂ as a catalyst and xantphos as a ligand (General procedure B1).

### Intermediate 26: 6-cyclopropyl-2-[[(3R)-oxan-3-yl]amino]pyridine-3-carbonitrile

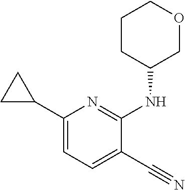

The title compound ([M+H]⁺ 244.3) was prepared from 2-chloro-6-cyclopropylnicotinonitrile (CAS [1198475-35-2]) by reaction with (R)-tetrahydro-2H-pyran-3-amine hydrochloride (CAS [1315500-31-2]) using NMP as solvent, DIPEA as a base at 210° C./MW (General procedure B2).

### Intermediate 27: 6-cyclopropyl-2-[[(3S)-oxan-3-yl]amino]pyridine-3-carbonitrile

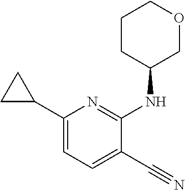 [SMILES: N#Cc1ccc(C2CC2)nc1N[C@H]1CCCOC1]

The title compound ([M+H]⁺ 244.3) was prepared from 2-chloro-6-cyclopropylnicotinonitrile (CAS [1198475-35-2]) by reaction with (S)-tetrahydro-2H-pyran-3-amine hydrochloride (CAS [1071829-81-6]) using NMP as solvent, DIPEA as a base at 210° C./MW (General procedure B2).

### Intermediate 28: 6-cyclopropyl-2-((4-methyltetrahydrofuran-3-yl)amino)nicotinonitrile

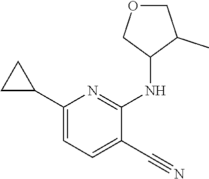 [SMILES: CC1COCC1Nc1nc(C2CC2)ccc1C#N]

The title compound ([M+H]⁺ 244.1) was prepared from 2-chloro-6-cyclopropylnicotinonitrile (CAS [1198475-35-2]) by reaction with 4-methyltetrahydrofuran-3-amine (CAS [1527863-66-6]) using NMP as solvent, DIPEA as a base at 120° C. (General procedure B2).

### Intermediate 29: 2-[(2-methylpyridin-3-yl)amino]-4-(trifluoromethyl)benzonitrile

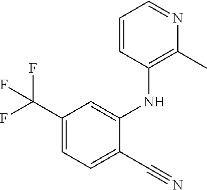 [SMILES: Cc1ncccc1Nc1cc(C(F)(F)F)ccc1C#N]

The title compound ([M+H]⁺ 278.2) was prepared from 2-bromo-4-(trifluoromethyl)benzonitrile (CAS [35764-15-9]) by reaction with 3-amino-2-methylpyridine (CAS [3430-10-2]) at 120° C. using Pd(OAc)₂ as catalyst, DPPF as a ligand and KOtBu as a base in toluene (General procedure I1).

### Intermediate 30: 6-cyclopropyl-2-[[(2SR,3RS)-2-methyltetrahydrofuran-3-yl]amino]pyridine-3-carbonitrile

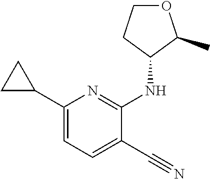 [SMILES: C[C@@H]1OCC[C@H]1Nc1nc(C2CC2)ccc1C#N]

### Step 1: (2SR,3SR)-2-methyltetrahydrofuran-3-yl 4-methylbenzenesulfonate

To a colorless solution of 2-methyldihydrofuran-3 (2H)-one (967 μl, 10.0 mmol) in THF (40 ml) at −78° C. was added L-selectride (1M in THF, 13 ml, 13.0 mmol) dropwise. The resulting reaction mixture was stirred at −78° C. for 1 h, before NaOH 1M was added and the mixture was allowed to warm up to room temperature. The aqueous phase was washed with DCM before it was carefully concentrated in vacuo. The remaining semisolid was suspended in DCM/MeOH 9:1 and filtered. Filtrate was concentrated in vacuo, resuspended in DCM and filtered over Dicalite and concentrated to afford crude (2SR,3SR)-2-methyltetrahydrofuran-3-ol used directly. At 0° C. 4-methylbenzenesulfonyl chloride (853 mg, 4.47 mmol) was added to a solution of (2SR,3SR)-2-methyltetrahydrofuran-3-ol (457 mg, 4.5 mmol) and TEA (1.25 ml, 9.0 mmol) in DCM (16 ml) and the resulting reaction mixture was stirred at room temperature until TLC showed complete consumption of starting material. The reaction mixture was diluted with DCM and washed 2× with water. The combined organic layers were dried with Na₂SO₄, filtered and concentrated in vacuo.

The crude material was purified by flash column chromatography to afford (2SR,3SR)-2-methyltetrahydrofuran-3-yl 4-methylbenzenesulfonate (374 mg, 33%) as a colorless oil. ¹H NMR (CDCl₃, 300 MHz) δ 7.8-7.8 (m, 2H), 7.35 (dd, 2H, J=0.6, 8.5 Hz), 4.9-5.0 (m, 1H, J=2.0, 3.7, 5.6 Hz), 3.9-4.0 (m, 1H), 3.86 (dq, 1H, J=3.7, 6.3 Hz), 3.72 (dt, 1H, J=5.4, 8.7 Hz), 2.46 (s, 3H), 2.0-2.3 (m, 2H), 1.20 (d, 3H, J=6.2 Hz))

### Step 2: (2SR,3RS)-2-methyltetrahydrofuran-3-amine acetate

To a solution of (2SR,3SR)-2-methyltetrahydrofuran-3-yl 4-methylbenzenesulfonate (374 mg, 1.46 mmol) in DMF (2 ml) sodium azide (285 mg, 4.38 mmol) was added. The mixture was heated to 80° C. for 72 h until TLC showed complete consumption of starting material. The reaction was diluted with Et₂O and washed twice with a minimal amount of water. 20 ml of MeOH was added to the mixture and the Et₂O mostly evaporated at 600 mbar (40° C.). The methanolic solution was acidified with acetic acid (418 μl, 7.3 mmol) and 10% Pd—C(16 mg, 146 μmol) was added and the mixture was set under H₂ atmosphere (balloon) and stirred for 16 h until TLC and LCMS showed complete consumption of starting material. The reaction mixture was filtered and concentrated to afford (2SR,3RS)-2-methyltetrahydrofuran-3-amine acetate (224 mg, 91%) as a colorless gum. ([M+H]⁺ 102.1)

### Step 3: 6-cyclopropyl-2-[[(2SR,3RS)-2-methyltetrahydrofuran-3-yl]amino]pyridine-3-carbonitrile

The title compound ([M+H]⁺ 244.2) was prepared from 2-chloro-6-cyclopropylnicotinonitrile (CAS [1198475-35-2]) by reaction with (2SR,3RS)-2-methyltetrahydrofuran-3-amine acetate in NMP at 210° C. using DIPEA as a base (General procedure B2).

### Intermediate 31: 4-cyclopropyloxy-2-[(2-methylpyridin-3-yl)amino]benzonitrile

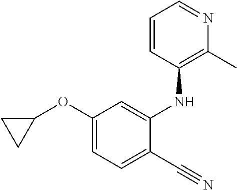

The title compound ([M+H]⁺ 266.2) was prepared from 2-bromo-4-cyclopropoxybenzonitrile (CAS [1237130-18-5]) by reaction with 3-amino-2-methylpyridine (CAS [3430-10-2]) at 100° C. using Pd₂(dba)₃ as catalyst, xantphos as a ligand and Cs₂CO₃ as a base in dioxane (General procedure I1).

### Intermediate 32: 2-((2-methylpyridin-3-yl)amino)-6-(trifluoromethyl)nicotinonitrile

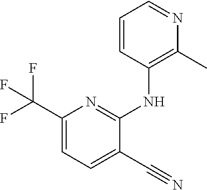 [SMILES: Cc1ncccc1Nc1nc(C(F)(F)F)ccc1C#N]

The title compound ([M+H]⁺ 279.2) was prepared from 2-chloro-6-(trifluoromethyl)nicotinonitrile (CAS [386704-06-9]) by reaction with 3-amino-2-methylpyridine (CAS [3430-10-2]) using Pd(OAc)₂ as a catalyst and xantphos as a ligand (General procedure B1).

### Intermediate 33: 6-cyclopropyl-2-[[rac-(1S)-3-triethylsilyloxycyclopentyl]amino]pyridine-3-carbonitrile

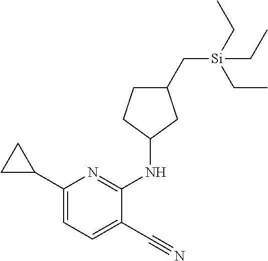 [SMILES: CC[Si](CC)(CC)CC1CCC(Nc2nc(C3CC3)ccc2C#N)C1]

### Step 1: 6-cyclopropyl-2-[[3-hydroxycyclopentyl]amino]pyridine-3-carbonitrile

The title compound ([M+H]⁺ 244.3) was prepared from 2-chloro-6-cyclopropylnicotinonitrile (CAS [1198475-35-2]) by reaction with 3-aminocyclopentan-1-ol (CAS [1279032-31-3]) using NMP as solvent, DIPEA as a base at 210° C. (General procedure B2).

### Step 2: 6-cyclopropyl-2-[[rac-(1S)-3-triethylsilyloxycyclopentyl]amino]pyridine-3-carbonitrile

To a solution of 6-cyclopropyl-2-((3-hydroxycyclopentyl)amino)nicotinonitrile (50 mg, 205 μmol) in DCE (1.3 ml) were added TEA (286 μl, 2.05 mmol), chlorotriethylsilane (55.2 μl, 329 μmol) and DMAP (30 mg, 247 μmol) and the reaction mixture was stirred at rt for 1 h before it was diluted with DCM and washed 2× with water. The combined organic layers were dried with Na₂SO₄, filtered and concentrated in vacuo. The crude material was purified by flash column chromatography to afford the desired product (74 mg, 100%) as a colorless oil. ([M+H]⁺ 358.4)

### Intermediate 34: 4-(difluoromethoxy)-2-[(2-methylpyridin-3-yl)amino]benzonitrile

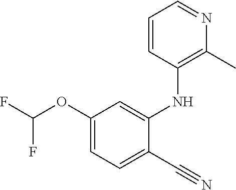 [SMILES: Cc1ncccc1Nc1cc(OC(F)F)ccc1C#N]

The title compound ([M+H]⁺ 276.3) was prepared from 2-bromo-4-(difluoromethoxy)benzonitrile (CAS [1261818-72-7]) by reaction with 3-amino-2-methylpyridine (CAS [3430-10-2]) at 100° C. using Pd₂(dba)₃ as catalyst, xantphos as a ligand and Cs₂CO₃ as a base in dioxane (General procedure I1).

### Intermediate 35: 4-(difluoromethyl)-2-((2-methylpyridin-3-yl)amino)benzonitrile

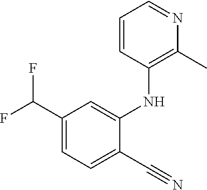 [SMILES: Cc1ncccc1Nc1cc(C(F)F)ccc1C#N]

### Step 1: 2-bromo-4-(difluoromethyl)benzonitrile

To a solution of 2-bromo-4-formylbenzonitrile (218 mg, 1.04 mmol) in DCM (5 ml) was added 1 M DAST in DCM (302 μl, 2.28 mmol) at room temperature. After stirring for 2 h, the reaction was quenched by sat. NaHCO₃ solution (3 mL) and extracted with DCM. The combined organic layers were washed with brine, dried over MgSO₄, and concentrated to give 2-bromo-4-(difluoromethyl)benzonitrile (202 mg 80% yield) as a brown oil. (¹H NMR (DMSO-d6, 300 MHz) dppm 8.09-8.15 (m, 2H) 7.73-7.86 (m, 1H) 6.90-7.37 (m, 1H))

### Step 2: 4-(difluoromethyl)-2-((2-methylpyridin-3-yl)amino)benzonitrile

The title compound ([M+H]⁺ 260.2) was prepared from 2-bromo-4-(difluoromethyl)benzonitrile (CAS [1261580-17-9]) by reaction with 3-amino-2-methylpyridine (CAS [3430-10-2]) at 100° C. using Pd₂(dba)₃ as catalyst, xantphos as a ligand and Cs₂CO₃ as a base in dioxane (General procedure I1).

### Intermediate 36: 6-[(1RS,2SR)-2-fluorocyclopropyl]-2-[(2-methyl-3-pyridyl)amino]pyridine-3-carbonitrile

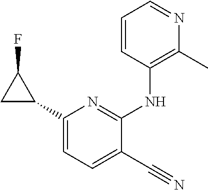 [SMILES: Cc1ncccc1Nc1nc([C@@H]2C[C@H]2F)ccc1C#N]

### Step 1: 6-((trans)-2-fluorocyclopropyl)-2-hydroxynicotinonitrile

6-((trans)-2-fluorocyclopropyl)-2-hydroxynicotinonitrile ([M+H]⁺ 179.0) was prepared by reaction of 3-(dimethylamino)-1-((trans)-2-fluorocyclopropyl)prop-2-en-1-one (prepared by a modified procedure from J. Med. Chem. 2011, 54, 7974-7985) and 2-cyanoacetamide (CAS [107-91-5]) using NaOMe as a base (General procedure G and H).

### Step 2: 2-chloro-6-((trans)-2-fluorocyclopropyl)nicotinonitrile

2-chloro-6-((trans)-2-fluorocyclopropyl)nicotinonitrile (¹H NMR (CDCl₃, 300 MHz) δ 7.8-7.9 (m, 1H), 7.3-7.3 (m, 1H), 4.8-5.1 (m, 1H), 2.49 (d, 1H, J=1.6 Hz), 1.6-1.8 (m, 1H), 1.5-1.6 (m, 1H)) was prepared by reaction of 6-((trans)-2-fluorocyclopropyl)-2-hydroxynicotinonitrile with POCl₃ (General procedure G and H).

### Step 3: 6-[(1RS,2SR)-2-fluorocyclopropyl]-2-[(2-methyl-3-pyridyl)amino]pyridine-3-carbonitrile

The title compound ([M+H]⁺ 269.0) was prepared from 2-chloro-6-((trans)-2-fluorocyclopropyl)nicotinonitrile by reaction with 3-amino-2-methylpyridine (CAS [3430-10-2]) at 80° C. using Pd(OAc)₂ as a catalyst and xantphos as a ligand (General procedure B1).

### Intermediate 37: 2-cyclopropyl-6-((2-methylpyridin-3-yl)amino)pyridine-3,5-dicarbonitrile

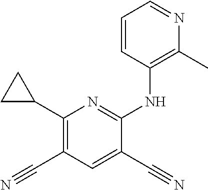 [SMILES: Cc1ncccc1Nc1nc(C2CC2)c(C#N)cc1C#N]

### Step 1: 2-amino-6-cyclopropylnicotinonitrile

The title compound ([M+H]⁺ 160.1) was prepared from 2-chloro-6-cyclopropylnicotinonitrile (CAS [1198475-35-2]) using General procedure D.

### Step 2: 2-amino-5-bromo-6-cyclopropylnicotinonitrile

The title compound ([M+H]⁺ 238.0) was prepared from 2-amino-6-cyclopropylnicotinonitrile and NBS in CHCl₃ (General procedure E).

### Step 3: 2-amino-6-cyclopropylpyridine-3,5-dicarbonitrile

To a solution of 2-amino-5-bromo-6-cyclopropylnicotinonitrile (21 mg, 0.088 mmol) in DMF (0.5 mL) were added Zn(CN)₂ (11 mg, 0.088 mmol) and Pd(PPh₃)₄ (3 mg, 0.0026 mmol). The reaction was heated in microwave at 150° C. for 1 h before additional portion of Zn(CN)₂ (11 mg, 0.088 mmol) and Pd(PPh₃)₄ (10 mg, 0.0088 mmol) were added. The reaction was allowed to stir for additional 30 min in microwave at 150° C. The reaction was quenched with water and extracted with EtOAc. The combined organic layers were washed with brine, dried over MgSO₄, and concentrated. 2-amino-6-cyclopropylpyridine-3,5-dicarbonitrile was purified by flash column chromatography to give 2-amino-6-cyclopropylpyridine-3,5-dicarbonitrile as a white solid (12 mg, 74%). ([M+H]⁺ 185.2)

### Step 4: 2-cyclopropyl-6-((2-methylpyridin-3-yl)amino)pyridine-3,5-dicarbonitrile

The title compound ([M+H]⁺ 276.3) was prepared from 2-amino-6-cyclopropylpyridine-3,5-dicarbonitrile by reaction with 3-bromo-2-methylpyridine (CAS [38749-79-0]) using Pd₂(dba)₃ as a catalyst and xantphos as a ligand (General procedure F).

### Intermediate 38: 2-((3-cyano-2-methoxyphenyl)amino)-6-cyclopropylnicotinonitrile

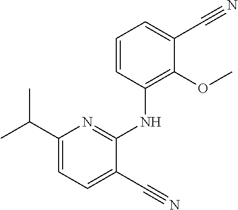 [SMILES: COc1c(C#N)cccc1Nc1nc(C(C)C)ccc1C#N]

The title compound ([M+H]⁺ 291.2) was prepared from 2-chloro-6-(propan-2-yl)pyridine-3-carbonitrile (CAS [108244-44-6]) by reaction with 3-amino-2-methoxybenzonitrile (CAS [725718-10-5]) using Pd(OAc)₂ as a catalyst and xphos as a ligand (General procedure B1).

### Intermediate 39: 4-methoxy-2-((2-methylpyridin-3-yl)amino)benzonitrile

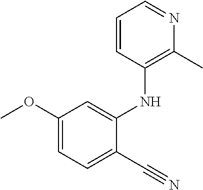

The title compound ([M+H]⁺ 240.2) was prepared from 2-bromo-4-methoxybenzonitrile (CAS [140860-51-1]) by reaction with 3-amino-2-methylpyridine (CAS [3430-10-2]) at 120° C. using Pd(OAc)₂ as catalyst, DPPF as a ligand and KO^{t}Bu as a base in toluene (General procedure I1).

### Intermediate 40: 2-((2-methylpyridin-3-yl)amino)-4-(trifluoromethoxy)benzonitrile

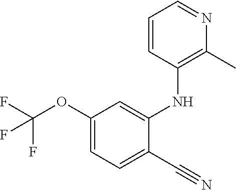 [SMILES: Cc1ncccc1Nc1cc(OC(F)(F)F)ccc1C#N]

The title compound ([M+H]⁺ 294.2) was prepared from 2-bromo-4-(trifluoromethoxy)benzonitrile (CAS [1214334-83-4]) by reaction with 3-amino-2-methylpyridine (CAS [3430-10-2]) at 100° C. using Pd(OAc)₂ as catalyst, xantphos as a ligand and Cs₂CO₃ as a base in dioxane (General procedure I1).

### Intermediate 41: 6-(4,5-dihydrofuran-3-yl)-2-((2-methylpyridin-3-yl)amino)nicotinonitrile

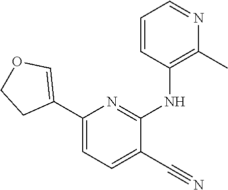 [SMILES: Cc1ncccc1Nc1nc(C2=COCC2)ccc1C#N]

### Step 1: 2-chloro-6-(2,3-dihydrofuran-4-yl)pyridine-3-carbonitrile

The title compound ([M+H]⁺ 207.1) was prepared from by reaction of 2,6-dichloronicotinonitrile (CAS [40381-90-6]) and 2-(4,5-dihydrofuran-3-yl)-4,4,5,5-tetramethyl-1,3,2-dioxaborolane (CAS [1046812-03-6]) using Pd(dppf)₂Cl₂·CH₂Cl₂ complex as a catalyst and K₂CO₃ as a base at 80° C. (General procedure A1).

### Step 2: 6-(4,5-dihydrofuran-3-yl)-2-((2-methylpyridin-3-yl)amino)nicotinonitrile

The title compound ([M+H]⁺ 279.2) was prepared from 2-chloro-6-(4,5-dihydrofuran-3-yl)nicotinonitrile by reaction with 3-amino-2-methylpyridine (CAS [3430-10-2]) using Pd(OAc)₂ as a catalyst and xantphos as a ligand (General procedure B1).

### Intermediate 42: 6-cyclopropyl-2-((4-methylpyrimidin-5-yl)amino)nicotinonitrile

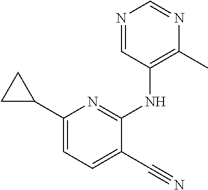 [SMILES: Cc1ncncc1Nc1nc(C2CC2)ccc1C#N]

The title compound ([M+H]⁺ 252.2) was prepared from 2-chloro-6-cyclopropylnicotinonitrile (CAS [1198475-35-2]) by reaction with 5-amino-4-methylpyrimidine (CAS [3438-61-7]) using Pd(OAc)₂ as a catalyst and xantphos as a ligand (General procedure B1).

### Intermediate 43: 6-cyclopropyl-2-((2-(trifluoromethyl)pyridin-3-yl)amino)nicotinonitrile

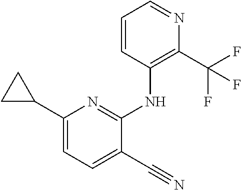 [SMILES: N#Cc1ccc(C2CC2)nc1Nc1cccnc1C(F)(F)F]

The title compound ([M+H]⁺ 305.2) was prepared from 2-chloro-6-cyclopropylnicotinonitrile (CAS [1198475-35-2]) by reaction with 2-(trifluoromethyl)pyridin-3-amine (CAS [106877-32-1]) using Pd(OAc)₂ as a catalyst and xantphos as a ligand (General procedure B1).

### Intermediate 44: 6-cyclopropyl-2-[(2-methylpyrazol-3-yl)amino]pyridine-3-carbonitrile

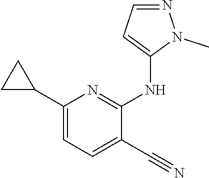 [SMILES: Cn1nccc1Nc1nc(C2CC2)ccc1C#N]

The title compound ([M+H]⁺ 240.1) was prepared from 2-chloro-6-cyclopropylnicotinonitrile (CAS [1198475-35-2]) by reaction with 1-methyl-1H-pyrazol-5-ylamine (CAS [1192-21-8]) using Pd(OAc)₂ as a catalyst and xantphos as a ligand (General procedure B1).

### Intermediate 45: 4-cyclopropyl-2-((2,3-dihydrobenzofuran-4-yl)amino)benzonitrile

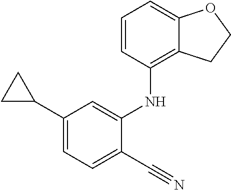 [SMILES: N#Cc1ccc(C2CC2)cc1Nc1cccc2c1CCO2]

The title compound ([M+H]⁺ 277.2) was prepared from 2-bromo-4-cyclopropylbenzonitrile (CAS [1237130-18-5]) by reaction with 2,3-dihydrobenzofuran-4-amine (CAS [61090-37-7]) at 100° C. using Pd(OAc)₂ as catalyst and xantphos as a ligand (General procedure I1).

### Intermediate 46: (R)-2-((tetrahydro-2H-pyran-3-yl)amino)-4-(trifluoromethyl)benzonitrile

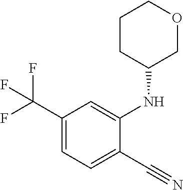

The title compound ([M+H]⁺ 271.2) was prepared from 2-bromo-4-(trifluoromethyl)benzonitrile (CAS [35764-15-9]) by reaction with (R)-tetrahydro-2H-pyran-3-amine hydrochloride (CAS 10 [1315500-31-2]) at 90° C. using Pd(OAc)₂ as catalyst and xantphos as a ligand (General procedure I1).

### Intermediate 47: 6-cyclopropyl-2-((4-methylpyridin-3-yl)amino)nicotinonitrile

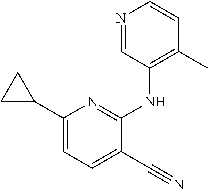 [SMILES: Cc1ccncc1Nc1nc(C2CC2)ccc1C#N]

The title compound ([M+H]⁺ 251.2) was prepared from 2-chloro-6-cyclopropylnicotinonitrile (CAS [1198475-35-2]) by reaction with 3-amino-4-methylpyridine (CAS [3430-27-1]) using Pd(OAc)₂ as a catalyst and xantphos as a ligand (General procedure B1).

### Intermediate 48: 4-ethyl-2-((2-methylpyridin-3-yl)amino)benzonitrile

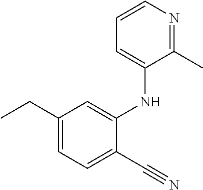 [SMILES: CCc1ccc(C#N)c(Nc2cccnc2C)c1]

The title compound ([M+H]⁺ 238.2) was prepared from 2-bromo-4-ethylbenzonitrile (CAS [38678-87-4]) by reaction with 3-amino-2-methylpyridine (CAS [3430-10-2]) at 100° C. using Pd(OAc)₂ as catalyst and xantphos as a ligand (General procedure I1).

### Intermediate 49: 6-[(1SR,2RS)-2-fluorocyclopropyl]-2-[[(3R)-tetrahydropyran-3-yl]amino]pyridine-3-carbonitrile

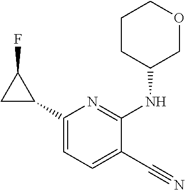

### Step 1: 6-((trans)-2-fluorocyclopropyl)-2-hydroxynicotinonitrile

6-((trans)-2-fluorocyclopropyl)-2-hydroxynicotinonitrile ([M+H]⁺ 179.0) was prepared by reaction of 3-(dimethylamino)-1-((trans)-2-fluorocyclopropyl)prop-2-en-1-one (prepared by a modified procedure from J. Med. Chem. 2011, 54, 7974-7985) and 2-cyanoacetamide (CAS [107-91-5]) using NaOMe as a base (General procedure G and H).

### Step 2: 2-chloro-6-((trans)-2-fluorocyclopropyl)nicotinonitrile

2-chloro-6-((trans)-2-fluorocyclopropyl)nicotinonitrile (1H NMR (CDCl3, 300 MHz) δ 7.8-7.9 (m, 1H), 7.3-7.3 (m, 1H), 4.8-5.1 (m, 1H), 2.49 (d, 1H, J=1.6 Hz), 1.6-1.8 (m, 1H), 1.5-1.6 (m, 1H)) was prepared by reaction of 6-((trans)-2-fluorocyclopropyl)-2-hydroxynicotinonitrile with POCl₃ (General procedure G and H).

### Step 3: 6-[(1SR,2RS)-2-fluorocyclopropyl]-2-[[(3R)-tetrahydropyran-3-yl]amino]pyridine-3-carbonitrile

The title compound ([M+H]⁺ 260.0) was prepared from 2-chloro-6-((trans)-2-fluorocyclopropyl)nicotinonitrile by reaction with (R)-tetrahydro-2H-pyran-3-amine hydrochloride (CAS [1315500-31-2]) at 80° C. using Pd(OAc)₂ as catalyst and xantphos as a ligand (General procedure I1).

### Intermediate 50: 6-cyclopropyl-2-[(4-methyloxazol-5-yl)amino]pyridine-3-carbonitrile

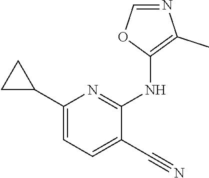 [SMILES: Cc1ncoc1Nc1nc(C2CC2)ccc1C#N]

### Step 1: tert-butyl N-(3-cyano-6-cyclopropyl-2-pyridyl)-N-(4-methyloxazol-5-yl)carbamate

The title compound ([M+H]⁺ 341.1) was prepared from 2-chloro-6-cyclopropylnicotinonitrile (CAS [1198475-35-2]) by reaction with tert-butyl N-(4-methyloxazol-5-yl)carbamate (CAS [3403-45-0]) at 80° C. using Pd(OAc)₂ as a catalyst and xantphos as a ligand (General procedure B1).

### Step 2: 6-cyclopropyl-2-[(4-methyloxazol-5-yl)amino]pyridine-3-carbonitrile

A mixture of tert-butyl N-(3-cyano-6-cyclopropyl-2-pyridyl)-N-(4-methyloxazol-5-yl)carbamate (50 mg, 0.150 mmol) in TFA:DCM=1:1 (3.0 mL) was stirred at 25° C. for 2 h before saturated aqueous NaHCO₃ was added. The reaction mixture was extracted with DCM (20 mL×2) and the combined organic layers were dried over Na₂SO₄, filtered and concentrated in vacuo to give crude 6-cyclopropyl-2-[(4-methyloxazol-5-yl)amino]pyridine-3-carbonitrile (40 mg 108%) as light yellow solid. ([M+H]⁺ 241.1)

### Intermediate 51: 6-cyclopropyl-2-[(4-methylthiazol-5-yl)amino]pyridine-3-carbonitrile

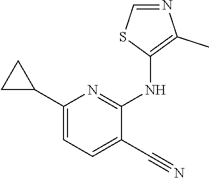 [SMILES: Cc1ncsc1Nc1nc(C2CC2)ccc1C#N]

### Step 1: tert-butyl N-(4-methylthiazol-5-yl)carbamate

To a solution of 4-methylthiazole-5-carboxylic acid (600 mg, 4.19 mmol) in tBuOH (15 mL) were added TEA (2.34 mL, 16.76 mmol) and diphenylphosphorylazide (1.73 g, 6.29 mmol). The reaction mixture was stirred at 20° C. for 15 min and then heated to 80° C. for 2 h. The reaction mixture was poured into saturated aqueous NaHCO₃ (30 mL) and extracted with EtOAc (50 mL×3). The combined organic layers were washed with brine (30 mL), dried over Na₂SO₄, filtered and concentrated in vacuo to give crude tert-butyl N-(4-methylthiazol-5-yl)carbamate (400 mg, 44%) as a yellow solid. ([M+H]⁺ 215.1)

### Step 2: 4-methylthiazol-5-amine hydrochloride

To a solution of tert-butyl N-(4-methylthiazol-5-yl)carbamate (400 mg, 1.87 mmol) in DCM (5 mL) HCl (4 M in dioxane, 2.0 mL, 8 mmol) was added. The reaction was stirred at 25° C. for 12 h until TCL and LCMS showed full consumption of starting material. The reaction mixture was concentrated in vacuo to give crude 4-methylthiazol-5-amine hydrochloride (200 mg, 71%) as a yellow solid. ([M+H]⁺ 115.1)

### Step 3: 6-cyclopropyl-2-[(4-methylthiazol-5-yl)amino]pyridine-3-carbonitrile

The title compound ([M+H]⁺ 256.9) was prepared from 2-chloro-6-cyclopropylnicotinonitrile (CAS [1198475-35-2]) by reaction with 4-methylthiazol-5-amine hydrochloride at 100° C. using Pd(OAc)₂ as a catalyst and xantphos as a ligand (General procedure B1).

### Intermediate 52: 6-cyclopropyl-4-(o-tolylamino)nicotinonitrile

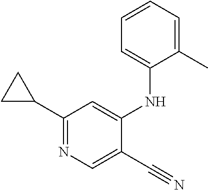 [SMILES: Cc1ccccc1Nc1cc(C2CC2)ncc1C#N]

### Step 1: 4-chloro-6-cyclopropylnicotinonitrile

The title compound ([M+H]⁺ 179.1) was prepared by reaction of 4,6-dichloronicotinonitrile (CAS [166526-03-0]) and cyclopropylboronic acid (CAS [411235-57-9]) using Pd(dppf)₂Cl₂·CH₂Cl₂ complex as a catalyst and K₂CO₃ as a base at 90° C. (in analogy to General procedure A1).

### Step 2: 6-cyclopropyl-4-(o-tolylamino)nicotinonitrile

The title compound ([M+H]⁺ 250.2) was prepared from 4-chloro-6-cyclopropylnicotinonitrile by reaction with o-toluidine (CAS [95-35-4]) at 100° C. using Pd(OAc)₂ as a catalyst and xphos as a ligand (in analogy to General procedure B1).

### Intermediate 53: 6-cyclobutyl-2-[(2-methyl-3-pyridyl)amino]pyridine-3-carbonitrile

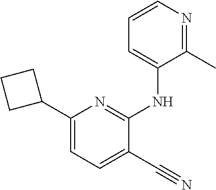 [SMILES: Cc1ncccc1Nc1nc(C2CCC2)ccc1C#N]

### Step 1: 2-chloro-6-cyclobutyl-pyridine-3-carbonitrile

To a 40 mL vial equipped with a stirrer bar were added bromocyclobutane (1171 mg, 8.67 mmol), 2,6-dichloronicotinonitrile (1000 mg, 5.78 mmol), Ir[dF(CF₃)ppy]₂(dtbpy)(PF₆) (65 mg, 0.060 mmol), NiCl₂·dtbbpy (12 mg, 0.030 mmol), tris(trimethylsilyl)silane (1437 mg, 5.78 mmol) and Na₂CO₃ (1225 mg, 11.56 mmol) in DME (30 mL). The vial was sealed and placed under nitrogen. The reaction was stirred and irradiated with a 34 W blue LED lamp (7 cm away), with cooling fan to keep the reaction temperature at 25° C. for 14 h. Ethyl acetate (30 mL) and brine (20 mL) were added and layers were separated. The aqueous phase was extracted with ethyl acetate (30 mL×2). Combined organic layers were dried over Na₂SO₄, filtered and concentrated in vacuo. Purification by reversed phase prep-HPLC afforded 2-chloro-6-cyclobutyl-pyridine-3-carbonitrile (100 mg 9%) as white solid. ([M+H]⁺ 193.0)

### Step 2: cyclobutyl-2-[(2-methyl-3-pyridyl)amino]pyridine-3-carbonitrile

The title compound ([M+H]⁺ 265.2) was prepared from 2-chloro-6-cyclobutyl-pyridine-3-carbonitrile by reaction with 3-amino-2-methylpyridine (CAS [3430-10-2]) using Pd(OAc)₂ as a catalyst and xphos as a ligand (General procedure B1).

### Intermediate 54: 2-[(2-methoxy-3-pyridyl)amino]-6-tetrahydropyran-2-yl-pyridine-3-carbonitrile

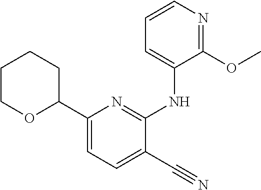 [SMILES: COc1ncccc1Nc1nc(C2CCCCO2)ccc1C#N]

### Step 1: 2-chloro-6-(3,4-dihydro-2H-pyran-6-yl)pyridine-3-carbonitrile

The title compound (¹H NMR (DMSO-d₆, 400 MHz) δ=8.43 (d, J=8.2 Hz, 1H), 7.61 (d, J=8.2 Hz, 1H), 6.24 (t, J=4.3 Hz, 1H), 4.20-4.16 (m, 2H), 2.30-2.24 (m, 2H), 1.90-1.83 (m, 2H)) was prepared from by reaction of 2,6-dichloronicotinonitrile (CAS [40381-90-6]) and 2-(3,4-dihydro-2H-pyran-6-yl)-4,4,5,5-tetramethyl-1,3,2-dioxaborolane (CAS [1025707-93-0]) using Pd(dppf)₂Cl₂·CH₂Cl₂ complex as a catalyst and K₂CO₃ as a base at 80° C. (General procedure A1).

### Step 2: 6-(3,4-dihydro-2H-pyran-6-yl)-2-[(2-methoxy-3-pyridyl)amino]pyridine-3-carbonitrile

The title compound ([M+H]⁺ 309.1) was prepared from 2-chloro-6-(3,4-dihydro-2H-pyran-6-yl)pyridine-3-carbonitrile by reaction with o-anisidine (CAS [90-04-0]) at 80° C. using Pd(OAc)₂ as a catalyst and xantphos as a ligand (General procedure B1).

### Step 3: 2-[(2-methoxy-3-pyridyl)amino]-6-tetrahydropyran-2-yl-pyridine-3-carbonitrile

A mixture of 6-(3,4-dihydro-2H-pyran-6-yl)-2-[(2-methoxy-3-pyridyl)amino]pyridine-3-carbonitrile (600 mg, 1.95 mmol) and 10% Pd/C (1.95 mmol) in THF (120 mL) and was stirred at 30° C. for 1 h under H₂ balloon before it was filtered and concentrated under reduced pressure. The residue was purified by prep-HPLC to give 2-[(2-methoxy-3-pyridyl)amino]-6-tetrahydropyran-2-yl-pyridine-3-carbonitrile (150 mg 25%) as white solid. (¹H NMR (DMSO-d₆, 400 MHz) δ=8.25 (s, 1H), 8.10 (d, J=7.9 Hz, 1H), 7.89 (dd, J=1.7, 5.0 Hz, 1H), 7.02 (dd, J=4.9, 7.7 Hz, 1H), 6.98 (d, J=7.9 Hz, 1H), 4.28 (dd, J=2.3, 11.1 Hz, 1H), 4.07-4.00 (m, 1H), 3.92 (s, 3H), 3.54 (br d, J=3.2 Hz, 1H), 1.95 (br dd, J=2.6, 13.1 Hz, 1H), 1.84 (br s, 1H), 1.71-1.58 (m, 1H), 1.57-1.48 (m, 2H), 1.36 (br s, 1H))

### Intermediate 55: 6-cyclopentyl-2-[(4-methylpyrimidin-5-yl)amino]pyridine-3-carbonitrile

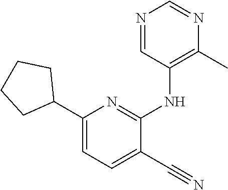 [SMILES: Cc1ncncc1Nc1nc(C2CCCC2)ccc1C#N]

### Step 1: 2-chloro-6-(cyclopenten-1-yl)pyridine-3-carbonitrile

The title compound ([M+H]⁺ 205.0) was prepared from by reaction of 2,6-dichloronicotinonitrile (CAS [40381-90-6]) and 1-cyclopentenyl boronic acid pinacol ester (CAS [287944-10-9]) using Pd(dppf)₂Cl₂·CH₂Cl₂ complex as a catalyst and K₂CO₃ as a base at 80° C. (General procedure A1).

### Step 2: 6-(cyclopenten-1-yl)-2-[(4-methylpyrimidin-5-yl)amino]pyridine-3-carbonitrile

The title compound (¹H NMR (DMSO-d₆, 400 MHz) δ=9.03 (s, 1H), 8.85 (s, 1H), 8.69 (s, 1H), 8.05 (d, J=8.1 Hz, 1H), 7.05 (d, J=7.9 Hz, 1H), 6.53 (d, J=1.7 Hz, 1H), 4.03 (q, J=7.1 Hz, 5H), 2.48-2.44 (m, 3H), 2.34 (s, 3H), 1.99 (s, 7H), 1.92-1.81 (m, 2H), 1.17 (t, J=7.1 Hz, 8H)) was prepared from 2-chloro-6-(cyclopenten-1-yl)pyridine-3-carbonitrile by reaction with 5-amino-4-methylpyrimidine (CAS [3438-61-7]) at 80° C. using Pd(OAc)₂ as a catalyst and xantphos as a ligand (General procedure B1).

### Step 3: 6-cyclopentyl-2-[(4-methylpyrimidin-5-yl)amino]pyridine-3-carbonitrile

To a solution of 6-(cyclopenten-1-yl)-2-[(4-methylpyrimidin-5-yl)amino]pyridine-3-carbonitrile (350 mg, 1.26 mmol) in THF (70 mL) was added 10% Pd/C (0.26 mmol). The mixture was stirred at 20° C. for 1 h under H₂ atmosphere (balloon) before it was filtered and concentrated under reduced pressure to give crude 6-cyclopentyl-2-[(4-methylpyrimidin-5-yl)amino]pyridine-3-carbonitrile (480 mg, quant.) as a white solid. (M+H]⁺280.0)

### Intermediate 56: 6-cyclopentyl-2-[(2-methyl-3-pyridyl)amino]pyridine-3-carbonitrile

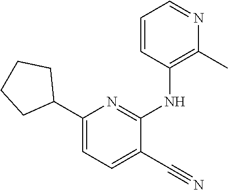 [SMILES: Cc1ncccc1Nc1nc(C2CCCC2)ccc1C#N]

### Step 1: 2-chloro-6-(cyclopenten-1-yl)pyridine-3-carbonitrile

The title compound ([M+H]⁺ 205.0) was prepared from by reaction of 2,6-dichloronicotinonitrile (CAS [40381-90-6]) and 1-cyclopentenyl boronic acid pinacol ester (CAS [287944-10-9]) using Pd(dppf)₂Cl₂·CH₂Cl₂ complex as a catalyst and K₂CO₃ as a base at 80° C. (General procedure A1).

### Step 2: 6-(cyclopenten-1-yl)-2-[(2-methyl-3-pyridyl)amino]pyridine-3-carbonitrile

The title compound ([M+H]⁺ 277.2) was prepared from 2-chloro-6-(cyclopenten-1-yl)pyridine-3-carbonitrile by reaction with 3-amino-2-methylpyridine (CAS [3430-10-2]) at 80° C. using Pd(OAc)₂ as a catalyst and xantphos as a ligand (General procedure B1).

### Step 3: 6-cyclopentyl-2-[(2-methyl-3-pyridyl)amino]pyridine-3-carbonitrile

To a solution of 6-(cyclopenten-1-yl)-2-[(2-methyl-3-pyridyl)amino]pyridine-3-carbonitrile (130.0 mg, 0.470 mmol) in THF (26 mL) was added 10% Pd/C (0.26 mmol). The mixture was stirred at 20° C. for 1 h under H₂ balloon before it was filtered and concentrated under reduced pressure to give 6-cyclopentyl-2-[(2-methyl-3-pyridyl)amino]pyridine-3-carbonitrile (150 mg, 100% yield) as off-white solid. ([M+H]⁺ 279.2)

### Intermediate 57: 6-(3-azabicyclo[2.2.1]heptan-3-yl)-2-[(2-methylpyrazol-3-yl)amino]pyridine-3-carbonitrile

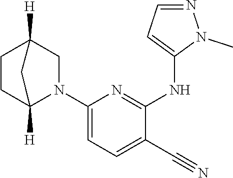 [SMILES: Cn1nccc1Nc1nc(N2C[C@H]3CC[C@@H]2C3)ccc1C#N]

### Step 1: 6-(7-azabicyclo[2.2.1]heptan-7-yl)-2-chloro-pyridine-3-carbonitrile

To a solution of 2,6-dichloronicotinonitrile (800 mg, 4.62 mmol) in THF (50 mL) and ACN (50 mL) were added 7-azabicyclo[2.2.1]heptane hydrochloride (618 mg, 4.62 mmol) and N,N-diisopropylethylamine (2.42 mL, 13.87 mmol). The reaction mixture was stirred at 30° C. for 16 h before it was concentrated in vacuo and purified by flash column chromatography to give 6-(7-azabicyclo[2.2.1]heptan-7-yl)-2-chloro-pyridine-3-carbonitrile (600 mg, 50%) as a yellow solid. (¹H NMR (DMSO-d₆, 400 MHz) δ=7.93-7.71 (m, 2H), 6.72 (br d, J=8.8 Hz, 1H), 6.37 (br d, J=8.8 Hz, 1H), 4.79-4.62 (m, 1H), 4.57-4.40 (m, 1H), 3.42-3.35 (m, 2H), 3.21-3.12 (m, 1H), 3.02 (br d, J=9.6 Hz, 1H), 2.70-2.61 (m, 2H), 1.76-1.62 (m, 6H), 1.57-1.35 (m, 6H))

### Step 2: 6-(3-azabicyclo[2.2.1]heptan-3-yl)-2-[(2-methylpyrazol-3-yl)amino]pyridine-3-carbonitrile

The title compound (¹H NMR (CDCl₃, 400 MHz) δ=7.38 (br s, 1H), 7.36-7.31 (m, 1H), 6.47-6.31 (m, 1H), 6.23-6.10 (m, 1H), 5.96-5.55 (m, 1H), 4.57-4.36 (m, 1H), 3.76-3.61 (m, 3H), 3.32-3.23 (m, 1H), 3.08-2.76 (m, 1H), 2.63-2.44 (m, 1H), 1.74-1.57 (m, 4H), 1.42-1.25 (m, 2H)) was prepared from 6-(7-azabicyclo[2.2.1]heptan-7-yl)-2-chloro-pyridine-3-carbonitrile by reaction with 1-methyl-1H-pyrazol-5-ylamine (CAS [1192-21-8]) at 100° C. using Pd(OAc)₂ as a catalyst and xantphos as a ligand (General procedure B1).

### Intermediate 58: 4-(2-fluoropropan-2-yl)-2-[(2-methylpyridin-3-yl)amino]benzonitrile

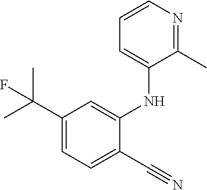 [SMILES: Cc1ncccc1Nc1cc(C(C)(C)F)ccc1C#N]

### Step 1: 2-bromo-4-(2-hydroxypropan-2-yl)benzonitrile

To a solution of 4-acetyl-2-bromobenzonitrile (50 mg, 223 μmol, CAS [93273-63-3]) in DCM (2 ml) was added methylmagnesium bromide solution (3 M in diethyl ether, 89 μl, 268 μmol) at 0° C. After 45 min the reaction was diluted with sat. aq. NH₄Cl solution and extracted 3× with ethyl acetate. The combined organic layers were washed with water and brine, dried over magnesium sulfate, filtered and concentrated in vacuo. The crude product was purified by flash column chromatography to afford 2-bromo-4-(2-hydroxypropan-2-yl)benzonitrile (37 mg, 68%) as a yellow viscous oil. ([GCMS: M]⁺239.0)

### Step 2: 2-bromo-4-(2-fluoropropan-2-yl)benzonitrile

To a solution of 2-bromo-4-(2-hydroxypropan-2-yl)benzonitrile (36 mg, 150 μmol) in DCM (1 ml) at −70° C. DAST (23.8 μl, 180 μmol) was added. The ice-bath was removed and after 2 h the reaction was diluted with sat. aq. NaHCO₃ solution and extracted with DCM. The combined organic layers were washed with brine, dried over MgSO₄, filtered and concentrated in vacuo. The crude product was purified by flash column chromatography to yield 2-bromo-4-(2-fluoropropan-2-yl)benzonitrile (20 mg, 54%) as a yellow viscous oil. ([GCMS: M]⁺241.0)

### Step 3: 4-(2-fluoropropan-2-yl)-2-[(2-methylpyridin-3-yl)amino]benzonitrile

The title compound ([M+H]⁺ 270.2) was prepared from 2-bromo-4-(2-fluoropropan-2-yl)benzonitrile by reaction with 3-amino-2-methylpyridine (CAS [3430-10-2]) at 100° C. using Pd₂(dba)₃ as catalyst and xantphos as a ligand (General procedure I1).

### Intermediate 59: 2-fluoro-6-((2-methylpyridin-3-yl)amino)-4-(trifluoromethyl)benzonitrile

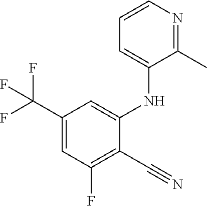 [SMILES: Cc1ncccc1Nc1cc(C(F)(F)F)cc(F)c1C#N]

The title compound ([M+H]⁺ 296.2) was prepared from 2,6-difluoro-4-(trifluoromethyl)benzonitrile (CAS [1803828-56-9]) by reaction with 3-amino-2-methylpyridine (CAS [3430-10-2]) using NMP as solvent, KOtBu as a base at room temperature (General procedure I2).

### Intermediate 60: 6-cyclopropyl-2-((4-fluoro-2-methoxypyridin-3-yl)amino)nicotinonitrile

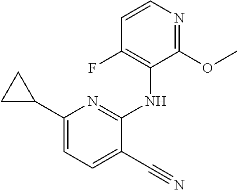 [SMILES: COc1nccc(F)c1Nc1nc(C2CC2)ccc1C#N]

### Step 1: tert-butyl (4-fluoro-2-methoxypyridin-3-yl)carbamate

To a solution of tert-butyl (2-methoxypyridin-3-yl)carbamate (500 mg, 2.23 mmol) and TMEDA (518 mg, 4.46 mmol) in dry THF (20 ml) at −35° C. nBuLi (1.6 M in hexane, 5.57 ml, 8.92 mmol) was added dropwise via syringe. After reaching −20° C., the mixture was stirred for 2 h at −20° C., cooled again to −35° C. and a solution of N-fluorobenzenesulfonimide (1 M in THF, 2.45 mmol) was added. The resulting reaction mixture was allowed to reach −20° C. and was quenched with sat. aq. NH₄Cl and extracted with EtOAc. The crude product was purified by flash column chromatography to give tert-butyl (4-fluoro-2-methoxypyridin-3-yl)carbamate (232 mg, 43%) as a colorless oil. ([M+H]⁺ 243.2).

### Step 2: 4-fluoro-2-methoxypyridin-3-amine

tert-butyl (4-fluoro-2-methoxypyridin-3-yl)carbamate (230 mg, 949 μmol) was dissolved in HCl (4 M in dioxane, 13 ml, 52.2 mmol) and stirred for 20 h at room temperature before quenched with sat. aq. NaHCO₃ and diluted with EtOAc. The layers were separated and the organic phase was dried over Na₂SO₄, filtered and concentrated in vacuo to afford the crude title product (130 mg, 91%). (¹H NMR (CDCl₃, 300 MHz) δ 7.52 (dd, 1H, J=5.7, 8.0 Hz), 6.65 (dd, 1H, J=5.7, 9.4 Hz), 4.00 (s, 3H), 3.6-3.8 (m, 2H)).

### Step 3: 6-cyclopropyl-2-((4-fluoro-2-methoxypyridin-3-yl)amino)nicotinonitrile

The title compound ([M+H]⁺ 285.2) was prepared from 2-chloro-6-cyclopropylnicotinonitrile (CAS [1198475-35-2]) by reaction with 4-fluoro-2-methoxypyridin-3-amine hydrochloride using Pd(OAc)₂ as a catalyst and xantphos as a ligand (General procedure B1).

### Intermediate 61: 6-cyclopropyl-2-[(2-ethylpyrazol-3-yl)amino]pyridine-3-carbonitrile

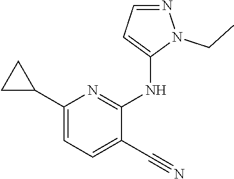 [SMILES: CCn1nccc1Nc1nc(C2CC2)ccc1C#N]

The title compound ([M+H]⁺ 254.3) was prepared from 2-chloro-6-cyclopropylnicotinonitrile (CAS [1198475-35-2]) by reaction with 5-amino-1-ethylpyrazole (CAS [3528-58-3]) using Pd(OAc)₂ as a catalyst and xantphos as a ligand (General procedure B1).

### Intermediate 62: 6-chloro-2-(o-tolylamino)nicotinonitrile

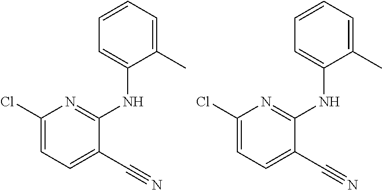

### Step 1: 6-chloro-2-(o-tolylamino)nicotinamide

2,6-dichloronicotinamide (1.03 g, 5.39 mmol), o-toluidine (867 mg, 8.09 mmol) and DIPEA (4.71 ml, 27 mmol) were dissolved in NMP (10 ml) and heated to 140° C. for 100 h. The crude mixture was quenched with water and extracted with EtOAc. The layers were separated and the organic phase was dried over Na₂SO₄, filtered and concentrated in vacuo. The crude product was purified by flash column chromatography to yield -chloro-2-(o-tolylamino)nicotinamide (1.00 g, 68%) as a white solid. ([M+H]⁺ 262.2)

### Step 2: 6-chloro-2-(o-tolylamino)nicotinonitrile

To a solution of 6-chloro-2-(o-tolylamino)nicotinamide (19 mg, 74.5 μmol) and pyridine (48.2 μl, 596 μmol) in acetonitrile (1 ml) at 0° C. was added POCl₃ (28 μl, 298 μmol) and the reaction mixture was stirred at 50° C. for 45 min. The reaction mixture was quenched with water, basified until pH 10 using 1M NaOH and extracted with EtOAc. The layers were separated and the organic phase was dried over Na₂SO₄, filtered and concentrated in vacuo. The crude product was purified by flash column chromatography to yield 6-chloro-2-(o-tolylamino)nicotinonitrile (14 mg, 73%) as a white solid. ([M+H]⁺ 244.2)

### Intermediate 63: 6-cyclopropyl-2-((2,3-dihydrobenzofuran-7-yl)amino)nicotinonitrile

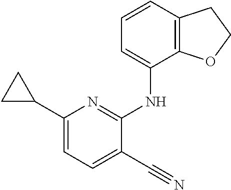 [SMILES: N#Cc1ccc(C2CC2)nc1Nc1cccc2c1OCC2]

The title compound ([M+H]⁺ 278.2) was prepared from 2-chloro-6-cyclopropylnicotinonitrile (CAS [1198475-35-2]) by reaction with 2,3-dihydrobenzofuran-7-amine (CAS [13414-56-7]) using Pd(OAc)₂ as a catalyst and xantphos as a ligand (General procedure B1).

### Intermediate 64: 2-((2-chlorophenyl)amino)-6-cyclopropylnicotinonitrile

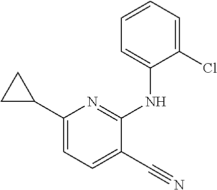 [SMILES: N#Cc1ccc(C2CC2)nc1Nc1ccccc1Cl]

The title compound ([M+H]⁺ 270.1) was prepared from 2-chloro-6-cyclopropylnicotinonitrile (CAS [1198475-35-2]) by reaction with 2-chloroaniline (CAS [95-51-2]) using Pd₂(dba)₃ as a catalyst and xantphos as a ligand (General procedure B1).

### Intermediate 65: 5-chloro-2-((3-cyano-2-methoxyphenyl)amino)-6-cyclopropylnicotinonitrile

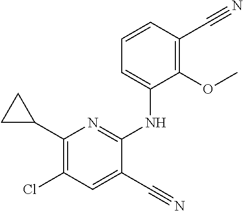 [SMILES: COc1c(C#N)cccc1Nc1nc(C2CC2)c(Cl)cc1C#N]

### Step 1: 2-amino-6-cyclopropylnicotinonitrile

The title compound ([M+H]⁺ 160.1) was prepared from 2-chloro-6-cyclopropylnicotinonitrile (CAS [1198475-35-2]) using General procedure D.

### Step 2: 2-amino-5-chloro-6-cyclopropylnicotinonitrile

The title compound ([M+H]⁺ 194.0) was prepared from 2-amino-6-cyclopropylnicotinonitrile and NCS in MeCN at 80° C. (General procedure E).

### Step 3: 5-chloro-2-((3-cyano-2-methoxyphenyl)amino)-6-cyclopropylnicotinonitrile

The title compound ([M+H]⁺ 325.2) was prepared from 2-amino-5-chloro-6-cyclopropylnicotinonitrile by reaction with 3-bromo-2-methoxybenzonitrile (CAS [874472-98-7]) using Pd₂(dba)₃ as a catalyst and xantphos as a ligand (General procedure F).

### Intermediate 66: 5-chloro-6-cyclopropyl-2-((2-methoxypyridin-3-yl)amino)nicotinonitrile

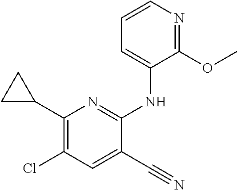 [SMILES: COc1ncccc1Nc1nc(C2CC2)c(Cl)cc1C#N]

### Step 1: 2-amino-6-cyclopropylnicotinonitrile

The title compound ([M+H]⁺ 160.1) was prepared from 2-chloro-6-cyclopropylnicotinonitrile (CAS [1198475-35-2]) using General procedure D.

### Step 2: 2-amino-5-chloro-6-cyclopropylnicotinonitrile

The title compound ([M+H]⁺ 194.0) was prepared from 2-amino-6-cyclopropylnicotinonitrile and NCS in MeCN at 80° C. (General procedure E).

### Step 3: 5-chloro-6-cyclopropyl-2-((2-methoxypyridin-3-yl)amino)nicotinonitrile

The title compound ([M+H]⁺ 301.2) was prepared from 2-amino-5-chloro-6-cyclopropylnicotinonitrile by reaction with 3-bromo-2-methoxypyridine (CAS [13472-59-8]) using Pd₂(dba)₃ as a catalyst and xantphos as a ligand (General procedure F).

### Intermediate 67: 6-cyclopropyl-2-(4-fluoro-2-methylanilino)pyridine-3-carbonitrile

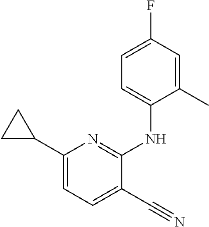 [SMILES: Cc1cc(F)ccc1Nc1nc(C2CC2)ccc1C#N]

The title compound ([M+H]⁺ 268.2) was prepared from 2-chloro-6-cyclopropylnicotinonitrile (CAS [1198475-35-2]) by reaction with 4-fluoro-2-methylaniline (CAS [452-71-1]) using Pd₂(dba)₃ as a catalyst and xantphos as a ligand (General procedure B1).

### Intermediate 68: 6-cyclopropyl-2-((3-ethylphenyl)amino)nicotinonitrile

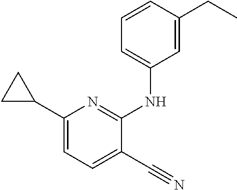 [SMILES: CCc1cccc(Nc2nc(C3CC3)ccc2C#N)c1]

The title compound ([M+H]⁺ 264.2) was prepared from 2-chloro-6-cyclopropylnicotinonitrile (CAS [1198475-35-2]) by reaction with 3-ethylaniline (CAS [587-02-0]) using Pd(OAc)₂ as a catalyst and xantphos as a ligand (General procedure B1).

### Intermediate 69: 6-cyclopropyl-2-(m-tolylamino)nicotinonitrile

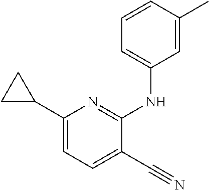 [SMILES: Cc1cccc(Nc2nc(C3CC3)ccc2C#N)c1]

The title compound ([M+H]⁺ 250.2) was prepared from 2-chloro-6-cyclopropylnicotinonitrile (CAS [1198475-35-2]) by reaction with 3-methylaniline (CAS [108-44-1]) using Pd(OAc)₂ as a catalyst and xantphos as a ligand (General procedure B1).

### Intermediate 70: 6-cyclopropyl-2-((3,5-difluorophenyl)amino)nicotinonitrile

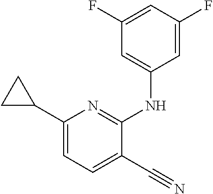 [SMILES: N#Cc1ccc(C2CC2)nc1Nc1cc(F)cc(F)c1]

The title compound ([M+H]⁺ 272.2) was prepared from 2-chloro-6-cyclopropylnicotinonitrile (CAS [1198475-35-2]) by reaction with 3,5-difluoroaniline (CAS [372-39-4]) using Pd(OAc)₂ as a catalyst and xantphos as a ligand (General procedure B1).

### Intermediate 71: 6-cyclopropyl-2-((3-methoxyphenyl)amino)nicotinonitrile

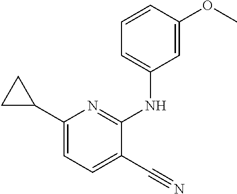 [SMILES: COc1cccc(Nc2nc(C3CC3)ccc2C#N)c1]

The title compound ([M+H]⁺ 266.2) was prepared from 2-chloro-6-cyclopropylnicotinonitrile (CAS [1198475-35-2]) by reaction with m-anisidine (CAS [536-90-3]) using Pd(OAc)₂ as a catalyst and xantphos as a ligand (General procedure B1).

### Intermediate 72: 6-cyclopropyl-2-((6-methoxypyridin-2-yl)amino)nicotinonitrile

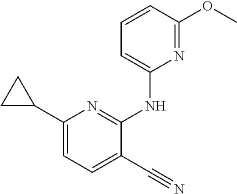 [SMILES: COc1cccc(Nc2nc(C3CC3)ccc2C#N)n1]

The title compound ([M+H]⁺ 267.2) was prepared from 2-chloro-6-cyclopropylnicotinonitrile (CAS [1198475-35-2]) by reaction with 2-amino-6-methoxypyridine (CAS [17920-35-3]) using Pd(OAc)₂ as a catalyst and xantphos as a ligand (General procedure B1).

### Intermediate 73: 6-cyclopropyl-2-(2,3-difluoroanilino)pyridine-3-carbonitrile

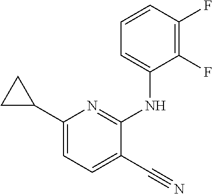 [SMILES: N#Cc1ccc(C2CC2)nc1Nc1cccc(F)c1F]

The title compound ([M+H]⁺ 272.2) was prepared from 2-chloro-6-cyclopropylnicotinonitrile (CAS [1198475-35-2]) by reaction with 2,3-difluoroaniline (CAS [4519-40-8]) using Pd₂(dba)₃ as a catalyst and xantphos as a ligand (General procedure B1).

### Intermediate 74: 6-cyclopropyl-2-(phenylamino)nicotinonitrile

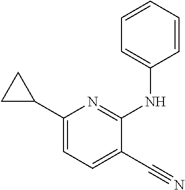 [SMILES: N#Cc1ccc(C2CC2)nc1Nc1ccccc1]

The title compound ([M+H]⁺ 236.3) was prepared from 2-chloro-6-cyclopropylnicotinonitrile (CAS [1198475-35-2]) by reaction with aniline (CAS [62-53-3]) using Pd(OAc)₂ as a catalyst and xantphos as a ligand (General procedure B1).

### Intermediate 75: 6-cyclopropyl-2-(1-oxazol-5-ylethylamino)pyridine-3-carbonitrile

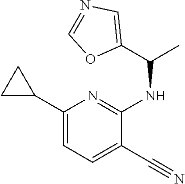

### Step 1: (R)-2-methyl-N—((R)-1-(oxazol-5-yl)ethyl)propane-2-sulfinamide

To a solution of 1-(oxazol-5-yl)ethan-1-one (150 mg, 1.35 mmol, CAS [1263378-07-9]) in THF (3 ml) (R)-2-methylpropane-2-sulfinamide (180 mg, 1.49 mmol) followed by titanium ethoxide (1.15 g, 4.05 mmol) were added. The resulting reaction mixture was heated to 60° C. for 2 h before it was cooled to −15° C. NaBH₄ (61.3 mg, 1.62 mmol) was added at −15° C. and the reaction mixture was stirred for 3 h at this temperature. The reaction was quenched with 1N HCl to ca. pH 5 and extracted with EtOAc. The combined organic layers were dried over Na₂SO₄, filtered and evaporated to dryness. The crude product was purified by flash column chromatography to afford (R)-2-methyl-N—((R)-1-(oxazol-5-yl)ethyl)propane-2-sulfinamide (156 mg, 53%) as light yellow oil. ([M+H]⁺ 217.2)

### Step 2: (R)-1-(oxazol-5-yl)ethan-1-amine hydrochloride

(R)-2-methyl-N—((R)-1-(oxazol-5-yl)ethyl)propane-2-sulfinamide (150 mg, 693 μmol) was dissolved in MeOH (3 ml) and HCl (4M in dioxane, 347 μl, 1.39 mmol) was added. The reaction mixture was stirred for 2 h at rt. The reaction mixture was evaporated to dryness, the crude product was suspended in Et₂O and the organic layer was removed. The remaining solid was dried under reduced pressure to afford (R)-1-(oxazol-5-yl)ethan-1-amine hydrochloride (84 mg, 81%) as a yellow gum. ([M+H]⁺ 113.1)

### Step: 6-cyclopropyl-2-(1-oxazol-5-ylethylamino)pyridine-3-carbonitrile

The title compound ([M+H]⁺ 255.2) was prepared from 2-chloro-6-cyclopropylnicotinonitrile (CAS [1198475-35-2]) by reaction with (R)-1-(oxazol-5-yl)ethan-1-amine hydrochloride using Pd(OAc)₂ as a catalyst and xantphos as a ligand (General procedure B1).

### Intermediate 76: 3-((2-cyano-5-(trifluoromethyl)phenyl)amino)-2-methylbenzonitrile

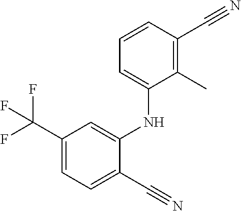 [SMILES: Cc1c(C#N)cccc1Nc1cc(C(F)(F)F)ccc1C#N]

The title compound ([M+H]⁺ 300.2) was prepared from 2-bromo-4-(trifluoromethyl)benzonitrile (CAS [35764-15-9]) by reaction with 3-amino-2-methylbenzonitrile (CAS [69022-35-1]) using Pd₂(dba)₃ as a catalyst and xantphos as a ligand (General procedure I1).

### Intermediate 77: 2-((3-cyano-2-methylphenyl)amino)-6-(trifluoromethyl)nicotinonitrile

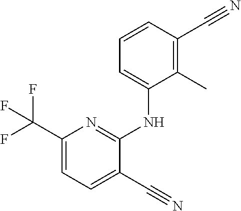 [SMILES: Cc1c(C#N)cccc1Nc1nc(C(F)(F)F)ccc1C#N]

The title compound ([M−H] 301.2) was prepared from 2-chloro-6-(trifluoromethyl)nicotinonitrile (CAS [386704-06-9]) by reaction with 3-amino-2-methylbenzonitrile (CAS [69022-35-1]) using Pd₂(dba)₃ as a catalyst and xantphos as a ligand (General procedure B1).

### Intermediate 78: 6-cyclopropyl-2-((2,6-difluorophenyl)amino)nicotinonitrile

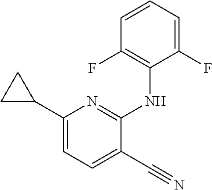 [SMILES: N#Cc1ccc(C2CC2)nc1Nc1c(F)cccc1F]

The title compound ([M+H]⁺ 272.2) was prepared from 2-chloro-6-cyclopropylnicotinonitrile (CAS [1198475-35-2]) by reaction with 2,6-difluoroaniline (CAS [5509-65-9]) using Pd(OAc)₂ as a catalyst and xphos as a ligand (General procedure B1).

### Intermediate 79: 6-cyclopropyl-2-((2-fluorophenyl)amino)nicotinonitrile

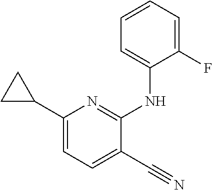 [SMILES: N#Cc1ccc(C2CC2)nc1Nc1ccccc1F]

The title compound ([M+H]⁺ 254.1) was prepared from 2-chloro-6-cyclopropylnicotinonitrile (CAS [1198475-35-2]) by reaction with 2-fluoroaniline (CAS [348-54-9]) using Pd(OAc)₂ as a catalyst and xphos as a ligand (General procedure B1).

### Intermediate 80: 6-cyclopropyl-2-((3-fluorophenyl)amino)nicotinonitrile

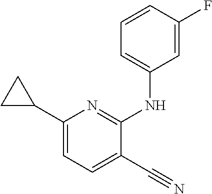 [SMILES: N#Cc1ccc(C2CC2)nc1Nc1cccc(F)c1]

The title compound ([M+H]⁺ 254.1) was prepared from 2-chloro-6-cyclopropylnicotinonitrile (CAS [1198475-35-2]) by reaction with 3-fluoroaniline (CAS [372-19-0]) using Pd(OAc)₂ as a catalyst and xphos as a ligand (General procedure B1).

### Intermediate 81: 6-cyclopropyl-2-[1-(oxetan-3-yl)ethylamino]pyridine-3-carbonitrile

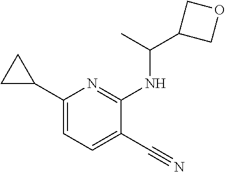 [SMILES: CC(Nc1nc(C2CC2)ccc1C#N)C1COC1]

The title compound ([M+H]⁺ 244.2) was prepared from 2-chloro-6-cyclopropylnicotinonitrile (CAS [1198475-35-2]) by reaction with 1-(oxetan-3-yl)ethan-1-amine (CAS [1544892-89-8]) using NMP as solvent, DIPEA as a base at 135° C./microwave (General procedure B2).

### Intermediate 82: 6-cyclopropyl-2-((1-(pyridin-2-yl)ethyl)amino)nicotinonitrile

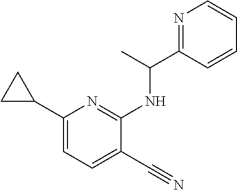 [SMILES: CC(Nc1nc(C2CC2)ccc1C#N)c1ccccn1]

The title compound ([M+H]⁺ 265.5) was prepared from 2-chloro-6-cyclopropylnicotinonitrile (CAS [1198475-35-2]) by reaction with 1-(pyridin-2-yl)ethan-1-amine (CAS [40154-81-2]) using NMP as solvent, TEA as a base at 135° C. (General procedure B2).

### Intermediate 83: 2-[(2-methyl-3-pyridyl)amino]-4-(2,2,2-trifluoroethyl)benzonitrile

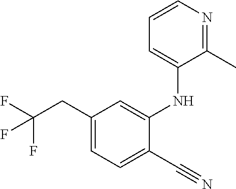 [SMILES: Cc1ncccc1Nc1cc(CC(F)(F)F)ccc1C#N]

### Step 1: 2-bromo-4-(2,2,2-trifluoroethyl)benzonitrile

To a solution of 2-bromo-4-(bromomethyl)benzonitrile (1.0 g, 3.64 mmol) in NMP (15 mL) was added methyl 2,2-difluoro-2-(fluorosulfonyl)acetate (1.4 g, 7.27 mmol) and copper(I) iodide (0.02 g, 0.730 mmol). The reaction mixture was purged with nitrogen 3 times and stirred under nitrogen atmosphere at 80° C. for 16 h. The resulting mixture was cooled, diluted with saturated aqueous NH₄Cl solution and extracted with DCM. The combined organic layers were washed with brine) and dried over Na₂SO₄. The solution was concentrated and purified by flash column chromatography to afford 2-bromo-4-(2,2,2-trifluoroethyl)benzonitrile (580 mg, 49%) as a white solid. ([M+H]⁺ 264.0)

### Step 2: 2-[(2-methyl-3-pyridyl)amino]-4-(2,2,2-trifluoroethyl)benzonitrile

The title compound ([M+H]⁺ 292.0) was prepared from 2-bromo-4-(2,2,2-trifluoroethyl)benzonitrile by reaction with 3-amino-2-methylpyridine (CAS [3430-10-2]) using using Pd(OAc)₂ as a catalyst and xantphos as a ligand (General procedure B1).

### Intermediate 84: 6-cyclopropyl-2-((tetrahydro-2H-pyran-4-yl)amino)nicotinonitrile

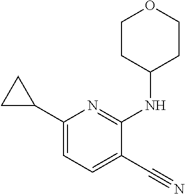 [SMILES: N#Cc1ccc(C2CC2)nc1NC1CCOCC1]

The title compound ([M+H]⁺ 244.2) was prepared from 2-chloro-6-cyclopropylnicotinonitrile (CAS [1198475-35-2]) by reaction with tetrahydro-2H-pyran-4-amine (CAS [38041-19-9]) using DMA as solvent, DIPEA as a base at 150° C./microwave (General procedure B2).

### Intermediate 85: 2-((2-chloropyridin-3-yl)amino)-6-cyclopropylnicotinonitrile

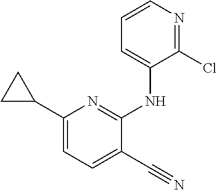 [SMILES: N#Cc1ccc(C2CC2)nc1Nc1cccnc1Cl]

### Step 1: 2-amino-6-cyclopropylnicotinonitrile

The title compound ([M+H]⁺ 160.1) was prepared from 2-chloro-6-cyclopropylnicotinonitrile (CAS [1198475-35-2]) using General procedure D.

### Step 2: 2-((2-chloropyridin-3-yl)amino)-6-cyclopropylnicotinonitrile

The title compound ([M+H]⁺ 271.1) was prepared from 2-amino-6-cyclopropylnicotinonitrile by reaction with 2-chloro-3-iodopyridine (CAS [78607-36-0]) using Pd₂(dba)₃ as a catalyst and xantphos as a ligand (General procedure B1).

### Intermediate 86: 4-cyclopropyl-2-[(2-methylpyridin-3-yl)amino]benzonitrile

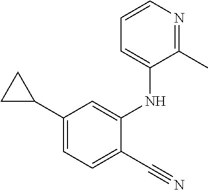 [SMILES: Cc1ncccc1Nc1cc(C2CC2)ccc1C#N]

The title compound ([M+H]⁺ 250.2) was prepared from 2-bromo-4-cyclopropylbenzonitrile (CAS [1237130-18-5]) by reaction with 3-amino-2-methylpyridine (CAS [3430-10-2]) at 120° C. using Pd(OAc)₂ as catalyst, DPPF as a ligand and KOtBu as a base in toluene (General procedure I1).

### Intermediate 87: 2-chloro-6-((2-methylpyridin-3-yl)amino)-4-(trifluoromethyl)benzonitrile

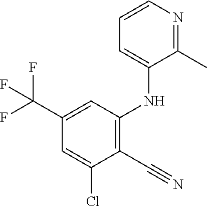 [SMILES: Cc1ncccc1Nc1cc(C(F)(F)F)cc(Cl)c1C#N]

The title compound ([M+H]⁺ 312.1) was prepared from 2,6-dichloro-4-(trifluoromethyl)benzonitrile (CAS [157021-61-9]) by reaction with 3-amino-2-methylpyridine (CAS [3430-10-2]) using Pd₂(dba)₃ as catalyst and xantphos as a ligand (General procedure I1).

### Intermediate 88: 6-cyclopropyl-2-(4-oxaspiro[2.5]octan-8-ylamino)pyridine-3-carbonitrile

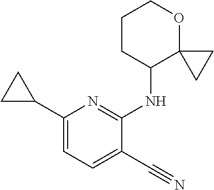 [SMILES: N#Cc1ccc(C2CC2)nc1NC1CCCOC12CC2]

### Step 1: methyl 1-(allyloxy)cyclopropane-1-carboxylate

To a solution of methyl 1-hydroxycyclopropane-1-carboxylate (10 g, 86.1 mmol) in THF (220 ml) at 0° C. was added sodium hydride (60% dispersion in mineral oil, 4.13 g, 103 mmol) over 30 min portion wise. Resulting yellow reaction mixture stirred for 15 min at 0° C. before a solution of allyl bromide (9.69 ml, 112 mmol) in THF (50 ml) was added over 30 min. The reaction mixture was stirred overnight at rt. It was cooled in an ice-bath and quenched with saturated NH₄Cl followed by addition of water and extracted with t-BME Combined organic layers were dried over Na₂SO₄, filtered and concentrated in vacuo at 20° C./100 mbar. The crude product was purified by vacuum distillation (short Vigreux-column) to afford methyl 1-(allyloxy)cyclopropane-1-carboxylate (6.25 g, 44%, bp: 79-82° C./12 mbar) as colorless oil. ([M+H]⁺ 157.1)

### Step 2: 1-(allyloxy)-N-methoxy-N-methylcyclopropane-1-carboxamide

To a suspension of N,O-dimethylhydroxylamine hydrochloride (6.56 g, 67.2 mmol) in DCM (63 ml) at 0° C. were added AlMe₃ (2 M in toluene, 33.6 ml, 67.2 mmol) over 45 min keeping temperature below 5° C. The resulting reaction mixture was stirred for 1 h at 0° C. before a solution of methyl 1-(allyloxy)cyclopropane-1-carboxylate (5.25 g, 33.6 mmol) in DCM (32 ml) was added over 30 min. The reaction solution was stirred overnight at rt and cooled in an ice-bath to carefully quench it with water (60 mL). HCl (4 M in water, ˜50 mL) were added and the reaction was stirred for 20 min. Reaction was diluted with DCM. After extraction with DCM, the organic layers were separated, dried over Na₂SO₄, filtered off and concentrated in vacuo at 20° C. The crude product was purified by flash column chromatography to afford 1-(allyloxy)-N-methoxy-N-methylcyclopropane-1-carboxamide (2.81 g, 45%) as colorless oil. ([M+H]⁺ 186.1)

### Step 3: 1-(1-(allyloxy)cyclopropyl)prop-2-en-1-one

To a solution of 1-(allyloxy)-N-methoxy-N-methylcyclopropane-1-carboxamide (3.11 g, 16.8 mmol) in THF (60 ml) at −75° C. vinylmagnesium bromide (1 M in THF, 35.3 ml, 35.3 mmol) was added over 15 minutes. The resulting yellow reaction mixture was stirred for 1 h at −75° C. and slowly warmed to 0° C. over 90 minutes. Reaction was cooled back to −75° C. and quenched with 4 N aqueous HCl followed by addition of water (100 mL). The reaction was repeatedly extracted with tBME and the combined organic layers were washed with brine, dried over Na₂SO₄, filtered and concentrated in vacuo at 20° C. to yield 1-(1-(allyloxy)cyclopropyl)prop-2-en-1-one (2.43 g, 91%) as a yellow oil. ([M+H]⁺ 153.2)

### Step 4: 4-oxaspiro[2.5]oct-6-en-8-one

To a solution of 1-(1-(allyloxy)cyclopropyl)prop-2-en-1-one (2.7 g, 17.7 mmol) in DCM (324 ml) was added zhan catalyst-1B (130 mg, 177 μmol) and resulting light greenish solution was stirred for 3 h at before it was concentrated in vacuo at 20° C. Crude product was purified by flash column chromatography to afford 4-oxaspiro[2.5]oct-6-en-8-one (1.85 g, 83%) as a colourless oil. ([M+H]⁺ 125.1)

### Step 5: (E)-4-oxaspiro[2.5]octan-8-one oxime

Step A: 4-oxaspiro[2.5]oct-6-en-8-one (520 mg, 4.19 mmol) was combined with 10% Pd/C (25 mg, 23.5 μmol) in MeOH (25 mL) at 20-25° C. for 30 min under a H₂ atmosphere (balloon). After completion the reaction was filtered over Decalite. Step B: Hydroxylamine-HCl (582 mg, 8.38 mmol) and KOAc (1.64 g, 16.8 mmol) were added to the reaction mixture obtained in step A and heated at 70° C. for 1 h. The reaction mixture was concentrated in vacuo and the product was isolated after extraction from water using EtOAc. The combined organic layers were dried over Na₂SO₄, filtered and concentrated in vacuo to afford (E)-4-oxaspiro[2.5]octan-8-one oxime (550 mg, 84%) as colorless oil which solidified on standing. ([M+H]⁺ 142.1)

### Step 6: 4-oxaspiro[2.5]octan-8-amine hydrochloride

(E)-4-oxaspiro[2.5]octan-8-one oxime (50 mg, 354 μmol) and Raney-Nickel (200 mg, 354 μmol) were combined in 7 M NH₃/MeOH at 25° C. under H₂ atmosphere (balloon). The reaction mixture was stirred for 75 min before catalyst was removed by filtration. Filtrate was evaporated in vacuo, the residue was dissolved in HCl (4 M in dioxane, 0.8 mL) and subsequently evaporated in vacuo.

White solid was suspended in MeCN/Et₂O, filtered off and washed with Et₂O. Filter cake was dried in vacuo at 45° C. to yield, 4-oxaspiro[2.5]octan-8-amine hydrochloride (37 mg, 61%) as a white solid. ([M-NH₄]⁺111.1) as white solid

### Step 7: 6-cyclopropyl-2-(4-oxaspiro[2.5]octan-8-ylamino)pyridine-3-carbonitrile

The title compound ([M+H]⁺ 270.3) was prepared from 2-chloro-6-cyclopropylnicotinonitrile (CAS [1198475-35-2]) by reaction with 4-oxaspiro[2.5]octan-8-amine hydrochloride using NMP as solvent, TEA as a base at 210° C. (General procedure B2).

### Intermediate 89: 6-cyclopropyl-2-[[rac-(2S,3S)-2-methyltetrahydrofuran-3-yl]amino]pyridine-3-carbonitrile

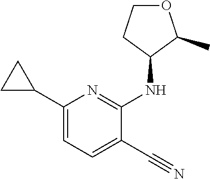 [SMILES: C[C@@H]1OCC[C@@H]1Nc1nc(C2CC2)ccc1C#N]

### Step 1: (cis)-N-benzyl-2-methyltetrahydrofuran-3-amine

To 2-methyldihydrofuran-3 (2H)-one (200 mg, 193 μl, 2 mmol, CAS [3188-00-9]) and benzylamine (235 mg, 240 μl, 2.2 mmol) in DCM (5 ml) were added acetic acid (144 mg, 137 μl, 2.4 mmol) and sodium triacetoxyborohydride (635 mg, 3 mmol) at 0° C. The reaction mixture was stirred at rt for 1 h before it was diluted with 1 M aqueous NaOH. The mixture was extracted twice with DCM, dried over Na₂SO₄ and concentrated under reduced pressure. The crude product was purified by flash column chromatography to afford (cis)-N-benzyl-2-methyltetrahydrofuran-3-amine (320 mg, 75%) as a yellow oil. ([M+H]⁺ 192.2)

### Step 2: cis-2-methyloxolan-3-amine

To (cis)-N-benzyl-2-methyltetrahydrofuran-3-amine (100 mg, 523 μmol) in THF (2 ml) was added acetic acid (59.9 μl, 1.05 mmol) and 10% Pd/C (111 mg, 105 μmol). H₂ was bubbled through the solution for 5 min and stirred under hydrogen atmosphere (balloon) for 2 h. The reaction mixture was filtered over Decalite and concentrated in vacuo to afford crude cis-2-methyloxolan-3-amine. (¹H NMR (DMSO-d6, 300 MHz) δ 3.8-3.9 (m, 1H), 3.7-3.8 (m, 1H), 3.53 (br d, 1H, J=6.0 Hz), 3.3-3.4 (m, 1H), 2.08 (s, 2H), 1.6-1.7 (m, 1H), 1.0-1.1 (m, 3H))

### Step 3: 6-cyclopropyl-2-[[(2SR,3SR)-2-methyltetrahydrofuran-3-yl]amino]pyridine-3-carbonitrile

The title compound ([M+H]⁺ 244.2) was prepared from 2-chloro-6-cyclopropylnicotinonitrile (CAS [1198475-35-2]) by reaction with cis-2-methyloxolan-3-amine using DMSO as solvent, DIPEA as a base at 120° C. (General procedure B2).

### Intermediate 90: 6-(7-azabicyclo[2.2.1]heptan-7-yl)-2-[(4-methylthiazol-5-yl)amino]pyridine-3-carbonitrile

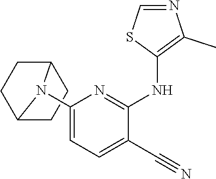 [SMILES: Cc1ncsc1Nc1nc(N2C3CCC2CC3)ccc1C#N]

### Step 1: 6-(7-azabicyclo[2.2.1]heptan-7-yl)-2-chloro-pyridine-3-carbonitrile

The title compound ([M+H]⁺ 234.2) was prepared from 2,6-dichloronicotinonitrile (CAS [40381-90-6]) and 7-azabicyclo[2.2.1]heptane hydrochloride (CAS [27514-07-4]) using DIPEA as a base in THF/ACN at rt (General procedure A2).

### Step 2: tert-butyl N-(4-methylthiazol-5-yl)carbamate

To a solution of 4-methylthiazole-5-carboxylic acid (600 mg, 4.19 mmol) in tBuOH (15 mL) were added TEA (2.34 mL, 16.76 mmol) and diphenylphosphorylazide (1.73 g, 6.29 mmol). The reaction mixture was stirred at 20° C. for 15 min and then heated to 80° C. for 2 h. The reaction mixture was poured into saturated aqueous NaHCO₃ solution (30 mL) and extracted with EtOAc. The combined organic layers were washed with brine, dried over Na₂SO₄, filtered and concentrated in vacuo to give crude tert-butyl N-(4-methylthiazol-5-yl)carbamate (400 mg, 44%) as a yellow solid. ([M+H]⁺ 215.1)

### Step 3: 4-methylthiazol-5-amine hydrochloride

A mixture of tert-butyl N-(4-methylthiazol-5-yl)carbamate (400.0 mg, 1.87 mmol) and HCl (4 M in dioxane, 2.0 mL, 8 mmol) in DCM (5 mL) was stirred at 25° C. for 12 h before it was concentrated in vacuo to crude give 4-methylthiazol-5-amine hydrochloride (200 mg, 71%) as a yellow solid. ([M+H]⁺ 115.1)

### Step 4: 6-(7-azabicyclo[2.2.1]heptan-7-yl)-2-[(4-methylthiazol-5-yl)amino]pyridine-3-carbonitrile

The title compound ([M+H]⁺ 312.1) was prepared from 6-(7-azabicyclo[2.2.1]heptan-7-yl)-2-chloro-pyridine-3-carbonitrile by reaction with 4-methylthiazol-5-amine hydrochloride using Pd(OAc)₂ as a catalyst and xantphos as a ligand (General procedure B1).

### EXAMPLES

| | | | Molecular | | |
|---|---|---|---|---|---|
| | | | weight | From | |
| Ex. | | | found | Inter- | |
| No. | Structure | Product Name | (M + H)⁺ | mediate | Prep. |
| 1 | 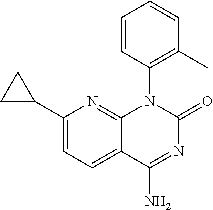 [SMILES: Cc1ccccc1-n1c(=O)nc(N)c2ccc(C3CC3)nc21] | 4-amino-7- cyclopropyl-1-(o- tolyl)pyrido[2,3- d]pyrimidin-2(1H)- one | 293.1 | 1 | C |
| 2 | 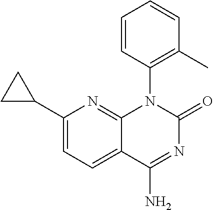 [SMILES: Cc1ccccc1-n1c(=O)nc(N)c2ccc(C3CC3)nc21] | 4-amino-7- cyclopropyl-1-(2- methoxyphenyl) pyrido[2,3- d]pyrimidin-2(1H)- one | 309.2 | 2 | C |
| 3 | 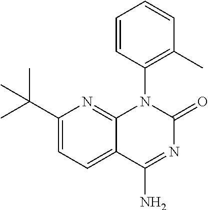 [SMILES: Cc1ccccc1-n1c(=O)nc(N)c2ccc(C(C)(C)C)nc21] | 4-amino-7-(tert- butyl)-1-(o- tolyl)pyrido[2,3- d]pyrimidin-2(1H)- one | 309.2 | 3 | C |
| 4 | 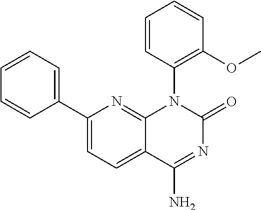 [SMILES: COc1ccccc1-n1c(=O)nc(N)c2ccc(-c3ccccc3)nc21] | 4-amino-1-(2- methoxyphenyl)-7- phenylpyrido[2,3- d]pyrimidin-2(1H)- one | 345.2 | 4 | C |
| 5 | 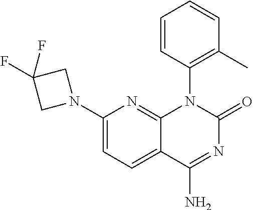 [SMILES: Cc1ccccc1-n1c(=O)nc(N)c2ccc(N3CC(F)(F)C3)nc21] | 4-amino-7-(3,3- difluoroazetidin-1- yl)-1-(o- tolyl)pyrido[2,3- d]pyrimidin-2(1H)- one | 344.1 | 5 | C |
| 6 | 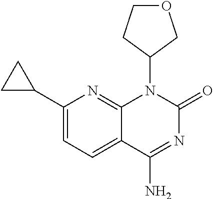 [SMILES: Nc1nc(=O)n(C2CCOC2)c2nc(C3CC3)ccc12] | 4-amino-7- cyclopropyl-1- (tetrahydrofuran-3- yl)pyrido[2,3- d]pyrimidin-2(1H)- one | 273.2 | 6 | C |
| 7 | 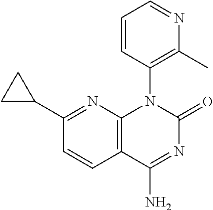 [SMILES: Cc1ncccc1-n1c(=O)nc(N)c2ccc(C3CC3)nc21] | 4-amino-7- cyclopropyl-1-(2- methylpyridin-3- yl)pyrido[2,3- d]pyrimidin-2(1H)- one | 294.2 | 7 | C |
| 8 | 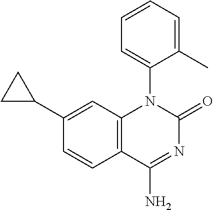 [SMILES: Cc1ccccc1-n1c(=O)nc(N)c2ccc(C3CC3)cc21] | 4-amino-7- cyclopropyl-1-(2- methylphenyl) quinazolin-2-one | 292.2 | 8 | C |
| 9 | 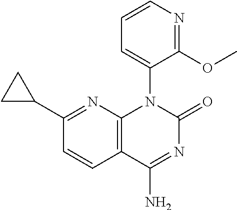 [SMILES: COc1ncccc1-n1c(=O)nc(N)c2ccc(C3CC3)nc21] | 4-amino-7- cyclopropyl-1-(2- methoxypyridin-3- yl)pyrido[2,3- d]pyrimidin-2(1H)- one | 310.2 | 9 | C |
| 10 | 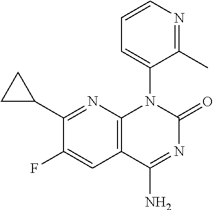 [SMILES: Cc1ncccc1-n1c(=O)nc(N)c2cc(F)c(C3CC3)nc21] | 4-amino-7- cyclopropyl-6- fluoro-1-(2- methylpyridin-3- yl)pyrido[2,3- d]pyrimidin-2(1H)- one | 312.3 | 10 | C |
| 11 | 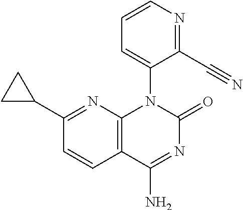 [SMILES: N#Cc1ncccc1-n1c(=O)nc(N)c2ccc(C3CC3)nc21] | 3-(4-amino-7- cyclopropyl-2- oxopyrido[2,3- d]pyrimidin-1(2H)- yl)picolinonitrile | 305.2 | 11 | C |
| 12 | 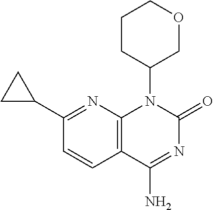 [SMILES: Nc1nc(=O)n(C2CCCOC2)c2nc(C3CC3)ccc12] | 4-amino-7- cyclopropyl-1- (oxan-3- yl)pyrido[2,3- d]pyrimidin-2-one | 287.2 | 12 | C |
| 13 | 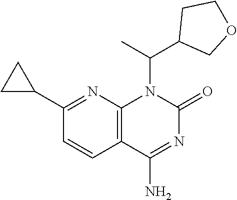 [SMILES: CC(C1CCOC1)n1c(=O)nc(N)c2ccc(C3CC3)nc21] | 4-amino-7- cyclopropyl-1-[1- (oxolan-3- yl)ethyl]pyrido[2,3- d]pyrimidin-2-one | 301.2 | 13 | C |
| 14 | 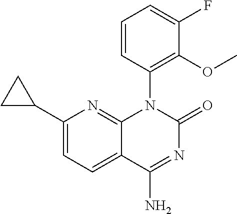 [SMILES: COc1c(F)cccc1-n1c(=O)nc(N)c2ccc(C3CC3)nc21] | 4-amino-7- cyclopropyl-1-(3- fluoro-2- methoxyphenyl) pyrido[2,3- d]pyrimidin-2(1H)- one | 327.2 | 14 | C |
| 15 | 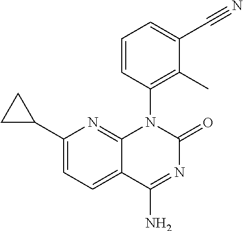 [SMILES: Cc1c(C#N)cccc1-n1c(=O)nc(N)c2ccc(C3CC3)nc21] | 3-(4-amino-7- cyclopropyl-2- oxopyrido[2,3- d]pyrimidin-1(2H)- yl)-2- methylbenzonitrile | 318.3 | 15 | C |
| 16 | 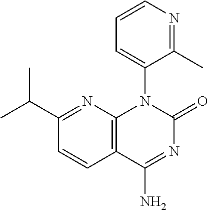 [SMILES: Cc1ncccc1-n1c(=O)nc(N)c2ccc(C(C)C)nc21] | 4-amino-1-(2- methylpyridin-3- yl)-7-propan-2- ylpyrido[2,3- d]pyrimidin-2-one | 296.2 | 16 | C |
| 17 | 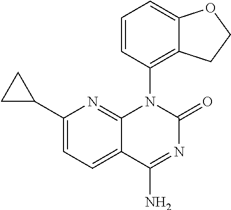 [SMILES: Nc1nc(=O)n(-c2cccc3c2CCO3)c2nc(C3CC3)ccc12] | 4-amino-7- cyclopropyl-1-(2,3- dihydrobenzofuran- 4-yl)pyrido[2,3- d]pyrimidin-2(1H)- one | 321.2 | 17 | C |
| 18 | 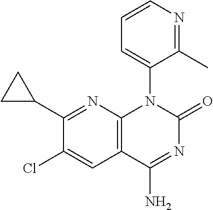 [SMILES: Cc1ncccc1-n1c(=O)nc(N)c2cc(Cl)c(C3CC3)nc21] | 4-amino-6-chloro- 7-cyclopropyl-1-(2- methylpyridin-3- yl)pyrido[2,3- d]pyrimidin-2(1H)- one | 326.2 | 18 | C |
| 19 | 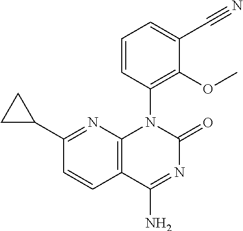 [SMILES: COc1c(C#N)cccc1-n1c(=O)nc(N)c2ccc(C3CC3)nc21] | 3-(4-amino-7- cyclopropyl-2- oxopyrido[2,3- d]pyrimidin-1(2H)- yl)-2- methoxybenzonitrile | 334.3 | 19 | C |
| 20 | 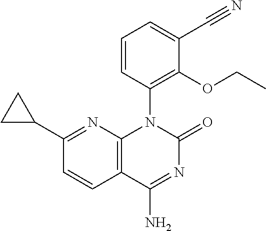 [SMILES: CCOc1c(C#N)cccc1-n1c(=O)nc(N)c2ccc(C3CC3)nc21] | 3-(4-amino-7- cyclopropyl-2- oxopyrido[2,3- d]pyrimidin-1(2H)- yl)-2- ethoxybenzonitrile | 348.3 | 20 | C |
| 21 | 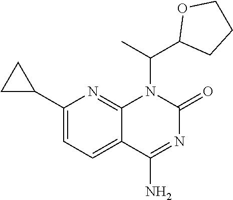 [SMILES: CC(C1CCCO1)n1c(=O)nc(N)c2ccc(C3CC3)nc21] | 4-amino-7- cyclopropyl-1-(1- (tetrahydrofuran-2- yl)ethyl)pyrido[2,3- d]pyrimidin- 2(1H)-one | 301.3 | 21 | C |
| 22 | 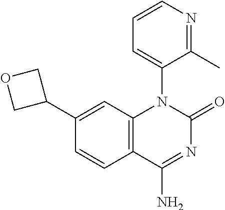 [SMILES: Cc1ncccc1-n1c(=O)nc(N)c2ccc(C3COC3)cc21] | 4-amino-1-(2- methylpyridin-3- yl)-7-(oxetan-3- yl)quinazolin- 2(1H)-one | 307.3 | 22 | C |
| 23 | 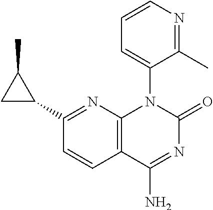 [SMILES: Cc1ncccc1-n1c(=O)nc(N)c2ccc([C@@H]3C[C@H]3C)nc21] | 4-amino-7- ((1RS,2RS)-2- methylcyclopropyl)- 1-(2- methylpyridin-3- yl)pyrido[2,3- d]pyrimidin-2(1H)- one | 308.3 | 23 | C |
| 24 | 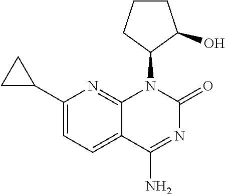 [SMILES: Nc1nc(=O)n([C@H]2CCC[C@H]2O)c2nc(C3CC3)ccc12] | 4-amino-7- cyclopropyl-1- ((1SR,2RS)-2- hydroxycyclopentyl) pyrido[2,3- d]pyrimidin-2(1H)- one | 287.3 | 24 | C |
| 25 | 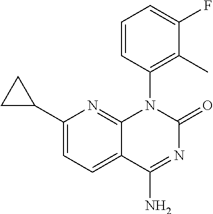 [SMILES: Cc1c(F)cccc1-n1c(=O)nc(N)c2ccc(C3CC3)nc21] | 4-amino-7- cyclopropyl-1-(3- fluoro-2- methylphenyl)pyrido [2,3-d]pyrimidin- 2(1H)-one | 311.2 | 25 | C |
| 26 | 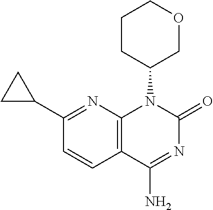 [SMILES: Nc1nc(=O)n([C@@H]2CCCOC2)c2nc(C3CC3)ccc12] | 4-amino-7- cyclopropyl-1- [(3R)-oxan-3- yl]pyrido[2,3- d]pyrimidin-2-one | 287.2 | 26 | C |
| 27 | 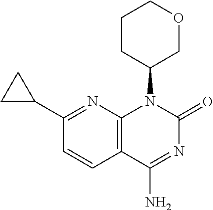 [SMILES: Nc1nc(=O)n([C@H]2CCCOC2)c2nc(C3CC3)ccc12] | 4-amino-7- cyclopropyl-1- [(3S)-oxan-3- yl]pyrido[2,3- d]pyrimidin-2-one | 287.2 | 27 | C |
| 28 | 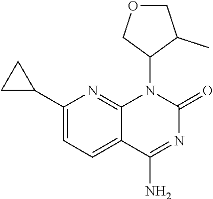 [SMILES: CC1COCC1n1c(=O)nc(N)c2ccc(C3CC3)nc21] | 4-amino-7- cyclopropyl-1-(4- methyltetrahydro- furan-3- yl)pyrido[2,3- d]pyrimidin-2(1H)- one | 287.2 | 28 | C |
| 29 | 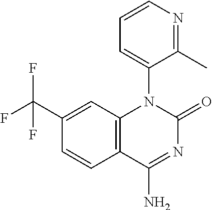 [SMILES: Cc1ncccc1-n1c(=O)nc(N)c2ccc(C(F)(F)F)cc21] | 4-amino-1-(2- methylpyridin-3- yl)-7- (trifluoromethyl) quinazolin-2-one | 321.2 | 29 | C |
| 30 | 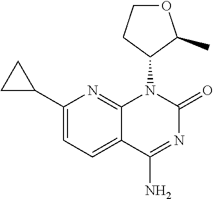 [SMILES: C[C@@H]1OCC[C@H]1n1c(=O)nc(N)c2ccc(C3CC3)nc21] | 4-amino-7- cyclopropyl-1-[rac- (2RS,3SR)-2- methyloxolan-3- yl]pyrido[2,3- d]pyrimidin-2-one | 287.3 | 30 | C |
| 31 | 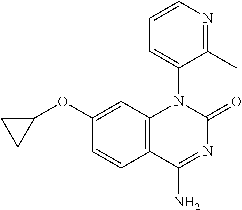 [SMILES: Cc1ncccc1-n1c(=O)nc(N)c2ccc(OC3CC3)cc21] | 4-amino-7- cyclopropyloxy-1- (2-methylpyridin- 3-yl)quinazolin-2- one | 309.2 | 31 | C |
| 32 | 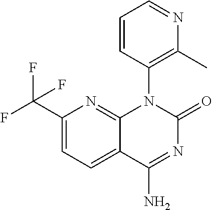 [SMILES: Cc1ncccc1-n1c(=O)nc(N)c2ccc(C(F)(F)F)nc21] | 4-amino-1-(2- methylpyridin-3- yl)-7- (trifluoromethyl) pyrido[2,3- d]pyrimidin-2(1H)- one | 322.3 | 32 | C |
| 33 | 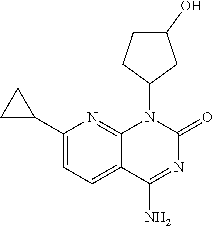 [SMILES: Nc1nc(=O)n(C2CCC(O)C2)c2nc(C3CC3)ccc12] | 4-amino-7- cyclopropyl-1-(3- hydroxycyclopentyl) pyrido[2,3- d]pyrimidin-2(1H)- one | 287.2 | 33 | C |
| 34 | 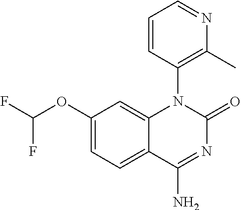 [SMILES: Cc1ncccc1-n1c(=O)nc(N)c2ccc(OC(F)F)cc21] | 4-amino-7- (difluoromethoxy)- 1-2- methylpyridin-3- yl)quinazolin- 2(1H)-one | 319.2 | 34 | C |
| 35 | 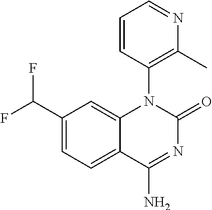 [SMILES: Cc1ncccc1-n1c(=O)nc(N)c2ccc(C(F)F)cc21] | 4-amino-7- (difluoromethyl)-1- (2-methylpyridin- 3-yl)quinazolin- 2(1H)-one | 303.2 | 35 | C |
| 36 | 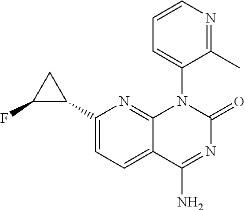 [SMILES: Cc1ncccc1-n1c(=O)nc(N)c2ccc([C@H]3C[C@@H]3F)nc21] | 4-amino-7- [(1RS,2SR)-2- fluorocyclopropyl]- 1-(2-methyl-3- pyridyl)pyrido[2,3- d]pyrimidin-2-one | 312.0 | 36 | C |
| 37 | 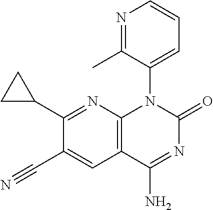 [SMILES: Cc1ncccc1-n1c(=O)nc(N)c2cc(C#N)c(C3CC3)nc21] | 4-amino-7- cyclopropyl-1-(2- methylpyridin-3- yl)-2-oxo-1,2- dihydropyrido[2,3- d]pyrimidine-6- carbonitrile | 319.3 | 37 | C |
| 38 | 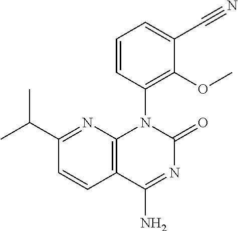 [SMILES: COc1c(C#N)cccc1-n1c(=O)nc(N)c2ccc(C(C)C)nc21] | 3-(4-amino-7- isopropyl-2- oxopyrido[2,3- d]pyrimidin-1(2H)- yl)-2- methoxybenzonitrile | 336.3 | 38 | C |
| 39 | 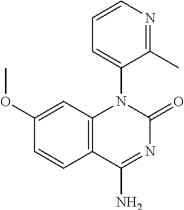 [SMILES: COc1ccc2c(N)nc(=O)n(-c3cccnc3C)c2c1] | 4-amino-7- methoxy-1-(2- methylpyridin-3- yl)quinazolin- 2(1H)-one | 283.2 | 39 | C |
| 40 | 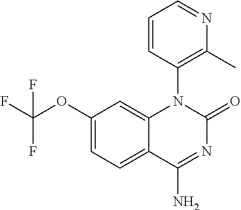 [SMILES: Cc1ncccc1-n1c(=O)nc(N)c2ccc(OC(F)(F)F)cc21] | 4-amino-1-(2- methylpyridin-3- yl)-7- (trifluoromethoxy) quinazolin-2(1H)- one | 337.2 | 40 | C |
| 41 | 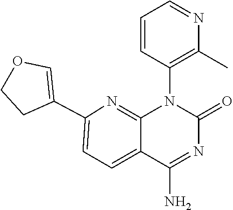 [SMILES: Cc1ncccc1-n1c(=O)nc(N)c2ccc(C3=COCC3)nc21] | 4-amino-7-(4,5- dihydrofuran-3-yl)- 1-(2- methylpyridin-3- yl)pyrido[2,3- d]pyrimidin-2(1H)- one | 322.2 | 41 | C |
| 42 | 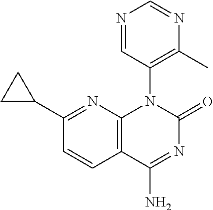 [SMILES: Cc1ncncc1-n1c(=O)nc(N)c2ccc(C3CC3)nc21] | 4-amino-7- cyclopropyl-1-(4- methylpyrimidin-5- yl)pyrido[2,3- d]pyrimidin-2(1H)- one | 295.2 | 42 | C |
| 43 | 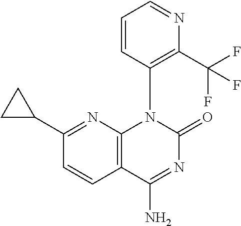 [SMILES: Nc1nc(=O)n(-c2cccnc2C(F)(F)F)c2nc(C3CC3)ccc12] | 4-amino-7- cyclopropyl-1-(2- (trifluoromethyl) pyridin-3- yl)pyrido[2,3- d]pyrimidin-2(1H)- one | 348.2 | 43 | C |
| 44 | 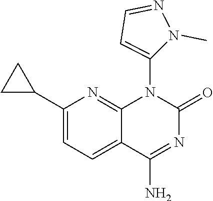 [SMILES: Cn1nccc1-n1c(=O)nc(N)c2ccc(C3CC3)nc21] | 4-amino-7- cyclopropyl-1-(2- methylpyrazol-3- yl)pyrido[2,3- d]pyrimidin-2-one | 283.2 | 44 | C |
| 45 | 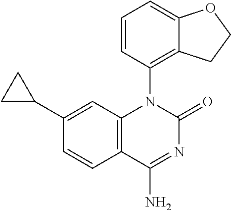 [SMILES: Nc1nc(=O)n(-c2cccc3c2CCO3)c2cc(C3CC3)ccc12] | 4-amino-7- cyclopropyl-1-(2,3- dihydrobenzofuran- 4-yl)quinazolin- 2(1H)-one | 320.2 | 45 | C |
| 46 | 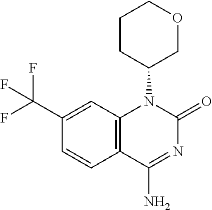 [SMILES: Nc1nc(=O)n([C@@H]2CCCOC2)c2cc(C(F)(F)F)ccc12] | (R)-4-amino-1- (tetrahydro-2H- pyran-3-yl)-7- (trifluoromethyl) quinazolin-2(1H)-one | 314.2 | 46 | C |
| 47 | 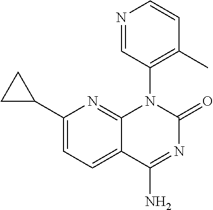 [SMILES: Cc1ccncc1-n1c(=O)nc(N)c2ccc(C3CC3)nc21] | 4-amino-7- cyclopropyl-1-(4- methylpyridin-3- yl)pyrido[2,3- d]pyrimidin-2(1H)- one | 294.2 | 47 | C |
| 48 | 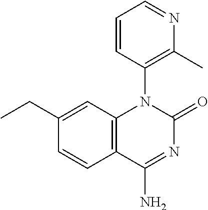 [SMILES: CCc1ccc2c(N)nc(=O)n(-c3cccnc3C)c2c1] | 4-amino-7-ethyl-1- (2-methylpyridin- 3-yl)quinazolin- 2(1H)-one | 281.2 | 48 | C |
| 49 | 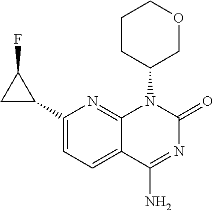 [SMILES: Nc1nc(=O)n([C@@H]2CCCOC2)c2nc([C@@H]3C[C@H]3F)ccc12] | 4-amino-7- [(1SR,2RS)-2- fluorocyclopropyl]- 1-[(3R)- tetrahydropyran-3- yl]pyrido[2,3- d]pyrimidin-2-one | 305.1 | 49 | C |
| 50 | 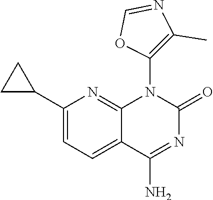 [SMILES: Cc1ncoc1-n1c(=O)nc(N)c2ccc(C3CC3)nc21] | 4-amino-7- cyclopropyl-1-(4- methyloxazol-5- yl)pyrido[2,3- d]pyrimidin-2-one | 284.1 | 50 | C |
| 51 | 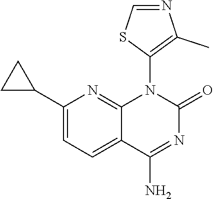 [SMILES: Cc1ncsc1-n1c(=O)nc(N)c2ccc(C3CC3)nc21] | 4-amino-7- cyclopropyl-1-(4- methylthiazol-5- yl)pyrido[2,3- d]pyrimidin-2-one | 299.9 | 51 | C |
| 52 | 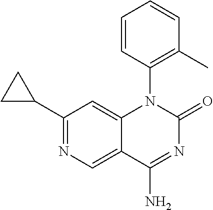 [SMILES: Cc1ccccc1-n1c(=O)nc(N)c2cnc(C3CC3)cc21] | 4-amino-7- cyclopropyl-1-(o- tolyl)pyrido[4,3- d]pyrimidin-2(1H)- one | 293.2 | 52 | C |
| 53 | 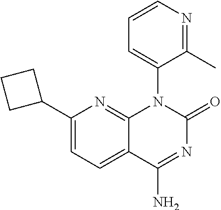 [SMILES: Cc1ncccc1-n1c(=O)nc(N)c2ccc(C3CCC3)nc21] | 4-amino-7- cyclobutyl-1-(2- methyl-3- pyridyl)pyrido[2,3- d]pyrimidin-2-one | 308.2 | 53 | C |
| 54 | 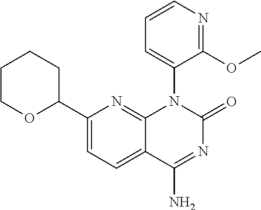 [SMILES: COc1ncccc1-n1c(=O)nc(N)c2ccc(C3CCCCO3)nc21] | 4-amino-1-(2- methoxy-3- pyridyl)-7- tetrahydropyran-2- yl-pyrido[2,3- d]pyrimidin-2-one | 354.2 | 54 | C |
| 55 | 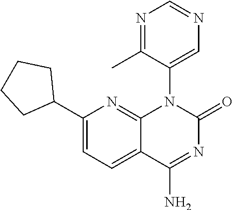 [SMILES: Cc1ncncc1-n1c(=O)nc(N)c2ccc(C3CCCC3)nc21] | 4-amino-7- cyclopentyl-1-(4- methylpyrimidin-5- yl)pyrido[2,3- d]pyrimidin-2-one | 323.2 | 55 | C |
| 56 | 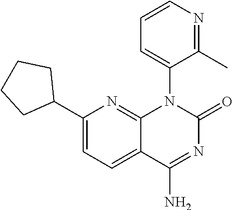 [SMILES: Cc1ncccc1-n1c(=O)nc(N)c2ccc(C3CCCC3)nc21] | 4-amino-7- cyclopentyl-1-(2- methyl-3- pyridyl)pyrido[2,3- d]pyrimidin-2-one | 322.2 | 56 | C |
| 57 | 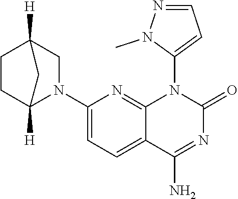 [SMILES: Cn1nccc1-n1c(=O)nc(N)c2ccc(N3C[C@H]4CC[C@@H]3C4)nc21] | 4-amino-7- [(1SR,4RS)-3- azabicyclo[2.2.1] heptan-3-yl]-1-(2- methylpyrazol-3- yl)pyrido[2,3- d]pyrimidin-2-one | 338.1 | 57 | C |
| 58 | 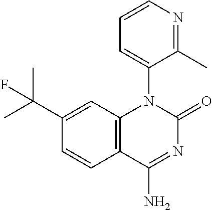 [SMILES: Cc1ncccc1-n1c(=O)nc(N)c2ccc(C(C)(C)F)cc21] | 4-amino-7-(2- fluoropropan-2-yl)- 1-(2- methylpyridin-3- yl)quinazolin-2-one | 311.3 | 58 | C |
| 59 | 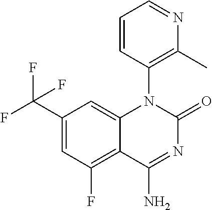 [SMILES: Cc1ncccc1-n1c(=O)nc(N)c2c(F)cc(C(F)(F)F)cc21] | 4-amino-5-fluoro- 1-(2- methylpyridin-3- yl)-7- (trifluoromethyl) quinazolin-2(1H)-one | 339.1 | 59 | C |
| 60 | 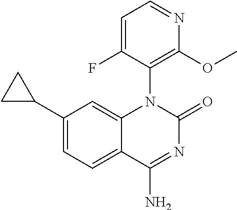 [SMILES: COc1nccc(F)c1-n1c(=O)nc(N)c2ccc(C3CC3)cc21] | 4-amino-7- cyclopropyl-1-(4- fluoro-2- methoxypyridin-3- yl)pyrido[2,3- d]pyrimidin-2(1H)- one | 328.2 | 60 | C |
| 61 | 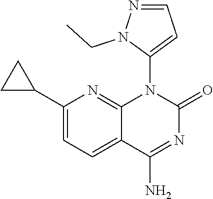 [SMILES: CCn1nccc1-n1c(=O)nc(N)c2ccc(C3CC3)nc21] | 4-amino-7- cyclopropyl-1-(1- ethyl-1H-pyrazol- 5-yl)pyrido[2,3- d]pyrimidin-2(1H)- one | 297.2 | 61 | C |
| 62 | 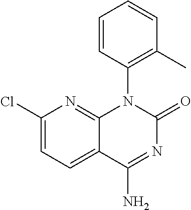 [SMILES: Cc1ccccc1-n1c(=O)nc(N)c2ccc(Cl)nc21] | 4-amino-7-chloro- 1-(o- tolyl)pyrido[2,3- d]pyrimidin-2(1H)- one | 287.2 | 62 | C |
| 63 | 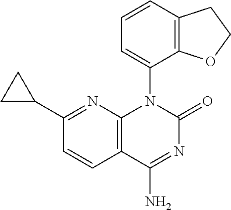 [SMILES: Nc1nc(=O)n(-c2cccc3c2OCC3)c2nc(C3CC3)ccc12] | 4-amino-7- cyclopropyl-1-(2,3- dihydrobenzofuran- 7-yl)pyrido[2,3- d]pyrimidin-2(1H)- one | 321.3 | 63 | C |
| 64 | 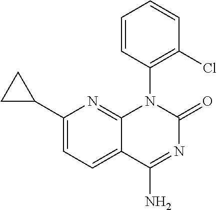 [SMILES: Nc1nc(=O)n(-c2ccccc2Cl)c2nc(C3CC3)ccc12] | 4-amino-1-(2- chlorophenyl)-7- cyclopropylpyrido [2,3-d]pyrimidin-2- one | 313.1 | 64 | C |
| 65 | 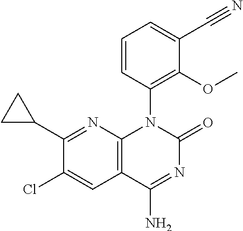 [SMILES: COc1c(C#N)cccc1-n1c(=O)nc(N)c2cc(Cl)c(C3CC3)nc21] | 3-(4-amino-6- chloro-7- cyclopropyl-2- oxopyrido[2,3- d]pyrimidin-1(2H)- yl)-2- methoxybenzonitrile | 368.2 | 65 | C |
| 66 | 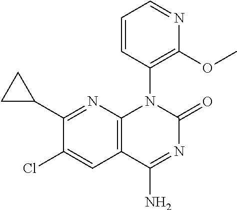 [SMILES: COc1ncccc1-n1c(=O)nc(N)c2cc(Cl)c(C3CC3)nc21] | 4-amino-6-chloro- 7-cyclopropyl-1-(2- methoxypyridin-3- yl)pyrido[2,3- d]pyrimidin-2(1H)- one | 344.2 | 66 | C |
| 67 | 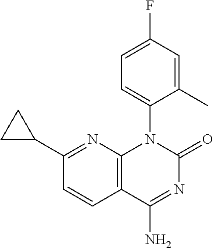 [SMILES: Cc1cc(F)ccc1-n1c(=O)nc(N)c2ccc(C3CC3)nc21] | 4-amino-7- cyclopropyl-1-(4- fluoro-2- methylphenyl)pyrido [2,3-d]pyrimidin- 2-one | 311.1 | 67 | C |
| 68 | 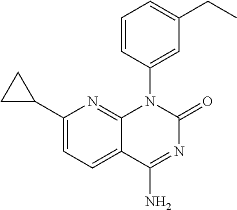 [SMILES: CCc1cccc(-n2c(=O)nc(N)c3ccc(C4CC4)nc32)c1] | 4-amino-7- cyclopropyl-1-(3- ethylphenyl)pyrido [2,3-d]pyrimidin- 2(1H)-one | 307.2 | 68 | C |
| 69 | 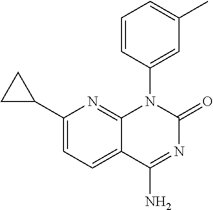 [SMILES: Cc1cccc(-n2c(=O)nc(N)c3ccc(C4CC4)nc32)c1] | 4-amino-7- cyclopropyl-1-(m- tolyl)pyrido[2,3- d]pyrimidin-2(1H)- one | 293.2 | 69 | C |
| 70 | 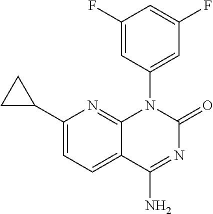 [SMILES: Nc1nc(=O)n(-c2cc(F)cc(F)c2)c2nc(C3CC3)ccc12] | 4-amino-7- cyclopropyl-1-(3,5- difluorophenyl) pyrido[2,3- d]pyrimidin-2(1H)- one | 315.1 | 70 | C |
| 71 | 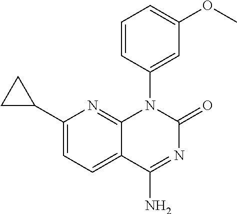 [SMILES: COc1cccc(-n2c(=O)nc(N)c3ccc(C4CC4)nc32)c1] | 4-amino-7- cyclopropyl-1-(3- methoxyphenyl) pyrido[2,3- d]pyrimidin-2(1H)- one | 309.2 | 71 | C |
| 72 | 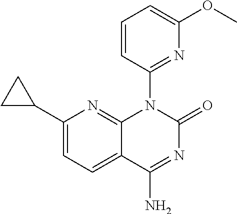 [SMILES: COc1cccc(-n2c(=O)nc(N)c3ccc(C4CC4)nc32)n1] | 4-amino-7- cyclopropyl-1-(6- methoxypyridin-2- yl)pyrido[2,3- d]pyrimidin-2(1H)- one | 310.2 | 72 | C |
| 73 | 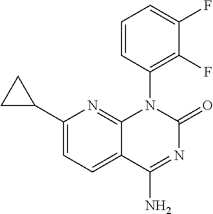 [SMILES: Nc1nc(=O)n(-c2cccc(F)c2F)c2nc(C3CC3)ccc12] | 4-amino-7- cyclopropyl-1-(2,3- difluorophenyl) pyrido[2,3- d]pyrimidin-2(1H)- one | 315.2 | 73 | C |
| 74 | 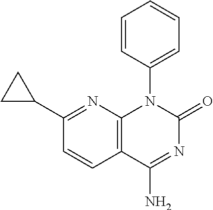 [SMILES: Nc1nc(=O)n(-c2ccccc2)c2nc(C3CC3)ccc12] | 4-amino-7- cyclopropyl-1- phenylpyrido[2,3- d]pyrimidin-2(1H)- one | 279.2 | 74 | C |
| 75 | 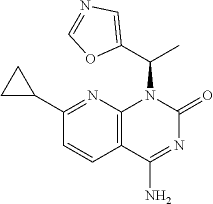 [SMILES: C[C@H](c1cnco1)n1c(=O)nc(N)c2ccc(C3CC3)nc21] | 4-amino-7- cyclopropyl-1-(1- (oxazol-5- yl)ethyl)pyrido[2,3-d] pyrimidin- 2(1H)-one | 298.2 | 75 | C |
| 76 | 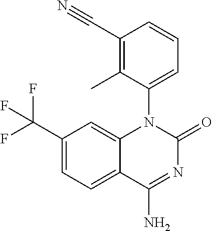 [SMILES: Cc1c(C#N)cccc1-n1c(=O)nc(N)c2ccc(C(F)(F)F)cc21] | 3-(4-amino-2-oxo- 7- (trifluoromethyl) quinazolin-1(2H)-yl)- 2- methylbenzonitrile | 345.2 | 76 | C |
| 77 | 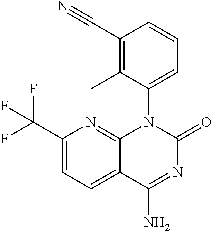 [SMILES: Cc1c(C#N)cccc1-n1c(=O)nc(N)c2ccc(C(F)(F)F)nc21] | 3-(4-amino-2-oxo- 7- (trifluoromethyl) pyrido[2,3- d]pyrimidin-1(2H)- yl)-2- methylbenzonitrile | 346.2 | 77 | C |
| 78 | 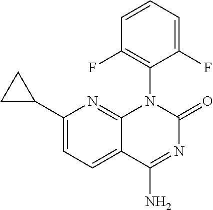 [SMILES: Nc1nc(=O)n(-c2c(F)cccc2F)c2nc(C3CC3)ccc12] | 4-amino-7- cyclopropyl-1-(2,6- difluorophenyl) pyrido[2,3- d]pyrimidin-2(1H)- one | 315.1 | 78 | C |
| 79 | 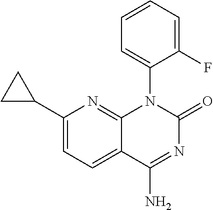 [SMILES: Nc1nc(=O)n(-c2ccccc2F)c2nc(C3CC3)ccc12] | 4-amino-7- cyclopropyl-1-(2- fluorophenyl) pyrido[2,3-d]pyrimidin- 2(1H)-one | 297.1 | 79 | C |
| 80 | 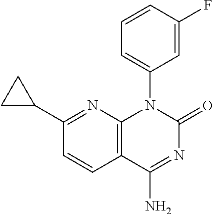 [SMILES: Nc1nc(=O)n(-c2cccc(F)c2)c2nc(C3CC3)ccc12] | 4-amino-7- cyclopropyl-1-(3- fluorophenyl) pyrido[2,3-d]pyrimidin- 2(1H)-one | 297.2 | 80 | C |
| 81 | 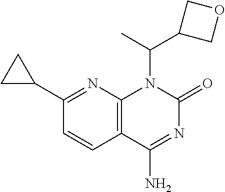 [SMILES: CC(C1COC1)n1c(=O)nc(N)c2ccc(C3CC3)nc21] | 4-amino-7- cyclopropyl-1-[1- (oxetan-3- yl)ethyl]pyrido[2,3- d]pyrimidin-2-one | 287.2 | 81 | C |
| 82 | 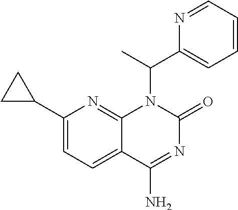 [SMILES: CC(c1ccccn1)n1c(=O)nc(N)c2ccc(C3CC3)nc21] | 4-amino-7- cyclopropyl-1-(1- pyridin-2- ylethyl)pyrido[2,3- d]pyrimidin-2-one | 308.2 | 82 | C |
| 83 | 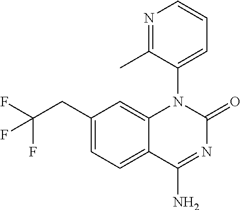 [SMILES: Cc1ncccc1-n1c(=O)nc(N)c2ccc(CC(F)(F)F)cc21] | 4-amino-1-(2- methyl-3-pyridyl)- 7-(2,2,2- trifluoroethyl) quinazolin-2-one hydrochloride | 335.0 | 83 | C |
| 84 | 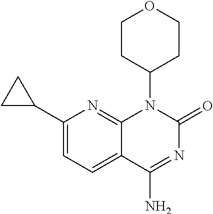 [SMILES: Nc1nc(=O)n(C2CCOCC2)c2nc(C3CC3)ccc12] | 4-amino-7- cyclopropyl-1- (tetrahydro-2H- pyran-4- yl)pyrido[2,3- d]pyrimidin-2(1H)- one | 287.2 | 84 | C |
| 85 | 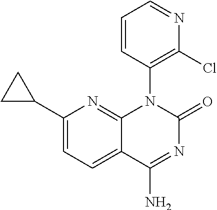 [SMILES: Nc1nc(=O)n(-c2cccnc2Cl)c2nc(C3CC3)ccc12] | 4-amino-1-(2- chloropyridin-3- yl)-7- cyclopropylpyrido [2,3-d]pyrimidin- 2(1H)-one | 314.2 | 85 | C |
| 86 | 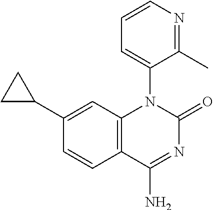 [SMILES: Cc1ncccc1-n1c(=O)nc(N)c2ccc(C3CC3)cc21] | 4-amino-7- cyclopropyl-1-(2- methylpyridin-3- yl)quinazolin-2-one | 293.2 | 86 | C |
| 87 | 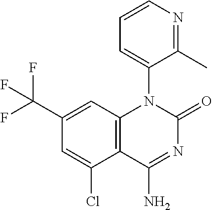 [SMILES: Cc1ncccc1-n1c(=O)nc(N)c2c(Cl)cc(C(F)(F)F)cc21] | 4-amino-5-chloro- 1-(2- methylpyridin-3- yl)-7- (trifluoromethyl) quinazolin-2(1H)-one | 355.1 | 87 | C |
| 88 | 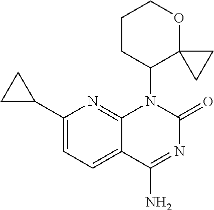 [SMILES: Nc1nc(=O)n(C2CCCOC23CC3)c2nc(C3CC3)ccc12] | 4-amino-7- cyclopropyl-1-(4- oxaspiro[2.5]octan- 8-yl)pyrido[2,3- d]pyrimidin-2(1H)- one | 313.3 | 88 | C |
| 89 | 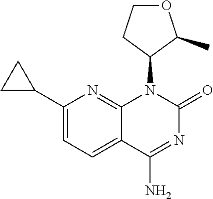 [SMILES: C[C@@H]1OCC[C@@H]1n1c(=O)nc(N)c2ccc(C3CC3)nc21] | 4-amino-7- cyclopropyl-1- ((cis)-2- methyltetrahydrofuran- 3- yl)pyrido[2,3- d]pyrimidin-2(1H)- one | 287.2 | 89 | C |
| 90 | 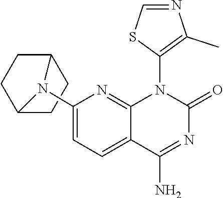 [SMILES: Cc1ncsc1-n1c(=O)nc(N)c2ccc(N3C4CCC3CC4)nc21] | 4-amino-7-(7- azabicyclo[2.2.1] heptan-7-yl)-1-(4- methylthiazol-5- yl)pyrido[2,3- d]pyrimidin-2-one | 354.9 | 90 | C |

### Example 91: 4-amino-7-(difluoromethoxy)-1-(2-methylphenyl)pyrido[2,3-d]pyrimidin-2-one

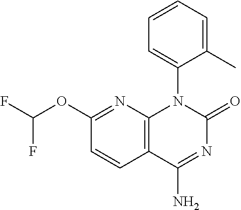 [SMILES: Cc1ccccc1-n1c(=O)nc(N)c2ccc(OC(F)F)nc21]

### Step 1: 4-amino-7-((4-methoxybenzyl)oxy)-1-(o-tolyl)pyrido[2,3-d]pyrimidin-2 (1H)-one

To a solution of 4-methoxybenzyl alcohol (59 μl, 474 μmol) in NMP (1 ml) at 0° C. sodium hydride (33 mg, 60% dispersion in mineral oil, 837 μmol) was added and the reaction mixture was stirred for 15 min. 4-amino-7-chloro-1-(o-tolyl)pyrido[2,3-d]pyrimidin-2 (1H)-one (80 mg, 279 μmol) (example 62) was added and the mixture was heated to 150° C. until LCMS showed full conversion. The reaction mixture was quenched with water and extracted with EtOAc. The combined organic layers were dried over Na₂SO₄, filtered and concentrated in vacuo. The crude product was purified by flash column chromatography to afford 4-amino-7-((4-methoxybenzyl)oxy)-1-(o-tolyl)pyrido[2,3-d]pyrimidin-2 (1H)-one (100 mg, 83%). ([M+H]⁺ 389.2)

### Step 2: 4-amino-7-hydroxy-1-(o-tolyl)pyrido[2,3-d]pyrimidin-2 (1H)-one

To a solution of 4-amino-7-((4-methoxybenzyl)oxy)-1-(o-tolyl)pyrido[2,3-d]pyrimidin-2 (1H)-one (670 mg, 1.55 mmol) in DCM (10 ml) and TFA (957 μl, 12.4 mmol) was added. The resulting solution was stirred for 2.5 h at room temperature before it was quenched with water. The aqueous layer was washed with DCM and evaporated to dryness. The crude product was purified by reversed phase preparative HPLC and yielded 4-amino-7-hydroxy-1-(o-tolyl)pyrido[2,3-d]pyrimidin-2 (1H)-one as a white solid (400 mg, 96%). ([M+H]⁺ 269.2)

### Step 3: 4-amino-7-(difluoromethoxy)-1-(2-methylphenyl)pyrido[2,3-d]pyrimidin-2-one

To a solution of 4-amino-7-hydroxy-1-(o-tolyl)pyrido[2,3-d]pyrimidin-2 (1H)-one (14 mg, 51.1 μmol) in NMP (2 ml) sodium chlorodifluoroacetate (156 mg, 102 μmol) and K₂CO₃ (21 mg, 153 μmol) were added. The mixture was heated to 80° C. for 25 min before it was quenched with water and extracted with EtOAc. The combined organic layers were dried over Na₂SO₄, filtered and concentrated in vacuo. The crude product was purified by flash column chromatography to afford 4-amino-7-(difluoromethoxy)-1-(2-methylphenyl)pyrido[2,3-d]pyrimidin-2-one (8 mg, 49%). ([M+H]⁺ 319.1)

### Example 92: 1-amino-4-(2-methoxyphenyl)-6-(trifluoromethyl)-3H-pyrido[1,2-c]pyrimidin-3-one

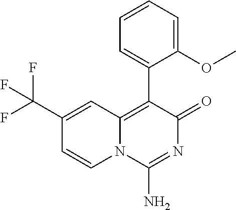 [SMILES: COc1ccccc1-c1c(=O)nc(N)n2ccc(C(F)(F)F)cc12]

### Step 1: 2-(2-methoxyphenyl)-2-(4-(trifluoromethyl)pyridin-2-yl)acetonitrile

To a solution of 2-chloro-4-(trifluoromethyl)pyridine (200 mg, 1.1 mmol) and 2-(2-methoxyphenyl)acetonitrile (162 mg, 1.1 mmol) in DMF (4 mL) was added NaH (60% dispersion in mineral oil, 88 mg, 2.2 mmol) and reaction was stirred at rt for 1 h before it was quenched with sat. NH₄Cl solution. Reaction mixture was extracted with EtOAc. The combined organic layers were washed with brine, dried over Na₂SO₄ and evaporated to give crude yellow oil. Compound was purified by flash column chromatography to afford 2-(2-methoxyphenyl)-2-(4-(trifluoromethyl)pyridin-2-yl)acetonitrile as light brown oil (221 mg, 69%). ([M+H]⁺ 293.2)

### Step 2: 2-(2-methoxyphenyl)-2-(4-(trifluoromethyl)pyridin-2-yl)acetamide

To a solution of 2-(2-methoxyphenyl)-2-(4-(trifluoromethyl)pyridin-2-yl)acetonitrile (50 mg, 0.17 mmol) in AcOH (0.7 mL) was added 95% H₂SO₄ (0.3 mL) and mixture was stirred for 2 days at 40° C. The reaction mixture was cooled to rt and poured onto ice followed by extraction with EtOAc. The organic layers were washed with sat. NaHCO₃ solution and brine, dried over Na₂SO₄ and concentrated. Purification by flash column chromatography afforded 2-(2-methoxyphenyl)-2-(4-(trifluoromethyl)pyridin-2-yl)acetamide (41 mg, 77%) as white solid. ([M+H]⁺ 311.2)

### Step 3: sodium 4-(2-methoxyphenyl)-3-oxo-6-(trifluoromethyl)-3H-pyrido[1,2-c]pyrimidine-1-thiolate

To a mixture of 2-(2-methoxyphenyl)-2-(4-(trifluoromethyl)pyridin-2-yl)acetamide (100 mg, 0.32 mmol) in EtOH (0.5 mL) was added sodium ethoxide (0.96 mL, 21% in EtOH, 2.58 mmol) followed by dropwise addition of thiophosgene (50 μL, 0.65 mmol) keeping the temperature below 40° C. The mixture was stirred in a sealed tube at 85° C. for 2 h before it was cooled to rt and quenched with ˜3 mL water. The resulting precipitate was filtered, washed with water and dried in vacuo to give sodium 4-(2-methoxyphenyl)-3-oxo-6-(trifluoromethyl)-3H-pyrido[1,2-c]pyrimidine-1-thiolate (85 mg, 64%) as yellow solid. ([M+H]⁺ 353.2)

### Step 4: 4-(2-methoxyphenyl)-1-(methylthio)-6-(trifluoromethyl)-3H-pyrido[1,2-c]pyrimidin-3-one

To a solution of sodium 4-(2-methoxyphenyl)-3-oxo-6-(trifluoromethyl)-3H-pyrido[1,2-c]pyrimidine-1-thiolate (80 mg, 0.21 mmol) in EtOH (2 mL) was added iodomethane (15 μL, 0.24 mmol). The reaction was stirred at rt for 7 h. An additional portion of iodomethane (˜10 μL) was added and stirring at rt was continued for 16 h. The reaction mixture was evaporated and the residue was diluted with EtOAc/water. The organic layers were washed with brine, dried over Na₂SO₄ and concentrated. Purification by flash column chromatography gave 4-(2-methoxyphenyl)-1-(methylthio)-6-(trifluoromethyl)-3H-pyrido[1,2-c]pyrimidin-3-one (58 mg, 72%) as yellow foam. ([M+H]⁺ 367.2)

### Step 5: 1-amino-4-(2-methoxyphenyl)-6-(trifluoromethyl)-3H-pyrido[1,2-c]pyrimidin-3-one

To a mixture of 4-(2-methoxyphenyl)-1-(methylthio)-6-(trifluoromethyl)-3H-pyrido[1,2-c]pyrimidin-3-one (51 mg, 0.14 mmol) and ammonium hydroxide solution (1.5 mL, 9.24 mmol) was added THF (0.5 mL). The reaction was stirred at rt for 3 days before it was concentrated. Purification by flash column chromatography afforded 1-amino-4-(2-methoxyphenyl)-6-(trifluoromethyl)-3H-pyrido[1,2-c]pyrimidin-3-one (23 mg, 49%) as yellow solid ([M+H]⁺ 336.3)

### Example 93: 4-amino-1-(2-methylpyridin-3-yl)-7-(tetrahydrofuran-3-yl)pyrido[2,3-d]pyrimidin-2 (1H)-one

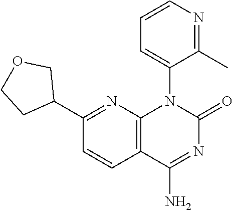 [SMILES: Cc1ncccc1-n1c(=O)nc(N)c2ccc(C3CCOC3)nc21]

4-amino-7-(4,5-dihydrofuran-3-yl)-1-(2-methylpyridin-3-yl)pyrido[2,3-d]pyrimidin-2 (1H)-one (20 mg, 62 μmol) (example 41) and Pd/C (7.0 mg, 62 μmol) were stirred at rt under hydrogen atmosphere for 16 h. The reaction mixture was filtered and concentrated in vacuo. Purification by flash column chromatography afforded 4-amino-1-(2-methylpyridin-3-yl)-7-(tetrahydrofuran-3-yl)pyrido[2,3-d]pyrimidin-2 (1H)-one (8 mg, 37%) as a white solid. ([M+H]⁺ 322.3)

### Example 94: 4-amino-7-cyclopropyl-2-oxo-1-(o-tolyl)-1,2-dihydropyrido[2,3-d]pyrimidine-5-carboxamide

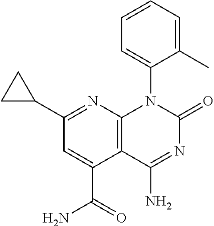 [SMILES: Cc1ccccc1-n1c(=O)nc(N)c2c(C(N)=O)cc(C3CC3)nc21]

### Step 1: ethyl 3-cyano-6-cyclopropyl-2-hydroxyisonicotinate

To a suspension of KOtBu (734 mg, 6.54 mmol) in THF (5 ml) at 0° C. were added dropwise over 10 min a mixture of diethyl oxalate (806 μl, 5.94 mmol) and 1-cyclopropylethan-1-one (589 μl, 5.94 mmol). The reaction mixture was stirred at 0° C. for 40 min, quenched with aq. dilute HCl and diluted with water. It was extracted with DCM, dried over Na₂SO₄, filtered and concentrated in vacuo. The crude product was added to a solution of 2-cyanoacetamide (500 mg, 5.94 mmol) and sodium methoxide (321 mg, 5.94 mmol) in MeOH (5 ml) and heated to 65° C. After 90 min the reaction mixture was cooled to rt and acidified with 6M HCl, extracted with EtOAc, dried over Na₂SO₄, filtered and concentrated in vacuo. Purification by flash column chromatography afforded ethyl 3-cyano-6-cyclopropyl-2-hydroxyisonicotinate (540 mg, 27%) as a light brown solid. ([M+H]⁺ 233.2)

### Step 2: ethyl 2-chloro-3-cyano-6-cyclopropylisonicotinate

Ethyl 3-cyano-6-cyclopropyl-2-hydroxyisonicotinate (60 mg, 258 μmol) was dissolved in POCl₃ (300 μl, 3.22 mmol) and the reaction mixture was heated to 100° C. for 2.5 h. The POCl₃ was removed in vacuo and the crude product was purified using flash column chromatography to afford ethyl 2-chloro-3-cyano-6-cyclopropylisonicotinate (22 mg, 32%) as a white solid. ([M+H]⁺ 251.2)

### Step 3: ethyl 3-cyano-6-cyclopropyl-2-(o-tolylamino)isonicotinate

Pd(OAc)₂ (2 mg, 8.0 μmol) was added to a degassed solution of ethyl 2-chloro-3-cyano-6-cyclopropylisonicotinate (20 mg, 79.8 μmol), o-toluidine (13 μl, 120 μmol), xphos (6 mg, 12 μmol) and Cs₂CO₃ (78 mg, 239 μmol) and the resulting reaction mixture was heated to 100° C. After 2 h it was cooled to rt, filtered through Decalite® and concentrated in vacuo. The crude product was purified using flash column chromatography to afford ethyl 3-cyano-6-cyclopropyl-2-(o-tolylamino)isonicotinate (8 mg, 31%) as a yellow solid. ([M+H]⁺ 322.3)

### Step 4: 4-amino-7-cyclopropyl-2-oxo-1-(o-tolyl)-1,2-dihydropyrido[2,3-d]pyrimidine-5-carboxamide

To a solution of ethyl 3-cyano-6-cyclopropyl-2-(o-tolylamino)isonicotinate (8 mg, 25 μmol) in DCM (0.5 ml) trichloroacetyl isocyanate (6 μl, 50 μmol) was added and the reaction mixture was stirred at rt until disappearance of starting material. After ammonia (7 M in MeOH, 1 ml, 7 mmol) was added and reaction was stirred until LCMS indicated full conversion to product. The crude product was purified by reversed phase preparative HPLC yielding 4-amino-7-cyclopropyl-2-oxo-1-(o-tolyl)-1,2-dihydropyrido[2,3-d]pyrimidine-5-carboxamide (5 mg, 57%) as a white solid. ([M+H]⁺ 336.1)

### Example 95: 4-amino-7-cyclopropyl-2-oxo-1-(o-tolyl)-1,2-dihydropyrido[2,3-d]pyrimidine-5-carbonitrile

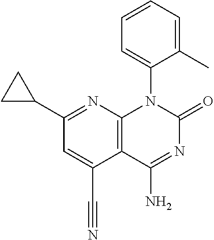 [SMILES: Cc1ccccc1-n1c(=O)nc(N)c2c(C#N)cc(C3CC3)nc21]

To a solution of 4-amino-7-cyclopropyl-2-oxo-1-(o-tolyl)-1,2-dihydropyrido[2,3-d]pyrimidine-5-carboxamide (23 mg, 68 μmol) (example 94) and TEA (33 μl, 239 μmol) in DCM (0.5 ml) at 0° C. trifluoroacetic anhydride (15 μl, 102 μmol) was added and the reaction mixture was stirred at rt. Three additional portions of TEA (33 μl, 239 μmol) and trifluoroacetic anhydride (15 μl, 102 μmol) was added every 30 min to afford complete conversion to product. The orange solution was adsorbed on silica and purified by flash column chromatography to afford 4-amino-7-cyclopropyl-2-oxo-1-(o-tolyl)-1,2-dihydropyrido[2,3-d]pyrimidine-5-carbonitrile (7 mg, 31%) as a white solid. ([M+H]⁺ 318.3)

### Example 96: 4-amino-7-cyclopropyl-1-(o-tolyl)pyrimido[4,5-d]pyrimidin-2 (1H)-one

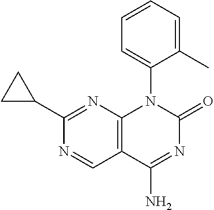 [SMILES: Cc1ccccc1-n1c(=O)nc(N)c2cnc(C3CC3)nc21]

### Step 1: 2-cyclopropyl-4-hydroxypyrimidine-5-carbonitrile

To a solution of cyclopropanecarboximidamide hydrochloride (200 mg, 1.58 mmol) and ethyl (E)-2-cyano-3-ethoxyacrylate (272 mg, 1.58 mmol) in EtOH (3.5 ml) at 0° C. was added KOtBu (442 mg, 3.94 mmol) and the suspension was stirred at 0° C. for 10 minutes and at reflux for 2 h. The mixture was poured on water and was acidified to pH 3 using aq. 25% HCl followed by extraction with EtOAc. The combined organic layers were dried over Na₂SO₄, filtered and concentrated in vacuo to give crude 2-cyclopropyl-4-hydroxypyrimidine-5-carbonitrile (196.5 mg, 77%) as a light yellow solid. ([M+H]⁺ 162.1)

### Step 2: 4-chloro-2-cyclopropylpyrimidine-5-carbonitrile

2-cyclopropyl-4-hydroxypyrimidine-5-carbonitrile (196 mg, 1.22 mmol) was combined with phosphorus oxychloride (1.42 ml, 15.2 mmol) to give an orange suspension. The reaction mixture was stirred at 110° C. for 1 h. The mixture was cooled to rt and was added dropwise to a well stirred mix of ice/water/EtOAc and washed with sat. aq. NaHCO₃. The aq. layer was extracted with EtOAc and combined organic layers were washed once with sat. aq. NaHCO₃. The combined organic layers were dried over Na₂SO₄, filtered and concentrated in vacuo to yield crude 4-chloro-2-cyclopropylpyrimidine-5-carbonitrile (122 mg, 56%). ([M+H]⁺ 180.1)

### Step 3: 2-cyclopropyl-4-(o-tolylamino)pyrimidine-5-carbonitrile

To a degassed solution of 4-chloro-2-cyclopropylpyrimidine-5-carbonitrile (122 mg, 679 μmol), o-toluidine (108 μl, 1.02 mmol) and Cs₂CO₃ (664 mg, 2.04 mmol) in dioxane (2.5 ml) were added xphos (49 mg, 102 μmol) and Pd(OAc)₂ (15 mg, 68 μmol) and the reaction mixture was stirred at 80° C. overnight. The reaction mixture was diluted with EtOAc and washed 3× with water. The combined organic layers were dried over Na₂SO₄, filtered and concentrated in vacuo. Purification by flash column chromatography afforded 2-cyclopropyl-4-(o-tolylamino)pyrimidine-5-carbonitrile (50 mg, 29%) as a off-white solid. ([M+H]⁺ 251.3)

### Step 4: 4-amino-7-cyclopropyl-1-(o-tolyl)pyrimido[4,5-d]pyrimidin-2 (1H)-one

To a solution of 2-cyclopropyl-4-(o-tolylamino)pyrimidine-5-carbonitrile (50 mg, 200 μmol) in DCE (1 ml) trichloroacetyl isocyanate (52 μl, 439 μmol) was added and the reaction mixture was stirred at at 80° C. overnight. After concentration, ammonia (7 M in MeOH, 6.67 ml, 46.7 mmol) was added and reaction was stirred for 1 h and the reaction concentrated to dryness Purification by flash column chromatography followed by suspension in EtOAc and filtration to yielded 4-amino-7-cyclopropyl-1-(o-tolyl)pyrimido[4,5-d]pyrimidin-2 (1H)-one (19 mg, 31%) as a white solid. ([M+H]⁺ 294.3)

### Example 97: 4-amino-7-cyclopropyl-1-(2-oxopiperidin-4-yl)pyrido[2,3-d]pyrimidin-2 (1H)-one

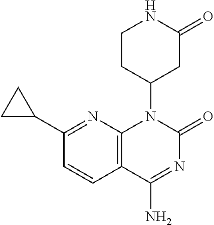 [SMILES: Nc1nc(=O)n(C2CCNC(=O)C2)c2nc(C3CC3)ccc12]

### Step 1: 6-cyclopropyl-2-((2-oxopiperidin-4-yl)amino)nicotinonitrile

To a solution of 2-chloro-6-cyclopropylnicotinonitrile (200 mg, 1.12 mmol) in DMSO (4 ml) were added DIPEA (978 μl, 5.6 mmol) and 4-aminopiperidin-2-one TFA salt (509 mg, 2.24 mmol). The reaction mixture was heated to 120° C. for 48 h after which time it was diluted with water and extracted twice with EtOAc. The organic layers were dried over Na₂SO₄, filtered and concentrated in vacuo. The crude material was purified by flash column chromatography to afford 6-cyclopropyl-2-((2-oxopiperidin-4-yl)amino)nicotinonitrile (192 mg, 55%) as a off-white solid. ([M+H]⁺ 257.2)

### Step 2: 6-cyclopropyl-2-((1-(4-methoxybenzyl)-2-oxopiperidin-4-yl)amino)nicotinonitrile

To a solution of 6-cyclopropyl-2-((2-oxopiperidin-4-yl)amino)nicotinonitrile (50 mg, 195 μmol) in THF (1 ml) at 0° C. were added 4-methoxybenzyl bromide (34 μl, 234 μmol) and KOtBu (43.8 mg, 390 μmol). The reaction mixture was stirred at rt. 4-methoxybenzyl bromide (34 μl, 234 μmol) was added once again after 2 h and the reaction mixture was stirred for additional 7 h. The reaction mixture was quenched with water and extracted twice with DCM. The organic layers were dried over Na₂SO₄, filtered and concentrated in vacuo. The crude material was purified by flash column chromatography to afford 6-cyclopropyl-2-((1-(4-methoxybenzyl)-2-oxopiperidin-4-yl)amino)nicotinonitrile (33 mg, 36%) as a yellow oil. ([M+H]⁺ 377.4)

### Step 3: 4-amino-7-cyclopropyl-1-(1-(4-methoxybenzyl)-2-oxopiperidin-4-yl)pyrido[2,3-d]pyrimidin-2 (1H)-one

To a solution of 6-cyclopropyl-2-((1-(4-methoxybenzyl)-2-oxopiperidin-4-yl)amino)nicotinonitrile (47 mg, 125 μmol) in DCM (1.5 ml) trichloroacetyl isocyanate (33 μl, 275 μmol) was added and the reaction mixture was stirred at rt until disappearance of starting material. After ammonia (7 M in MeOH, 4 ml, 28 mmol) was added and reaction was stirred until LCMS indicated full conversion to product. The crude product was purified by flash column chromatography to yield 4-amino-7-cyclopropyl-1-(1-(4-methoxybenzyl)-2-oxopiperidin-4-yl)pyrido[2,3-d]pyrimidin-2 (1H)-one (27 mg, 48%) as a white solid. ([M+H]⁺ 420.4)

### Step 4: 4-amino-7-cyclopropyl-1-(2-oxopiperidin-4-yl)pyrido[2,3-d]pyrimidin-2 (1H)-one

4-amino-7-cyclopropyl-1-(1-(4-methoxybenzyl)-2-oxopiperidin-4-yl)pyrido[2,3-d]pyrimidin-2 (1H)-one (27 mg, 64 μmol) was dissolved in TFA (2 ml, 26 mmol). The reaction mixture was stirred at 75° C. for 67 h before it was concentrated in vacuo and purified by reverse-phase HPLC to yield 4-amino-7-cyclopropyl-1-(2-oxopiperidin-4-yl)pyrido[2,3-d]pyrimidin-2 (1H)-one (9 mg, 46%) as a white solid. ([M+H]⁺ 300.2)

### Example 98: 7-cyclopropyl-1-(2-methylpyridin-3-yl)quinazoline-2,4-dione

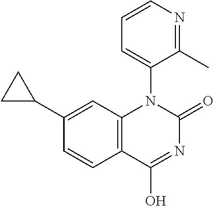 [SMILES: Cc1ncccc1-n1c(=O)nc(O)c2ccc(C3CC3)cc21]

4-amino-7-cyclopropyl-1-(2-methylpyridin-3-yl)quinazolin-2 (1H)-one (55 mg, 188 μmol) (Example 86) was suspended in KOH (2M aqueous, 941 μl, 1.88 mmol) and heated to 110° C. After 4 h the reaction mixture was cooled to room temperature, diluted with water and extracted 3 times with EtOAc. The combined organic layers were dried over magnesium sulfate, filtered and evaporated in vacuo. The crude product was purified by flash column chromatography to yield 4-amino-7-cyclopropyl-1-(1-(4-methoxybenzyl)-2-oxopiperidin-4-yl)pyrido[2,3-d]pyrimidin-2 (1H)-one (25 mg, 43%) as a white solid. ([M+H]⁺ 294.3)

### Example 99: 7-cyclopropyl-1-(2-methylphenyl)pyrido[2,3-d]pyrimidine-2,4-dione

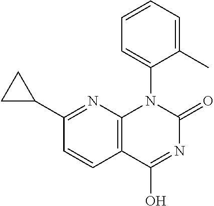 [SMILES: Cc1ccccc1-n1c(=O)nc(O)c2ccc(C3CC3)nc21]

### Step 1: methyl 2-chloro-6-cyclopropylpyridine-3-carboxylate

To a degassed solution of methyl 6-bromo-2-chloronicotinate (700 mg, 2.65 mmol) in dioxane (12 ml) were added K₂CO₃ (734 mg, 5.31 mmol), cyclopropylboronic acid (1.14 g, 13.3 mmol) and Pd(dppf)₂Cl₂·CH₂Cl₂ (217 mg, 265 μmol). The reaction mixture was heated in the microwave at 80° C. for 1 h before it was poured into water and extracted 3 times with EtOAc. The combined organic layers were washed with brine, dried over magnesium sulfate, filtered and evaporated. The crude product was purified by flash column chromatography to afford the title compound (498 mg, 87%) as a yellow crystalline solid. ([M+H]⁺ 212.1)

### Step 2: 2-chloro-6-cyclopropylpyridine-3-carboxylic acid

To a solution of methyl 2-chloro-6-cyclopropylnicotinate (489 mg, 2.31 mmol) in THF (5 ml) and MeOH (5 ml) was added LiOH (1M in water, 4.62 ml, 4.62 mmol). The reaction mixture was stirred at rt over night before volatiles were removed in vacuo. The residue was diluted with water, acidified with 1 M HCl and extracted 3 times with EtOAc. The combined organic layers were washed with brine, dried over magnesium sulfate, filtered and evaporated to afford the title compound (470 mg, 100%) as a white solid. ([M+H]⁺ 198.1)

### Step 3: 2-chloro-6-cyclopropylpyridine-3-carboxamide

To a solution of 2-chloro-6-cyclopropylnicotinic acid (220 mg, 1.11 mmol) in DMF (2 ml) was added CDI (271 mg, 1.67 mmol). The reaction was heated to 50° C. for 2.5 h before ammonia (25% solution in water, 1.21 g, 1.33 ml, 17.8 mmol) was added at rt and the reaction was allowed to stir for 3 days. The reaction was diluted with water and extracted 3 times with EtOAc. The combined organic layers were dried over magnesium sulfate, filtered and evaporated to dryness. The crude product was purified by flash column chromatography to afford the desired compound (165 mg, 74%) as a white solid. ([M+H]⁺ 197.2)

### Step 4: 6-cyclopropyl-2-(2-methylanilino)pyridine-3-carboxamide

A solution of 2-chloro-6-cyclopropylnicotinamide (79 mg, 402 μmol) and o-toluidine (42.9 μl, 402 μmol) in AcOH (0.5 ml) was heated to 120° C. overnight. The reaction was cooled to rt and basified with 2N NaOH and extracted 3 times with EtOAc. The combined organic layers were washed with water and brine, dried over magnesium sulfate, filtered and evaporated to dryness. The crude product was purified by flash column chromatography to afford 6-cyclopropyl-2-(2-methylanilino)pyridine-3-carboxamide (50 mg, 46%) as a light yellow solid. ([M+H]⁺ 268.2)

### Step 5: 7-cyclopropyl-1-(2-methylphenyl)pyrido[2,3-d]pyrimidine-2,4-dione

A light yellow solution of 6-cyclopropyl-2-(2-methylanilino)pyridine-3-carboxamide (47 mg, 176 μmol), CDI (43 mg, 264 μmol) and DBU (53 μl, 352 μmol) in THF (1 ml) was heated to 70° C. for 1 h before it was cooled to rt, diluted with water and extracted 3 times with EtOAc. The combined organic layers were washed with brine, dried over magnesium sulfate, filtered and evaporated to dryness. The crude product was purified by flash column chromatography to afford 7-cyclopropyl-1-(2-methylphenyl)pyrido[2,3-d]pyrimidine-2,4-dione (33 mg, 63%) as a white solid. ([M+H]⁺ 294.2)

### Example 100: 7-cyclopropyl-1-(2-methylphenyl)quinazoline-2,4-dione

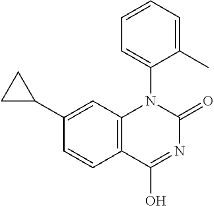 [SMILES: Cc1ccccc1-n1c(=O)nc(O)c2ccc(C3CC3)cc21]

4-amino-7-cyclopropyl-1-(o-tolyl)quinazolin-2 (1H)-one (60 mg, 206 μmol) (example 8) was suspended in KOH (2M in water, 1.03 ml, 2.06 mmol) and heated to 110° C. for 4 h before it was cooled to rt, diluted with water and extracted 3 times with EtOAc. The combined organic layers were dried over magnesium sulfate, filtered and evaporated to dryness to afford the title compound (56 mg, 90%) as a white solid. ([M+H]⁺ 293.2)

### Example 101: 7-cyclopropyl-1-(2-methylpyridin-3-yl)pyrido[2,3-d]pyrimidine-2,4-dione

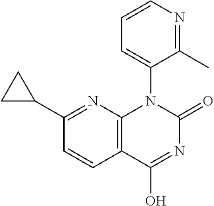 [SMILES: Cc1ncccc1-n1c(=O)nc(O)c2ccc(C3CC3)nc21]

To a mixture of Example 7 (20 mg, 680 μmol) in TI-IF (1 mL) was added tert-butyl nitrite (17 μL, 136 μmol). Reaction was stirred at 60° C. for 2 h before more tert-butyl nitrite (17 μL, 136 μmol) was added and mixture was stirred at 60° C. for additional 5 h. Reaction was cooled to rt and extracted with EtOAc. The organic layers were washed with brine, dried over Na₂SO₄ and evaporated in vacuo. Compound was purified by flash chromatography to give 7-cyclopropyl-1-(2-methylpyridin-3-yl)pyrido[2,3-d]pyrimidine-2,4-dione (14 mg, 70%) as white solid. ([M+H]⁺ 295.1)

### Example 102: 4-amino-5-methoxy-1-(2-methylpyridin-3-yl)-7-(trifluoromethyl)quinazolin-2 (1H)-one

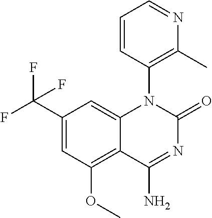 [SMILES: COc1cc(C(F)(F)F)cc2c1c(N)nc(=O)n2-c1cccnc1C]

4-amino-5-fluoro-1-(2-methylpyridin-3-yl)-7-(trifluoromethyl)quinazolin-2 (1H)-one (61 mg. 180 μmol) (example 59) and sodium methoxide (15 mg, 270 μmol) were stirred in MeOH (3 ml) for 48 h at rt The solvent was evaporated and the crude product was purified by flash column chromatography to afford 4-amino-5-methoxy-1-(2-methylpyridin-3-yl)-7-(trifluoromethyl)quinazolin-2 (1H)-one (42 mg, 63%) as a white solid. ([M+H]⁺ 351.2)

### Example 103 and Example 104: (+)-4-amino-7-cyclopropyl-1-(3-fluoro-2-methylphenyl)pyrido[2,3-d]pyrimidin-2 (1H)-one and (−)-4-amino-7-cyclopropyl-1-(3-fluoro-2-methylphenyl)pyrido[2,3-d]pyrimidin-2 (1H)-one

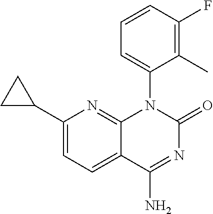 [SMILES: Cc1c(F)cccc1-n1c(=O)nc(N)c2ccc(C3CC3)nc21]

### Step 1: 6-cyclopropyl-2-((3-fluoro-2-methylphenyl)amino)nicotinonitrile

The title compound ([M+H]⁺ 268.3) was prepared from 2-chloro-6-cyclopropylnicotinonitrile (CAS [1198475-35-2]) by reaction with 3-fluoro-2-methylaniline (CAS [443-86-7]) using Pd(OAc)₂ as a catalyst and xantphos as a ligand (General procedure B1).

### Step 2: 4-amino-7-cyclopropyl-1-(3-fluoro-2-methylphenyl)pyrido[2,3-d]pyrimidin-2 (1H)-one

To a solution of 6-cyclopropyl-2-((3-fluoro-2-methylphenyl)amino)nicotinonitrile (57 mg, 213 μmol) in DCE (3 ml) trichloroacetyl isocyanate (56 μl, 469 μmol) was added and the reaction mixture was stirred at rt until disappearance of starting material. After, ammonia (7 M in MeOH, 3.81 ml, 26.6 mmol) was added and reaction was stirred until LCMS indicated full conversion to product. The crude product was purified by flash column chromatography to yield 4-amino-7-cyclopropyl-1-(3-fluoro-2-methylphenyl)pyrido[2,3-d]pyrimidin-2 (1H)-one (61 mg, 91%) as a white solid. ([M+H]⁺ 311.2)

### Step 3: (+)-4-amino-7-cyclopropyl-1-(3-fluoro-2-methylphenyl)pyrido[2,3-d]pyrimidin-2 (1H)-one and (−)-4-amino-7-cyclopropyl-1-(3-fluoro-2-methylphenyl)pyrido[2,3-d]pyrimidin-2 (1H)-one

4-amino-7-cyclopropyl-1-(3-fluoro-2-methylphenyl)pyrido[2,3-d]pyrimidin-2 (1H)-one was separated by chiral reversed phase prep-HPLC to afford (+)-4-amino-7-cyclopropyl-1-(3-fluoro-2-methylphenyl)pyrido[2,3-d]pyrimidin-2 (1H)-one as a white solid and (−)-4-amino-7-cyclopropyl-1-(3-fluoro-2-methylphenyl)pyrido[2,3-d]pyrimidin-2 (1H)-one as a white solid. ([M+H]⁺ 420.4) and ([M+H]⁺ 311.2)

### Example 105: 3-(4-amino-6-chloro-7-isopropyl-2-oxopyrido[2,3-d]pyrimidin-1 (2H)-yl)₂ methoxybenzonitrile

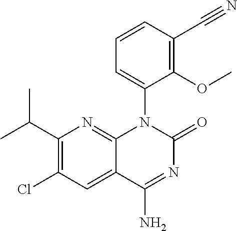 [SMILES: COc1c(C#N)cccc1-n1c(=O)nc(N)c2cc(Cl)c(C(C)C)nc21]

### Step 1: 2-amino-6-isopropylnicotinonitrile

The title compound ([M+H]+ 162.2) was prepared from 2-chloro-6-isopropylnicotinonitrile (CAS [108244-44-6]) using General procedure D.

### Step 2: 2-amino-5-chloro-6-isopropylnicotinonitrile

To a solution of 2-amino-6-isopropylnicotmonitrile (130 mg, 806 μmol) in CHCl3 (5 ml) was added NCS (118 mg, 887 μmol) and reaction was stirred in the dark at 60° C. for 6 h. The mixture was diluted with water and extracted with EtOAc. The organic layer was washed 2× with water and the combined organic layers were dried and evaporated to dryness to yield crude product (180 mg, 114%) as an orange solid. ([M+H]+ 196.1)

### Step 3: 5-chloro-2-((3-cyano-2-methoxyphenyl)amino)-6-isopropylnicotinonitrile

2-amino-5-chloro-6-isopropylnicotinonitrile (90 mg, 460 μmol), 3-bromo-2-methoxybenzonitrile (127 mg, 598 μmol), xantphos (26.6 mg, 46 μmol) and Cs₂CO₃ (450 mg, 1.38 mmol) were mixed in dioxane (3 ml) and degassed under argon. Pd(OAc)₂ (5.16 mg, 23 μmol) was added and the mixture was stirred over night at 90° C. On the next day again xantphos (27 mg, 46 μmol) and Pd(OAc)₂ (5.2 mg, 23 μmol) were added and the reaction was stirred for additional 3 h at 90° C. The crude product was purified by flash column chromatography to yield 5-chloro-2-((3-cyano-2-methoxyphenyl)amino)-6-isopropylnicotinonitrile (75 mg, 50%) as a yellow solid. ([M+H]⁺ 327.2)

### Step 4: 3-(4-amino-6-chloro-7-isopropyl-2-oxopyrido[2,3-d]pyrimidin-1 (2H)-yl)-2-methoxybenzonitrile

5-chloro-2-((3-cyano-2-methoxyphenyl)amino)-6-isopropylnicotinonitrile (70 mg, 214 μmol) was dissolved in DCM (1 ml) and trichloroacetyl isocyanate (121 mg, 76.1 μl, 643 μmol) was added. The mixture was stirred over night at rt before an additional trichloroacetyl isocyanate (121 mg, 76.1 μl, 643 μmol) was added and stirred at rt. After 3 h ammonia (7 M in MeOH, 5 ml, 35 mmol) was added and stirred over night at rt. The reaction mixture was evaporated and the residue was purified by by flash column chromatography to yield 3-(4-amino-6-chloro-7-isopropyl-2-oxopyrido[2,3-d]pyrimidin-1 (2H)-yl)-2-methoxybenzonitrile (76 mg, 96%) as a white solid. ([M+H]+ 370.2)

### Example 106: 4-amino-7-cyclopropyl-1-(3-fluoro-2-methylphenyl)quinazolin-2-one

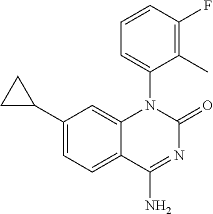 [SMILES: Cc1c(F)cccc1-n1c(=O)nc(N)c2ccc(C3CC3)cc21]

### Step 1: 4-cyclopropyl-2-((3-fluoro-2-methylphenyl)amino)benzonitrile

The title compound ([M+H]⁺ 267.2) was prepared from 2-chloro-4-cyclopropylbenzonitrile (Angew. Chem. 2018, 12573) by reaction with 3-fluoro-2-methylaniline (CAS [443-86-7]) using Pd(OAc)₂ as a catalyst and X-phos as a ligand (General procedure B1).

### Step 2: 4-amino-7-cyclopropyl-1-(3-fluoro-2-methylphenyl)quinazolin-2-one

To a solution of 4-cyclopropyl-2-((3-fluoro-2-methylphenyl)amino)benzonitrile (76 mg, 284 μmol) in DCM (3 ml) trichloroacetyl isocyanate (75 μl, 625 μmol) was added and the reaction mixture was stirred at rt until disappearance of starting material. After, ammonia (7 M in MeOH, 6.1 ml, 42.6 mmol) was added and reaction was stirred until LCMS indicated full conversion to product. The crude product was purified by flash column chromatography to yield 4-amino-7-cyclopropyl-1-(3-fluoro-2-methylphenyl)quinazolin-2-one (70 mg, 80%) as a white solid. ([M+H]⁺ 308.2)

### Example 107: 4-amino-7-cyclopropyl-1-(1,4-dioxepan-6-yl)pyrido[2,3˜d]pyrimidin-2-one

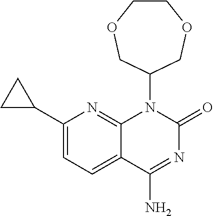 [SMILES: Nc1nc(=O)n(C2COCCOC2)c2nc(C3CC3)ccc12]

### Step 1: 6-cyclopropyl-2-(1,4-dioxepan-6-ylamino)pyridine-3-carbonitrile

The title compound ([M+H]⁺ 260.2) was prepared from 2-chloro-6-cyclopropylnicotinonitrile (CAS [1198475-35-2]) by reaction with 1,4-dioxepan-6-amine (EP1958666 A1) using Pd(OAc)₂ as a catalyst and xanthphos as a ligand (General procedure B1).

### Step 2: 4-amino-7-cyclopropyl-1-(1,4-dioxepan-6-yl)pyrido[2,3-d]pyrimidin-2-one

To a solution of 6-cyclopropyl-2-(1,4-dioxepan-6-ylamino)pyridine-3-carbonitrile (15 mg, 60 μmol) in DCM (1 ml) trichloroacetyl isocyanate (35 μl, 290 μmol) was added and the reaction mixture was stirred at rt until disappearance of starting material. After, ammonia (7 M in MeOH, 1.0 ml, 0.06 mmol) was added and reaction was stirred until LCMS indicated full conversion to product. The crude product was purified by flash column chromatography to 4-amino-7-cyclopropyl-1-(1,4-dioxepan-6-yl)pyrido[2,3-d]pyrimidin-2-one (9 mg, 78%) as a white solid. ([M+H]⁺ 303.1)

### Example 108: 4-amino-7-cyclopropyl-1-(6-(difluoromethoxy)pyridin-2-yl)pyrido[2,3-d]pyrimidin-2 (1H)-one

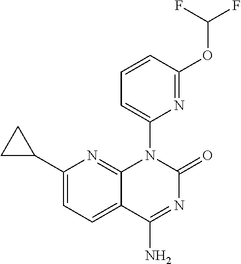 [SMILES: Nc1nc(=O)n(-c2cccc(OC(F)F)n2)c2nc(C3CC3)ccc12]

### Step 1: 6-cyclopropyl-2-((6-(difluoromethoxy)pyridin-2-yl)amino)nicotinonitrile

The title compound ([M+H]⁺ 303.1) was prepared from 2-chloro-6-cyclopropylnicotinonitrile (CAS [1198475-35-2]) by reaction with 6-(difluoromethoxy)pyridin-2-amine (CAS [1131007-43-6]) using Pd(OAc)₂ as a catalyst and xanthphos as a ligand (General procedure B1).

### Step 2: 4-amino-7-cyclopropyl-1-(6-(difluoromethoxy)pyridin-2-yl)pyrido[2,3-d]pyrimidin-2 (1H)-one

To a solution of 6-cyclopropyl-2-((6-(difluoromethoxy)pyridin-2-yl)amino)nicotinonitrile (87 mg, 210 μmol) in DCE (2 ml) trichloroacetyl isocyanate (62 μl, 525 μmol) was added and the reaction mixture was stirred at rt until disappearance of starting material. The reaction was concentrated to dryness and redissolved in ammonia (7 M in MeOH, 12 ml, 84 mmol) was added and reaction was stirred until LCMS indicated full conversion to product. Evaporation of the methanol and trituration with ethyl aceate afforded 4-amino-7-cyclopropyl-1-(6-(difluoromethoxy)pyridin-2-yl)pyrido[2,3-d]pyrimidin-2 (1H)-one (35 mg, 48%) as a white solid. ([M+H]⁺ 346.2)

### Example 109: 4-amino-1-(2-chloro-3-pyridyl)-7-(trifluoromethoxy)quinazolin-2-one

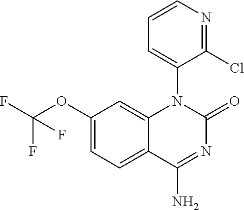 [SMILES: Nc1nc(=O)n(-c2cccnc2Cl)c2cc(OC(F)(F)F)ccc12]

### Step 1: 2-[(2-chloro-3-pyridyl)amino]-4-(trifluoromethoxy)benzonitrile

The title compound ([M+H]⁺ 313.8) was prepared from 2-bromo-4-(trifluoromethoxy)benzonitrile (CAS [1214334-83-4]) by reaction with 3-amino-2-chloro-pyridine (CAS [6298-19-7]) using Pd(OAc)₂ as a catalyst and xanthphos as a ligand (General procedure B1).

### Step 2: 4-amino-1-(2-chloro-3-pyridyl)-7-(trifluoromethoxy)quinazolin-2-one

To a solution of 2-[(2-chloro-3-pyridyl)amino]-4-(trifluoromethoxy)benzonitrile (100 mg, 320 μmol) in DCM (5 ml) trichloroacetyl isocyanate (187 μl, 1.5 mmol) was added and the reaction mixture was stirred at rt until disappearance of starting material. Ammonia (7 M in MeOH, 5 ml, 35 mmol) was added and reaction was stirred until LCMS indicated full conversion to product.

The crude product was purified by flash column chromatography to 4-amino-1-(2-chloro-3-pyridyl)-7-(trifluoromethoxy)quinazolin-2-one (33 mg, 26%) as a white solid. ([M+H]⁺ 357.1)

### Example 110: 4-amino-7-cyclopropyl-1-[2-(trifluoromethoxy)phenyl]pyrido[2,3-d]pyrimidin-2-one

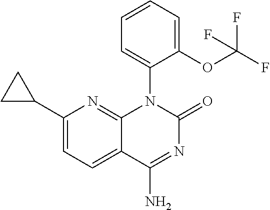 [SMILES: Nc1nc(=O)n(-c2ccccc2OC(F)(F)F)c2nc(C3CC3)ccc12]

### Step 1: 6-cyclopropyl-2-[2-(trifluoromethoxy)anilino]pyridine-3-carbonitrile

The title compound ([M+H]⁺ 320.0) was prepared from 2-chloro-6-cyclopropylnicotinonitrile (CAS [1198475-35-2]) by reaction with 2-(trifluoromethoxy)aniline (CAS [175205-77-3]) using Pd(OAc)₂ as a catalyst and xanthphos as a ligand (General procedure B1).

### Step 2: 4-amino-7-cyclopropyl-1-[2-(trifluoromethoxy)phenyl]pyrido[2,3-d]pyrimidin-2-one

The title compound ([M+H]⁺ 363.0) was prepared from 6-cyclopropyl-2-[2-(trifluoromethoxy)anilino]pyridine-3-carbonitrile using General procedure C.

### Example 111: 4-(2-chlorophenyl)-6-cyclopropyl-1-imino-pyrido[1,2-c]pyrimidin-3-one

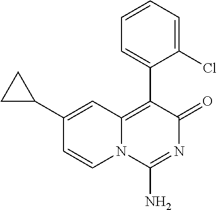 [SMILES: Nc1nc(=O)c(-c2ccccc2Cl)c2cc(C3CC3)ccn12]

### Step 1: (4-bromo-2-pyridyl)-(2-chlorophenyl)methanol

To a solution of 2,4-dibromopyridine (500 mg, 2.11 mmol) in toluene (25 mL) was added dropwise n-BuLi/(1.6M in hexanes, 1.01 mL, 2.53 mmol) at −78° C. and the reaction mixture was stirred for 1 h before addition of 2-chlorobenzaldehyde (326 mg, 2.32 mmol) and the mixture stirred for another 1 h before the reaction mixture was poured into sat. NH₄Cl, extracted with ethyl acetate, the combined extracts were washed with brine and concentrated. Purification by flash column chromatography gave product (4-bromo-2-pyridyl)-(2-chlorophenyl)methanol (500 mg, 71%) as a yellow oil. ([M+H]⁺ 298.0)

### Step 2: (4-bromo-2-pyridyl)-(2-chlorophenyl)methanone

To a solution of (4-bromo-2-pyridyl)-(2-chlorophenyl)methanol (500 mg, 1.67 mmol) in chloroform (20 mL) was added MnO₂ (1455 mg, 16.75 mmol) at 25° C., the reaction mixture was stirred at 50° C. for 2 h. The mixture was filtered through Celite® and concentrated. Purification by flash column chromatography gave product (4-bromo-2-pyridyl)-(2-chlorophenyl)methanone (450 mg, 91%) as a yellow oil. ([M+H]⁺ 295.9)

### Step 3: (2-chlorophenyl)-(4-cyclopropyl-2-pyridyl)methanone

A mixture of (4-bromo-2-pyridyl)-(2-chlorophenyl)methanone (400 mg, 1.35 mmol), potassium cyclopropyltrifluoroborate (399 mg, 2.7 mmol), K₂CO₃ (558 mg, 4.05 mmol), Pd(dppf)Cl₂ (40.0 mg, 0.130 mmol) in 1,4-dioxane (4 mL) and water (1 mL) stirred at 80° C. for 12 h under inert atmosphere. The reaction mixture was concentrated under reduced pressure and the residue was purified by flash column chromatography to give product (2-chlorophenyl)-(4-cyclopropyl-2-pyridyl)methanone (300 mg, 69%) as a yellow oil. ([M+H]⁺ 258.1)

### Step 4: 2-(2-chlorophenyl)-2-(4-cyclopropyl-2-pyridyl)acetonitrile

To an ice-cold solution of (2-chlorophenyl)-(4-cyclopropyl-2-pyridyl)methanone (500 mg, 191 mmol) and Tosmic (568 mg, 2.9 mmol) in DME (10 mL) was added potassium tert-butylate (1 M in tBuOH, 4.85 ml, 4.85 mmol) and reaction was then heated to 50° C. for 12 h before it was quenched by addition of water. The reaction mixture was extracted with EtOAc. The combined organic layers were washed with brine, dried (Na₂SO₄) and concentrated. The product was purified by flash column chromatography to afford 2-(2-chlorophenyl)-2-(4-cyclopropyl-2-pyridyl)acetonitrile as a light yellow oil (500 mg, 67%). ([M+H]⁺ 269.1)

### Step 5: 2-(2-chlorophenyl)-2-(4-cyclopropyl-2-pyridyl)acetamide

To a solution of 2-(2-chlorophenyl)-2-(4-cyclopropyl-2-pyridyl)acetonitrile (450 mg, 1.67 mmol) in AcOH (15 mL) was added 95% H₂SO₄ (5 mL) and the mixture was stirred for 2 days at 40° C. The reaction mixture was cooled to rt and poured onto ice followed by extraction with EtOAc. The organic layers were washed with sat. NaHCO₃ solution and brine, dried (Na₂SO₄) and concentrated. Purification by flash column chromatography afforded 2-(2-chlorophenyl)-2-(4-cyclopropyl-2-pyridyl)acetamide (400 mg, 83%) as yellow solid. ([M+H]⁺ 287.1)

### Step 6: 4-(2-chlorophenyl)-6-cyclopropyl-1-thioxo-pyrido[1,2-c]pyrimidin-3-one

To a mixture of 2-(2-chlorophenyl)-2-(4-cyclopropyl-2-pyridyl)acetamide (400 mg, 1.39 mmol) in EtOH (2.5 mL) was added sodium ethoxide (4.2 mL, 21% in EtOH, 11.6 mmol) followed by dropwise addition of thiophosgene (215 μL, 2.79 mmol) keeping the temperature below 40° C. The mixture was stirred in a sealed tube at 85° C. for 2 h before it was cooled to rt and quenched with ˜3 mL water. The mixture was extracted with ethyl acetate, the combined washings washed with brine and concentrated. Purification by flash column chromatography to gave 4-(2-chlorophenyl)-6-cyclopropyl-1-thioxo-pyrido[1,2-c]pyrimidin-3-one (250 mg, 44%) as yellow solid. ([M+H]⁺ 329.0)

### Step 7: 4-(2-chlorophenyl)-6-cyclopropyl-1-methylsulfanyl-pyrido[1,2-c]pyrimidin-3-one

To a solution of 4-(2-chlorophenyl)-6-cyclopropyl-1-thioxo-pyrido[1,2-c]pyrimidin-3-one (200 mg, 0.61 mmol) in DMF (2 mL) was added potassium carbonate (168 mg, 1.22 mmol) iodomethane (45 μL, 0.73 mmol). The reaction was stirred at rt for 7 h. The reaction mixture was evaporated and the residue was diluted with EtOAc/water. The organic layers were washed with brine, dried (Na₂SO₄) and concentrated. Purification by flash column chromatography gave 4-(2-chlorophenyl)-6-cyclopropyl-1-methylsulfanyl-pyrido[1,2-c]pyrimidin-3-one 4-(2-methoxyphenyl)-1-(methylthio)-6-(trifluoromethyl)-3H-pyrido[1,2-c]pyrimidin-3-one (100 mg, 43%) as yellow oil. ([M+H]⁺ 343.0)

### Step 8: 1-amino-4-(2-methoxyphenyl)-6-(trifluoromethyl)-3H-pyrido[1,2-c]pyrimidin-3-one

To a mixture of 4-(2-chlorophenyl)-6-cyclopropyl-1-methylsulfanyl-pyrido[1,2-c]pyrimidin-3-one (90 mg, 0.260 mmol) and ammonium hydroxide solution (1.5 mL, 9.24 mmol) was added THF (0.5 mL). The reaction was heated to 50° C. for 48 h after which time it was concentrated. Purification by flash column chromatography afforded 4-(2-chlorophenyl)-6-cyclopropyl-1-imino-pyrido[1,2-c]pyrimidin-3-one (8 mg, 9%) as yellow solid ([M+H]⁺ 312.2)

### Example 112: 4-amino-7-cyclopropyl-1-(6,7-dihydro-51-pyrrolo[1,2-c]imidazol-7-yl)pyrido[2,3-d]pyrimidin-2-one

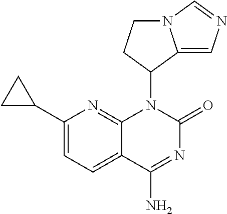 [SMILES: Nc1nc(=O)n(C2CCn3cncc32)c2nc(C3CC3)ccc12]

### Step 1: 6-cyclopropyl-2-(6,7-dihydro-5H-pyrrolo[1,2-c]imidazol-7-ylamino)pyridine-3-carbonitrile

The title compound ([M+H]⁺ 266.1) was prepared from 2-chloro-6-cyclopropylnicotinonitrile (CAS [1198475-35-2]) by reaction with 6,7-dihydro-5H-pyrrolo[1,2-c]imidazol-7-amine hydrochloride (CAS [272438-86-5]) using Pd(OAc)₂ as a catalyst and xanthphos as a ligand (General procedure B1).

### Step 2: 4-amino-7-cyclopropyl-1-(6,7-dihydro-5H-pyrrolo[1,2-c]imidazol-7-yl)pyrido[2,3-d]pyrimidin-2-one

The title compound ([M+H]⁺ 309.2) was prepared from 6-cyclopropyl-2-(6,7-dihydro-5H-pyrrolo[1,2-c]imidazol-7-ylamino)pyridine-3-carbonitrile using General procedure C.

### Example 113: 4-amino-7-cyclopropyl-6-(difluoromethoxy)-1-(o-tolyl)pyrido[2,3-d]pyrimidin-2-one

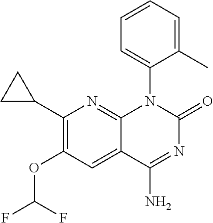 [SMILES: Cc1ccccc1-n1c(=O)nc(N)c2cc(OC(F)F)c(C3CC3)nc21]

### Step 1: 2-amino-5-bromo-6-cyclopropyl-pyridine-3-carbonitrile

To a solution of 2-amino-6-cyclopropyl-pyridine-3-carbonitrile (1000 mg, 6.28 mmol) in chloroform (20 mL) was added N-bromosuccinamide (1173 mg, 6.6 mmol). The mixture was stirred at 20° C. for 16 h in the dark after which time it was concentrated. Purification by flash column chromatography afforded 2-amino-5-bromo-6-cyclopropyl-pyridine-3-carbonitrile (1.4 g, 93% yield) as yellow solid. ([M+H]⁺ 238.0)

### Step 2: 2-[bis[(4-methoxyphenyl)methyl]amino]-5-bromo-6-cyclopropyl-pyridine-3-carbonitrile

A mixture of 2-amino-5-bromo-6-cyclopropyl-pyridine-3-carbonitrile (200 mg, 0.84 mmol) in THF (10 mL) was cooled to 0° C. NaH (141.13 mg, 3.53 mmol, 4.2 eq) was added and the mixture stirred for 0.5 h after which time was added 4-methoxybenzylchloride (0.46 mL, 3.36 mmol) and the mixture then stirred at rt for 12 h. The reaction was quenched by addition of sat.NH₄Cl and extracted with ethyl acetate and concentrated. Purification by flash column chromatography afforded 2-[bis[(4-methoxyphenyl)methyl]amino]-5-bromo-6-cyclopropyl-pyridine-3-carbonitrile (300 mg, 75%) as light yellow gum ([M+H]⁺ 480.2)

### Step 3: 2-[bis[(4-methoxyphenyl)methyl]amino]-6-cyclopropyl-5-hydroxy-pyridine-3-carbonitrile

A mixture of 2-[bis[(4-methoxyphenyl)methyl]amino]-5-bromo-6-cyclopropyl-pyridine-3-carbonitrile (300 mg, 0.63 mmol), potassium hydroxide (105 mg, 1.88 mmol, 3), t-BuBretPhos Pd G3 (107 mg, 0.13 mmol), and t-BubretPhos (61 mg, 0.13 mmol) in 1,4-dioxane (3 mL) and water (0.30 mL) was stirred at 80° C. for 2 h. The reaction was then diluted with water, extracted with ethyl acetate and the combined organic washed with brine and concentrated. Purification by flash column chromatography afforded 2-[bis[(4-methoxyphenyl)methyl]amino]-6-cyclopropyl-5-hydroxy-pyridine-3-carbonitrile (260 mg, 70% yield) as orange oil. ([M+H]⁺ 416.3)

### Step 4: 2-[bis[(4-methoxyphenyl)methyl]amino]-6-cyclopropyl-5-(difluoromethoxy)pyridine-3-carbonitrile

A mixture of 2-[bis[(4-methoxyphenyl)methyl]amino]-6-cyclopropyl-5-hydroxy-pyridine-3-carbonitrile (270 mg, 0.45 mmol), sodium chlorodifluoroaceate (138 mg, 0.91 mmol) and cesium carbonate (444 mg, 1.36 mmol) in DMF (2 mL) was stirred 80° C. for 2 h. The reaction was then diluted with water, extracted with ethyl acetate and the combined organic washed with brine and concentrated. Purification by flash column chromatography afforded 2-[bis[(4-methoxyphenyl)methyl]amino]-6-cyclopropyl-5-(difluoromethoxy)pyridine-3-carbonitrile (180 mg, 85%) as yellow oil. ([M+H]⁺ 466.3)

### Step 5: 2-amino-6-cyclopropyl-5-(difluoromethoxy)pyridine-3-carbonitrile

To 2-[bis[(4-methoxyphenyl)methyl]amino]-6-cyclopropyl-5-(difluoromethoxy)pyridine-3-carbonitrile (160 mg, 0.34 mmol) was added TFA (3.0 mL, 0.34 mmol) at rt and the reaction stirred for 1 h before it was quenched by addition of saturated sodium hydrogen carbonate solution. It was then extracted with ethyl aceate, concentrated and the residue purified by flash column chromatography to afford 2-amino-6-cyclopropyl-5-(difluoromethoxy)pyridine-3-carbonitrile (70 mg, 90%) as colorless oil. ([M+H]⁺ 226.2)

### Step 6: 6-cyclopropyl-5-(difluoromethoxy)-2-(2-methylanilino)pyridine-3-carbonitrile

The title compound ([M+H]⁺ 316.2) was prepared from 2-amino-6-cyclopropyl-5-(difluoromethoxy)pyridine-3-carbonitrile by reaction with 2-bromotoluene using Pd(OAc)₂ as a catalyst and xanthphos as a ligand (General procedure B1).

### Step 7: 4-amino-7-cyclopropyl-6-(difluoromethoxy)-1-(o-tolyl)pyrido[2,3-d]pyrimidin-2-one

The title compound ([M+H]⁺ 359.2) was prepared from 6-cyclopropyl-5-(difluoromethoxy)-2-(2-methylanilino)pyridine-3-carbonitrile using General procedure C.

### Example 114 &115: (+)-4-amino-1-(2˜chlorophenyl)˜7-cyclopropylpyrido[2,3-d]pyrimidin-2(H)-one & (−)-4-amino-1-(2-chlorophenyl)-7-cyclopropylpyrido[2,3-d]pyrimidin-2 (1H)-one

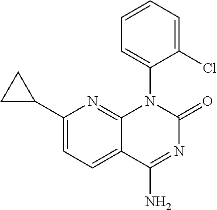 [SMILES: Nc1nc(=O)n(-c2ccccc2Cl)c2nc(C3CC3)ccc12]

### Step 1: 2-((2-chlorophenyl)amino)-6-cyclopropylnicotinonitrile

The title compound ([M+H]⁺ 270.1) was prepared from 2-chloro-6-cyclopropylnicotinonitrile (CAS [1198475-35-2]) by reaction with 2-chloroaniline (CAS [95-51-2]) using Pd(OAc)₂ as a catalyst and xanthphos as a ligand (General procedure B1).

### Step 2: (+)-4-amino-1-(2-chlorophenyl)-7-cyclopropylpyrido[2,3-d]pyrimidin-2 (1H)-one & (−)-4-amino-1-(2-chlorophenyl)-7-cyclopropylpyrido[2,3-d]pyrimidin-2 (1H)-one

The title compounds ([M+H]⁺ 313.1) were prepared from 2-((2-chlorophenyl)amino)-6-cyclopropylnicotinonitrile using General procedure C followed by separation using chiral HPLC.

### Example 116 & 117: (+)-4-amino-1-(2-chloro-3-fluorophenyl)-7-cyclopropylpyrido[2,3-d]pyrimidin-2 (1H)-one and (−)-4-amino-1-(2-chloro-3-fluorophenyl)-7-cyclopropylpyrido[2,3-d]pyrimidin-2 (1H)-one

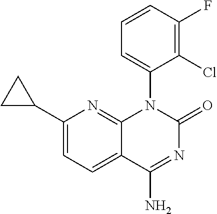 [SMILES: Nc1nc(=O)n(-c2cccc(F)c2Cl)c2nc(C3CC3)ccc12]

### Step 1: 2-((2-chloro-3-fluorophenyl)amino)-6-cyclopropylnicotinonitrile

The title compound ([M+H]⁺ 288.1) was prepared from 2-chloro-6-cyclopropylnicotinonitrile (CAS [1198475-35-2]) by reaction with 2-chloro-3-fluoroaniline (CAS [21397-08-0]) using Pd(OAc)₂ as a catalyst and xanthphos as a ligand (General procedure B1).

### Step 2: (+)-4-amino-1-(2-chloro-3-fluorophenyl)-7-cyclopropylpyrido[2,3-d]pyrimidin-2 (1H)-one and (−)-4-amino-1-(2-chloro-3-fluorophenyl)-7-cyclopropylpyrido[2,3-d]pyrimidin-2 (1H)-one

The title compounds ([M+H]⁺ 331.1 & 331.1) were prepared from 2-((2-chloro-3-fluorophenyl)amino)-6-cyclopropylnicotinonitrile using General procedure C followed by separation using chiral HPLC.

### Example 118 & 119: 4-amino-7-cyclopropyl-1-((2R,3S)-2-methyltetrahydro-2H-pyran-3-yl)pyrido[2,3-d]pyrimidin-2 (1H)-one (example 118) and 4-amino-7-cyclopropyl-1-((2S,3R)-2-methyltetrahydro-2H-pyran-3-yl)pyrido[2,3-d]pyrimidin-2 (1H)-one (example 119)

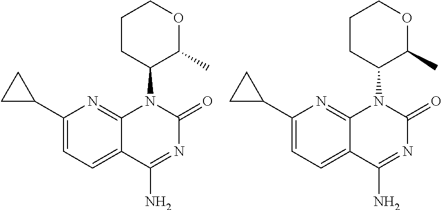

### Step 1: 2-methyldihydro-2H-pyran-3 (4H)-one

To a solution of 2-methyl-2H-pyran-3 (6H)-one (WO2010/96338 A1) (6.45 g, 57.5 mmol) in MeOH (250 ml) was added 10% palladium on activated charcoal (317 mg, 298 μmol) and the reaction stirred under an atmosphere of hydrogen (balloon) for 1 h. The reaction was filtered over Celite® and concentrated. Vacuum distillation over a short Vigreux column (Bp: 42-43 @10 mbar) afforded the title compound (4.75 g, 65%) as a colourless liquid. ([M+H]⁺ 115.1)

### Step 2: (2RS,3RS)-2-methyltetrahydro-2H-pyran-3-ol

To a cooled (−78° C.) solution of 2-methyldihydro-2H-pyran-3 (4H)-one (1.00 g, 8.76 mmol) in dry THF (25 ml) was added L-selectride® 1 M in THF (20 ml, 20 mmol) dropwise over 30 minutes and the mixture stirred for a further 3 h which time the reaction was allowed to come to −10° C. before ethanol (2.4 ml, 41.1 mmol) was added dropwise followed by dropwise addition of water (6 ml, 333 mmol) and finally NaOH 1 M in Water (6 ml, 6 mmol). The temperature was then raised to 0° C. for addition of 36% H₂O₂ (6 ml, 70.5 mmol) dropwise keeping the temperature below 10° C. after which time the mixture was stirred for a further 1 h at rt. The reaction was filtered over Celite®, washing with ethylacetate. The filtrate was washed with sat. NaHCO₃ and 10% sodium thiosulfate-solution. All aqueous layers were re-extracted with DCM:MeOH (9:1) and the organic phases combined, dried (Na₂SO₄) and concentrated. The residue was by flash column chromatography to afford the title compound (0.74 g, 72%) as a colorless oil. 1H NMR (300 MHz, CHLOROFORM-d) δ ppm 1.18-1.23 (m, 3H) 1.35-1.45 (m, 1H) 1.61-1.73 (m, 1H) 1.81-2.01 (m, 3H) 3.43-3.59 (m, 3H) 3.91-4.00 (m, 1H)

### Step 3: (2RS,3RS)-2-methyltetrahydro-2H-pyran-3-yl 4-methylbenzenesulfonate

To a solution of (2RS, 2RS)-2-methyltetrahydro-2H-pyran-3-ol (730 mg, 6.28 mmol) in dry DCM (25 ml) was added DABCO (1.41 g, 12.6 mmol). The solution was cooled in an ice bath and toluenesulfonyl chloride (1.8 g, 9.43 mmol) was added and the ice bath removed and the mixture stirred for 20 minutes at rt. The reaction was concentrated and the residue purified by flash column chromatography to afford the title compound (1.37 g, 80%) as white solid. 1H NMR (300 MHz, CHLOROFORM-d) δ ppm 1.04 (d, J=6.45 Hz, 3H) 1.31-1.41 (m, 1H) 1.62-1.74 (m, 1H) 1.83-1.99 (m, 1H) 2.05-2.16 (m, 1H) 2.45 (s, 3H) 3.40-3.54 (m, 2H) 3.91-3.99 (m, 1H) 4.50 (brs, 1H) 7.30-7.36 (m, 2H) 7.79-7.84 (m, 2H)

### Step 4: (2RS,3SR)-2-methyltetrahydro-2H-pyran-3-amine acetate

To a solution of (2RS,3RS)-2-methyltetrahydro-2H-pyran-3-yl 4-methylbenzenesulfonate (1.37 g, 5.07 mmol) in dry DMF (8 ml) was added sodium azide (1.65 g, 25.3 mmol) and the suspension heated to 65° C. for 94 h. The reaction was diluted with water and extracted with diethyl ether, the combined organic extracts washed with water and dried (Na₂SO₄), filtered and 30 mL of methanol added to the filtrate, which was then cautiously concentrated (p>250 mbar, water bath 25° C.) to remove the diethyl ether. Acetic acid (1.45 mL, 25.3 mmol) was then added to the methanolic solution followed by 10% palladium on charcoal (132 mg, 124 μmol) and the reaction placed under an atmosphere of hydrogen (balloon) and the mixture stirred for 3 h. The reaction was then filtered over Celite® and concentrated. The residue was suspended in diethyl ether, sonicated and filtered to afford the title compound (176 mg, 16%) as off-white solid. ([M+H]⁺ 116.1)

### Step 5: 6-cyclopropyl-2-(((2RS,3SR)-2-methyltetrahydro-2H-pyran-3-yl)amino)nicotinonitrile

The title compound ([M+H]⁺ 258.2) was prepared from 2-chloro-6-cyclopropylnicotinonitrile (CAS [1198475-35-2]) by reaction with (2SR,3RS)-2-methyltetrahydro-2H-pyran-3-amine acetate using DIPEA as base in NMP at 150° C. (General procedure B2).

### Step 6: 4-amino-7-cyclopropyl-1-((2R,3S)-2-methyltetrahydro-2H-pyran-3-yl)pyrido[2,3-d]pyrimidin-2 (1H)-one and 4-amino-7-cyclopropyl-1-((2S,3R)-2-methyltetrahydro-2H-pyran-3-yl)pyrido[2,3-d]pyrimidin-2 (1H)-one

The title compounds ([M+H]⁺ 301.2 & 301.2) were prepared from 6-cyclopropyl-2-(((2RS,3SR)-2-methyltetrahydro-2H-pyran-3-yl)amino)nicotinonitrile using General procedure C followed by separation using chiral HPLC.

### Example 120: 4-amino-1-(benzo[d]thiazol-7-yl-7-cyclopropylpyrido[2,3-d]pyrimidin-2 (1H)-one

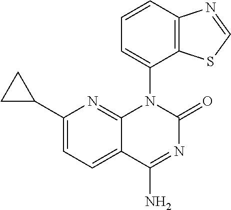 [SMILES: Nc1nc(=O)n(-c2cccc3ncsc23)c2nc(C3CC3)ccc12]

### Step 1: 2-(benzo[d]thiazol-7-ylamino)-6-cyclopropylnicotinonitrile

The title compound ([M+H]⁺ 293.1) was prepared from 2-chloro-6-cyclopropylnicotinonitrile (CAS [1198475-35-2]) by reaction with 6,7-dihydro-5H-pyrrolo[1,2-c]imidazol-7-amine hydrochloride (CAS [272438-86-5]) using Pd₂(dba)₃ as a catalyst and tBuXphos as a ligand (General procedure B1).

### Step 2: 4-amino-1-(benzo[d]thiazol-7-yl) 7-cyclopropylpyrido[2,3-d]pyrimidin-2 (1H)-one

The title compound ([M+H]⁺ 336.1) was prepared from 2-(benzo[d]thiazol-7-ylamino)-6-cyclopropylnicotinonitrile using General procedure C.

### Example 121: 4-amino-7-cyclopropyl-1-[(2SR,3SR)-2-methyltetrahydropyran-3-yl]pyrido[2,3-d]pyrimidin-2-one

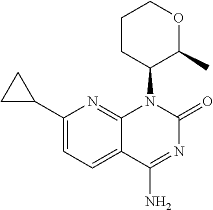 [SMILES: C[C@@H]1OCCC[C@@H]1n1c(=O)nc(N)c2ccc(C3CC3)nc21]

### Step 1: (2SR,3SR)—N-benzyl-2-methyltetrahydro-2H-pyran-3-amine

To a solution of 2-methyldihydro-2H-pyran-3 (4H)-one (Example 118, step 1) (500 mg, 4.38 mmol) and sodium triacetoxyborohydride (1.39 g, 6.57 mmol) in dry DCM (14 ml) was added phenylmethanamine (526 μl, 4.82 mmol) and acetic acid (301 μl, 5.26 mmol) at 0° C., the reaction was brought to rt and stirred for 1 h after which time it was diluted with DCM washed with TN NaOH solution, dried (Na₂SO₄) and concentrated. The residue was purified by flash column chromatography to afford the title compound (736 mg, 65%) as a colourless oil. ([M+H]⁺ 116.1)

### Step 1: (2SR,3SR)-2-methyltetrahydro-2H-pyran-3-amine acetate

To a solution of (2SR,3SR)—N-benzyl-2-methyltetrahydro-2H-pyran-3-amine (730 mg, 3.56 mmol) in THF dry (14 ml) and acetic acid (407 μl, 7.11 mmol) was added 10% palladium on activate charcoal (378 mg, 356 μmol) and the reaction set under an atmosphere of hydrogen (balloon) and stirred for 24 h. The reaction was filtered over Celite® washing with MeOH and concentrated to afford the title compound (628 mg, 75%) as an off-white solid. ([M+H]⁺ 116.1)

### Step 1: 6-cyclopropyl-2-(((2SR,3SR)-2-methyltetrahydro-2H-pyran-3-yl)amino)nicotinonitrile

The title compound ([M+H]⁺ 258.2) was prepared from 2-chloro-6-cyclopropylnicotinonitrile (CAS [1198475-35-2]) by reaction with (2SR,3SR)-2-methyltetrahydro-2H-pyran-3-amine acetate using Pd₂(dba)₃ as a catalyst and tBuXphos as a ligand (General procedure B1).

### Step 2: 4-amino-1-(benzo[d]thiazol-7-yl)-7-cyclopropylpyrido[2,3-d]pyrimidin-2 (1H)-one

The title compound ([M+H]⁺ 301.2) was prepared from 6-cyclopropyl-2-(((2SR,3SR)-2-methyltetrahydro-2H-pyran-3-yl)amino)nicotinonitrile using General procedure C.

### Example 122: 4˜amino-7-(difluoromethoxy)-1-(3-fluoro-2-methylphenyl)quinazolin-2 (1H)-one

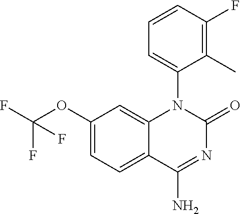 [SMILES: Cc1c(F)cccc1-n1c(=O)nc(N)c2ccc(OC(F)(F)F)cc21]

### Step 1: 4-(difluoromethoxy)-2-((3-fluoro-2-methylphenyl)amino)benzonitrile

The title compound ([M+H]⁺ 293.1) was prepared from 2-bromo-4-(trifluoromethoxy)benzonitrile (CAS [1214334-83-4]) by reaction with 3-fluoro-2-methylaniline using Pd(OAc)₂ as a catalyst and xanthphos as a ligand (General procedure B1).

### Step 2: 4-amino-7-(difluoromethoxy)-1-(3-fluoro-2-methylphenyl)quinazolin-2 (1H)-one

The title compound ([M+H]⁺ 336.2) was prepared from 4-(difluoromethoxy)-2-((3-fluoro-2-methylphenyl)amino)benzonitrile using General procedure C.

### Example 123: 4-amino-7-cyclopropyl-1-(3-hydroxy-2-methylphenyl)pyrido[2,3-d]pyrimidin-2 (1H)-one

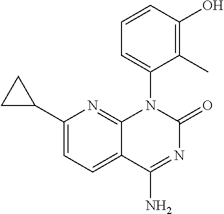 [SMILES: Cc1c(O)cccc1-n1c(=O)nc(N)c2ccc(C3CC3)nc21]

### Step 1: 2-((3-((tert-butyldimethylsilyl)oxy)-2-methylphenyl)amino)-6-cyclopropylnicotinonitrile

The title compound ([M+H]⁺ 380.6) was prepared from 2-chloro-6-cyclopropylnicotinonitrile (CAS [1198475-35-2]) by reaction with 3-((tert-butyldimethylsilyl)oxy)-2-methylaniline using Pd(OAc)₂ as a catalyst and xanthphos as a ligand (General procedure B1).

### Step 2: 4-amino-1-(3-((tert-butyldimethylsilyl)oxy)-2-methylphenyl)-7-cyclopropylpyrido[2,3-d]pyrimidin-2 (1H)-one

The title compound ([M+H]⁺ 423.8) was prepared from 2-((3-((tert-butyldimethylsilyl)oxy)-2-methylphenyl)amino)-6-cyclopropylnicotinonitrile using General procedure C.

### Step 3: 4-amino-7-cyclopropyl-1-(3-hydroxy-2-methylphenyl)pyrido[2,3-d]pyrimidin-2 (1H)-one

To a suspension of 4-amino-1-(3-((tert-butyldimethylsilyl)oxy)-2-methylphenyl)-7-cyclopropylpyrido[2,3-d]pyrimidin-2 (1H)-one (47 mg, 111 μmol) in HCl 4 M in dioxane (1 ml) was added MeOH (0.5 ml) was added and the reaction was stirred at rt for 4 h. 1 ml water was then added and mixture was stirred for 30 min after which time the title product (22 mg, 64%) was isolated by filtration as a white solid. ([M+H]⁺ 309.2)

### Example 124 & 125: (R)-4-amino-7-cyclopropyl-1-(oxepan-3-yl)pyrido[2,3-d]pyrimidin-2 (1H)-one and (S)-4-amino-7-cyclopropyl-1-(oxepan-3-yl)pyrido[2,3-d]pyrimidin-2 (1H)-one

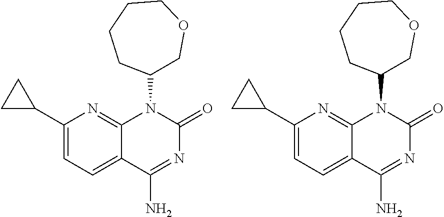

### Step 3: (E/Z)-oxepan-3-one oxime

To a solution of 6,7-dihydrooxepin-3 (2H)-one (1.35 g, 12 mmol, CAS: 497063-30-6) in MeOH (50 ml) was added 10% palladium on activated charcoal (100 mg, 94 μmol) and the reaction ste under an atmosphere of hydrogen (balloon) and the mixture stirred for 30 minutes. The reaction was then filtered over Celite®, washing with methanol and the filtrate partially concentrated (p>100 mbar@20° C.). Hydroxylamine hydrochloride (1.67 g, 24.1 mmol) and potassium acetate (4.73 g, 48.2 mmol) were then added and the reaction heated to 70° C. for 1 hour after which time the reaction was concentrated to dryness and then partioned between water and ethyl actetate. The layers were separated and the aqueous fraction re-extracted with ethylacetate. The combined organic layers were washed with brine and concentrated. The residue was by flash column chromatography to afford the title compounds (1.21 g, 74%) as a colourless oil. ([M+H]⁺ 130.0)

### Step 4: oxepan-3-amine hydrochloride

To a solution of oxepan-3-one oxime (1.21 g, 9.37 mmol) in 7M ammonia in methanol (150 ml) was added Raney®-Nickel (6.2 g, 9.37 mmol) and the mixture stirred under an atmosphere of hydrogen (balloon) for 90 minutes. The reaction was then filtered over Celite® and concentrated. Purification by flash column chromatography followed by precipitation from diethyl ether (made acidic by addition of 4N HCl in dioxane) afforded the title compound (1.05 g, 76%) as a white solid. ([M+H]⁺ 116.1)

### Step 5: 6-cyclopropyl-2-(oxepan-3-ylamino)nicotinonitrile

The title compound ([M+H]⁺ 258.2) was prepared from 2-chloro-6-cyclopropylnicotinonitrile (CAS [1198475-35-2]) by reaction with oxepan-3-amine hydrochloride using DIPEA as base in NMP at 150° C. (General procedure B2).

### Step 6: (R)-4-amino-7-cyclopropyl-1-(oxepan-3-yl)pyrido[2,3-d]pyrimidin-2 (1H)-one and (S)-4-amino-7-cyclopropyl-1-(oxepan-3-yl)pyrido[2,3-d]pyrimidin-2 (1H)-one

The title compounds ([M+H]⁺ 301.2 & 301.2) were prepared from 6-cyclopropyl-2-(oxepan-3-ylamino)nicotinonitrile using General procedure C followed by separation using chiral HPLC.

### Example 126: 3-(4-amino-7-cyclopropyl-2-oxopyrido[2,3-d]pyrimidin-1 (21H)-yl)-2-fluorobenzonitrile

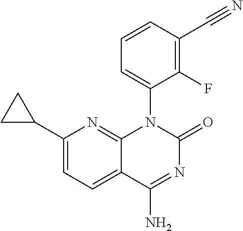 [SMILES: N#Cc1cccc(-n2c(=O)nc(N)c3ccc(C4CC4)nc32)c1F]

### Step 1: 2-((3-cyano-2-fluorophenyl)amino)-6-cyclopropylnicotinonitrile

The title compound ([M+H]⁺ 279.1) was prepared from 2-chloro-6-cyclopropylnicotinonitrile (CAS [1198475-35-2]) by reaction with 3-amino-2-fluorobenzonitrile using Pd(OAc)₂ as a catalyst and xanthphos as a ligand (General procedure B1).

### Step 2: 3-(4-amino-7-cyclopropyl-2-oxopyrido[2,3-d]pyrimidin-1 (2H)-yl)-2-fluorobenzonitrile

The title compound ([M+H]⁺ 322.1) was prepared from 2-((3-cyano-2-fluorophenyl)amino)-6-cyclopropylnicotinonitrile using General procedure C.

### Example 127: 4-amino-7-cyclopropyl-1-(2-fluoro-3-methylphenyl)pyrido[2,3-d]pyrimidin-2 (1H)-one

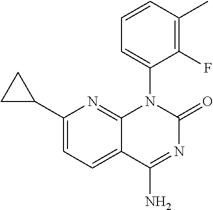 [SMILES: Cc1cccc(-n2c(=O)nc(N)c3ccc(C4CC4)nc32)c1F]

### Step 1: 6-cyclopropyl-2-((2-fluoro-3-methylphenyl)amino)nicotinonitrile

The title compound ([M+H]⁺ 268.2) was prepared from 2-chloro-6-cyclopropylnicotinonitrile (CAS [1198475-35-2]) by reaction with 2-fluoro-3-methylaniline using Pd(OAc)₂ as a catalyst and xanthphos as a ligand (General procedure B1).

### Step 2: 4-amino-7-cyclopropyl-1-(2-fluoro-3-methylphenyl)pyrido[2,3-d]pyrimidin-2 (1H)-one

The title compound ([M+H]⁺ 311.1) was prepared from 6-cyclopropyl-2-((2-fluoro-3-methylphenyl)amino)nicotinonitrile using General procedure C.

### Example 128: 4-amino-7-cyclopropyl-1-(2,3-dichlorophenyl)pyrido[2,3-d]pyrimidin-2 (1H)-one

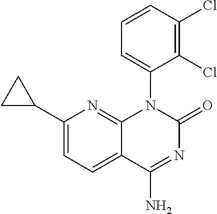 [SMILES: Nc1nc(=O)n(-c2cccc(Cl)c2Cl)c2nc(C3CC3)ccc12]

### Step 1: 6-cyclopropyl-2-((2,3-dichlorophenyl)amino)nicotinonitrile

The title compound ([M+H]⁺ 304.0) was prepared from 2-chloro-6-cyclopropylnicotinonitrile (CAS [1198475-35-2]) by reaction with 2,3-dichloroaniline using Pd(OAc)₂ as a catalyst and xanthphos as a ligand (General procedure B1).

### Step 2: 4-amino-7-cyclopropyl-1-(2,3-dichlorophenyl)pyrido[2,3-d]pyrimidin-2 (1H)-one

The title compound ([M+H]⁺ 347.1) was prepared from 6-cyclopropyl-2-((2,3-dichlorophenyl)amino)nicotinonitrile using General procedure C.

### Example 129: 4-amino-1-(3-chloro-2-methylphenyl)-7-cyclopropylpyrido[2,3-d]pyrimidin-2-one

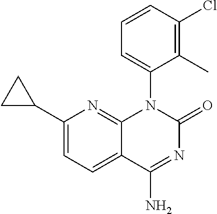 [SMILES: Cc1c(Cl)cccc1-n1c(=O)nc(N)c2ccc(C3CC3)nc21]

### Step 1: 2-((3-chloro-2-methylphenyl)amino)-6-cyclopropylnicotinonitrile

The title compound ([M+H]⁺ 284.1) was prepared from 2-chloro-6-cyclopropylnicotinonitrile (CAS [1198475-35-2]) by reaction with 3-chloro-2-methylaniline using Pd(OAc)₂ as a catalyst and xanthphos as a ligand (General procedure B1).

### Step 2: 4-amino-1-(3-chloro-2-methylphenyl)-7-cyclopropylpyrido[2,3-d]pyrimidin-2-one

The title compound ([M+H]⁺ 327.1) was prepared from 2-((3-chloro-2-methylphenyl)amino)-6-cyclopropylnicotinonitrile using General procedure C.

### Example 130: 4-amino-1-(2-chloro-3-methylphenyl)-7-cyclopropylpyrido[2,3-d]pyrimidin-2-one

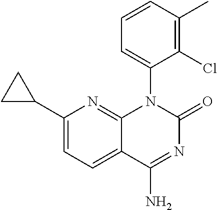 [SMILES: Cc1cccc(-n2c(=O)nc(N)c3ccc(C4CC4)nc32)c1Cl]

### Step 1: 2-((2-chloro-3-methylphenyl)amino)-6-cyclopropylnicotinonitrile

The title compound ([M+H]⁺ 284.1) was prepared from 2-chloro-6-cyclopropylnicotinonitrile (CAS [1198475-35-2]) by reaction with 2-chloro-3-methylaniline using Pd(OAc)₂ as a catalyst and xanthphos as a ligand (General procedure B1).

### Step 2: 4-amino-1-(2-chloro-3-methylphenyl)-7-cyclopropylpyrido[2,3-d]pyrimidin-2-one

The title compound ([M+H]⁺ 327.1) was prepared from 2-((2-chloro-3-methylphenyl)amino)-6-cyclopropylnicotinonitrile using General procedure C.

### Example 131: 4-amino-7-cyclopropyl-1-(3-(fluoromethyl)˜2-methylphenyl)pyrido[2,3˜d]pyrimidin-2 (1H)-one

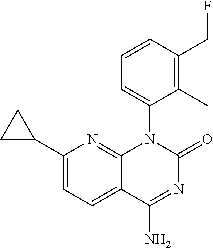 [SMILES: Cc1c(CF)cccc1-n1c(=O)nc(N)c2ccc(C3CC3)nc21]

### Step 1: 6-cyclopropyl-2-((3-(fluoromethyl)-2-methylphenyl)amino)nicotinonitrile

The title compound ([M+H]⁺ 282.3) was prepared from 2-chloro-6-cyclopropylnicotinonitrile (CAS [1198475-35-2]) by reaction with 3-(fluoromethyl)-2-methylaniline using Pd(OAc)₂ as a catalyst and xanthphos as a ligand (General procedure B1).

### Step 2: 4-amino-7-cyclopropyl-1-(3-(fluoromethyl)-2-methylphenyl)pyrido[2,3-d]pyrimidin-2 (1H)-one

The title compound ([M+H]⁺ 325.1) was prepared from 6-cyclopropyl-2-((3-(fluoromethyl)-2-methylphenyl)amino)nicotinonitrile using General procedure C.

### Example 132: 4-amino-7-cyclopropyl-1-(2-(trifluoromethyl)phenyl)pyrido[2,3-d]pyrimidin-2 (1H)-one

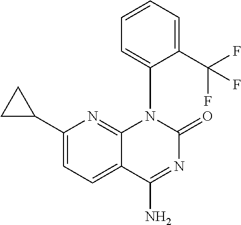 [SMILES: Nc1nc(=O)n(-c2ccccc2C(F)(F)F)c2nc(C3CC3)ccc12]

### Step 1: 6-cyclopropyl-2-((2-(trifluoromethyl)phenyl)amino)nicotinonitrile

The title compound ([M+H]⁺ 304.1) was prepared from 2-chloro-6-cyclopropylnicotinonitrile (CAS [1198475-35-2]) by reaction with 2-(trifluormethyl)aniline (CAS [88-17-5]) using Pd₂(dba)₃ as a catalyst and Xphos as a ligand (General procedure B1).

### Step 2: 4-amino-7-cyclopropyl-1-(2-(trifluoromethyl)phenyl)pyrido[2,3-d]pyrimidin-2 (1H)-one

The title compound ([M+H]⁺ 347.1) was prepared from 6-cyclopropyl-2-((2-(trifluoromethyl)phenyl)amino)nicotinonitrile using General procedure C.

### Example 133: 4-amino-7-(difluoromethoxy)-1-(2-fluoro-3-methylphenyl)quinazolin-2 (1H)-one

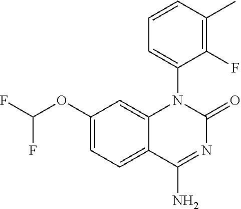 [SMILES: Cc1cccc(-n2c(=O)nc(N)c3ccc(OC(F)F)cc32)c1F]

### Step 1: bromo-4-(difluoromethoxy)benzonitrile

To a solution of 2-bromo-4-hydroxy-benzonitrile (30.2 g, 122 mmol) and cesium carbonate (119.3 g, 366 mmol) in DMF (302 mL) was added sodium 2-chloro-2,2-difluoroacetate (55.8 g, 366 mmol) and the reaction mixture was heated to 80° C. for 2 h after which time the reaction was filtered, diluted with ethyl acetate and washed with water, brine and concentrated. Purification by flash column chromatography afforded the title compound (6.0 g, 18%) as a white solid. 1H NMR (400 MHz, CHLOROFORM-d) δ=7.72-7.65 (m, 1H), 7.51-7.45 (m, 1H), 7.24-7.14 (m, 1H), 6.81-6.39 (m, 1H).

### Step 2: 4-(difluoromethoxy)-2-((2-fluoro-3-methylphenyl)amino)benzonitrile

The title compound ([M+H]⁺ 293.1) was prepared from 2-bromo-4-(difluoromethoxy)benzonitrile by reaction with 2-fluoro-3-methylaniline using Pd(OAc)₂ as a catalyst and xanthphos as a ligand (General procedure B1).

### Step 3: 4-amino-7-(difluoromethoxy)-2-(2-fluoro-3-methylphenyl)quinazolin-2 (1H)-one

The title compound ([M+H]⁺ 336.1) was prepared from 4-(difluoromethoxy)-2-((2-fluoro-3-methylphenyl)amino)benzonitrile using General procedure C.

### Example 134: 4-amino-7-(difluoromethoxy)-1-(m-tocly)quinazolin-2 (1H)-one

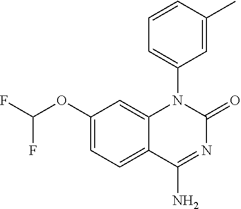 [SMILES: Cc1cccc(-n2c(=O)nc(N)c3ccc(OC(F)F)cc32)c1]

### Step 1: 4-(difluoromethoxy)-2-(m-tolylamino)benzonitrile

The title compound ([M+H]⁺ 275.1) was prepared from 2-bromo-4-(difluoromethoxy)benzonitrile (Example 133, step 1) by reaction with m-toluidine using Pd(OAc)₂ as a catalyst and xanthphos as a ligand (General procedure B1).

### Step 2: 4-amino-7-(difluoromethoxy)-1-(m-tolyl)quinazolin-2 (1H)-one

The title compound ([M+H]⁺ 318.1) was prepared from 4-(difluoromethoxy)-2-(m-tolylamino)benzonitrile using General procedure C.

### Example 135 & 136: (+)-4-amino-1-(2-chloropyridin-3-yl)-7-cyclopropylpyrido[2,3-d]pyrimidin-2 (1H)-one and (−)-4-amino-1-(2-chloropyridin-3-yl)-7-cyclopropylpyrido[2,3-d]pyrimidin-2 (1H)-one

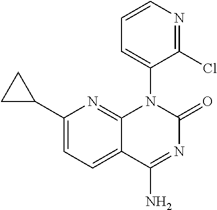 [SMILES: Nc1nc(=O)n(-c2cccnc2Cl)c2nc(C3CC3)ccc12]

### Step 1: 2-((2-chloropyridin-3-yl)amino)-6-cyclopropylnicotinonitrile

The title compound ([M+H]⁺ 271.1) was prepared from 2-amino-6-cyclopropylnicotinonitrile (CAS [1249836-67-6]) by reaction with 2-chloro-3-iodopyridine using Pd₂(dba)₃ as a catalyst and xanthphos as a ligand (General procedure B1).

### Step 2: (+)-4-amino-1-(2-chloropyridin-3-yl)-7-cyclopropylpyrido[2,3-d]pyrimidin-2 (1H)-one and (−)-4-amino-1-(2-chloropyridin-3-yl)-7-cyclopropylpyrido[2,3-d]pyrimidin-2 (1H)-one

The title compounds ([M+H]⁺ 314.2 & 314.2) were prepared from 6-2-((2-chloropyridin-3-yl)amino)-6-cyclopropylnicotinonitrile using General procedure C followed by separation using chiral HPLC.

### Example 137: 4-amino-1-(2-chloropyridin-3-yl)-7-(difluoromethoxy)quinazolin-2 (1H)-one

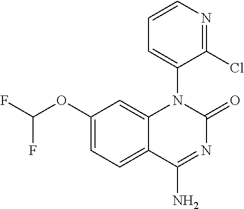 [SMILES: Nc1nc(=O)n(-c2cccnc2Cl)c2cc(OC(F)F)ccc12]

### Step 1: 2-((2-chloropyridin-3-yl)amino)-4-(difluoromethoxy)benzonitrile

The title compound ([M+H]⁺ 296.1) was prepared from 2-bromo-4-(difluoromethoxy)benzonitrile (CAS [1261818-72-7]) by reaction with 2-chloropyridin-3-amine using Pd₂(dba)₃ as a catalyst and xanthphos as a ligand (General procedure B1).

### Step 2: 4-amino-1-(2-chloropyridin-3-yl)-7-difluoromethoxy)quinazolin-2 (1H)-one

The title compound ([M+H]⁺ 339.1) was prepared from 2-((2-chloropyridin-3-yl)amino)-4-(difluoromethoxy)benzonitrile using General procedure C.

### Example 138: 4-amino-7-cyclopropyl-1-(2,3-dimethylphenyl)pyrido[2,3-d]pyrimidin-2 (1H)-one

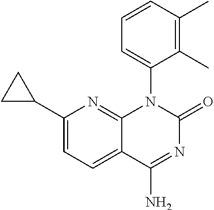 [SMILES: Cc1cccc(-n2c(=O)nc(N)c3ccc(C4CC4)nc32)c1C]

### Step 1: 6-cyclopropyl-2-((2,3-dimethylphenyl)amino)nicotinonitrile

The title compound ([M+H]⁺ 264.3) was prepared from 2-chloro-6-cyclopropylnicotinonitrile (CAS [1198475-35-2]) by reaction with 2,3-dimethylaniline using Pd₂(dba)₃ as a catalyst and xanthphos as a ligand (General procedure B1).

### Step 2: 4-amino-7-cyclopropyl-1-(2,3-dimethylphenyl)pyrido[2,3-d]pyrimidin-2 (1H)-one

The title compound ([M+H]⁺ 307.2) was prepared from 6-cyclopropyl-2-((2,3-dimethylphenyl)amino)nicotinonitrile using General procedure C.

### Example 139: 4-amino-7-cyclopropyl-1-(1H-indazol-4-yl)pyrido[2,3-d]pyrimidin-2 (1H)-one

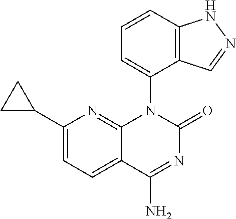 [SMILES: Nc1nc(=O)n(-c2cccc3[nH]ncc23)c2nc(C3CC3)ccc12]

### Step 1: tert-butyl 4-((3-cyano-6-cyclopropylpyridin-2-yl)amino)-1H-indazole-1-carboxylate

The title compound ([M+H]⁺ 264.3) was prepared from 2-amino-6-cyclopropylnicotinonitrile (CAS [1249836-67-6]) by reaction with tert-butyl 4-bromo-1H-indazole-1-carboxylate using Pd₂(dba)₃ as a catalyst and Xphos as a ligand (General procedure B1).

### Step 2: 4-amino-7-cyclopropyl-1-(1H-indazol-4-yl)pyrido[2,3-d]pyrimidin-2 (1H)-one

The title compound ([M+H]⁺ 319.1) was prepared from tert-butyl 4-((3-cyano-6-cyclopropylpyridin-2-yl)amino)-1H-indazole-1-carboxylate using General procedure C.

### Example 140: 4-amino-7-cyclopropyl-1-(1H-indazol-4-yl)pyrido[2,3-d]pyrimidin-2 (1H)-one

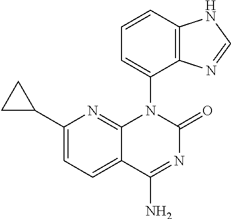 [SMILES: Nc1nc(=O)n(-c2cccc3[nH]cnc23)c2nc(C3CC3)ccc12]

### Step 1: tert-butyl 4-((3-cyano-6-cyclopropylpyridin-2-yl)amino)-1H-benzo[d]imidazole-1-carboxylate

The title compound ([M+H]⁺ 376.2) was prepared from 2-amino-6-cyclopropylnicotinonitrile (CAS [1249836-67-6]) by reaction with tert-butyl 4-bromo-1H-benzo[d]imidazole-1-carboxylate (WO2018/132372 A1) using Pd₂(dba)₃ as a catalyst and Xphos as a ligand (General procedure B1).

### Step 2: 4-amino-7-cyclopropyl-1-(1H-indazol-4-yl)pyrido[2,3-d]pyrimidin-2 (1H)-one

The title compound ([M+H]⁺ 317.2) was prepared from tert-butyl 4-((3-cyano-6-cyclopropylpyridin-2-yl)amino)-1H-indazole-1-carboxylate using General procedure C.

### Example 141: 4-amino-7-cyclopropyl-1-[(8R)-4-oxaspiro[2.5]octan-8-yl]pyrido[2,3-d]pyrimidin-2-one

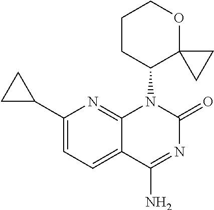 [SMILES: Nc1nc(=O)n([C@@H]2CCCOC23CC3)c2nc(C3CC3)ccc12]

### Step 1: methyl 1-(allyloxy)cyclopropane-1-carboxylate

To an ice cold solution of methyl 1-hydroxycyclopropane-1-carboxylate (10 g, 86.1 mmol) in dry THF (220 ml) was added sodium hydride, 60% dispersion in mineral oil (4.13 g, 103 mmol) and the mixture stirred for 15 minutes before the addition of allyl bromide (9.69 ml, 112 mmol) dissolved in dry THF (50 ml) over 30 min. The mixture was allowed to come to rt and stirred for 16 h after which time the reaction was quenched by addition of saturated aqueous ammonium chloride solution, extracted with TBME and the combined organics were dried (Na₂SO₄) and concentrated. Distillation (Bp 79-82° C.@12 mmbar) afforded the title compound (6.25 g, 44.1% yield) as a light yellow oil. ([M+H]⁺ 157.1)

### Step 2: 1-(allyloxy)-N-methoxy-N-methylcyclopropane-1-carboxamide

To a ice-cold suspension of N,O-dimethylhydroxylamine hydrochloride (1.25 g, 12.8 mmol) in dry DCM (12 ml) was added trimethylaluminum 2 M in toluene (6.4 ml, 12.8 mmol) and the mixture stirred for 1 h before a solution of methyl 1-(allyloxy)cyclopropane-1-carboxylate (1 g, 6.4 mmol) in dry DCM (6 ml) was added over 10 min. The ice bath was removed and the reaction stirred for 16 h at rt. The reaction was then cooled to 0° C., quenched by cautious addition of water, followed by 4N aq. HCl and extracted with DCM. The combined organics were dried (Na₂SO₄) and concentrated. The reside was by flash column chromatography to afford the title compound (768 mg, 65%) as a colourless oil. ([M+H]⁺ 186.1)

### Step 3: 1-(1-(allyloxy)cyclopropyl)prop-2-en-1-one

To a −78° C. solution of 1-(allyloxy)-N-methoxy-N-methylcyclopropane-1-carboxamide (463 mg, 2.5 mmol) in dry THF (8 ml) was added vinylmagnesium bromide 1 M in THF (2.75 ml, 2.75 mmol) over 10 minutes and the mixture stirred for 1 h. A second portion of vinylmagnesium bromide 1 M in THF (2.75 ml, 2.75 mmol) was added and the reaction warmed to 0° C. over 30 minutes. The reaction was recooled to −78° C. before addition of 4N aq HCl (10 ml) and the temperature raised to rt. The mixture was diluted with water, extracted with TBME, the combined organics were dried (Na₂SO₄) and concentrated to afford the title compound (357 mg, 89%) as a yellow oil.

1H NMR (300 MHz, CHLOROFORM-d) δ ppm 1.21-1.28 (m, 2H) 1.34-1.41 (m, 2H) 4.04 (dt, J=5.44, 1.51 Hz, 2H) 5.15-5.36 (m, 2H) 5.74 (dd, J=10.38, 1.91 Hz, 1H) 5.92 (ddt, J=17.33, 10.58, 5.39, 5.39 Hz, 1H) 6.40 (dd, J=17.33, 2.01 Hz, 1H) 7.02 (dd, J=17.23, 10.38 Hz, 1H)

### Step 4: 4-oxaspiro[2.5]oct-6-en-8-one

To a solution of 1-(1-(allyloxy)cyclopropyl)prop-2-en-1-one (2.7 g, 17.7 mmol) in DCM (324 ml) was added Zhan Catalyst-1B (130 mg, 177 μmol) and the mixture stirred at rt for 3 h. The reaction was concentrated and the residue purified by flash column chromatography to afford the title compound (1.9 g, 83%) as a colourless oil. ([M+H]⁺ 125.0)

### Step 5: 4-oxaspiro[2.5]octan-8-one

To a solution of 4-oxaspiro[2.5]oct-6-en-8-one (302 mg, 2.43 mmol) in THF (7 ml) was added 10% palladium on activated charcoal (12 mg, 11.3 μmol) and the mixture set under an atmosphere of hydrogen (balloon), stirred for 40 minutes. It was then filtered over Celite® and concentrated to afford the title compound (296 mg, 96%) as a colourless oil. ([M+H]⁺ 127.1)

### Step 6: (S,Z)-2-methyl-N-(4-oxaspiro[2.5]octan-8-ylidene)propane-2-sulfinamide

To a solution of TiOEt₄ (496 μl, 2.35 mmol) in THF (2 ml) was added 4-oxaspiro[2.5]octan-8-one (148 mg, 1.17 mmol) in THF (2 ml) followed by addition of (S)-(−)-2-methyl-2-propanesulfinamide (174 mg, 1.41 mmol) and the mixture heated for 68 h at 45° C. The reaction was diluted with ethyl acetate, brine was added resulting in a thick suspension which was then filtered over Celite®. The mixture was extracted with ethyl acetate the combined organics were dried (Na₂SO₄) and concentrated. The reside was by flash column chromatography to afford the title compound (120 mg, 44%) as a yellow oil. ([M+H]⁺ 230.2)

### Step 7: (S)-2-methyl-N—((R)-4-oxaspiro[2.5]octan-8-yl)propane-2-sulfinamide

To a solution of (S,Z)-2-methyl-N-(4-oxaspiro[2.5]octan-8-ylidene)propane-2-sulfinamide (120 mg, 523 μmol) in THF (2.0 ml) was added water (41 μl) and cooled to −50° C. before sodium borohydride (59.4 mg, 1.57 mmol) was added. It was allowed to come to 15° C. over 3 h. The reaction was quenched by addition of methanol (0.5 ml), water (2 ml) and 10% sodium carbonate solution (2 ml) and stirred for 30 min. The reaction was extracted with ethyl acetate, the combined organics were dried (Na₂SO₄) and concentrated. The reside was by flash column chromatography to afford the title compound (65 mg, 54%) as a white solid. ([M+H]⁺ 232.1)

### Step 8: (R)-4-oxaspiro[2.5]octan-8-amine hydrochloride

To a solution of (S)-2-methyl-N—((R)-4-oxaspiro[2.5]octan-8-yl)propane-2-sulfinamide (60 mg, 0.3 mmol) in dioxane (2 ml) was added 4M HCl in Dioxane (195 μl, 778 μmol) and the mixture stirred for 16 h at rt. The reaction was evaporated to dryness, suspended in diethyl ether and filtered to afford the titled compound (38 mg, 89%) as a white solid. ([M+H]⁺ 111.1)

### Step 9: 6-cyclopropyl-2-((2,3-dimethylphenyl)amino)nicotinonitrile

The title compound ([M+H]⁺ 270.2) was prepared from 2-chloro-6-cyclopropylnicotinonitrile (CAS [1198475-35-2]) by reaction with (R)-4-oxaspiro[2.5]octan-8-amine hydrochloride using Pd₂(dba)₃ as a catalyst and tBuXphos as a ligand (General procedure B1).

### Step 10: 4-amino-7-cyclopropyl-1-[(8R)-4-oxaspiro[2.5]octan-8-yl]pyrido[2,3-d]pyrimidin-2-one

The title compound ([M+H]⁺ 313.1) was prepared from (R)-2-((4-oxaspiro[2.5]octan-8-yl)amino)-6-cyclopropylnicotinonitrile using General procedure C.

### Example 142: 4-amino-7-cyclopropyl-1-[(8S)-4-oxaspiro[2.5]octan-8-yl]pyrido[2,3-d]pyrimidin-2-one

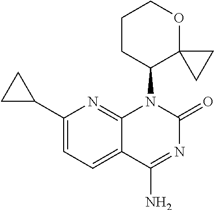 [SMILES: Nc1nc(=O)n([C@H]2CCCOC23CC3)c2nc(C3CC3)ccc12]

### Step 1: (S)-2-((4-oxaspiro[2.5]octan-8-yl)amino)-6-cyclopropylnicotinonitrile

The title compound ([M+H]⁺ 270.2) was prepared from 2-chloro-6-cyclopropylnicotinonitrile (CAS [1198475-35-2]) by reaction with (S)-4-oxaspiro[2.5]octan-8-amine hydrochloride (prepared in analogy to example 141 but using (R)-(−)-2-methyl-2-propanesulfinamide in step 6) using Pd₂(dba)₃ as a catalyst and tBuXphos as a ligand (General procedure B1).

### Step 2: 4-amino-7-cyclopropyl-1-[(8R)-4-oxaspiro[2.5]octan-8-yl]pyrido[2,3-d]pyrimidin-2-one

The title compound ([M+H]⁺ 313.1) was prepared from (S)-2-((4-oxaspiro[2.5]octan-8-yl)amino)-6-cyclopropylnicotinonitrile using General procedure C.

### Example 143: 3-(4-amino-7-cyclopropyl-2-oxopyrido[2,3-d]pyrimidin-1 (2H)-yl)-2-chlorobenzonitrile

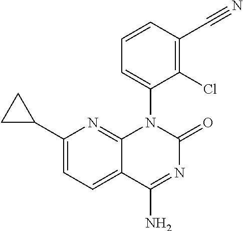 [SMILES: N#Cc1cccc(-n2c(=O)nc(N)c3ccc(C4CC4)nc32)c1Cl]

### Step 1: 3-amino-2-chlorobenzonitrile

To 2-chloro-3-nitrobenzonitrile (0.5 g, 2.74 mmol), iron powder (3.09 g, 54.8 mmol) and ammonium chloride (3.66 g, 68.5 mmol) were added EtOH (29 ml) and water (12 ml). The reaction was stirred at 70° C. for 3 h. The reaction was filtered over Decalite® washing with DCM, MeOH and EtOAc and the filtrate evaporated to dryness. Suspension in DCM and concentration of the filtrate afforded the titled compound (426 mg, 102%) as an off-white solid. ([M+H]⁺ 153.0)

### Step 2: 2-((2-chloro-3-cyanophenyl)amino)-6-cyclopropylnicotinonitrile

The title compound ([M+H]⁺ 295.1) was prepared from 2-amino-6-cyclopropylnicotinonitrile (CAS [1249836-67-6]) by reaction with 3-amino-2-chlorobenzonitrile using Pd₂(dba)₃ as a catalyst and xanthphos as a ligand (General procedure B1).

### Step 3: 3-(4-amino-7-cyclopropyl-2-oxopyrido[2,3-d]pyrimidin-1 (2H)-yl)-2-chlorobenzonitrile

The title compound ([M+H]⁺ 338.2) was prepared from 2-((2-chloro-3-cyanophenyl)amino)-6-cyclopropylnicotinonitrile using General procedure C.

### Example 144: 4-amino-7-(difluoromethoxy)-1-(o-tolyl)quinazolin-2 (1H)-one

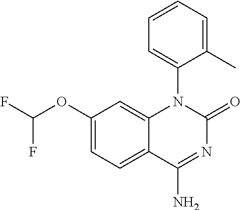 [SMILES: Cc1ccccc1-n1c(=O)nc(N)c2ccc(OC(F)F)cc21]

### Step 1: 4-(difluoromethoxy)-2-(o-tolylamino)benzonitrile

The title compound ([M+H]⁺ 275.2) was prepared from 2-bromo-4-(difluoromethoxy)benzonitrile (CAS [1261818-72-7]) by reaction with o-toluidine using Pd₂(dba)₃ as a catalyst and xanthphos as a ligand (General procedure B1).

### Step 2: 4-amino-1-(2-chloropyridin-3-yl)-7-(difluoromethoxy)quinazolin-2 (1H)-one

The title compound ([M+H]⁺ 318.2) was prepared from 4-(difluoromethoxy)-2-(o-tolylamino)benzonitrile using General procedure C.

### Example 145 & 146: (+)-3-(4-amino-7-cyclopropyl-2-oxopyrido[2,3-d]pyrimidin-1 (2H)-yl)-2-methylbenzonitrile and (−)-3-(4-amino-7-cyclopropyl-2-oxopyrido[2,3-d]pyrimidin-1 (2H)-yl)-2-methylbenzonitrile

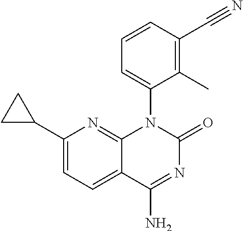 [SMILES: Cc1c(C#N)cccc1-n1c(=O)nc(N)c2ccc(C3CC3)nc21]

### Step 1: 2-((3-cyano-2-methylphenyl)amino)-6-cyclopropylnicotinonitrile

The title compound ([M+H]⁺ 275.3) was prepared from 2-chloro-6-cyclopropylnicotinonitrile (CAS [1198475-35-2]) by reaction with 3-amino-2-methylbenzonitrile (CAS [69022-35-1] using Pd(OAc)₂ as a catalyst and Xphos as a ligand (General procedure B1).

### Step 2: (+)-3-(4-amino-7-cyclopropyl-2-oxopyrido[2,3-d]pyrimidin-1 (2H)-yl)-2-methylbenzonitrile and (−)-3-(4-amino-7-cyclopropyl-2-oxopyrido[2,3-d]pyrimidin-1 (2H)-yl)-2-methylbenzonitrile

The title compounds ([M+H]⁺ 318.2 & 318.2) were prepared from 2-((3-cyano-2-methylphenyl)amino)-6-cyclopropylnicotinonitrile using General procedure C followed by separation using chiral HPLC.

### Example 147: 4-amino-7-cyclopropyl-1-(3,4-difluorophenyl)pyrido[2,3-d]pyrimidin-2 (1H)-one

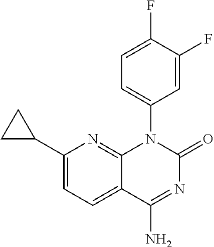 [SMILES: Nc1nc(=O)n(-c2ccc(F)c(F)c2)c2nc(C3CC3)ccc12]

### Step 1: 6-cyclopropyl-2-((3,4-difluorophenyl)amino)nicotinonitrile

The title compound ([M+H]⁺ 272.1) was prepared from 2-chloro-6-cyclopropylnicotinonitrile (CAS [1198475-35-2]) by reaction with 3,4-difluoroaniline using Pd₂(dba)₃ as a catalyst and xanthphos as a ligand (General procedure B1).

### Step 2: 4-amino-7-cyclopropyl-1-(3,4-difluorophenyl)pyrido[2,3-d]pyrimidin-2 (1H)-one

The title compound ([M+H]⁺ 315.1) was prepared from 6-cyclopropyl-2-((3,4-difluorophenyl)amino)nicotinonitrile using General procedure C.

### Example 148: 4-amino-1-(benzo[d]oxazol-4-yl)-7-cyclopropylpyrido[2,3-d]pyrimidin-2 (1H)-one

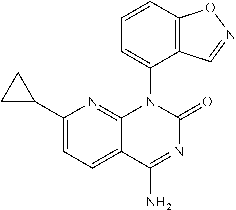 [SMILES: Nc1nc(=O)n(-c2cccc3oncc23)c2nc(C3CC3)ccc12]

### Step 1: 2-(benzo[d]oxazol-4-ylamino)-6-cyclopropylnicotinonitrile

The title compound ([M+H]⁺ 277.2) was prepared from 2-chloro-6-cyclopropylnicotinonitrile (CAS [1198475-35-2]) by reaction with benzo[d]oxazol-4-amine using Pd₂(dba)₃ as a catalyst and tBuXphos as a ligand (General procedure B1).

### Step 2: 4-amino-7-cyclopropyl-1-(3,4-difluorophenyl)pyrido[2,3-d]pyrimidin-2 (1H)-one

The title compound ([M+H]⁺ 320.1) was prepared from 2-(benzo[d]oxazol-4-ylamino)-6-cyclopropylnicotinonitrile using General procedure C.

### Example 149: 1-amino-4-(2-chlorophenyl)-6-(trifluoroethyl)pyrido[1,2-c]pyrimidin-3-one

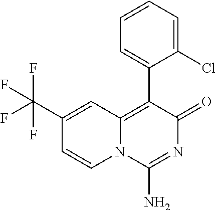

### Step 1: 2-(2-chlorophenyl)-2-[4-(trifluoromethyl)-2-pyridyl]acetonitrile

To a solution of 2-chloro-4-(trifluoromethyl)pyridine (3.35 g, 18.4 mmol) and 2-chlorobenzyl cyanide (2.00 g, 13.1 mmol) in DMF (50 mL) was added NaH (1.47 g, 36.9 mmol) and the reaction mixture was stirred for 20 minutes. The reaction mixture was poured into water, extracted with ethyl acetate, the combined extracts were washed with brine and concentrated. Purification by preparative TLC afforded the title compound (3.00 g, 69% yield) as a yellow oil. ([M+H]⁺ 297.1)

### Step 2: 2-(2-chlorophenyl)-2-[4-(trifluoromethyl)-2-pyridyl]acetamide

To a solution of 2-(2-chlorophenyl)-2-[4-(trifluoromethyl)-2-pyridyl]acetonitrile (2.0 g, 6.74 mmol) in AcOH (15 mL) was added 95% H₂SO₄ (5 mL) and mixture was stirred for 2 days at 40° C. The reaction mixture was cooled to rt and poured onto ice followed by extraction with EtOAc. The organic layers were washed with sat. NaHCO₃ solution and brine, dried over Na₂SO₄ and concentrated. Trituration from hexane afforded the title compound (1500 mg, 69%) as yellow solid. ([M+H]⁺ 315.1)

### Step 3: 4-(2-chlorophenyl)-1-thioxo-6-(trifluoromethyl)pyrido[1,2-c]pyrimidin-3-one

To a mixture of 2-(2-chlorophenyl)-2-[4-(trifluoromethyl)-2-pyridyl]acetamide (500 mg, 1.59 mmol) in EtOH (2.5 mL) was added sodium ethoxide (4.6 mL, 21% in EtOH, 12.7 mmol) followed by dropwise addition of thiophosgene (245 μL, 3.18 mmol) keeping the temperature below 40° C. The mixture was stirred in a sealed tube at 85° C. for 2 h before it was cooled to rt and quenched with ˜3 mL water. The mixture was extracted with ethyl acetate, the combined washings washed with brine and concentrated. Purification by flash column chromatography to gave the title compound (400 mg, 64%) as yellow solid. ([M+H]⁺ 357.0)

### Step 4: 4-(2-chlorophenyl)-1-methylsulfanyl-6-(trifluoromethyl)pyrido[1,2-c]pyrimidin-3-one

To a solution of 4-(2-chlorophenyl)-1-thioxo-6-(trifluoromethyl)pyrido[1,2-c]pyrimidin-3-one (250 mg, 0.70 mmol) in DMF (5 mL) was added potassium carbonate (193 mg, 1.40 mmol) iodomethane (51 μL, 0.84 mmol). The reaction was stirred at rt for 7 h. The reaction mixture was evaporated and the residue was diluted with EtOAc/water. The organic layers were washed with brine, dried over Na₂SO₄ and concentrated. Purification by flash column chromatography gave the titled compound (150 mg, 55%) as yellow solid. ([M+H]⁺ 371.0)

### Step 5: 1-amino-4-(2-chlorophenyl)-6-(trifluoromethyl)pyrido[1,2-c]pyrimidin-3-one

To a mixture of 4-(2-chlorophenyl)-1-methylsulfanyl-6-(trifluoromethyl)pyrido[1,2-c]pyrimidin-3-one (100 mg, 0.270 mmol) and ammonium hydroxide solution (1.5 mL, 0.270 mmol) was added THF (1 mL). The reaction was stirred at rt for 48 h after which time it was concentrated. Purification by reversed phase preparative HPLC afforded the titled product (65 mg, 68%) as yellow solid ([M+H]⁺ 340.0)

### Example 150: 4-amino-7-(difluoromethoxy)-1-(4-oxaspiro[2.5]octan-8-yl)quinazolin-2 (1H)-one

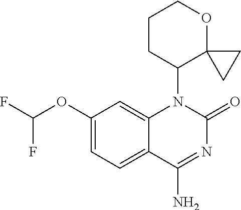 [SMILES: Nc1nc(=O)n(C2CCCOC23CC3)c2cc(OC(F)F)ccc12]

### Step 1: 2-((4-oxaspiro[2.5]octan-8-yl)amino)-4-(difluoromethoxy)benzonitrile

The title compound ([M+H]⁺ 295.1) was prepared from 2-bromo-4-(difluoromethoxy)benzonitrile (CAS [1261818-72-7]) by reaction with 4-oxaspiro[2.5]octan-8-amine hydrochloride using Pd₂(dba)₃ as a catalyst and xanthphos as a ligand (General procedure B1).

### Step 2: 4-amino-7-(difluoromethoxy)-1-(4-oxaspiro[2.5]octan-8-yl)quinazolin-2 (1H)-one

The title compound ([M+H]⁺ 338.2) was prepared from 2-((4-oxaspiro[2.5]octan-8-yl)amino)-4-(difluoromethoxy)benzonitrile using General procedure C.

### Example 151: 3-(4-amino-7-cyclopropyl-2-oxopyrido[2,3-d]pyrimidin-1 (2H)-yl)benzonitrile

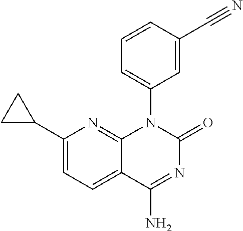 [SMILES: N#Cc1cccc(-n2c(=O)nc(N)c3ccc(C4CC4)nc32)c1]

### Step 1: 2-((3-cyanophenyl)amino)-6-cyclopropylnicotinonitrile

The title compound ([M+H]⁺ 261.1) was prepared from 2-chloro-6-cyclopropylnicotinonitrile (CAS [1198475-35-2]) by reaction with 3-aminobenzonitrile using Pd₂(dba)₃ as a catalyst and xanthphos as a ligand (General procedure B1).

### Step 2: 3-(4-amino-7-cyclopropyl-2-oxopyrido[2,3-d]pyrimidin-1 (2H)-yl)benzonitrile

The title compound ([M+H]⁺ 304.1) was prepared from 2-((3-cyanophenyl)amino)-6-cyclopropylnicotinonitrile using General procedure C.

### Example 152: 4-amino-1-(3-chlorophenyl)-7-cyclopropylpyrido[2,3-d]pyrimidin-2 (1H)-one

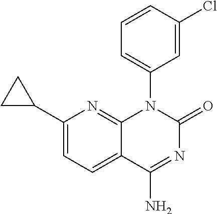 [SMILES: Nc1nc(=O)n(-c2cccc(Cl)c2)c2nc(C3CC3)ccc12]

### Step 1: 2-((3-chlorophenyl)amino)-6-cyclopropylnicotinonitrile

The title compound ([M+H]⁺ 270.1) was prepared from 2-chloro-6-cyclopropylnicotinonitrile (CAS [1198475-35-2]) by reaction with 3-chloroaniline using Pd(OAc)₂ as a catalyst and xanthphos as a ligand (General procedure B1).

### Step 2: 4-amino-1-(3-chlorophenyl)-7-cyclopropylpyrido[2,3-d]pyrimidin-2 (1H)-one

The title compound ([M+H]⁺ 313.1) was prepared from 2-((3-chlorophenyl)amino)-6-cyclopropylnicotinonitrile using General procedure C.

### Example 153: 4-amino-7-(ethylamino)-1-(o-tolyl)quinazolin-2-one

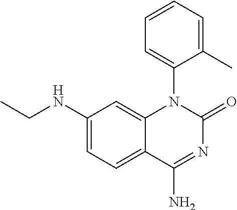 [SMILES: CCNc1ccc2c(N)nc(=O)n(-c3ccccc3C)c2c1]

### Step 1: 2-bromo-4-(ethylamino)benzonitrile

To a solution of 2-bromo-4-fluorobenzonitrile (700 mg, 3.5 mmol) in DMF (7 mL) were added ethylamine hydrochloride (571 mg, 7 mmol) and K₂CO₃ (967 mg, 7 mmol) and the reaction mixture heated to 90° C. for 6 h. The reaction was diluted with ethyl acetate and washed with water, brine and concentrated. Purification by flash column chromatography afforded the titled compound (850 mg, 75%) as yellow solid. ([M+H]⁺ 225.1)

### Step 2: tert-butyl N-(3-bromo-4-cyano-phenyl)-N-ethyl-carbamate

To a solution 2-bromo-4-(ethylamino)benzonitrile (750 mg, 2.70 mmol) of di-t-butyldicarbonate (1163 mg, 5.33 mmol) in DCM (15 mL) was added triethylamine (1.11 mL, 8 mmol) and DMAP (65.13 mg, 0.530 mmol). The reaction mixture was stirred at rt for 12 h after which time it was concentrated and the residue purified by flash column chromatography to yield the titled compound (700 mg, 810%) as a yellow oil. ([M+H-tBu]⁺269.1)

### Step 3: tert-butyl N-[4-cyano-3-(2-methylanilino)phenyl]-N-ethyl-carbamate

The title compound ([M+H-tBu]⁺296.1) was prepared from tert-butyl N-(3-bromo-4-cyano-phenyl)-N-ethyl-carbamate by reaction with o-toluidine using Pd(OAc)₂ as a catalyst and xanthphos as a ligand (General procedure B1).

### Step 4: tert-butyl N-[4-amino-1-(o-tolyl)-2-oxo-quinazolin-7-yl]-N-ethyl-carbamate

The title compound ([M+H]⁺ 395.3) was prepared from tert-butyl N-[4-cyano-3-(2-methylanilino)phenyl]-N-ethyl-carbamate using General procedure C.

### Step 5: 4-amino-7-(ethylamino)-1-(o-tolyl)quinazolin-2-one

To a solution of tert-butyl N-[4-amino-1-(o-tolyl)-2-oxo-quinazolin-7-yl]-N-ethyl-carbamate (350 mg, 0.89 mmol) in DCM (5 mL) was added TFA (3.0 mL, 0.89 mmol) and the mixture stirred at rt for 2 h. The reaction was quenched by addition of saturated sodium hydrogen carbonate solution, extracted with DCM and the combined organic concentrated. Purification by reversed phase preparative HPLC afforded the title compound (51 mg, 19%) as white solid. ([M+H]⁺ 295.2)

### Example 154: 4-amino-7-cyclopropyl-1-(2,5-difluorophenyl)pyrido[2,3-d]pyrimidin-2 (1H)-one

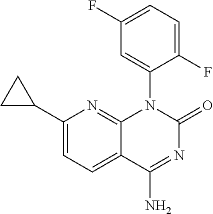 [SMILES: Nc1nc(=O)n(-c2cc(F)ccc2F)c2nc(C3CC3)ccc12]

### Step 1: 6-cyclopropyl-2-((2,5-difluorophenyl)amino)nicotinonitrile

The title compound ([M+H]⁺ 272.2) was prepared from 2-chloro-6-cyclopropylnicotinonitrile (CAS [1198475-35-2]) by reaction with 2,5-difluoroaniline using Pd(OAc)₂ as a catalyst and xanthphos as a ligand (General procedure B1).

### Step 2: 4-amino-7-cyclopropyl-1-(2,5-difluorophenyl)pyrido[2,3-d]pyrimidin-2 (1H)-one

The title compound ([M+H]⁺ 315.2) was prepared from 6-cyclopropyl-2-((2,5-difluorophenyl)amino)nicotinonitrile using General procedure C.

### Example 155: 4-amino-1-(2-chloro-4-fluorophenyl)-7-cyclopropylpyrido[2,3-d]pyrimidin-2 (1H)-one

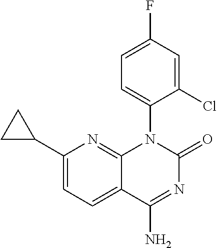 [SMILES: Nc1nc(=O)n(-c2ccc(F)cc2Cl)c2nc(C3CC3)ccc12]

### Step 1: 2-((2-chloro-4-fluorophenyl)amino)-6-cyclopropylnicotinonitrile

The title compound ([M+H]⁺ 288.1) was prepared from 2-chloro-6-cyclopropylnicotinonitrile (CAS [1198475-35-2]) by reaction with 2-chloro-4-fluoroaniline using Pd(OAc)₂ as a catalyst and xanthphos as a ligand (General procedure B1).

### Step 2: 4-amino-1-(2-chloro-4-fluorophenyl)-7-cyclopropylpyrido[2,3-d]pyrimidin-2 (1H)-one

The title compound ([M+H]⁺ 331.0) was prepared from 6-cyclopropyl-2-((2,5-difluorophenyl)amino)nicotinonitrile using General procedure C.

### Example 156: 4-amino-1-(2-chloro-5-fluorophenyl)-7-cyclopropylpyrido[2,3-d]pyrimidin-2-one

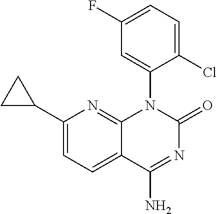 [SMILES: Nc1nc(=O)n(-c2cc(F)ccc2Cl)c2nc(C3CC3)ccc12]

### Step 1: 2-((2-chloro-5-fluorophenyl)amino)-6-cyclopropylnicotinonitrile

The title compound ([M+H]⁺ 288.0) was prepared from 2-chloro-6-cyclopropylnicotinonitrile (CAS [1198475-35-2]) by reaction with 2-chloro-5-fluoroaniline using Pd(OAc)₂ as a catalyst and xanthphos as a ligand (General procedure B1).

### Step 2: 4-amino-1-(2-chloro-5-fluorophenyl)-7-cyclopropylpyrido[2,3-d]pyrimidin-2-one

The title compound ([M+H]⁺ 331.0) was prepared from 2-((2-chloro-5-fluorophenyl)amino)-6-cyclopropylnicotinonitrile using General procedure C.

### Example 157: 4-amino-7-cyclopropyl-1-(2,3-dihydro-1H-indol-4-yl)pyrido[2,3-d]pyrimidin-2-one

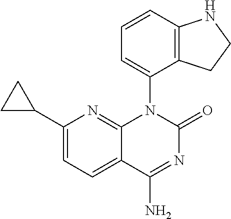 [SMILES: Nc1nc(=O)n(-c2cccc3c2CCN3)c2nc(C3CC3)ccc12]

### Step 1: tert-butyl 4-((3-cyano-6-cyclopropylpyridin-2-yl)amino)indoline-1-carboxylate

The title compound ([M+H]⁺ 377.2) was prepared from tert-butyl 4-aminoindoline-1-carboxylate (US2010/36123 A1) by reaction with 2-chloro-6-cyclopropylnicotinonitrile (CAS [1198475-35-2]) using Pd(OAc)₂ as a catalyst and xanthphos as a ligand (General procedure B1).

### Step 2: 4-amino-7-cyclopropyl-1-(2,3-dihydro-1H-indol-4-yl)pyrido[2,3-d]pyrimidin-2-one

tert-butyl 4-((3-cyano-6-cyclopropylpyridin-2-yl)amino)indoline-1-carboxylate was first converted to tert-butyl 4-(4-amino-7-cyclopropyl-2-oxo-pyrido[2,3-d]pyrimidin-1-yl)indoline-1-carboxylate using General procedure C and the crude material deprotected using TFA in DCM in analogy to Example 153 to afford the title compound. ([M+H]⁺ 320.1)

### Example 158: 3-[4-amino-2-oxo-7-(trifluoromethoxy)quinazolin-1-yl]-2-methylbenzonitrile

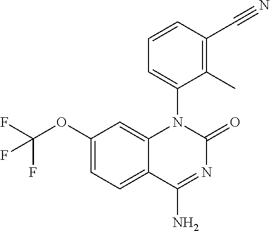 [SMILES: Cc1c(C#N)cccc1-n1c(=O)nc(N)c2ccc(OC(F)(F)F)cc21]

### Step 1: 3-((2-cyano-5-(trifluoromethoxy)phenyl)amino)-2-methylbenzonitrile

The title compound ([M+H]⁺ 316.1) was prepared from 2-amino-4-(trifluoromethoxy)benzonitrile (CAS [1260847-67-3]) by reaction with 3-chloro-2-methylbenzonitrile (CAS [54454-12-5]) using Pd(OAc)₂ as a catalyst and xanthphos as a ligand (General procedure B1).

### Step 2: 3-[4-amino-2-oxo-7-(trifluoromethoxy)quinazolin-1-yl]-2-methylbenzonitrile

The title compound ([M+H]⁺ 361.1) was prepared from 6-cyclopropyl-2-((2,5-difluorophenyl)amino)nicotinonitrile using General procedure C.

### Example 159: 4-amino-1-(3-fluoro-2-methylphenyl)-7-(trifluoromethoxy)quinazolin-2-one

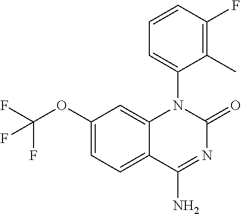 [SMILES: Cc1c(F)cccc1-n1c(=O)nc(N)c2ccc(OC(F)(F)F)cc21]

### Step 1: 2-((3-fluoro-2-methylphenyl)amino)-4-(trifluoromethoxy)benzonitrile

The title compound ([M+H]⁺ 311.1) was prepared from 2-bromo-4-(trifluoromethoxy)benzonitrile (CAS [1214334-83-4]) by reaction with 3-fluoro-2-methylaniline (CAS [54454-12-5]) using Pd(OAc)₂ as a catalyst and xanthphos as a ligand (General procedure B1).

### Step 2: 4-amino-1-(3-fluoro-2-methylphenyl)-7-(trifluoromethoxy)quinazolin-2-one

The title compound ([M+H]⁺ 354.1) was prepared from 6-cyclopropyl-2-((2,5-difluorophenyl)amino)nicotinonitrile using General procedure C.

### Example 160: 4-amino-1-(2,3-dihydro-1-benzofuran-4-yl)-7-(trifluoromethoxy)quinazolin-2-one

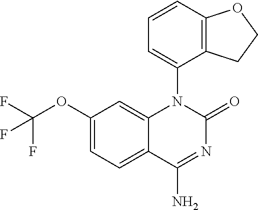 [SMILES: Nc1nc(=O)n(-c2cccc3c2CCO3)c2cc(OC(F)(F)F)ccc12]

### Step 1: 2-((2,3-dihydrobenzofuran-4-yl)amino)-4-(trifluoromethoxy)benzonitrile

The title compound ([M+H]⁺ 321.0) was prepared from 2-bromo-4-(trifluoromethoxy)benzonitrile (CAS [1214334-83-4]) by reaction with 2,3-dihydrobenzofuran-4-amine (CAS [61090-37-7]) using Pd(OAc)₂ as a catalyst and xanthphos as a ligand (General procedure B1).

### Step 2: 4-amino-1-(3-fluoro-2-methylphenyl)-7-(trifluoromethoxy)quinazolin-2-one

The title compound ([M+H]⁺ 364.2) was prepared from 2-((2,3-dihydrobenzofuran-4-yl)amino)-4-(trifluoromethoxy)benzonitrile using General procedure C.

### Example 161: 4-amino-1-(3-chloro-2-fluorophenyl)-7-cyclopropylpyrido[2,3-d]pyrimidin-2-one

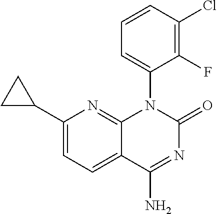 [SMILES: Nc1nc(=O)n(-c2cccc(Cl)c2F)c2nc(C3CC3)ccc12]

### Step 1: 2-((3-chloro-2-fluorophenyl)amino)-6-cyclopropylnicotinonitrile

The title compound ([M+H]⁺ 288.1) was prepared from 2-chloro-6-cyclopropylnicotinonitrile (CAS [1198475-35-2]) by reaction with 2-chloro-5-fluoroaniline (CAS [2106-04-9]) using Pd(OAc)₂ as a catalyst and xanthphos as a ligand (General procedure B1).

### Step 2: 4-amino-1-(3-chloro-2-fluorophenyl)-7-cyclopropylpyrido[2,3-d]pyrimidin-2-one

The title compound ([M+H]⁺ 331.0) was prepared from 4-amino-1-(3-chloro-2-fluorophenyl)-7-cyclopropylpyrido[2,3-d]pyrimidin-2-one using General procedure C.

### Example 162: 4-amino-1-(2,3-dihydrobenzofuran-4-yl)-7-(trifluoromethyl)pyrido[2,3-d]pyrimidin-2 (1H)-one

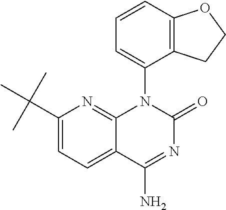 [SMILES: CC(C)(C)c1ccc2c(N)nc(=O)n(-c3cccc4c3CCO4)c2n1]

### Step 1: 2-((2,3-dihydrobenzofuran-4-yl)amino)-6-(trifluoromethyl)nicotinonitrile

The title compound ([M+H]⁺ 306.1) was prepared from 2-chloro-6-(trifluoromethyl)nicotinonitrile (CAS [1249836-67-6]) by reaction with 2,3-dihydrobenzofuran-4-amine using Pd(OAc)₂ as a catalyst and xanthphos as a ligand (General procedure B1).

### Step 2: 4-amino-1-(2,3-dihydrobenzofuran-4-yl)-7-(trifluoromethyl)pyrido[2,3-d]pyrimidin-2 (1H)-one

The title compound ([M+H]⁺ 349.1) was prepared from 2-((2,3-dihydrobenzofuran-4-yl)amino)-6-(trifluoromethyl)nicotinonitrile using General procedure C.

### Example 163: 4-amino-7-cyclopropyl-1-[6-(trifluoromethoxy)pyridin-2-yl]pyrido[2,3-d]pyrimidin-2-one

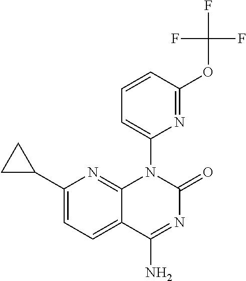 [SMILES: Nc1nc(=O)n(-c2cccc(OC(F)(F)F)n2)c2nc(C3CC3)ccc12]

### Step 1: 6-cyclopropyl-2-((6-(trifluoromethoxy)pyridin-2-yl)amino)nicotinonitrile

The title compound ([M+H]⁺ 321.1) was prepared from 2-chloro-6-cyclopropylnicotinonitrile (CAS [1198475-35-2]) by reaction with 6-(trifluoromethoxy)pyridin-2-amine (CAS [1131007-45-8]) using Pd(OAc)2 as a catalyst and xanthphos as a ligand (General procedure B1).

### Step 2: 4-amino-7-cyclopropyl-1-[6-(trifluoromethoxy)pyridin-2-yl]pyrido[2,3-d]pyrimidin-2-one

The title compound ([M+H]⁺ 364.1) was prepared from 6-cyclopropyl-2-((6-(trifluoromethoxy)pyridin-2-yl)amino)nicotinonitrile using General procedure C.

### Example 164: 4-amino-7-cyclopropyl-1-(1H-indol-4-yl)pyrido[2,3-d]pyrimidin-2-one

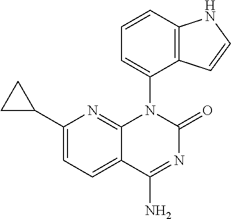 [SMILES: Nc1nc(=O)n(-c2cccc3[nH]ccc23)c2nc(C3CC3)ccc12]

### Step 1: tert-butyl 4-((3-cyano-6-cyclopropylpyridin-2-yl)amino)-1H-indole-1-carboxylate

The title compound ([M+H]⁺ 375.2) was prepared from tert-butyl 4-amino-1H-indole-1-carboxylate (US2009/227575 A1) by reaction with 2-chloro-6-cyclopropylnicotinonitrile (CAS [1198475-35-2]) using Pd(OAc)₂ as a catalyst and xanthphos as a ligand (General procedure B1).

### Step 2: 4-amino-7-cyclopropyl-1-(2,3-dihydro-1H-indol-4-yl)pyrido[2,3-d]pyrimidin-2-one

tert-butyl 4-((3-cyano-6-cyclopropylpyridin-2-yl)amino)-1H-indole-1-carboxylate was first converted to tert-butyl 4-(4-amino-7-cyclopropyl-2-oxo-pyrido[2,3-d]pyrimidin-1-yl)indoline-1-carboxylate using General procedure C and the crude material deprotected using TFA in DCM in analogy to Example 153 to afford the title compound. ([M+H]⁺ 318.1)

### Example 165: 4-amino-1-(3-fluoro-2-methylphenyl)-7-(trifluoromethyl)pyrido[2,3-d]pyrimidin-2 (1H)-one

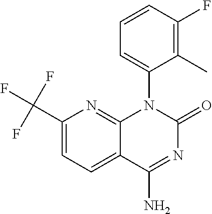 [SMILES: Cc1c(F)cccc1-n1c(=O)nc(N)c2ccc(C(F)(F)F)nc21]

### Step 1: 2-((3-fluoro-2-methylphenyl)amino)-6-(trifluoromethyl)nicotinonitrile

The title compound ([M+H]⁺ 296.1) was prepared from 2-chloro-6-(trifluoromethyl)nicotinonitrile (CAS [1249836-67-6]) by reaction with 3-fluoro-2-methylaniline using Pd(OAc)₂ as a catalyst and xanthphos as a ligand (General procedure B1).

### Step 2: 4-amino-1-(3-fluoro-2-methylphenyl)-7-(trifluoromethyl)pyrido[2,3-d]pyrimidin-2 (1H)-one

The title compound ([M+H]⁺ 339.1) was prepared from 2-((3-fluoro-2-methylphenyl)amino)-6-(trifluoromethyl)nicotinonitrile using General procedure C.

1. A compound formula I or II: 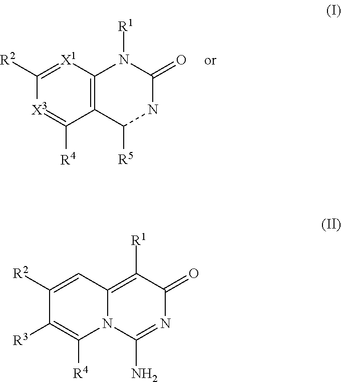
wherein
X¹ is either N or CH;
X³ is either N or CR³
the dotted line represents a single bond when R⁵ is oxo or a double bond when R⁵ is —NH₂,
R¹ is (C₁-C₆)alkyl optionally substituted with one or more, particularly one to three, more particularly one or two substituents R^{1a}, (C₁-C₆)alkoxy optionally substituted with one or more, particularly one to three, more particularly one or two substituents R^{1b}, halo(C₁-C₆)alkyl optionally substituted with one or more, particularly one to three, more particularly one or two substituents R^{1a}, halo(C₁-C₆)alkoxy optionally substituted with one or more, particularly one to three, more particularly one or two substituents R^{1b}, (C₃-C₈)cycloalkyl optionally substituted with one or more, particularly one to three, more particularly one or two substituents R^{1c}, heteroaryl optionally substituted with one or more, particularly one to three, more particularly one or two substituents R^{1d}, heterocycloalkyl optionally substituted with one or more, particularly one to three, more particularly one or two substituents R^{1e} or phenyl optionally substituted with one or more, particularly one to three, more particularly one or two substituents R^{1f};
R^{1a} and R^{1b} are each independently selected from (C₃-C₆)cycloalkyl, hydroxyl, heteroaryl, heterocycloalkyl and phenyl, wherein heteroaryl, heterocycloalkyl or phenyl are optionally substituted with one or more, particularly one to three, more particularly one or two substituents R^{1g};
R^{1c}, R^{1d}, R^{1e} and R^{1f} are each independently selected from halogen, oxo, cyano, hydroxyl, (C₁-C₆)alkyl, (C₁-C₆)alkoxy, (C₃-C₆)cycloalkyl, (C₃-C₆)cycloalkyl-(C₁-C₆)alkyl, halo(C₁-C₆)alkyl, halo(C₁-C₆)alkoxy, hydroxy(C₁-C₆)alkyl, (C₁-C₆)alkoxy-(C₁-C₆)alkyl, heteroaryl, heterocycloalkyl and phenyl;
R^{1g} are each independently selected from halogen, cyano, hydroxyl, (C₁-C₆)alkyl, (C₁-C₆)alkoxy, (C₃-C₆)cycloalkyl, (C₃-C₆)cycloalkyl-(C₁-C₆)alkyl, halo(C₁-C₆)alkyl, halo(C₁-C₆)alkoxy, hydroxy(C₁-C₆)alkyl and (C₁-C₆)alkoxy-(C₁-C₆)alkyl;
R² is hydrogen, halogen, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{2a}, (C₃-C₆)cycloalkyl-(C₁-C₆)alkyl optionally substituted with one or more, particularly one to three, more particularly one or two substituents R^{2b}, (C₃-C₆)cycloalkyl-(C₁-C₆)alkoxy optionally substituted with one or more, particularly one to three, more particularly one or two substituents R^{2c}, heterocycloalkyl optionally substituted with one or more, particularly one to three, more particularly one or two substituents R^{2d}NR^{2f}R^{2g} or phenyl optionally substituted with one or more, particularly one to three, more particularly one or two substituents R^{2e};
R^{2a}, R^{2b}, R^{2c}, R^{2d} and R^{2e} are each independently selected from halogen, (C₁-C₆)alkyl, (C₁-C₆)alkoxy, halo(C₁-C₆)alkyl and halo(C₁-C₆)alkoxy;
R^{2f} and R^{2g} are each independently selected from hydrogen or (C₁-C₆)alkyl;
R³ is hydrogen, halogen, cyano, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{3a}, (C₃-C₆)cycloalkyl-(C₁-C₆)alkyl optionally substituted with one or more, particularly one to three, more particularly one or two substituents R^{3b}, (C₃-C₆)cycloalkyl-(C₁-C₆)alkoxy optionally substituted with one or more, particularly one to three, more particularly one or two substituents R^{3c}, heterocycloalkyl optionally substituted with one or more, particularly one to three, more particularly one or two substituents R^{3d} or phenyl optionally substituted with one or more, particularly one to three, more particularly one or two substituents R^{3e};
R^{3a}, R^{3b}, R^{3c}, R^{3d} and R^{3e} are each independently selected from halogen, (C₁-C₆)alkyl, (C₁-C₆)alkoxy, halo(C₁-C₆)alkyl and halo(C₁-C₆)alkoxy;
R⁴ is hydrogen, cyano, hydroxy, halogen, amino, (C₁-C₆)alkyl, (C₁-C₆)alkoxy, halo(C₁-C₆)alkyl, halo(C₁-C₆)alkoxy, (C₁-C₆)alkoxy-(C₁-C₆)alkyl, (C₃-C₆)cycloalkyl, (C₃-C₆)cycloalkyl-(C₁-C₆)alkyl, (C₃-C₆)cycloalkyl optionally substituted with one or more, particularly one to three, more particularly one or two substituents R^{4a}, (C₃-C₆)cycloalkyl-(C₁-C₆)alkyl optionally substituted with one or more, particularly one to three, more particularly one or two substituents R^{4b}, (C₃-C₆)cycloalkyl-(C₁-C₆)alkoxy optionally substituted with one or more, particularly one to three, more particularly one or two substituents R^{4c}, heterocycloalkyl optionally substituted with one or more, particularly one to three, more particularly one or two substituents R^{4d}, —CO₂R^{4a} or —CONR^{4b}R^{4c};
R^{4a}, R^{4b}, R^{4c} and R^{4d}, R^{4e} are each independently selected from hydrogen and (C₁-C₆)alkyl; and
R⁵ is —NH₂ or oxo;
or pharmaceutically acceptable salts thereof.

2. (canceled)

3. The compound of claim 1, wherein the compound is of formula I′:

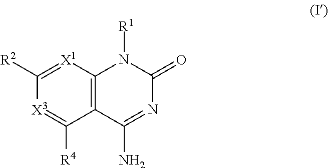

4. The compound of claim 1, wherein the compound is of formula I″:

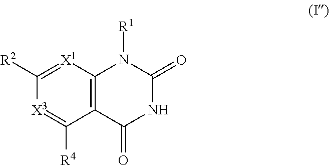

5. The compound of claim 1, wherein X³ is CR³.

6. The compound of claim 1, wherein X¹ is N.

7. The compound of claim 1, wherein R¹ is (C₁-C₆)alkyl optionally substituted with one R^{1a}, (C₃-C₆)cycloalkyl optionally substituted with one R″ °, heteroaryl optionally substituted with one or two R^{1d}, heterocycloalkyl optionally substituted with one R^{1e} or phenyl optionally substituted with one or two R^{1f}.

8. The compound of claim 1, wherein R¹ is (C₁-C₃)alkyl optionally substituted with one R^{1a}, (C₃-C₆)cycloalkyl optionally substituted with one R^{1c}, pyrazolyl optionally substituted with one R^{1d}, indazolyl optionally substituted with one R^{1d}, indolyl optionally substituted with one R^{1d}, benzo[d]oxazolyl optionally substituted with one R^{1d}, benzo[d]thiazolyl optionally substituted with one R^{1d}, benzo[d]imidazolyl optionally substituted with one R^{1d}, dioxepanyl optionally substituted with one R^{1d}, oxazolyl optionally substituted with one R^{1d}, thiazolyl optionally substituted with one R^{1d}, pyridinyl optionally substituted with one or two R^{1d}, pyrimidinyl optionally substituted with one R^{1d}, dihydropyrrolo[1,2-c]imidazolyl optionally substituted with one R^{1e}, oxepanyl optionally substituted with one R^{1e}, dihydro-indolyl optionally substituted with one R^{1e}, 1,4-dioxepanyl optionally substituted with one R^{1e}, tetrahydrofuranyl optionally substituted with one R^{1e}, tetrahydropyranyl optionally substituted with one R^{1e}, piperidinyl optionally substituted with one R^{1e}, oxaspiro[2.5]octanyl optionally substituted with one R^{1e}, dihydrobenzofuranyl optionally substituted with one R^{1e} or phenyl optionally substituted with one or two R^{1f}.

9. (canceled)

10. (canceled)

11. (canceled)

12. (canceled)

13. (canceled)

14. The compound of claim 1, wherein R¹ is tetrahydropyranyl optionally substituted with one (C₁-C₃)alkyl in alpha.

15. The compound of claim 1, wherein R¹ is heteroaryl substituted with one or two R^{1d} wherein at least of one R^{1d} is substituted in ortho, heterocycloalkyl substituted with one R^{1e} substituted in alpha or phenyl substituted with one or two R^{1f} wherein at least of one R^{1f} is substituted in ortho.

16. The compound of claim 1, wherein R¹ is pyridinyl substituted with one or two R^{1d} wherein at least of one R^{1d} is substituted in ortho, tetrahydrofuranyl substituted with one R^{1e} substituted in alpha, tetrahydropyranyl substituted with one R^{1e} substituted in alpha, oxaspiro[2.5]octanyl or 2,3-dihydrobenzofuranyl substituted with one R^{1e}.

17. (canceled)

18. (canceled)

19. The compound of claim 1, wherein R^{1a} and R^{1b} are each independently selected from heteroaryl, heterocycloalkyl and phenyl.

20. The compound of claim 1, wherein R^{1a} is selected from tetrahydrofuranyl, pyridinyl, oxetanyl or oxazolyl.

21. The compound of claim 1, wherein R^{1c}, R^{1d}, R^{1e} and R^{1f} are each independently selected from halogen, oxo, cyano, hydroxy, (C₁-C₆)alkyl, (C₁-C₆)alkoxy, halo(C₁-C₆)alkyl and halo(C₁-C₆)alkoxy.

22. (canceled)

23. (canceled)

24. (canceled)

25. The compound of claim 1, wherein R¹ is 2,3-dihydrobenzofuranyl, 2-hydroxycyclopentyl, 3-hydroxycyclopentyl, 1-(tetrahydrofuran-2-yl)ethyl, 1-tetrahydrofuran-3-yl-ethyl, 1-pyridin-2-yl-ethyl, oxepan-3-yl, 1,4-dioxepan-6-yl, dihydro-1H-indol-4-yl, 1-(oxetan-3-yl)ethyl, 1-(oxazol-5-yl)ethyl, indazol-4-yl, oxaspiro[2.5]octanyl, 4-methyloxazol-5-yl, 2-methoxy-phenyl, 3-methyl-phenyl, 2-methyl-phenyl, 3-fluoro-2-methoxyphenyl, 2-methylbenzonitrile, 2-methoxybenzonitrile, 2-ethoxybenzonitrile, 2-chlorophenyl, 3-chlorophenyl, 4-fluoro-2-methylphenyl, 3-fluoro-2-methylphenyl, 3-fluorophenyl, 2-fluorophenyl, 2,6-difluorophenyl, 2,3-dimethylphenyl, phenyl, 2,3-difluorophenyl, 2-fluoro-3-methylphenyl, 3-methoxyphenyl, 3,5-difluorophenyl, 3,4-difluorophenyl, 2-trifluoromethyl-phenyl, 3-(fluoromethyl)-2-methylphenyl, 3-ethylphenyl, 3-chloro-2-fluorophenyl, 2-chloro-5-fluorophenyl, 2-chloro-4-fluorophenyl, 2,3-dichlorophenyl, benzo[d]oxazol-4-yl, benzo[d]imidazolyl, benzo[d]thiazol-7-yl, 2-oxopiperidin-4-yl, 2-methylpyrazol-3-yl, 1-ethyl-1H-pyrazol-5-yl, 2-methylpyridin-3-yl, picolinonitrile, 2-methoxypyridin-3-yl, 2-(trifluoromethyl)pyridin-3-yl, 4-methylpyridin-3-yl, 4-fluoro-2-methoxypyridin-3-yl, indolyl, 2-chloropyridin-3-yl, 6-methoxypyridin-2-yl, 4-methylpyrimidin-5-yl, trifluoromethoxypyridin-2-yl, dihydrobenzofuranyl, tetrahydrofuranyl, 4-methyltetrahydrofuran-3-yl, methyl-tetrahydro-2H-pyran-3-yl, 6,7-dihydro-5H-pyrrolo[1,2-c]imidazol-7-yl, tetrahydro-2H-pyran-3-yl, tetrahydro-2H-pyran-4-yl or 4-methylthiazol-5-yl,

26. (canceled)

27. (canceled)

28. (canceled)

29. The compound of claim 1, wherein R² is hydrogen, halogen, amino, (C₁-C₆)alkyl, (C₁-C₆)alkoxy, halo(C₁-C₆)alkyl, halo(C₁-C₆)alkoxy, (C₃-C₆)cycloalkyl optionally substituted with one or more, particularly one to three, more particularly one or two substituents R^{2a}, (C₃-C₆)cycloalkyl-(C₁-C₆)alkyl optionally substituted with one or more, particularly one to three, more particularly one or two substituents R^{2b}, (C₃-C₆)cycloalkyl-(C₁-C₆)alkoxy optionally substituted with one or more, particularly one to three, more particularly one or two substituents R^{2e} heterocycloalkyl optionally substituted with one or more, particularly one to three, more particularly one or two substituents R^{2d} or phenyl optionally substituted with one or more, particularly one to three, more particularly one or two substituents R^{2e}; and
R^{2a}, R^{2b}, R^{2c}, R^{2d} and R^{2e} are each independently selected from halogen, (C₁-C₆)alkyl, (C₁-C₆)alkoxy, halo(C₁-C₆)alkyl and halo(C₁-C₆)alkoxy.

30. (canceled)

31. (canceled)

32. (canceled)

33. (canceled)

34. The compound of claim 1, wherein R² is trifluoromethyl, difluoromethoxy, trifluoromethoxy or cyclopropyl.

35. The compound of claim 1, wherein R^{2a}, R^{2b}, R^{2c}, R^{2d} and R^{2e} are each independently selected from halogen and (C₁-C₆)alkyl.

36. (canceled)

37. (canceled)

38. The compound of claim 1, wherein R^{2f} and R^{2g} are each independently selected from hydrogen or (C₁-C₃)alkyl, particularly wherein one of R^{2f} and R^{2g} is hydrogen while the other is (C₁-C₃)alkyl.

39. (canceled)

40. The compound of claim 1, wherein R³ is hydrogen, chloro, fluoro or cyano.

41. (canceled)

42. The compound of claim 1, wherein R^{3a}, R^{3b}, R^{3c}, R^{3d} and R^{3e} are each independently selected from halogen and (C₁-C₃)alkyl.

43. The compound of claim 1, wherein R⁴ is hydrogen, cyano, halogen, (C₁-C₆)alkyl, (C₁-C₆)alkoxy or —CONR^{4b}R^{4c}.

44. (canceled)

45. (canceled)

46. The compound of claim 1, wherein R^{4b} or R^{4c} are hydrogen.

47. The compound of claim 1, wherein R⁵ is —NH₂.

48. The compound of claim 1, selected from the group consisting of:
4-amino-7-cyclopropyl-1-(o-tolyl)pyrido[2,3-d]pyrimidin-2 (1H)-one;
4-amino-7-cyclopropyl-1-(2-methoxyphenyl)pyrido[2,3-d]pyrimidin-2 (1H)-one;
4-amino-7-(tert-butyl)-1-(o-tolyl)pyrido[2,3-d]pyrimidin-2 (1H)-one;
4-amino-1-(2-methoxyphenyl)-7-phenylpyrido[2,3-d]pyrimidin-2 (1H)-one;
4-amino-7-(3,3-difluoroazetidin-1-yl)-1-(o-tolyl)pyrido[2,3-d]pyrimidin-2 (1H)-one;
4-amino-7-cyclopropyl-1-(tetrahydrofuran-3-yl)pyrido[2,3-d]pyrimidin-2 (1H)-one;
4-amino-7-cyclopropyl-1-(2-methylpyridin-3-yl)pyrido[2,3-d]pyrimidin-2 (1H)-one;
4-amino-7-cyclopropyl-1-(2-oxopiperidin-4-yl)pyrido[2,3-d]pyrimidin-2 (1H)-one;
4-amino-7-cyclopropyl-1-((cis)-2-methyltetrahydrofuran-3-yl)pyrido[2,3-d]pyrimidin-2(1H)-one;
4-amino-7-cyclopropyl-1-(o-tolyl)pyrido[4,3-d]pyrimidin-2 (1H)-one;
4-amino-7-cyclopropyl-1-(2-methylpyridin-3-yl)quinazolin-2-one;
4-amino-7-cyclopropyl-1-(2-methylphenyl)quinazolin-2-one;
7-cyclopropyl-1-(2-methylphenyl)quinazoline-2,4-dione;
4-amino-7-cyclopropyl-1-(o-tolyl)pyrimido[4,5-d]pyrimidin-2 (1H)-one;
7-cyclopropyl-1-(2-methylpyridin-3-yl)quinazoline-2,4-dione;
4-amino-7-cyclopropyl-1-(2-methoxypyridin-3-yl)pyrido[2,3-d]pyrimidin-2 (1H)-one;
4-amino-7-cyclopropyl-6-fluoro-1-(2-methylpyridin-3-yl)pyrido[2,3-d]pyrimidin-2 (1H)-one;
7-cyclopropyl-1-(2-methylphenyl)pyrido[2,3-d]pyrimidine-2,4-dione;
3-(4-amino-7-cyclopropyl-2-oxopyrido[2,3-d]pyrimidin-1 (2H)-yl)picolinonitrile;
4-amino-7-cyclopropyl-1-(oxan-3-yl)pyrido[2,3-d]pyrimidin-2-one;
4-amino-7-cyclopropyl-1-[1-(oxolan-3-yl)ethyl]pyrido[2,3-d]pyrimidin-2-one;
4-amino-7-cyclopropyl-1-(3-fluoro-2-methoxyphenyl)pyrido[2,3-d]pyrimidin-2 (1H)-one;
3-(4-amino-7-cyclopropyl-2-oxopyrido[2,3-d]pyrimidin-1 (2H)-yl)-2-methylbenzonitrile;
4-amino-1-(2-methylpyridin-3-yl)-7-propan-2-ylpyrido[2,3-d]pyrimidin-2-one;
4-amino-7-cyclopropyl-1-(2,3-dihydrobenzofuran-4-yl)pyrido[2,3-d]pyrimidin-2 (1H)-one;
4-amino-6-chloro-7-cyclopropyl-1-(2-methylpyridin-3-yl)pyrido[2,3-d]pyrimidin-2 (1H)-one;
3-(4-amino-7-cyclopropyl-2-oxopyrido[2,3-d]pyrimidin-1 (2H)-yl)-2-methoxybenzonitrile;
3-(4-amino-7-cyclopropyl-2-oxopyrido[2,3-d]pyrimidin-1 (2H)-yl)-2-ethoxybenzonitrile;
4-amino-7-cyclopropyl-1-(1-(tetrahydrofuran-2-yl)ethyl)pyrido[2,3-d]pyrimidin-2 (1H)-one;
4-amino-1-(2-methylpyridin-3-yl)-7-(oxetan-3-yl)quinazolin-2 (1H)-one;
7-cyclopropyl-1-(2-methylpyridin-3-yl)pyrido[2,3-d]pyrimidine-2,4-dione;
4-amino-7-((1RS,2RS)-2-methylcyclopropyl)-1-(2-methylpyridin-3-yl)pyrido[2,3-d]pyrimidin-2 (1H)-one;
4-amino-7-cyclopentyl-1-(2-methyl-3-pyridyl)pyrido[2,3-d]pyrimidin-2-one;
4-amino-7-cyclopropyl-1-((1SR,2RS)-2-hydroxycyclopentyl)pyrido[2,3-d]pyrimidin-2 (1H)-one;
4-amino-2-cyclopentyl-7-(o-tolyl)pyrazolo[3,4-d]pyrimidin-6-one; formic acid;
4-amino-7-cyclopentyl-1-(4-methylpyrimidin-5-yl)pyrido[2,3-d]pyrimidin-2-one;
4-amino-7-cyclopropyl-1-(3-fluoro-2-methylphenyl)pyrido[2,3-d]pyrimidin-2 (1H)-one;
4-amino-7-cyclopropyl-1-[(3R)-oxan-3-yl]pyrido[2,3-d]pyrimidin-2-one;
4-amino-7-cyclopropyl-1-[(3S)-oxan-3-yl]pyrido[2,3-d]pyrimidin-2-one;
4-amino-7-cyclopropyl-1-(4-methyltetrahydrofuran-3-yl)pyrido[2,3-d]pyrimidin-2 (1H)-one;
4-amino-7-cyclopropyl-2-oxo-1-(0-tolyl)-1,2-dihydropyrido[2,3-d]pyrimidine-5-carboxamide;
4-amino-1-(2-methoxy-3-pyridyl)-7-tetrahydropyran-2-yl-pyrido[2,3-d]pyrimidin-2-one;
4-amino-7-[(1S,4R)-3-azabicyclo[2.2.1]heptan-3-yl]-1-(2-methylpyrazol-3-yl)pyrido[2,3-d]pyrimidin-2-one; formic acid;
4-amino-1-(2-methylpyridin-3-yl)-7-(trifluoromethyl)quinazolin-2-one;
4-amino-7-cyclopropyl-1-[rac-(2R,3 S)-2-methyloxolan-3-yl]pyrido[2,3-d]pyrimidin-2-one;
4-amino-7-cyclobutyl-1-(2-methyl-3-pyridyl)pyrido[2,3-d]pyrimidin-2-one; formic acid;
4-amino-7-cyclopropyloxy-1-(2-methylpyridin-3-yl)quinazolin-2-one;
4-amino-1-(2-methylpyridin-3-yl)-7-(trifluoromethyl)pyrido[2,3-d]pyrimidin-2 (1H)-one;
4-amino-7-(7-azabicyclo[2.2.1]heptan-7-yl)-1-(4-methylthiazol-5-yl)pyrido[2,3-d]pyrimidin-2-one; formic acid;
4-amino-7-cyclopropyl-1-(3-hydroxycyclopentyl)pyrido[2,3-d]pyrimidin-2 (1H)-one;
4-amino-7-(difluoromethoxy)-1-(2-methylpyridin-3-yl)quinazolin-2 (1H)-one;
4-amino-7-(difluoromethyl)-1-(2-methylpyridin-3-yl)quinazolin-2 (1H)-one;
4-amino-7-[(1R,2S)-2-fluorocyclopropyl]-1-(2-methyl-3-pyridyl)pyrido[2,3-d]pyrimidin-2-one;
4-amino-7-cyclopropyl-1-(2-methylpyridin-3-yl)-2-oxo-1,2-dihydropyrido[2,3-d]pyrimidine-6-carbonitrile;
3-(4-amino-7-isopropyl-2-oxopyrido[2,3-d]pyrimidin-1 (2H)-yl)-2-methoxybenzonitrile;
4-amino-7-cyclopropyl-2-oxo-1-(0-tolyl)-1,2-dihydropyrido[2,3-d]pyrimidine-5-carbonitrile;
4-amino-7-methoxy-1-(2-methylpyridin-3-yl)quinazolin-2 (1H)-one;
4-amino-1-(2-methylpyridin-3-yl)-7-(trifluoromethoxy)quinazolin-2 (1H)-one;
4-amino-7-(4,5-dihydrofuran-3-yl)-1-(2-methylpyridin-3-yl)pyrido[2,3-d]pyrimidin-2 (1H)-one;
4-amino-1-(2-methylpyridin-3-yl)-7-(tetrahydrofuran-3-yl)pyrido[2,3-d]pyrimidin-2 (1H)-one;
4-amino-7-cyclopropyl-1-(2-methylpyridin-3-yl)pyrido[2,3-d]pyrimidin-2-one;
4-amino-7-cyclopropyl-1-(2-methylpyridin-3-yl)pyrido[2,3-d]pyrimidin-2-one;
4-amino-7-cyclopropyl-1-(4-methylthiazol-5-yl)pyrido[2,3-d]pyrimidin-2-one;
4-amino-7-cyclopropyl-1-(4-methylpyrimidin-5-yl)pyrido[2,3-d]pyrimidin-2 (1H)-one,
4-amino-7-cyclopropyl-1-(2-(trifluoromethyl)pyridin-3-yl)pyrido[2,3-d]pyrimidin-2(1H)-one;
4-amino-7-cyclopropyl-1-(2-methylpyrazol-3-yl)pyrido[2,3-d]pyrimidin-2-one;
4-amino-7-cyclopropyl-1-(2,3-dihydrobenzofuran-4-yl)quinazolin-2 (1H)-one;
(R)-4-amino-1-(tetrahydro-2H-pyran-3-yl)-7-(trifluoromethyl)quinazolin-2 (1H)-one;
4-amino-7-cyclopropyl-1-(4-methylpyridin-3-yl)pyrido[2,3-d]pyrimidin-2 (1H)-one;
4-amino-7-ethyl-1-(2-methylpyridin-3-yl)quinazolin-2 (1H)-one;
4-amino-7-[(1S,2R)-2-fluorocyclopropyl]-1-[(3R)-tetrahydropyran-3-yl]pyrido[2,3-d]pyrimidin-2-one;
4-amino-7-cyclopropyl-1-(4-methyloxazol-5-yl)pyrido[2,3-d]pyrimidin-2-one;
3-(4-amino-6-chloro-7-isopropyl-2-oxopyrido[2,3-d]pyrimidin-1 (2H)-yl)-2-methoxybenzonitrile;
4-amino-7-cyclopropyl-1-((R)-1-((S)-tetrahydrofuran-3-yl)ethyl)pyrido[2,3-d]pyrimidin-2 (1H)-one;
4-amino-7-cyclopropyl-1-((R)-1-((R)-tetrahydrofuran-3-yl)ethyl)pyrido[2,3-d]pyrimidin-2 (1H)-one;
4-amino-7-(2-fluoropropan-2-yl)-1-(2-methylpyridin-3-yl)quinazolin-2-one;
4-amino-5-methoxy-1-(2-methylpyridin-3-yl)-7-(trifluoromethyl)quinazolin-2 (1H)-one;
4-amino-7-cyclopropyl-1-(4-fluoro-2-methoxypyridin-3-yl)pyrido[2,3-d]pyrimidin-2(1H)-one;
4-amino-5-fluoro-1-(2-methylpyridin-3-yl)-7-(trifluoromethyl)quinazolin-2 (1H)-one;
4-amino-7-cyclopropyl-1-(1-ethyl-1H-pyrazol-5-yl)pyrido[2,3-d]pyrimidin-2 (1H)-one;
4-amino-7-chloro-1-(o-tolyl)pyrido[2,3-d]pyrimidin-2 (1H)-one;
4-amino-7-cyclopropyl-1-(2,3-dihydrobenzofuran-7-yl)pyrido[2,3-d]pyrimidin-2 (1H)-one;
1-amino-4-(2-methoxyphenyl)-6-(trifluoromethyl)-3H-pyrido[1,2-c]pyrimidin-3-one;
4-amino-1-(2-chlorophenyl)-7-cyclopropylpyrido[2,3-d]pyrimidin-2-one;
4-amino-7-cyclopropyl-1-[(1R)-1-[(3S)-oxolan-3-yl]ethyl]pyrido[2,3-d]pyrimidin-2-one;
4-amino-7-cyclopropyl-1-[(1R)-1-[(3R)-oxolan-3-yl]ethyl]pyrido[2,3-d]pyrimidin-2-one;
3-(4-amino-6-chloro-7-cyclopropyl-2-oxopyrido[2,3-d]pyrimidin-1 (2H)-yl)-2-methoxybenzonitrile;
4-amino-7-cyclopropyl-1-(4-oxaspiro[2.5]octan-8-yl)pyrido[2,3-d]pyrimidin-2 (1H)-one;
4-amino-6-chloro-7-cyclopropyl-1-(2-methoxypyridin-3-yl)pyrido[2,3-d]pyrimidin-2(1H)-one;
4-amino-7-cyclopropyl-1-(4-fluoro-2-methylphenyl)pyrido[2,3-d]pyrimidin-2-one;
4-amino-7-cyclopropyl-1-(3-ethylphenyl)pyrido[2,3-d]pyrimidin-2 (1H)-one,
4-amino-7-cyclopropyl-1-(m-tolyl)pyrido[2,3-d]pyrimidin-2 (1H)-one;
4-amino-7-cyclopropyl-1-(3,5-difluorophenyl)pyrido[2,3-d]pyrimidin-2 (1H)-one;
4-amino-7-cyclopropyl-1-(6-methoxypyridin-2-yl)pyrido[2,3-d]pyrimidin-2 (1H)-one;
4-amino-7-cyclopropyl-1-(3-methoxyphenyl)pyrido[2,3-d]pyrimidin-2 (1H)-one;
4-amino-7-cyclopropyl-1-(2,3-difluorophenyl)pyrido[2,3-d]pyrimidin-2 (1H)-one;
4-amino-7-cyclopropyl-1-phenylpyrido[2,3-d]pyrimidin-2 (1H)-one;
4-amino-7-cyclopropyl-1-(1-(oxazol-5-yl)ethyl)pyrido[2,3-d]pyrimidin-2 (1H)-one;
3-(4-amino-2-oxo-7-(trifluoromethyl)quinazolin-1 (2H)-yl)-2-methylbenzonitrile;
3-(4-amino-2-oxo-7-(trifluoromethyl)pyrido[2,3-d]pyrimidin-1 (2H)-yl)-2-methylbenzonitrile;
4-amino-7-cyclopropyl-1-(2,6-difluorophenyl)pyrido[2,3-d]pyrimidin-2 (1H)-one;
4-amino-7-cyclopropyl-1-(2-fluorophenyl)pyrido[2,3-d]pyrimidin-2 (1H)-one;
4-amino-7-cyclopropyl-1-(3-fluorophenyl)pyrido[2,3-d]pyrimidin-2 (1H)-one;
4-amino-7-cyclopropyl-1-[1-(oxetan-3-yl)ethyl]pyrido[2,3-d]pyrimidin-2-one;
4-amino-7-(difluoromethoxy)-1-(2-methylphenyl)pyrido[2,3-d]pyrimidin-2-one;
4-amino-7-cyclopropyl-1-(1-pyridin-2-ylethyl)pyrido[2,3-d]pyrimidin-2-one;
4-amino-1-(2-methyl-3-pyridyl)-7-(2,2,2-trifluoroethyl)quinazolin-2-one hydrochloride;
4-amino-7-cyclopropyl-1-(tetrahydro-2H-pyran-4-yl)pyrido[2,3-d]pyrimidin-2 (1H)-one;
4-amino-1-(2-chloropyridin-3-yl)-7-cyclopropylpyrido[2,3-d]pyrimidin-2 (1H)-one;
4-amino-2-cyclopentyl-7-(2-methyl-3-pyridyl)pyrazolo[3,4-d]pyrimidin-6-one;
4-amino-5-chloro-1-(2-methylpyridin-3-yl)-7-(trifluoromethyl)quinazolin-2 (1H)-one;
4-amino-1-(3-fluoro-2-methylphenyl)-7-(trifluoromethyl)pyrido[2,3-d]pyrimidin-2 (1H)-one;
4-amino-7-cyclopropyl-1-(1H-indol-4-yl)pyrido[2,3-d]pyrimidin-2-one;
4-amino-7-cyclopropyl-1-[6-(trifluoromethoxy)pyridin-2-yl]pyrido[2,3-d]pyrimidin-2-one;
4-amino-1-(2,3-dihydrobenzofuran-4-yl)-7-(trifluoromethyl)pyrido[2,3-d]pyrimidin-2(1H)-one;
4-amino-1-(3-chloro-2-fluorophenyl)-7-cyclopropylpyrido[2,3-d]pyrimidin-2-one;
4-amino-1-(2,3-dihydro-1-benzofuran-4-yl)-7-(trifluoromethoxy)quinazolin-2-one;
4-amino-1-(3-fluoro-2-methylphenyl)-7-(trifluoromethoxy)quinazolin-2-one;
3-[4-amino-2-oxo-7-(trifluoromethoxy)quinazolin-1-yl]-2-methylbenzonitrile;
4-amino-7-cyclopropyl-1-(2,3-dihydro-1H-indol-4-yl)pyrido[2,3-d]pyrimidin-2-one;
4-amino-1-(2-chloro-5-fluorophenyl)-7-cyclopropylpyrido[2,3-d]pyrimidin-2-one;
4-amino-1-(2-chloro-4-fluorophenyl)-7-cyclopropylpyrido[2,3-d]pyrimidin-2 (1H)-one;
4-amino-7-cyclopropyl-1-(2,5-difluorophenyl)pyrido[2,3-d]pyrimidin-2 (1H)-one;
4-amino-7-(ethylamino)-1-(o-tolyl)quinazolin-2-one;
4-amino-1-(3-chlorophenyl)-7-cyclopropylpyrido[2,3-d]pyrimidin-2 (1H)-one;
3-(4-amino-7-cyclopropyl-2-oxopyrido[2,3-d]pyrimidin-1 (2H)-yl)benzonitrile;
4-amino-7-(difluoromethoxy)-1-(4-oxaspiro[2.5]octan-8-yl)quinazolin-2 (1H)-one;
1-amino-4-(2-chlorophenyl)-6-(trifluoromethyl)pyrido[1,2-c]pyrimidin-3-one;
4-amino-1-(benzo[d]oxazol-4-yl)-7-cyclopropylpyrido[2,3-d]pyrimidin-2 (1H)-one;
4-amino-7-cyclopropyl-1-(3,4-difluorophenyl)pyrido[2,3-d]pyrimidin-2 (1H)-one;
3-(4-amino-7-cyclopropyl-2-oxopyrido[2,3-d]pyrimidin-1 (2H)-yl)-2-methylbenzonitrile;
3-(4-amino-7-cyclopropyl-2-oxopyrido[2,3-d]pyrimidin-1 (2H)-yl)-2-methylbenzonitrile;
4-amino-7-(difluoromethoxy)-1-(o-tolyl)quinazolin-2 (1H)-one;
3-(4-amino-7-cyclopropyl-2-oxopyrido[2,3-d]pyrimidin-1(2H)-yl)-2-chlorobenzonitrile;
4-amino-7-cyclopropyl-1-[(8S)-4-oxaspiro[2.5]octan-8-yl]pyrido[2,3-d]pyrimidin-2-one;
4-amino-7-cyclopropyl-1-[(8R)-4-oxaspiro[2.5]octan-8-yl]pyrido[2,3-d]pyrimidin-2-one;
4-amino-1-(1H-benzo[d]imidazol-4-yl)-7-cyclopropylpyrido[2,3-d]pyrimidin-2 (1H)-one;
4-amino-7-cyclopropyl-1-(1H-indazol-4-yl)pyrido[2,3-d]pyrimidin-2 (1H)-one;
4-amino-7-cyclopropyl-1-(2,3-dimethylphenyl)pyrido[2,3-d]pyrimidin-2 (1H)-one;
4-amino-1-(2-chloropyridin-3-yl)-7-(difluoromethoxy)quinazolin-2 (1H)-one;
4-amino-1-(2-chloropyridin-3-yl)-7-cyclopropylpyrido[2,3-d]pyrimidin-2 (1H)-one;
4-amino-1-(2-chloropyridin-3-yl)-7-cyclopropylpyrido[2,3-d]pyrimidin-2 (1H)-one;
4-amino-7-(difluoromethoxy)-1-(m-tolyl)quinazolin-2 (1H)-one;
4-amino-7-(difluoromethoxy)-1-(2-fluoro-3-methylphenyl)quinazolin-2 (1H)-one;
4-amino-7-cyclopropyl-1-(2-(trifluoromethyl)phenyl)pyrido[2,3-d]pyrimidin-2 (1H)-one;
4-amino-7-cyclopropyl-1-(3-(fluoromethyl)-2-methylphenyl)pyrido[2,3-d]pyrimidin-2 (1H)-one;
4-amino-1-(2-chloro-3-methylphenyl)-7-cyclopropylpyrido[2,3-d]pyrimidin-2-one;
4-amino-1-(3-chloro-2-methylphenyl)-7-cyclopropylpyrido[2,3-d]pyrimidin-2-one;
4-amino-7-cyclopropyl-1-(2,3-dichlorophenyl)pyrido[2,3-d]pyrimidin-2 (1H)-one;
4-amino-7-cyclopropyl-1-(2-fluoro-3-methylphenyl)pyrido[2,3-d]pyrimidin-2 (1H)-one;
3-(4-amino-7-cyclopropyl-2-oxopyrido[2,3-d]pyrimidin-1 (2H)-yl)-2-fluorobenzonitrile;
(S)-4-amino-7-cyclopropyl-1-(oxepan-3-yl)pyrido[2,3-d]pyrimidin-2 (1H)-one;
(R)-4-amino-7-cyclopropyl-1-(oxepan-3-yl)pyrido[2,3-d]pyrimidin-2 (1H)-one;
4-amino-7-cyclopropyl-1-(3-hydroxy-2-methylphenyl)pyrido[2,3-d]pyrimidin-2 (1H)-one;
4-amino-7-(difluoromethoxy)-1-(3-fluoro-2-methylphenyl)quinazolin-2 (1H)-one;
4-amino-7-cyclopropyl-1-[rac-(2S,3S)-2-methyltetrahydropyran-3-yl]pyrido[2,3-d]pyrimidin-2-one;
4-amino-1-(benzo[d]thiazol-7-yl)-7-cyclopropylpyrido[2,3-d]pyrimidin-2 (1H)-one;
4-amino-7-cyclopropyl-1-((2S,3R)-2-methyltetrahydro-2H-pyran-3-yl)pyrido[2,3-d]pyrimidin-2 (1H)-one;
4-amino-7-cyclopropyl-1-((2R,3S)-2-methyltetrahydro-2H-pyran-3-yl)pyrido[2,3-d]pyrimidin-2 (1H)-one;
(−)-4-amino-1-(2-chloro-3-fluorophenyl)-7-cyclopropylpyrido[2,3-d]pyrimidin-2 (1H)-one;
(+)-4-amino-1-(2-chloro-3-fluorophenyl)-7-cyclopropylpyrido[2,3-d]pyrimidin-2 (1H)-one;
(−)-4-amino-1-(2-chlorophenyl)-7-cyclopropylpyrido[2,3-d]pyrimidin-2 (1H)-one;
(+)-4-amino-1-(2-chlorophenyl)-7-cyclopropylpyrido[2,3-d]pyrimidin-2 (1H)-one;
4-amino-7-cyclopropyl-6-(difluoromethoxy)-1-(o-tolyl)pyrido[2,3-d]pyrimidin-2-one;
4-amino-7-cyclopropyl-1-(6,7-dihydro-5H-pyrrolo[1,2-c]imidazol-7-yl)pyrido[2,3-d]pyrimidin-2-one; formic acid;
4-(2-chlorophenyl)-6-cyclopropyl-1-imino-pyrido[1,2-c]pyrimidin-3-one; formic acid;
4-amino-7-cyclopropyl-1-[2-(trifluoromethoxy)phenyl]pyrido[2,3-d]pyrimidin-2-one;
4-amino-1-(2-chloro-3-pyridyl)-7-(trifluoromethoxy)quinazolin-2-one;
4-amino-7-cyclopropyl-1-(6-(difluoromethoxy)pyridin-2-yl)pyrido[2,3-d]pyrimidin-2(1H)-one;
4-amino-7-cyclopropyl-1-(1,4-dioxepan-6-yl)pyrido[2,3-d]pyrimidin-2-one; and
4-amino-7-cyclopropyl-1-(3-fluoro-2-methylphenyl)quinazolin-2-one;
or pharmaceutically acceptable salts thereof.

49. The compound of claim 1, selected from the group consisting of:
4-amino-7-cyclopropyl-1-(o-tolyl)pyrido[2,3-d]pyrimidin-2 (1H)-one;
4-amino-7-cyclopropyl-1-(2-methylpyridin-3-yl)pyrido[2,3-d]pyrimidin-2 (1H)-one;
3-(4-amino-7-cyclopropyl-2-oxopyrido[2,3-d]pyrimidin-1 (2H)-yl)-2-methylbenzonitrile;
4-amino-7-cyclopropyl-1-(2,3-dihydrobenzofuran-4-yl)pyrido[2,3-d]pyrimidin-2 (1H)-one;
4-amino-7-cyclopropyl-1-(3-fluoro-2-methylphenyl)pyrido[2,3-d]pyrimidin-2 (1H)-one;
4-amino-7-cyclopropyl-1-(oxan-3-yl)pyrido[2,3-d]pyrimidin-2-one;
4-amino-7-(difluoromethoxy)-1-(2-methylpyridin-3-yl)quinazolin-2 (1H)-one;
4-amino-1-(2-methylpyridin-3-yl)-7-(trifluoromethoxy)quinazolin-2 (1H)-one;
4-amino-7-cyclopropyl-1-(2,3-dihydrobenzofuran-4-yl)quinazolin-2 (1H)-one;
4-amino-7-cyclopropyl-1-(2,3-dihydrobenzofuran-7-yl)pyrido[2,3-d]pyrimidin-2 (1H)-one;
4-amino-1-(2-chlorophenyl)-7-cyclopropylpyrido[2,3-d]pyrimidin-2-one;
4-amino-7-cyclopropyl-1-(4-oxaspiro[2.5]octan-8-yl)pyrido[2,3-d]pyrimidin-2 (1H)-one;
4-amino-1-(2-chloropyridin-3-yl)-7-cyclopropylpyrido[2,3-d]pyrimidin-2 (1H)-one;
4-amino-7-(difluoromethoxy)-1-(2-methylphenyl)pyrido[2,3-d]pyrimidin-2-one;
3-(4-amino-2-oxo-7-(trifluoromethyl)pyrido[2,3-d]pyrimidin-1 (2H)-yl)-2-methylbenzonitrile;
4-amino-7-cyclopropyl-1-(m-tolyl)pyrido[2,3-d]pyrimidin-2 (1H)-one;
4-amino-7-cyclopropyl-1-[(8S)-4-oxaspiro[2.5]octan-8-yl]pyrido[2,3-d]pyrimidin-2-one;
4-amino-7-cyclopropyl-1-[(8R)-4-oxaspiro[2.5]octan-8-yl]pyrido[2,3-d]pyrimidin-2-one;
(R)-4-amino-7-cyclopropyl-1-(oxepan-3-yl)pyrido[2,3-d]pyrimidin-2 (1H)-one; and
4-amino-7-cyclopropyl-1-((2S,3R)-2-methyltetrahydro-2H-pyran-3-yl)pyrido[2,3-d]pyrimidin-2(1H)-one;
or pharmaceutically acceptable salts thereof.

50. A compound of claim 1 for use as a therapeutically active substance.

51. Pharmaceutical compositions comprising compounds of formula I or II of claim 1 or their pharmaceutically acceptable salts and one or more pharmaceutically acceptable excipients.

52. Compounds of formula I or II of claim 1 or their pharmaceutically acceptable salts above for use as therapeutically active substances.

53. Compounds of formula I or II of claim 1 or their pharmaceutically acceptable salts for the use in the treatment, prevention and/or delay of progression of Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma.

54. (canceled)

55. A method for the treatment or prevention of Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma, which method comprises administering compounds of formula I of claim 1 or their pharmaceutically acceptable salts as defined above to a subject.

56. (canceled)

57. The use of compounds of formula of claim 1 or their pharmaceutically acceptable salts for the treatment, prevention and/or delay of progression of Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma.

58. (canceled)

59. The use of compounds of formula I of claim 1 or their pharmaceutically acceptable salts for the preparation of medicaments for the treatment or prevention of Lung Adenocarcinoma, Melanoma, Pancreatic Adenocarcinoma, Head and Neck Squamous Cell Carcinoma, Lung Squamous Cell Carcinoma, Esophageal Carcinoma, Glioblastmoa Multiforme, and Mesothelioma.

60. (canceled)

61. (canceled)
